(19) United States
(12) Patent Application Publication
(10) Pub. No.: US 2006/0106020 A1
(43) Pub. Date: May 18, 2006

(54) Tetracyclic inhibitors of Janus kinases

(76) Inventors: James D. Rodgers, Landenberg, PA (US); Darius J. Robinson, Wilmington, DE (US); Argrios G. Arvanitis, Kennett Square, PA (US); Thomas P. Maduskuie JR., Wilmington, DE (US); Stacey Shepard, Voorhees, NJ (US); Louis Storace, Middletown, DE (US); Haisheng Wang, Hockessin, DE (US); Maria Rafalski, Wilmington, DE (US); Ravi Kumar Jalluri, Avondale, PA (US); Andrew P. Combs, Kennett Square, PA (US); Matthew L. Crawley, Swathmore, PA (US)

Correspondence Address: COZEN O' CONNOR, P.C., 1900 MARKET STREET, PHILADELPHIA, PA, 19103-3508, US

(21) Appl. No.: 11/115,702

(22) Filed: Apr. 27, 2005

Related U.S. Application Data
(60) Provisional application No. 60/566,142, filed on Apr. 28, 2004.
(60) Provisional application No. 60/626,111, filed on Nov. 8, 2004.

(51) Int. Cl. A61K 31/503; A61K 31/519; C07D 487/14

(52) U.S. Cl. 514/248; 514/257; 544/233; 544/247

(57) ABSTRACT

The present invention provides compounds that modulate the activity of Janus kinases and are useful in the treatment of diseases related to activity of Janus kinases including, for example, immune-related diseases and cancer.

### CROSS REFERENCE TO RELATED APPLICATIONS

This application claims the benefit of U.S. Ser. Nos. 60/566,142, filed Apr. 28, 2004 and 60/626,111, filed Nov. 8, 2004, the disclosures of each of which are incorporated herein by reference in their entireties.

### FIELD OF THE INVENTION

The present invention relates to compounds that modulate the activity of Janus kinases and are useful in the treatment of diseases related to activity of Janus kinases including, for example, immune-related diseases and cancer.

### BACKGROUND OF THE INVENTION

The immune system responds to injury and threats from pathogens. Cytokines are low-molecular weight polypeptides or glycoproteins that stimulate biological responses in virtually all cell types. For example, cytokines regulate many of the pathways involved in the host inflammatory response to sepsis. Cytokines influence cell differentiation, proliferation and activation, and they can modulate both proinflammatory and anti-inflammatory responses to allow the host to react appropriately to pathogens.

Binding of a cytokine to its cell surface receptor initiates intracellular signaling cascades that transduce the extracellular signal to the nucleus, ultimately leading to changes in gene expression. The pathway involving the Janus kinase family of protein tyrosine kinases (JAKs) and Signal Transducers and Activators of Transcription (STATs) is engaged in the signaling of a wide range of cytokines. Generally, cytokine receptors do not have intrinsic tyrosine kinase activity, and thus require receptor-associated kinases to propagate a phosphorylation cascade. JAKs fulfill this function. Cytokines bind to their receptors, causing receptor dimerization, and this enables JAKs to phosphorylate each other as well as specific tyrosine motifs within the cytokine receptors. STATs that recognize these phosphotyrosine motifs are recruited to the receptor, and are then themselves activated by a JAK-dependent tyrosine phosphorylation event. Upon activation, STATs dissociate from the receptors, dimerize, and translocate to the nucleus to bind to specific DNA sites and alter transcription (Scott, M. J., C. J. Godshall, et al. (2002). “Jaks, STATs, Cytokines, and Sepsis.” Clin Diagn Lab Immunol 9 (6): 1153-9).

The JAK family plays a role in the cytokine-dependent regulation of proliferation and function of cells involved in immune response. Currently, there are four known mammalian JAK family members: JAK1 (also known as Janus kinase-1), JAK2 (also known as Janus kinase-2), JAK3 (also known as Janus kinase, leukocyte; JAK1; L-JAK and Janus kinase-3) and TYK2 (also known as protein-tyrosine kinase 2). The JAK proteins range in size from 120 to 140 kDa and comprise seven conserved JAK homology (JH) domains; one of these is a functional catalytic kinase domain, and another is a pseudokinase domain potentially serving a regulatory function and/or serving as a docking site for STATs (Scott, Godshall et al. 2002, supra).

While JAK1, JAK2 and TYK2 are ubiquitously expressed, JAK3 is reported to be preferentially expressed in natural killer (NK) cells and not resting T cells, suggesting a role in lymphoid activation (Kawamura, M., D. W. McVicar, et al. (1994). “Molecular cloning of L-JAK, a Janus family protein-tyrosine kinase expressed in natural killer cells and activated leukocytes.” Proc Natl Acad Sci USA 91 (14): 6374-8).

Not only do the cytokine-stimulated immune and inflammatory responses contribute to normal host defense, they also play roles in the pathogenesis of diseases: pathologies such as severe combined immunodeficiency (SCID) arise from hypoactivity and suppression of the immune system, and a hyperactive or inappropriate immune/inflammatory response contributes to the pathology of autoimmune diseases such as rheumatoid and psoriatic arthritis, asthma and systemic lupus erythematosus, as well as illnesses such as scleroderma and osteoarthritis (Ortmann, R. A., T. Cheng, et al. (2000). “Janus kinases and signal transducers and activators of transcription: their roles in cytokine signaling, development and immunoregulation.” Arthritis Res 2 (1): 16-32). Furthermore, syndromes with a mixed presentation of autoimmune and immunodeficiency disease are quite common (Candotti, F., L. Notarangelo, et al. (2002). “Molecular aspects of primary immunodeficiencies: lessons from cytokine and other signaling pathways.” J Clin Invest 109 (10): 1261-9). Thus, therapeutic agents are typically aimed at augmentation or suppression of the immune and inflammatory pathways, accordingly.

Deficiencies in expression of JAK family members are associated with disease states. Jak1−/− mice are runted at birth, fail to nurse, and die perinatally (Rodig, S. J., M. A. Meraz, et al. (1998). “Disruption of the Jak1 gene demonstrates obligatory and nonredundant roles of the Jaks in cytokine-induced biologic responses.” Cell 93 (3): 373-83). Jak2−/− mouse embryos are anemic and die around day 12.5 postcoitum due to the absence of definitive erythropoiesis. JAK2-deficient fibroblasts do not respond to IFNgamma, although responses to IFNalpha/beta and IL-6 are unaffected. JAK2 functions in signal transduction of a specific group of cytokine receptors required in definitive erythropoiesis (Neubauer, H., A. Cumano, et al. (1998). Cell 93 (3): 397-409; Parganas, E., D. Wang, et al. (1998). Cell 93 (3): 385-95.). JAK3 appears to play a role in normal development and function of B and T lymphocytes. Mutations of JAK3 are reported to be responsible for autosomal recessive severe combined immunodeficiency (SCID) in humans (Candotti, F., S. A. Oakes, et al. (1997). “Structural and functional basis for JAK3-deficient severe combined immunodeficiency.” Blood 90 (10): 3996-4003).

The JAK/STAT pathway, and in particular all four members of the JAK family, are believed to play a role in the pathogenesis of the asthmatic response. The inappropriate immune responses that characterize asthma are orchestrated by a subset of CD4+ T helper cells termed T helper 2 (Th2) cells. Signaling through the cytokine receptor IL-4 stimulates JAK1 and JAK3 to activate STAT6, and signaling through IL-12 stimulates activation of JAK2 and TYK2, and subsequent phosphorylation of STAT4. STAT4 and STAT6 control multiple aspects of CD4+ T helper cell differentiation (Pernis, A. B. and P. B. Rothman (2002). “JAK-STAT signaling in asthma.” J Clin Invest 109 (10): 1279-83). Furthermore, TYK2-deficient mice were found to have enhanced Th2 cell-mediated allergic airway inflammation (Seto, Y., H. Nakajima, et al. (2003). “Enhanced Th2 cell-mediated allergic inflammation in Tyk2-deficient mice.” J Immunol 170 (2): 1077-83).

The JAK/STAT pathway, and in particular, JAK3, also plays a role in cancers of the immune system. In adult T cell leukemia/lymphoma (ATLL), human CD4+ T cells acquire a transformed phenotype, an event that correlates with acquisition of constitutive phosphorylation of JAKs and STATs. Furthermore, an association between JAK3 and STAT-1, STAT-3, and STAT-5 activation and cell-cycle progression was demonstrated by both propidium iodide staining and bromodeoxyuridine incorporation in cells of four ATLL patients tested. These results imply that JAK/STAT activation is associated with replication of leukemic cells and that therapeutic approaches aimed at JAK/STAT inhibition may be considered to halt neoplastic growth (Takemoto, S., J. C. Mulloy, et al. (1997). “Proliferation of adult T cell leukemia/lymphoma cells is associated with the constitutive activation of JAK/STAT proteins.” Proc Natl Acad Sci USA 94 (25): 13897-902).

Blocking signal transduction at the level of the JAK kinases holds promise for developing treatments for human cancers. Cytokines of the interleukin 6 (IL-6) family, which activate the signal transducer gp130, are major survival and growth factors for human multiple myeloma (MM) cells. The signal transduction of gp130 is believed to involve JAK1, JAK2 and Tyk2 and the downstream effectors STAT3 and the mitogen-activated protein kinase (MAPK) pathways. In IL-6-dependent MM cell lines treated with the JAK2 inhibitor tyrphostin AG490, JAK2 kinase activity and ERK2 and STAT3 phosphorylation were inhibited. Furthermore, cell proliferation was suppressed and apoptosis was induced (De Vos, J., M. Jourdan, et al. (2000). “JAK2 tyrosine kinase inhibitor tyrphostin AG490 downregulates the mitogen-activated protein kinase (MAPK) and signal transducer and activator of transcription (STAT) pathways and induces apoptosis in myeloma cells.” Br J Haematol 109 (4): 823-8). However, in some cases, AG490 can induce dormancy of tumor cells and actually then protect them from death.

Pharmacological targeting of Janus kinase 3 (JAK3) has been employed successfully to control allograft rejection and graft versus host disease (GVHD). In addition to its involvement in signaling of cytokine receptors, JAK3 is also engaged in the CD40 signaling pathway of peripheral blood monocytes. During CD40-induced maturation of myeloid dendritic cells (DCs), JAK3 activity is induced, and increases in costimulatory molecule expression, IL-12 production, and potent allogeneic stimulatory capacity are observed. A rationally designed JAK3 inhibitor WHI-P-154 prevented these effects arresting the DCs at an immature level, suggesting that immunosuppressive therapies targeting the tyrosine kinase JAK3 may also affect the function of myeloid cells (Saemann, M. D., C. Diakos, et al. (2003). “Prevention of CD40-triggered dendritic cell maturation and induction of T-cell hyporeactivity by targeting of Janus kinase 3.” Am J Transplant 3 (11): 1341-9). In the mouse model system, JAK3 was also shown to be an important molecular target for treatment of autoimmune insulin-dependent (type 1) diabetes mellitus. The rationally designed JAK3 inhibitor JANEX-1 exhibited potent immunomodulatory activity and delayed the onset of diabetes in the NOD mouse model of autoimmune type 1 diabetes (Cetkovic-Cvrlje, M., A. L. Dragt, et al. (2003). “Targeting JAK3 with JANEX-1 for prevention of autoimmune type 1 diabetes in NOD mice.” Clin Immunol 106 (3): 213-25).

Thus, new or improved agents which inhibit Janus kinases are continually needed that act as immunosuppressive agents for organ transplants, as well as agents for the prevention and treatment of autoimmune diseases (e.g., multiple sclerosis, rheumatoid arthritis, asthma, type I diabetes, inflammatory bowel disease, Crohn's disease, autoimmune thyroid disorders, Alzheimer's disease), diseases involving a hyperactive inflammatory response (e.g., eczema), allergies and cancer (e.g., prostate, leukemia, multiple myeloma). The compounds, compositions and methods described herein are directed toward this end.

### SUMMARY OF THE INVENTION

The present invention provides, inter alia, compounds of Formula I: 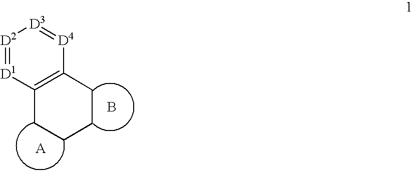
or pharmaceutically acceptable salts or prodrugs thereof, wherein constituent members are defined herein.

The present invention further provides compositions comprising a compound of Formula I and a pharmaceutically acceptable carrier.

The present invention further provides a method of modulating an activity of JAK comprising contacting JAK with a compound of Formula I.

The present invention further provides a method of treating a disease in a patient, where the disease is associated with JAK activity, by administering to the patient a therapeutically effective amount of a compound of Formula I.

### DETAILED DESCRIPTION

The present invention provides, inter alia, compounds of Formula I: 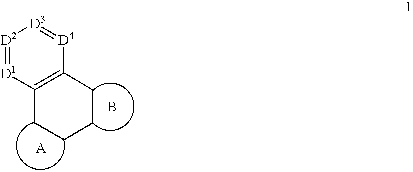
or pharmaceutically acceptable salt or prodrug thereof, wherein:
D¹ is N, NO, or CR^{1a};
D² is N, NO, or CR^{1b};
D³ is N, NO, or CR^{1c};
D⁴ is N, NO or CR^{1d};
Ring A is 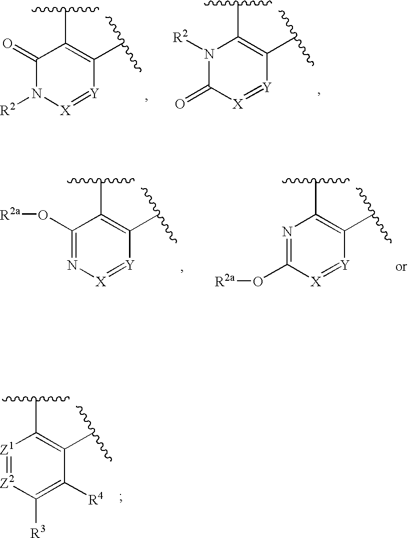
X and Y are each, independently, N or CR⁵;
Z¹ and Z² are each, independently, N, CR⁶, or NO; wherein at least one of Z¹ and Z² is other than CR⁶;
Ring B is 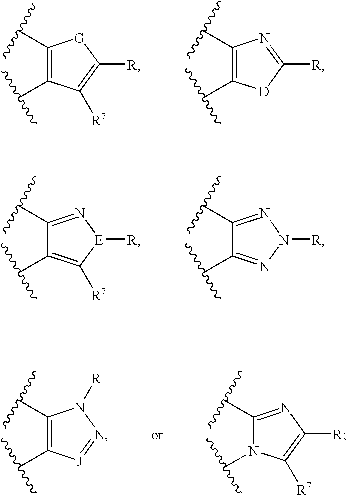
D is O, S, or NR⁸;
E is N or CR⁹;
G is O, S, or NR⁸;
J is N or CR⁷;
R is —W¹—W²—W³—W⁴;
W¹ is absent, C₁₋₆ alkyl, C₂₋₆ alkenyl, C₂₋₆ alkynyl, O, S, NR¹¹, CO, COO, CONR¹¹, SO, SO₂, SONR¹¹, SO₂NR¹¹, or NR¹¹CONR¹², wherein said C₁₋₆alkyl, C₂₋₆alkenyl, C₂₋₆alkynyl are each optionally substituted by 1, 2 or 3 halo, OH, C₁₋₄alkoxy, C₁₋₄ haloalkoxy, amino, C₁₋₄ alkylamino or C₂₋₈ dialkylamino;
W² is absent, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by one or more halo, CN, NO₂, OH, ═NH, ═NOH, ═NO—(C₁₋₄alkyl), C₁₋₄ haloalkyl, C₁₋₄ alkoxy, C₁₋₄ haloalkoxy, amino, C₁₋₄ alkylamino or C₂₋₈ dialkylamino;
W³ is absent, C₁₋₆ alkyl, C₂₋₆ alkenyl, C₂₋₆ alkynyl, O, S, NR¹⁰, ═N—, ═N—O—, ═N—O—(C₁₋₄ alkyl), O—(C₁₋₄ alkyl), S—(C₁₋₄ alkyl), NR¹⁰—(C₁₋₄ alkyl), (C₁₋₄ alkyl)-O—(C₁₋₄ alkyl), (C₁₋₄ alkyl)-S—(C₁₋₄ alkyl), (C₁₋₄ alkyl)-NR¹⁰—(C₁₋₄ alkyl), CO, COO, C(O)—(C₁₋₄ alkyl), C(O)O—(C₁₋₄ alkyl), C(O)—(C₁₋₄ alkyl)-C(O), NR¹⁰C(O)—(C₁₋₄ alkyl), C(O)NR¹⁰—(C₁₋₄ alkyl), NR¹⁰C(O)O—(C₁₋₄ alkyl), NR¹⁰C(O)O, CONR¹⁰, SO, SO₂, SONR¹⁰, SO₂NR¹⁰, or NR¹⁰CONR¹¹, wherein said C₁₋₆ alkyl, C₂₋₆ alkenyl, C₂₋₆ alkynyl are each optionally substituted by 1, 2 or 3 halo, OH, CN, C₁₋₄ alkoxy, C₁₋₄ haloalkoxy, amino, C₁₋₄ alkylamino or C₂₋₈ dialkylamino;
W⁴ is H, NR¹⁰R¹¹, CN, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2, 3, 4 or 5 halo, OH, CN, C₄ alkoxy, ═NH, ═NOH, ═NO—(C₄ alkyl), C₁₋₄ haloalkyl, C₁₋₄ haloalkoxy, COOH, COO—(C₁₋₄ alkyl), amino, C₁₋₄ alkylamino or C₂₋₈ dialkylamino;
R^{1a}, R^{1b}, R^{1c} and R^{1d} are each, independently, H, halo, C₁₋₄ alkyl, C₂₋₄ alkenyl, C₂₋₄alkynyl, C₁₋₄ haloalkyl, OH, C₁₋₄ alkoxy, C₁₋₄ haloalkoxy, CN, NO₂, C(O)—(C₁₋₄ alkyl), C(O)OH, C(O)O—(C₁₋₄ alkyl), C(O)NH₂, C(O)NH(C₁₋₄ alkyl), C(O)N(C₁₋₄ alkyl)₂, S(O)₂NH₂, S(O)₂NH(C₁₋₄ alkyl), S(O)₂N(C₄ alkyl)₂, S(O)₂—(C₁₋₄ alkyl), NH₂, NH(C₁₋₄alkyl), or N(C₁₋₄alkyl)₂;
R² is H, OH, C₁₋₆ alkyl, C₂₋₆alkenyl, C₂₋₆ alkynyl, carbocyclyl, heterocyclyl, carbocyclylalkyl or heterocyclylalkyl;
R^{2a} is C₁₋₆ alkyl, C₂₋₆ alkenyl, C₂₋₆ alkynyl, aryl, heteroaryl, arylalkyl, heteroarylalkyl, cycloalkyl, cycloalkylalkyl, heterocycloalkyl or heterocycloalkylalkyl;
R³, R⁴, R⁵, and R⁶ are each, independently, H, C₁₋₆ alkyl, C₂₋₆ alkenyl, C₂₋₆ alkynyl, halo, C₁₋₄ haloalkyl, CN, NO₂, OR¹², SR¹², C(O)R¹³, C(O)OR¹², C(O)NR¹⁴R¹⁵, NR¹⁴R¹⁵, NR¹⁴CONHR¹⁵, NR¹⁴C(O)R¹³, NR¹⁴C(O)OR¹², S(O)R¹³, S(O)₂R¹³, S(O)NR¹⁴R¹⁵, SO₂NR¹⁴R¹⁵;
R⁷ is H, C₁₋₆ alkyl, C₂₋₆ alkenyl, C₂₋₆ alkynyl, halo, C₁₋₄ haloalkyl, OH, C₁₋₄ alkoxy, C₁₋₄ haloalkoxy, CN, NO₂, C(O)—(C₁₋₄ alkyl), C(O)OH, C(O)O—(C₁₋₄ alkyl), C(O)NH₂, C(O)NH(C₁₋₄ alkyl), C(O)N(C₁₋₄ alkyl)₂, S(O)₂NH₂, S(O)₂NH(C₁₋₄ alkyl), S(O)₂N(C₁₋₄ alkyl)₂, S(O)₂—(C₁₋₄ alkyl), NH₂, NH(C₁₋₄ alkyl), or N(C₁₋₄ alkyl)₂;
R⁸ is H, C₁₋₄ alkyl, C₂₋₄ alkenyl, C₂₋₄ alkynyl, OH or C₁₋₄ alkoxy;
R⁹ is H, halo, C₁₋₄ alkyl, C₁₋₄ haloalkyl, C₂₋₄ alkenyl, C₂₋₄ alkynyl, OH, C₁₋₄ alkoxy or C₁₋₄ haloalkoxy;
R¹⁰ and R¹¹ are each, independently, H, C₁₋₆ alkyl, C₂₋₆ alkenyl, C₂₋₆ alkynyl, aryl, cycloalkyl, arylalkyl, cycloalkylalkyl, COR^{a}, SOR^{a}, or SO₂R^{a} wherein each of said C₁₋₆ alkyl, C₂₋₆ alkenyl, C₂₋₆ alkynyl, aryl, cycloalkyl, arylalkyl, or cycloalkylalkyl is optionally substituted by 1, 2 or 3 substitutents slected from halo, C₁₋₄ alkyl, C₁₋₄ haloalkyl, OH, C₁₋₄ alkoxy, C₁₋₄ haloalkoxy, amino, C₁₋₄ alkylamino, C₂₋₈ dialkylamino, aminocarbonyl, C₁₋₄ alkylaminocarbonyl, or C₂₋₈ dialkylaminocarbonyl, CN and NO₂;
or R¹⁰ and R¹¹ together with the N atom to which they are attached form a heterocycloalkyl group optionally substituted by 1, 2 or 3 substitutents slected from halo, C₁₋₄ alkyl, C₁₋₄ haloalkyl, OH, C₁₋₄ alkoxy, C₁₋₄ haloalkoxy, amino, C₁₋₄ alkylamino, C₂₋₈ dialkylamino, aminocarbonyl, C₁₋₄ alkylaminocarbonyl, or C₂₋₈ dialkylaminocarbonyl;
R¹² and R¹³ are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
R¹⁴ and R¹⁵ are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R¹⁴ and R¹⁵ together with the N atom to which they are attached form a heterocyclyl group;
R^{a} is H, C₁₋₆ alkyl, C₁₋₆ haloalkyl, C₂₋₆ alkenyl, C₂₋₆ alkynyl, aryl, cycloalkyl, arylalkyl, cycloalkylalkyl, heteroaryl, heterocycloalkyl, heteroarylalkyl, heterocycloalkylalkyl, NH₂, NH(C₁₋₆ alkyl), N(C₁₋₆ alkyl)₂, NH(carbocyclyl), N(carbocyclyl)₂, NH(carbocyclylalkyl) or N(carbocyclylalkyl)₂;
   with the proviso that when Ring A is: 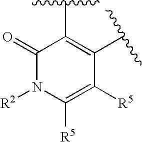
Ring B is: 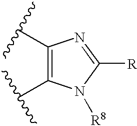
D¹ is CR^{1a};
D² is N or CR^{1b};
D³ is CR^{1c}; and
D⁴ is CR^{1d};
   then W¹ is O, S, NR¹¹, SO, SO₂, SONR¹¹, SO₂NR¹¹, or NR¹¹CONR¹².

According to some embodiments, Ring A is

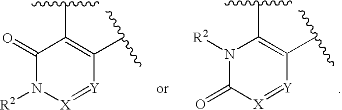

In some embodiments, both X and Y are CR⁵.

In some embodiments, both X and Y are N.

In some embodiments, one of X and Y is N and the other is CR⁵.

In some embodiments, X is CR⁵ and Y is N.

In some embodiments, X is N and Y is CR⁵.

In some embodiments, R² is H.

In some embodiments, R² is H, X is CH and Y is CH.

In some embodiments, Ring A is

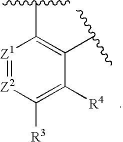

In some embodiments, Z¹ is NO or Z² is NO.

In some embodiments, Z¹ is NO and Z² is CR⁶.

In some embodiments, Z² is NO and Z¹ is CR⁶.

In some embodiments, Ring A is

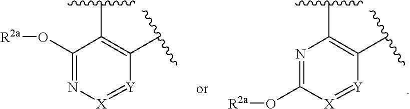

In some embodiments, R^{2a} is C₁₋₆ alkyl.

In some embodiments, R^{2a} is methyl.

In some embodiments, at least one of X and Y is N.

In some embodiments, Ring B is

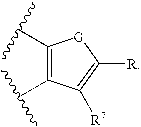

In some embodiments, G is O or S.

In some embodiments, G is NR⁸.

In some embodiments, G is NH.

In some embodiments, R is H, C₁₋₆ alkyl, aryl, heteroaryl, cycloalkyl, heterocycloalkyl, or NR¹⁰R¹¹

In some embodiments, R is H, C₁₋₆ alkyl or NR¹⁰R¹¹.

In some embodiments, R is O—W²—W³—W⁴, S—W²—W³—W⁴ or NR¹¹—W²—W³—W⁴.

In some embodiments, Ring B is

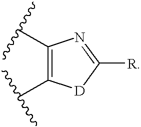

In some embodiments, D is S.

In some embodiments, D is O.

In some embodiments, D is NR⁸.

In some embodiments, R is H, C₁₋₆ alkyl, aryl, heteroaryl, cycloalkyl, heterocycloalkyl, or NR¹⁰R¹¹

In some embodiments, R is H, C₁₋₆ alkyl or NR¹⁰R¹¹.

In some embodiments, R is (C₁₋₆alkyl)-W²—W³—W⁴, O—W²—W³—W⁴, S—W²—W³—W⁴, NR¹¹—W²—W³—W⁴, or —W²—W³—W⁴.

In some embodiments, D is S or O and R is O—W²—W³—W⁴, S—W²—W³—W⁴ or NR¹¹—W²—W³—W⁴.

In some embodiments, D is S and R is O—W²—W³—W⁴, S—W²—W³—W⁴ or NR¹¹—W²—W³—W⁴.

In some embodiments, Ring B is

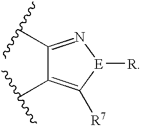

In some embodiments, E is N.

In some embodiments, R⁷ is H.

In some embodiments, R is H, C₁₋₆ alkyl, aryl, heteroaryl, cycloalkyl, heterocycloalkyl, or NR¹⁰R¹¹

In some embodiments, R is H, C₁₋₆ alkyl or NR¹⁰R¹¹.

In some embodiments, E is CR⁹ and R is O—W²—W³—W⁴, S—W²—W³—W⁴ or NR¹¹—W²—W³—W⁴.

In some embodiments, Ring B is

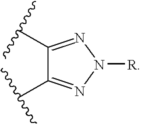

In some embodiments, R is H, C₁₋₆ alkyl, aryl, heteroaryl, cycloalkyl, heterocycloalkyl.

In some embodiments, R is H or C₁₋₆ alkyl.

In some embodiments, R is (C₁₋₆ alkyl)-W²—W³—W⁴, CO—W²—W³—W⁴, COO—W²—W³—W⁴, CONR¹¹—W²—W³—W⁴ or SO₂—W²—W³—W⁴.

In some embodiments, Ring B is:

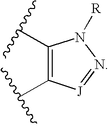

In some embodiments, J is N.

In some embodiments, J is CR⁷.

In some embodiments, R is H, C₁₋₆ alkyl, aryl, heteroaryl, cycloalkyl, heterocycloalkyl.

In some embodiments, R is H or C₁₋₆ alkyl.

In some embodiments, R is (C₁₋₆ alkyl)-W²—W³—W⁴, CO—W²—W³—W⁴, COO—W²—W³—W⁴, CONR¹—W²—W³—W⁴ or SO₂—W²—W³—W⁴.

In some embodiments, Ring B is

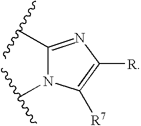

In some embodiments, R is C₁₋₆ alkyl, C₂₋₆ alkenyl, C₂₋₆ alkynyl, cycloalkyl, or heterocycloalkyl, each optionally substituted by 1, 2, 3, 4 or 5 halo, OH, CN, C₁₋₄ alkoxy, ═NH, ═NOH, ═NO—(C₁₋₄ alkyl), C₁₋₄ haloalkyl, C₁₋₄ haloalkoxy, COOH, COO—(C₁₋₄ alkyl), amino, C₁₋₄ alkylamino or C₂₋₈ dialkylamino;
In some embodiments, R is cycloalkyl or heterocycloalkyl, each optionally substituted by 1, 2, 3, 4 or 5 halo, OH, CN, C₁₋₄ alkoxy, C₁₋₄ haloalkyl, C₁₋₄ haloalkoxy, COOH, COO—(C₁₋₄ alkyl), amino, C₁₋₄ alkylamino or C₂₋₈ dialkylamino.

In some embodiments, R is 5-, 6-, or 7-membered cycloalkyl or 5-, 6-, or 7-membered heterocycloalkyl, each optionally substituted by 1 or 2 halo, OH, CN, C₁₋₄ alkoxy, C₁₋₄ haloalkyl, or C₁₋₄ haloalkoxy.

In some embodiments, D¹ is CR^{1a}, D² is CR^{1b}, D³ is CR^{1c} and D⁴ is CR^{1d}.

In some embodiments, D² is CR^{1b}.

In some embodiments, D² is CR^{1b} and CR^{1b} is H, C₁₋₄ alkyl or halo.

In some embodiments, D is CR^{1b} and CR^{1b} is H or halo.

In some embodiments, D² is CR^{1b} and CR^{1b} is F, Cl, Br or I.

In some embodiments, D² is CR^{1b}; CR^{1b} is F, Cl, Br or I; D¹ is CH, D³ is CH; and D⁴ is CH.

In some embodiments, D² is CF; D¹ is CH, D³ is CH; and D⁴ is CH.

In some embodiments, at least one of D¹, D², D³, and D⁴ is N.

In some embodiments, at least one of D¹, D³, and D⁴ is N.

In some embodiments, not more than 2 of D¹, D², D³, and D⁴ are N.

In some embodiments, at least one of D¹, D², D³, and D⁴ is NO.

In some embodiments, at least one of D¹, D³, and D⁴ is NO.

In some embodiments, compounds of the invention the Formula Ia:

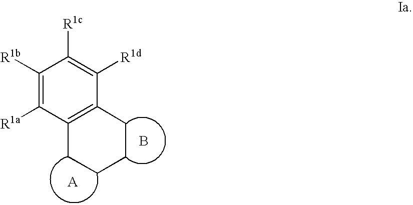

In some embodiments, R^{1a}, R^{1b}, R^{1c} and R^{1d} are each, independently, H, C₁₋₄ alkyl, C₂₋₄ alkenyl, C₂₋₄ alkynyl, halo, C₁₋₄ haloalkyl, OH, C₁₋₄ alkoxy, C₁₋₄ haloalkoxy, CN, NO₂, NH₂, NH(C₁₋₄ alkyl), or N(C₁₋₄ alkyl)₂.

In some embodiments, R is other than H.

In some embodiments, R is —W¹—W²—W³—W⁴; and W¹ is absent, C., alkyl, O, S, NR¹¹, SO, or SO₂.

In some embodiments, R is —W¹—W²—W³—W⁴; and W¹ is absent, and W² is aryl, cycloalkyl, heteroaryl or heterocycloalkyl, each optionally substituted by 1, 2, 3 or 4 halo, CN, NO₂, OH, ═NH, ═NOH, ═NO—(C₁₋₄ alkyl), C₁₋₄ haloalkyl, C₁₋₄ alkoxy, C₁₋₄ haloalkoxy, amino, C₁₋₄ alkylamino or C₂₋₈ dialkylamino.

In some embodiments:
R is —W¹—W²—W³—W⁴;
W¹ is absent or C₁₋₆ alkyl optionally substituted by 1, 2 or 3 halo, OH, C₁₋₄ alkoxy, C₁₋₄ haloalkoxy, amino, C₁₋₄ alkylamino or C₂₋₈ dialkylamino;
W² is absent; and
W³ is O, S, NR¹⁰, CO, or COO.

In some embodiments, R is H, C₁₋₆ alkyl, C₂₋₆ alkenyl, C₂₋₆ alkynyl, O—W²—W³—W⁴, S—W²—W³—W⁴, or NR¹¹—W²—W³—W⁴, wherein said C₁₋₆ alkyl, C₂₋₆ alkenyl, C₂₋₆ alkynyl are each optionally substituted by 1, 2 or 3 halo, OH, C₁₋₄ alkoxy, C₁₋₄ haloalkoxy, amino, C₁₋₄ alkylamino or C₂₋₈ dialkylamino.

In some embodiments, R is W⁴.

In some embodiments, R is —W³—W⁴.

In some embodiments, R is —W²—W³—W⁴.

In some embodiments, R is —W¹—W⁴.

In some embodiments, R is —O—W²—W³—W⁴.

In some embodiments, R is —S—W²—W³—W⁴.

In some embodiments, R is —NR¹¹—W²—W³—W⁴.

In some embodiments, R is NR¹⁰R¹¹.

In some embodiments, R is aryl, cycloalkyl, heteroaryl or heterocycloalkyl each optionally substituted by 1, 2, 3, 4 or 5 halo, OH, CN, C₁₋₄ alkoxy, ═NH, ═NOH, ═NO—(C₁₋₄ alkyl), C₁₋₄ haloalkyl, C₁₋₄ haloalkoxy, COOH, COO—(C₁₋₄ alkyl), amino, C₁₋₄ alkylamino or C₂₋₈ dialkylamino.

In some embodiments, W¹ is O, S, NR¹¹, CO, COO, CONR¹¹, SO, SO₂, SONR¹¹, SO₂NR¹¹, or NR¹¹CONR¹².

In some embodiments, W¹ is C₁₋₆ alkyl optionally substituted by one or more halo, CN, NO₂, OH, ═NH, ═NOH, ═NO—(C₁₋₄ alkyl), C₁₋₄ haloalkyl, C₁₋₄alkoxy, CIA haloalkoxy, amino, C₁₋₄ alkylamino or C₂₋₈ dialkylamino.

In some embodiments, W¹ is absent.

In some embodiments, W² is aryl, cycloalkyl, heteroaryl or heterocycloalkyl, each optionally substituted by one or more halo, CN, NO₂, OH, ═NH, NOH, ═NO—(C₁₋₄ alkyl), C₁₋₄ haloalkyl, C₁₋₄ alkoxy, C₁₋₄ haloalkoxy, amino, C₁₋₄ alkylamino or C₂₋₈ dialkylamino.

In some embodiments, W² is absent.

In some embodiments, W³ is O, S, NR¹⁰, ═N—, ═N—O—, ═N—O—(C₁₋₄ alkyl), O—(C₁₋₄ alkyl), S—(C₁₋₄ alkyl), NR¹⁰—(C₁₋₄ alkyl), (C₁₋₄ alkyl)-O—(C₁₋₄ alkyl), (C₁₋₄ alkyl)-S—(C₁₋₄ alkyl), (C₁₋₄ alkyl)-NR¹¹—(C₁₋₄ alkyl), CO, COO, C(O)—(C₁₋₄ alkyl), C(O)O—(C₁₋₄ alkyl), C(O)—(C₁₋₄ alkyl)-C(O), NR¹⁰C(O)—(C₁₋₄ alkyl), C(O)NR¹⁰—(C₁₋₄ alkyl), NR¹⁰C(O)O—(C₁₋₄ alkyl), NR¹⁰C(O)O, CONR¹⁰, SO, SO₂, SONR¹⁰, SO₂NR¹⁰, or NR¹⁰CONR¹¹.

In some embodiments, W³ is C₁₋₆ alkyl optionally substituted by 1, 2 or 3 halo, OH, CN, C₁₋₄ alkoxy, C₁₋₄ haloalkoxy, amino, C₁₋₄ alkylamino or C₂₋₈ dialkylamino.

In some embodiments, W³ is absent.

In some embodiments, W⁴ is aryl, cycloalkyl, heteroaryl or heterocycloalkyl, each optionally substituted by 1, 2, 3, 4 or 5 halo, OH, CN, C₁₋₄ alkoxy, ═NH, ═NOH, ═NO—(C₄ alkyl), C₁₋₄ haloalkyl, C₁₋₄ haloalkoxy, COOH, COO—(C₁₋₄alkyl), amino, C₁₋₄alkylamino or C₂₋₈ dialkylamino.

In some embodiments, W⁴ is C₁₋₆ alkyl optionally substituted by 1, 2, 3, 4 or 5 halo, OH, CN, C₁₋₄alkoxy, ═NH, ═NOH, ═NO—(C₁₋₄alkyl), C₁₋₄ haloalkyl, C₁₋₄ haloalkoxy, COOH, COO—(C₁₋₄ alkyl), amino, C₁₋₄ alkylamino or C₂₋₈ dialkylamino.

In some embodiments, W⁴ is H, NR¹⁰R¹ or CN.

In some embodiments:
Ring B is 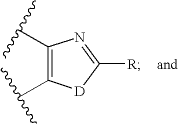
R is H, C₁₋₆ alkyl, C₂₋₆ alkenyl, C₂₋₆ alkynyl, (C₁₋₆ alkyl)-W²—W³—W⁴, O—W²—W³—W⁴, S—W²—W³—W⁴, NR¹¹—W²—W³—W⁴, or —W²—W³—W⁴, wherein said C₁₋₆ alkyl, C₂₋₆ alkenyl, C₂₋₆ alkynyl are each optionally substituted by 1, 2 or 3 halo, OH, C₁₋₄alkoxy, CIA haloalkoxy, amino, C₁₋₄ alkylamino or C₂₋₈ dialkylamino.

In some embodiments:
Ring B is 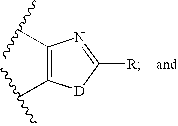
R is S_W²—W³—W⁴, S(O)—W²—W³—W⁴ or S(O)₂—W²—W³—W⁴.

In some embodiments:
Ring B is 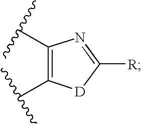
D is NR⁸; and
R is S—W²—W³—W⁴, S(O)—W²—W³—W⁴ or S(O)₂—W²—W¹—W⁴.

In some embodiments:
Ring B is 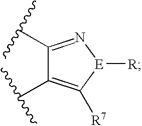
E is N; and
R is H, (C₁₋₆ alkyl)-W²—W³—W⁴, (C₂₋₆ alkenyl)-W²—W³—W⁴ or (C₂₋₆ alkynyl)-W²—W³—W⁴.

In some embodiments:
Ring B is 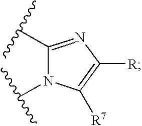
R⁷ is H; and
R is C₁₋₆ alkyl, C₂₋₆ alkenyl, C₂₋₆ alkynyl, cycloalkyl, or heterocycloalkyl, each optionally substituted by 1, 2, 3, 4 or 5 halo, OH, CN, C₁₋₄ alkoxy, ═NH, ═NOH, ═NO—(C₁₋₄ alkyl), C₁₋₄ haloalkyl, C₁₋₄ haloalkoxy, COOH, COO—(C₁₋₄ alkyl), amino, C₁₋₄ alkylamino or C₂₋₈ dialkylamino.

In some embodiments:
Ring B is 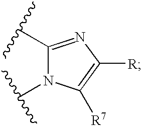
R⁷ is H; and
R is cycloalkyl, or heterocycloalkyl, each optionally substituted by 1, 2, 3, 4 or 5 halo, OH, CN, C₁₋₄ alkoxy, C₁₋₄ haloalkyl, C₁₋₄ haloalkoxy, COOH, COO—(C₁₋₄ alkyl), amino, C₁₋₄ alkylamino or C₂₋₈ dialkylamino.

In some embodiments, the compounds of the invention have Formula II:

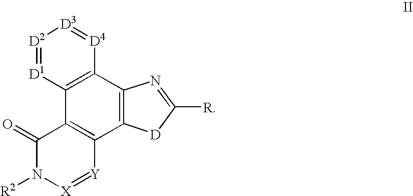

In some embodiments, the compounds of the invention have Formula III:

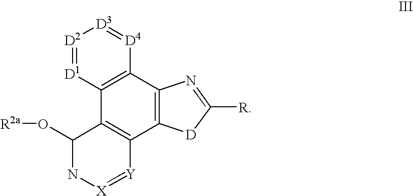

In some embodiments, the compounds of the invention have Formula IV:

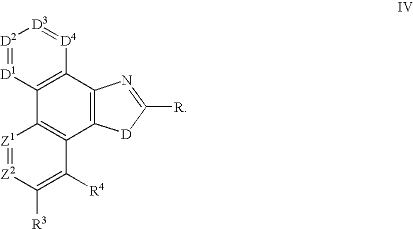

In some embodiments, the compounds of the invention have Formula V:

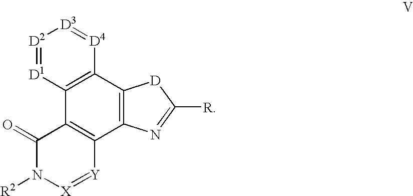

In some embodiments, the compounds of the invention have Formula VI:

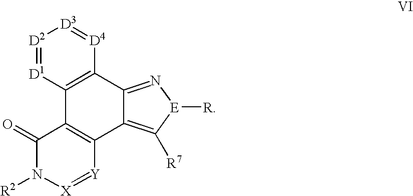

In some embodiments, the compounds of the invention have Formula VII:

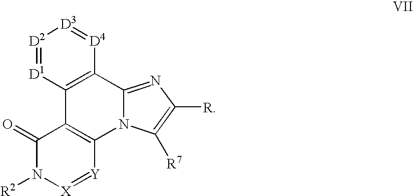

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

For compounds of the invention in which a variable appears more than once, each variable can be a different moiety selected from the Markush group defining the variable. For example, where a structure is described having two R groups that are simultaneously present on the same compound; the two R groups can represent different moieties selected from the Markush group defined for R.

It is further intended that where a group is depicted in a certain direction or orientation, all other possible orientations are included. For example, it is intended that the defining groups of ring A and ring B are meant to include all orientations, such that when rings A and B are asymmetric they can be combined with the core structure in at least two possible orientations.

It is further intended with respect to the moiety —W¹—W²—W³—W⁴, that the bond(s) connecting each component (e.g., bonds between W¹ and W², between W² and W³, etc.) can be single, double, or normalized.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms. The term “alkyl” is further used in the case of bivalent (linker) alkyl groups.

As used herein, “alkenyl” refers to an alkyl group having one or more double carbon-carbon bonds. Example alkenyl groups include ethenyl, propenyl, cyclohexenyl, and the like. The term “alkenyl” is further used herein in the case of bivalent (linker) alkenyl groups.

As used herein, “alkynyl” refers to an alkyl group having one or more triple carbon-carbon bonds. Example alkynyl groups include ethynyl, propynyl, and the like. The term “alkynyl” is further used herein in the case of bivalent (linker) alkynyl groups.

As used herein, “haloalkyl” refers to an alkyl group having one or more halogen substituents. Example haloalkyl groups include CF₃, C₂F₅, CHF₂, CCl₃, CHCl₂, C₂Cl₅, and the like.

As used herein, “carbocyclyl” groups are saturated (i.e., containing no double or triple bonds) or unsaturated (i.e., containing one or more double or triple bonds) cyclic hydrocarbon moieties. Carbocyclyl groups can be mono- or polycyclic (e.g., having 2, 3 or 4 fused rings). Example carbocyclyl groups include cyclopropyl, cyclobutyl, cyclopentyl, cyclohexyl, cycloheptyl, cyclopentenyl, 1,3-cyclopentadienyl, cyclohexenyl, norbornyl, norpinyl, norcarnyl, adamantyl, phenyl, and the like. Carbocyclyl groups can be aromatic (e.g., “aryl”) or non-aromatic (e.g., “cycloalkyl”). In some embodiments, carbocyclyl groups can have from about 3 to about 30 carbon atoms, about 3 to about 20, about 3 to about 10, or about 3 to about 7 carbon atoms.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spiro systems. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of pentane, pentene, hexane, and the like. One or more ring-forming carbon atoms of a cycloalkyl group can be oxidized, for example, having an oxo or sulfide substituent. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like.

As used herein, “heterocyclyl” or “heterocycle” refers to a saturated or unsaturated cyclic group wherein one or more of the ring-forming atoms is a heteroatom such as O, S, or N. Heterocyclyl groups include mono- or polycyclic ring systems. Heterocyclyl groups can be aromatic (e.g., “heteroaryl”) or non-aromatic (e.g., “heterocycloalkyl”). Heterocyclyl groups can be characterized as having 3-14 ring-forming atoms. In some embodiments, heterocyclyl groups can contain, in addition to at least one heteroatom, from about 1 to about 13, about 2 to about 10, or about 2 to about 7 carbon atoms and can be attached through a carbon atom or heteroatom. In further embodiments, the heteroatom can be oxidized (e.g., have an oxo or sulfido substituent) or a nitrogen atom can be quaternized. Examples of heterocyclyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like, as well as any of the groups listed below for “heteroaryl” and “heterocycloalkyl.” Further example heterocycles include pyrimidinyl, phenanthridinyl, phenanthrolinyl, phenazinyl, phenothiazinyl, phenoxathiinyl, phenoxazinyl, phthalazinyl, piperazinyl, piperidinyl, 3,6-dihydropyridyl, 1,2,3,6-tetrahydropyridyl, 1,2,5,6-tetrahydropyridyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, tetrahydrofuranyl, tetrahydroisoquinolinyl, tetrahydroquinolinyl, tetrazolyl, 6H-1,2,5-thia-diazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl, octahydro-isoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, quinazolinyl, quinolinyl, 4H-quinolizinyl, quinoxalinyl, quinuclidinyl, acridinyl, azocinyl, benzimidazolyl, benzofuranyl, benzothiofuranyl, benzo-thiophenyl, benzoxazolyl, benzthiazolyl, benztriazolyl, benztetrazolyl, benzisoxazolyl, benzisothiazolyl, benzimidazolinyl, methylenedioxyphenyl, morpholinyl, naphthyridinyl, deca-hydroquinolinyl, 2H,6H-1,5,2dithiazinyl, dihydrofuro[2,3-b]tetrahydrofuran, furanyl, furazanyl, carbazolyl, 4aH-carbazolyl, carbolinyl, chromanyl, chromenyl, cinnolinyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl and isoxazolyl. Further examples of heterocycles include azetidin-1-yl, 2,5-dihydro-1H-pyrrol-1-yl, piperindin-lyl, piperazin-1-yl, pyrrolidin-1-yl, isoquinol-2-yl, pyridin-1-yl, 3,6-dihydropyridin-1-yl, 2,3-dihydroindol-1-yl, 1,3,4,9-tetrahydrocarbolin-2-yl, thieno[2,3-c]pyridin-6-yl, 3,4,10,10a-tetrahydro-1H-pyrazino[1,2-a) indol-2-yl, 1,2,4,4a,5,6-hexahydro-pyrazino[1,2-a]quinolin-3-yl, pyrazino[1,2-a]quinolin-3-yl, diazepan-1-yl, 1,4,5,6-tetrahydro-2H-benzo[f]isoquinolin-3-yl, 1,4,4a,5,6,10b-hexahydro-2H-benzo[f]isoquinolin-3-yl, 3,3a,8,8a-tetrahydro-1H-2-aza-cyclopenta[a]inden-2-yl, and 2,3,4,7-tetrahydro-1H-azepin-1-yl, azepan-1-yl.

As used herein, “heteroaryl” groups refer to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heterocycloalkyl” refers to non-aromatic heterocycles including cyclized alkyl, alkenyl, and alkynyl groups where one or more of the ring-forming atoms is a heteroatom such as an O, N, or S atom. Heterocycloalkyl groups can include mono- or polycyclic (e.g., having 2, 3 or 4 fused rings) ring systems as well as spiro systems. Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles such as indolene and isoindolene groups. In some embodiments, the heterocycloalkyl group has from 1 to about 20 carbon atoms, and in further embodiments from about 3 to about 20 carbon atoms. In some embodiments, the heterocycloalkyl group contains 3 to about 20, 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

As used herein, “aryloxy” refers to an —O-aryl group. An example aryloxy group is phenoxy.

As used here, “haloalkoxy” refers to an —O-haloalkyl group. An example haloalkoxy group is OCF₃.

As used herein, “carbocyclylalkyl” refers to an alkyl moiety substituted by a carbocyclyl group. Example carbocyclylalkyl groups include “aralkyl” (alkyl substituted by aryl (“arylalkyl”)) and “cycloalkylalkyl” (alkyl substituted by cycloalkyl). In some embodiments, carbocyclylalkyl groups have from 4 to 24 carbon atoms.

As used herein, “heterocyclylalkyl” refers to an alkyl moiety substituted by a heterocarbocyclyl group. Example heterocarbocyclylalkyl groups include “heteroarylalkyl” (alkyl substituted by heteroaryl) and “heterocycloalkylalkyl” (alkyl substituted by heterocycloalkyl). In some embodiments, heterocyclylalkyl groups have from 3 to 24 carbon atoms in addition to at least one ring-forming heteroatom.

As used herein, “amino” refers to NH₂.

As used herein, “alkylamino” refers to an amino group substituted by an alkyl group.

As used herein, “dialkylamino” refers to an amino group substituted by two alkyl groups.

As used herein, “aminocarbonyl” refers to a carbonyl group substituted by an amino group.

As used herein, “alkylaminocarbonyl” refers to a carbonyl group substituted by an alkylamino group.

As used herein, “dialkylaminocarbonyl” refers to a carbonyl group substituted by a dialkylamino group.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. An example method includes fractional recrystallizaion using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as O-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention also include tautomeric forms, such as keto-enol tautomers.

Compounds of the invention can also include all isotopes of atoms occurring in the intermediates or final compounds. Isotopes include those atoms having the same atomic number but different mass numbers. For example, isotopes of hydrogen include tritium and deuterium.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts or the quaternary ammonium salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The present invention also includes prodrugs of the compounds described herein. As used herein, “prodrugs” refer to any covalently bonded carriers which release the active parent drug when administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Synthesis

Compounds of the invention, including salts, hydrates, and solvates thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Preparation of compounds of the invention can involve the protection and deprotection of various chemical groups. The need for protection and deprotection, and the selection of appropriate protecting groups can be readily determined by one skilled in the art. The chemistry of protecting groups can be found, for example, in T. W. Green and P. G. M. Wuts, Protective Groups in Organic Synthesis, 3^{rd}. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

Compounds of the invention can be prepared according to numerous preparatory routes known in the literature such as those reported in WO 03/011285 which is incorporated herein by reference in its entirety. Example synthetic methods for preparing compounds of the invention are provided in the Schemes below.

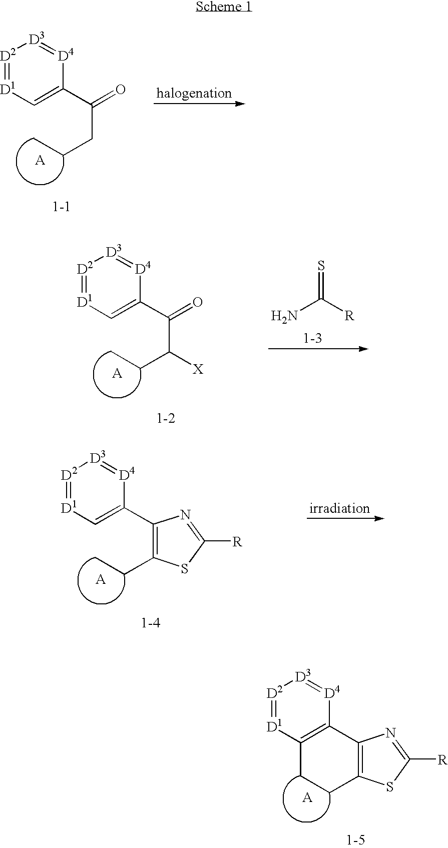

Scheme 1 provides an example preparatory route to thiazole compounds of the invention. Compounds having the formula 1-1, which can be prepared according to methods described in WO 03/011285, can be reacted with a halogenating reagent such as N-bromosuccinimide (NBS), bromine (Br₂) and the like in an appropriate solvent such as dimethylformamide (DMF), acetic acid, mixtures thereof and the like to produce the halogenated compound 1-2 (X is F, Cl, Br or I). The halogenated compound 1-2 can be treated with thioamide 1-3 in a suitable solvent such as acetic acid, THF, DMF, mixtures thereof and the like and optionally at elevated temperature to render thiazole compound 1-4. Irradiation of the thiazole compound with ultraviolet (UV) light results in the tetracyclic thiazole 1-5.

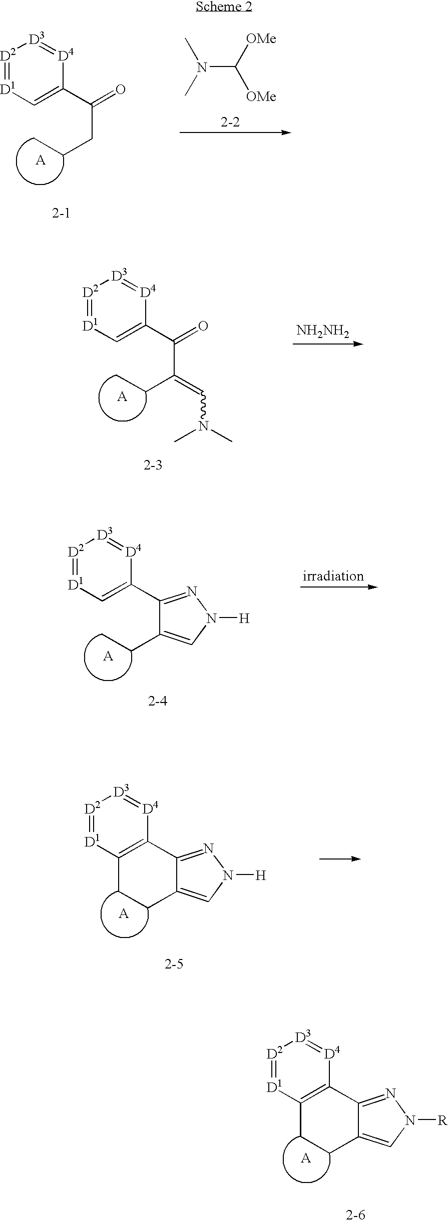

Scheme 2 provides an example preparatory route to pyrazole compounds of the invention. Compounds of having the formula 2-1 can be treated with at least one molar equivalent of aminoacetal 2-2 or similar reagent in appropriate solvent such as an ether (e.g., THF, diethyl ether, etc.) to yield amine 2-3. The amine 2-3 can be reacted with hydrazine in a protic solvent such as an alcohol (e.g., methoanol, ethanol, etc.) to provide pyrazole 2-4. Irradiation of pyrazole 2-4 yields tetracyclic compound 2-5 which can be further derivatized by substitution of the pyrazole proton with —R according to routine methods to yield a variety of compounds with formula 2-6.

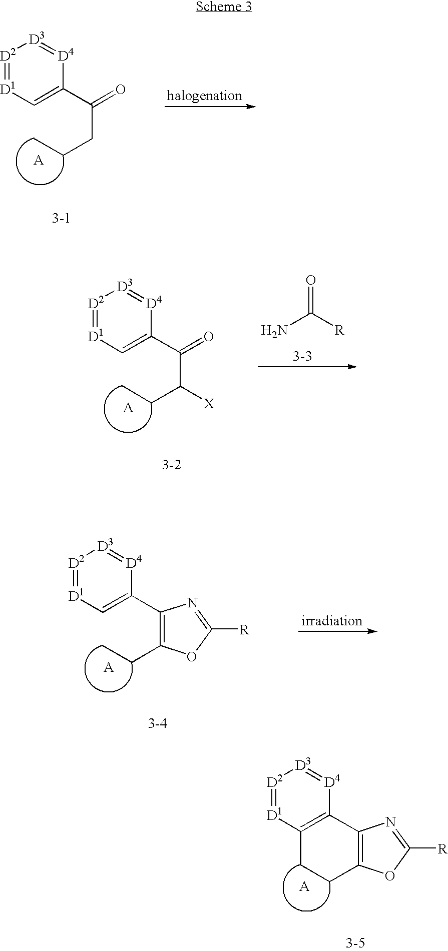

Scheme 3 provides an example preparatory route to oxazole compounds of the invention. Compounds having the formula 3-1 can be reacted with a halogenating reagent such as N-bromosuccinimide (NBS), bromine (Br₂) or the like in an appropriate solvent such as dimethylformamide (DMF), acetic acid, mixtures thereof and the like to produce the halogenated compound 3-2 (X is F, Cl, Br or I). The halogenated compound 3-2 can be treated with amide 3-3 in a suitable solvent such as DMF and optionally at elevated temperature to render oxazole compound 3-4. Irradiation of the oxazole compound with ultraviolet (UV) light results in the tetracyclic oxazole 3-5.

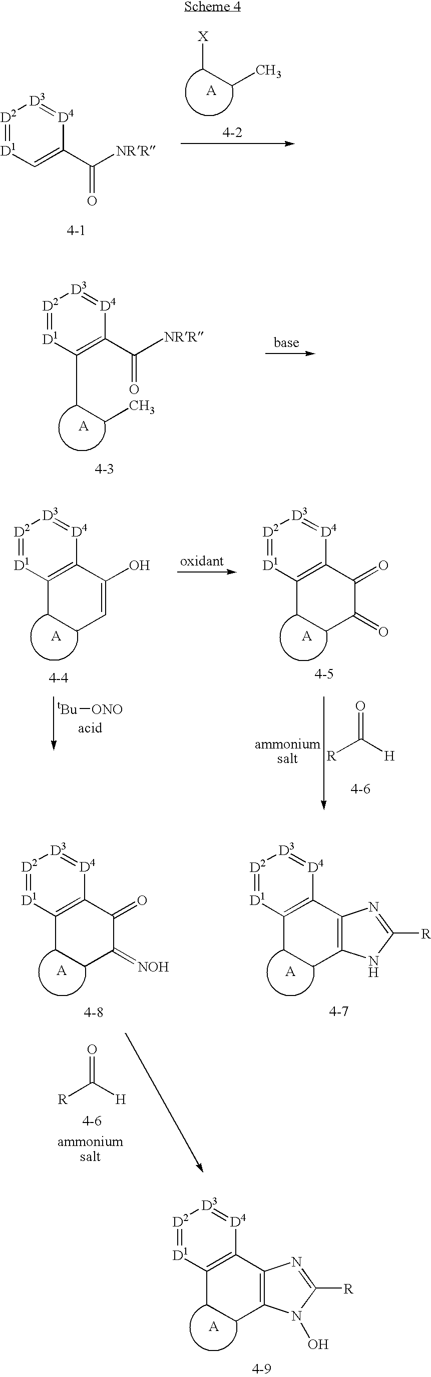

Scheme 4 provides an example preparatory route to imidazole compounds of the invention. Compounds having the formula 4-1 (R′ and R″ can be H, alkyl, etc.) can be treated with a strong base (e.g., about one equivalent) such as an alkyllithium reagent (e.g., sec-butyllithium, t-butlylithium, etc.) in the presence of about 1 equivalent of a tetraalkylethylenediamine reagent (e.g., tetramethylethylenediamine (TMEDA)). Halogenated heterocycle 4-2 can be combined with the resulting mixture in the presence of a metal catalyst (e.g., Pd) and optionally in the presence of heat to provide the benzamide compound of formula 4-3. The benzamde compound 4-3 can be treated with strong base such as lithium diisopropylamide (LDA), LTMP or the like to yield alcohol 4-4 which can be treated with an oxidant such as Cr(VI) in a suitable solvent such as an ether solvent to provide dione 4-5. Dione 4-5 can be treated with aldehyde 4-6 in the presence of an ammonium salt (e.g., ammonium hydroxide, ammonium acetate, etc.) optionally at elevated temperatures to yield tetracyclic imidazoles of formula 4-7. Alternatively, compound 4-4 can be treated with tBuONO in the presence of acid to yield the oxime 4-8 which, when treated with aldehyde 4-6 in the presence of an ammonium salt (e.g., ammonium hydroxide, ammonium acetate, etc.) optionally at elevated temperatures yields hydroxyimidizoles 4-9.

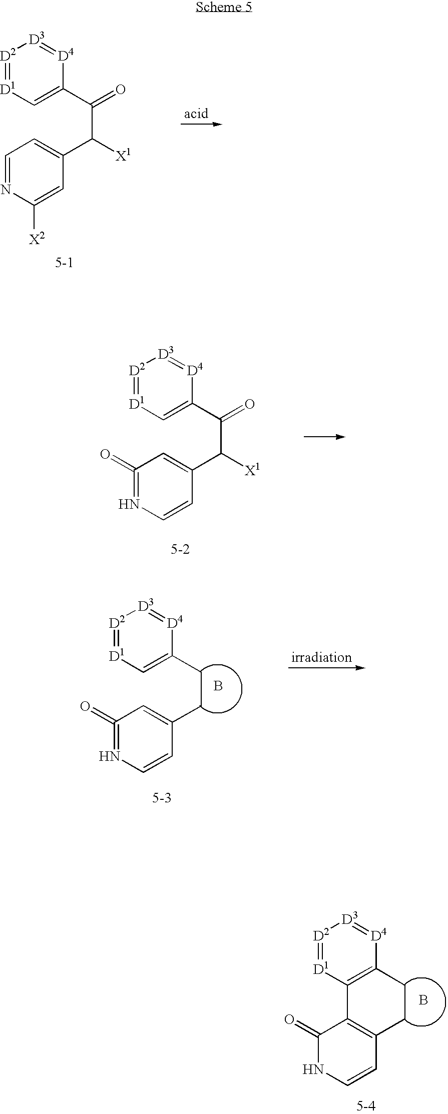

Scheme 5 (X¹ and X² are, independently, F, Cl, Br or I) provides an example preparatory route to pyridone compounds of the invention. Compounds having the formula 5-1 (prepared, for example, according to Scheme 1) can be treated with acid optionally at elevated temperatures to form the corresponding pyridone 5-2. The pyridone can be treated as described, for example, in Schemes 1 and 3 to yield intermediates 5-3 and tetracyclic pyridones 5-4.

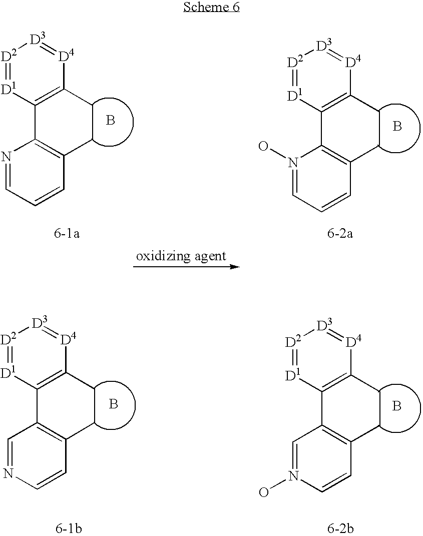

Scheme 6 provides an example preparatory route to N-oxo pyridine compounds of the invention. Compounds having formulas 6-1a or 6-1b, prepared according to certain Schemes provided herein, can be treated with an oxidizing agent such as, for example, BO₃⁻, 3-chloroperoxybenzoic acid (MCPBA), dimethyldioxirane and the like to yield the oxidized compounds of formula 6-2a and 6-2b.

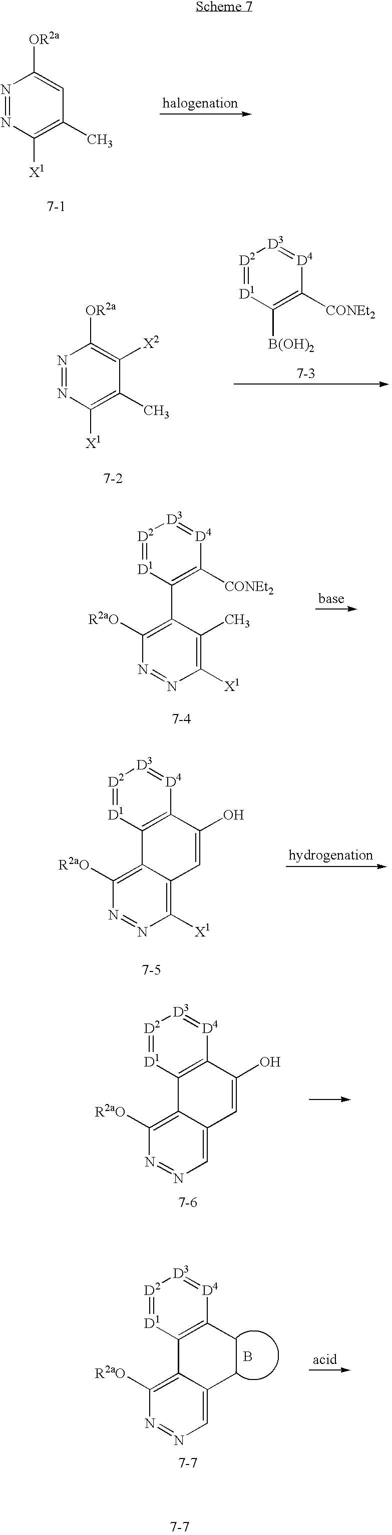

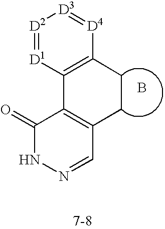

Scheme 7 (X¹ and X² are, independently, F, Cl, Br or I) provides an example preparatory route to pyridazine and pyridazone compounds of the invention. Compounds having formula 7-1 can be halogenated with a suitable halogenating agent such as I₂, NIS and the like optionally in the presence of base to yield halogenated intermediates of formula 7-2. The halogentated intermediates of formula 7-2 can be coupled to phenyl boronic acid reagents of formula 7-3 under, for example, Suzuki type reaction conditions to form coupled compounds of formula 7-4. In the presence of a strong base such as LDA, LTMP and the like, the compounds of formula 7-4 cyclize to form tricyclic compounds of formula 7-5 which can be hydrogenated (e.g., H₂, Pd/C) to form the dehalogenated pyridazines of formula 7-6. The dehalogenated pyridazines of formula 7-6 can be converted to tetracyclic compounds according to, for example, Scheme 4 which then can be converted under acidic conditions (e.g., HCl) to the corresponding pyridizones of formula 7-8.

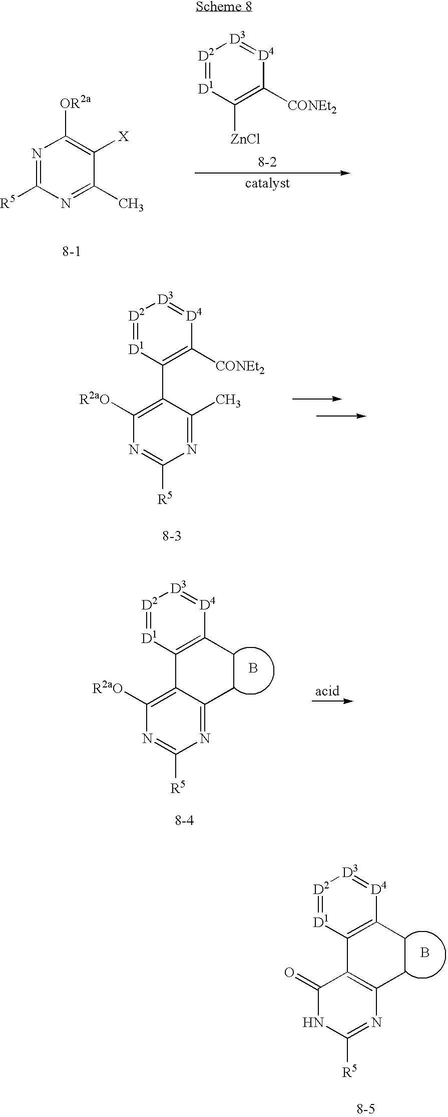

Scheme 8 (X is F, Cl, Br or I) provides an example preparatory route to pyrimidine and pyrimidone compounds of the invention. Compounds having formula 8-1 can be coupled with phenyl derivatives of formula 8-2 in the presence of a suitable catalyst (e.g., Pd) and optionally at elevated temperatures to form coupled compounds of formula 8-3. The coupled compounds of formula 8-3 can be converted to their respective tetracyclic pyrimidines of formula 8-4 according to, for example, Scheme 4 which can then be treated with acid (e.g., acetic acid, hydrochloric acid, etc.) optionally at elevated temperatures to form the corresponding pyrimidones of formula 8-4.

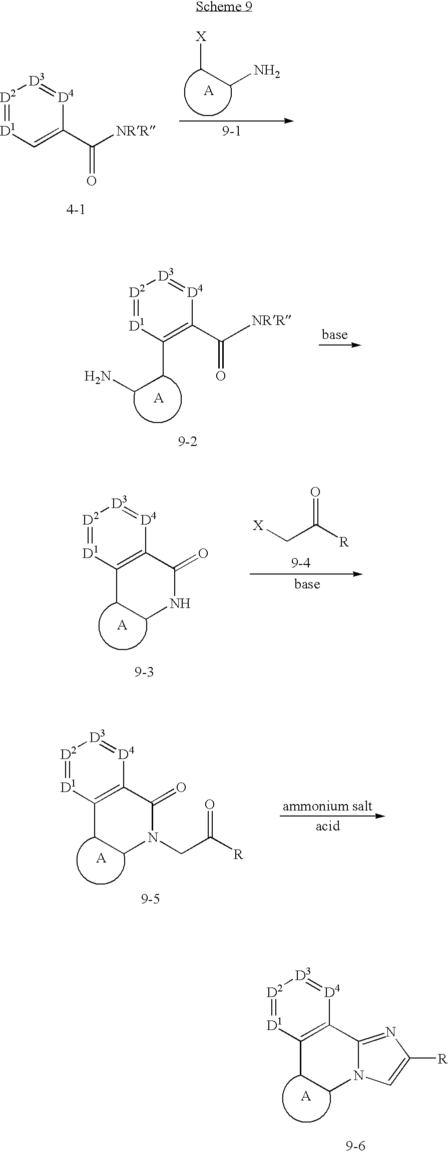

Scheme 9 provides a route for the isoimidazole compounds of Formula 9-6. Compounds having the formula 4-1 can be treated with a strong base (e.g., about one equivalent) such as an alkyllithium reagent (e.g., sec-butyllithium, t-butlylithium, etc.) in the presence of about 1 equivalent of a tetraalkylethylenediamine reagent (e.g., tetramethylethylenediamine (TMEDA)). Halogenated heterocycle 9-1 (where X is halo) can be combined with the resulting mixture in the presence of a metal catalyst (e.g., Pd) and optionally in the presence of heat to provide the benzamide compound of formula 9-2. The benzamide compound 9-2 can be treated with strong base such as lithium diisopropylamide (LDA), LTMP, lithium, sodium or potassium hexamethyldisilazide or the like to yield the tricyclic compound 9-3. Compound 9-3 can be treated with a strong base such as potassium t-butoxide or NaH in an aprotic solvent to give the corresponding metal salt which is alkylated with a haloketone 9-4 (X is halo) to give 9-5. Compound 9-5 can be cyclized to the tetracylic compound 9-6 by heating with an ammonium salt in the presence of an acid.

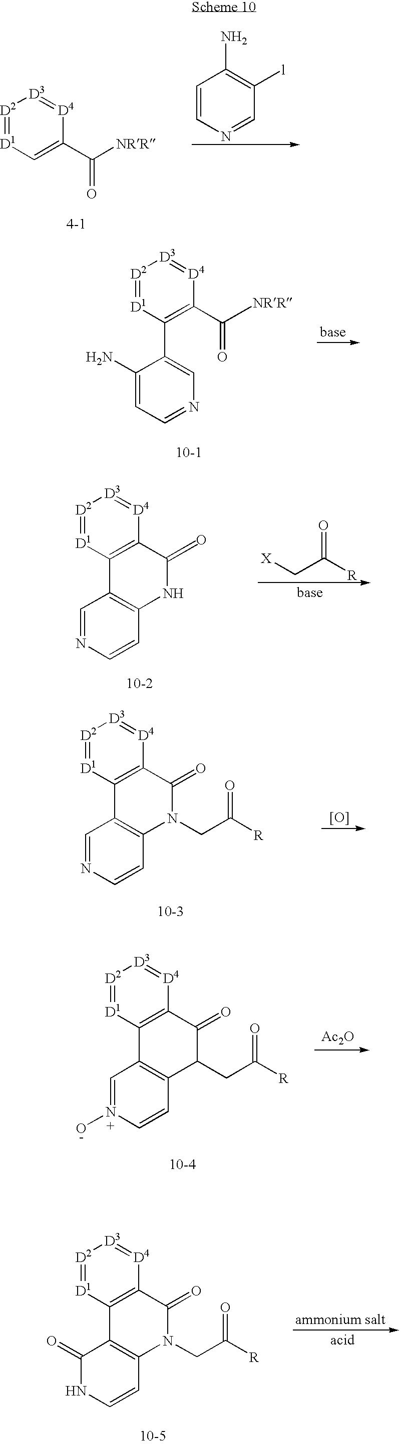

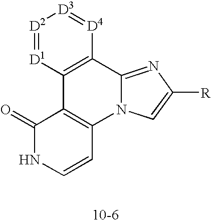

Scheme 10 provides a route for the tetracyclic pyridones 10-6. 4-Amino-3-iodopyridine can be synthesized by the as described in WO 2001/007436. Compounds 10-1, 10-2 and 10-3 can be synthesized by the methods described in Scheme 9. Compounds 10-3 can be oxidized to the corresponding pyridine oxides 10-4 by the action of an oxidizing agent such as m-chloroperbenzoic acid. The pyridine oxide was heated in an acid anhydride and the resulting 2-acyl pyridone was hydrolyzed to 10-5. This compound can be cyclized to the tetracyclic compound 10-6 by heating with an ammonium salt in the presence of an acid.

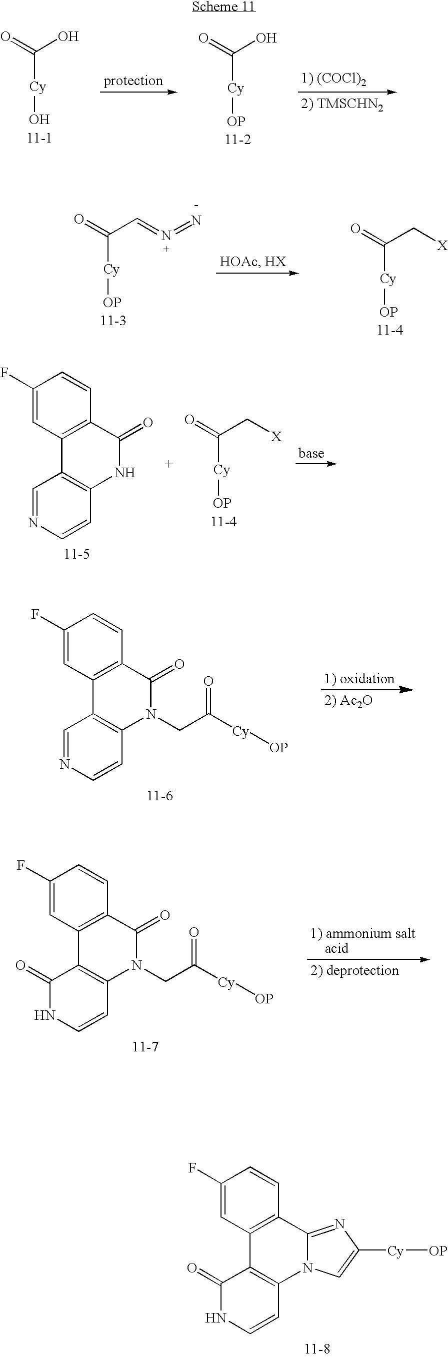

A hydroxyl substituted carboxylic acid 11-1 (Cy is, e.g., cyclolalkyl or heterocycloalkyl) can be protected with an appropriate protecting group to produce 11-2. Acid 11-2 can be subsequently converted to the corresponding acid chloride by treatment with an agent such as oxalyl chloride and to the corresponding diazomethylketone 11-3 with a diazomethane reagent. The diazomethylketone 11-3 can be converted to the halomethylketone 11-4 (X is halo) by treatment with HCl, HBr or HI and 11-4 was reacted with the tricyclic core 11-5 to give 11-6 which was converted to 11-7 under the conditions described in scheme 10. Pyridone 11-7 could be converted to the fused isoimidazole 11-8 under similar procedures described in Scheme 10.

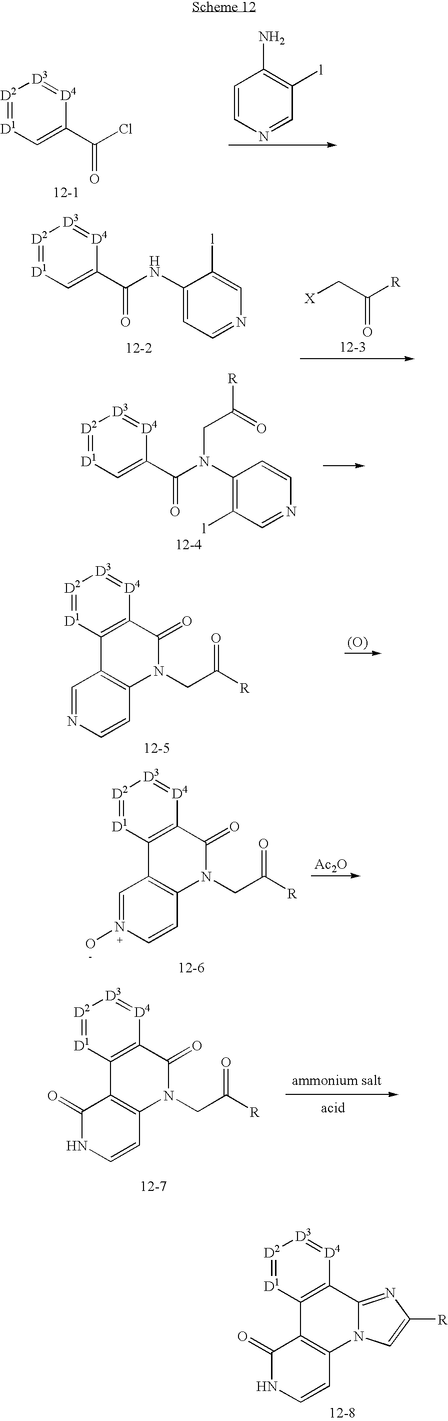

An alternative route to isoimidazoles is provided in Scheme 12. Acid chlorides of formula 12-1 can be reacted with a 4-amino-3-halohalopyridine such as 4-amino-3-iodopyridine to form the amide of formula 12-2. This could be alkylated with a haloketone 12-3 (X is halo) to give the alkyl amide 12-4. Amide 12-4 could then be cyclized by the use of an appropriate catalyst, such as Pd(PPh₃)₄, or Pd(OAc)₂ and P(o-tol)₃ in the presence of a base such as Na₂CO₃ or Ag₂CO₃ to give the tricyclic intermediate 12-5. This could be carried through the sequence described in Scheme 10, an oxidation to the pyridine oxide 12-6, followed by rearrangement and hydrolysis to 12-7, which can be cyclized to give compounds of formula 12-8.

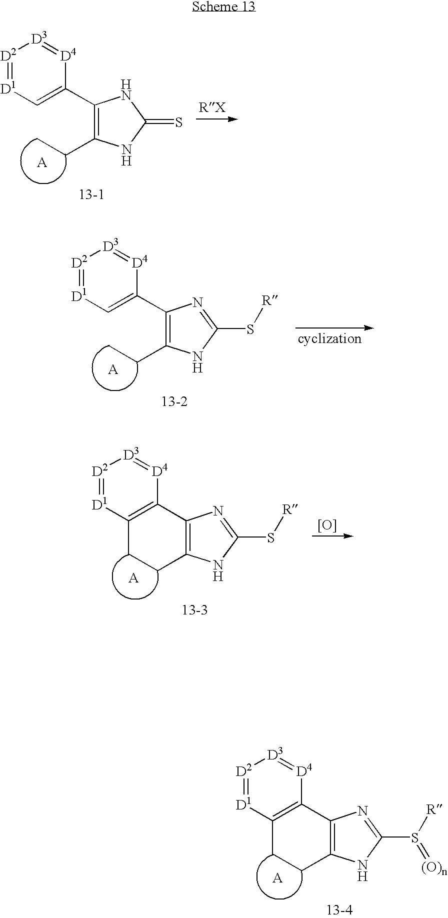

Scheme 13 provides an example preparatory route to thioimidazoles compounds (e.g., 13-3 and 13-4) of the invention. Certain compounds having formula 13-1 can be prepared by methods described in the literature, e.g., Laufer, et al, J Med Chem 2003, 46, 3230-3244. Compounds of formula 13-2 (where R″ is, e.g., alkyl, cycloalkyl, heterocycloalkyl, aryl or heteroaryl) can be prepared by the reaction of the thione formula 13-1 with an appropriate reagent such as R″X where X is a leaving group such as halogen, mesylate, tosylate or other leaving group. Other suitable reagents include epoxides or a unsaturated esters, nitriles or amides, in an appropriate solvent such as DMF, acetonitrile, THF and optionally in the presence of a base like sodium hydride, potassium carbonate, bicarbonate or lithium alkyl, at a temperature compatible for the reaction. Compounds of formula 13-3 can be prepared from 13-2 by known methods for photocyclization. Compounds of formula 13-4 can be prepared from compounds of formula 13-3 by reaction with an oxidizing reagent such as m-chloroperbenzoic acid or hydrogen peroxide in an appropriate solvent and at an appropriate temperature.

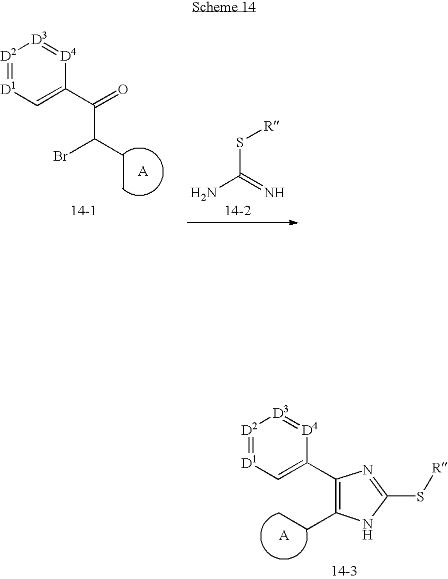

Compounds of formula 14-1 can be prepared as previously described herein. Thioimidazole compounds 14-3 can be prepared from 14-1 by reaction with an appropriately substituted isothiourea compound of formula 14-2 (R″ is, e,g., alkyl, cycloalkyl, heterocycloalkyl, aryl, heteroaryl, etc.).

The compounds of formula 14-3 can be cyclized according to routine methods such as any of those described herein to form thioimidazole compounds of the invention.

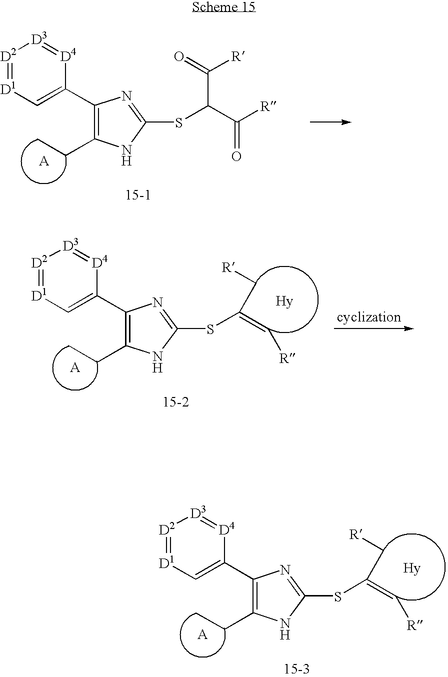

The compounds of formula 15-1 can be prepared by reaction of compounds of formula 13-1 (see Scheme 13) with an appropriately substituted 2-bromo or 2-chloro dicarbonyl reagent like malonaldehyde, pentane-2,4-dione, methyl 3-oxopropanoate. The compounds of formula 15-2 (Hy is a heterocyclic ringn system) can be prepared by reaction of the dicabonyl compound of formula 15-1 with a reagent such as hydrazine, alkyl hydrazines, hydroxylamines, formamidines, alkyl amidines, urea, O-alkyl ureas or guainidines, where the reaction can be carried out in a solvent such as DMF, DMSO, or acetic acid at an appropriate temperature. Transformations such as these are well known in the literature for preparing of a variety of 5 and 6 member heterocyclic rings.

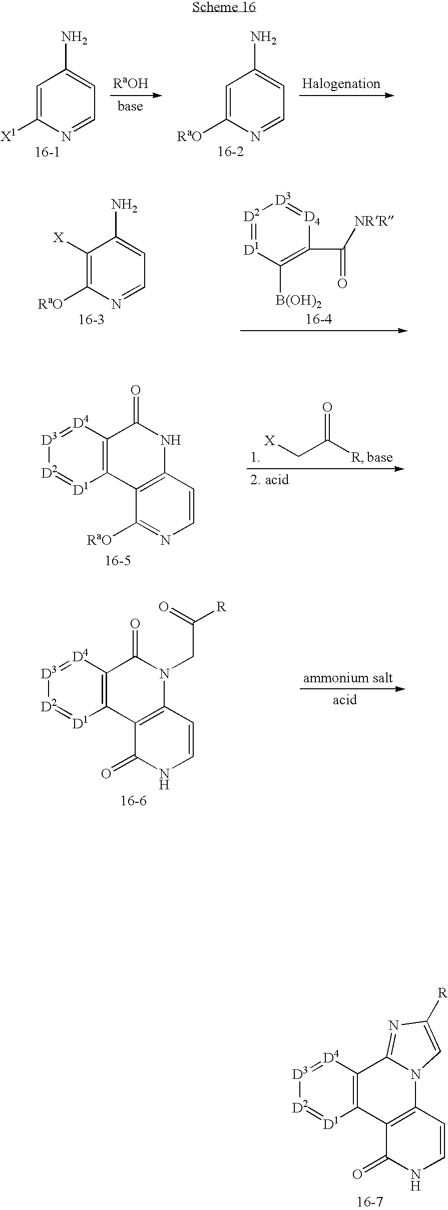

Scheme 16 provides a synthetic route for compounds of Formula 16-7. A compound of Formula 16-1 wherein X¹ is a leaving group such as chloride can be treated with an alcohol R^{a}OH under basic condition to afford a compound of Formula 16-2. The compound of Formula 16-2 can be halogenated with an appropriate reagent such as NIS to yield a halogenated intermediate of Formula 16-3. The halogenated intermediate of Formula 16-3 can be coupled to a phenyl boronic acid reagents of Formula 16-4 under, for example, Suzuki type reaction conditions to form a coupled and cyclized compound of Formula 16-5. The amide moiety of the compound of Formula 16-5 can be alkylated with a haloketone under a basic condition, followed by a subsequent acid condition to covert the alkoxypridine moiety to pyridinone, to afford a compound of Formula 16-6. The compound of Formula 16-6 can be treated with an ammonium salt in the presence of an acid to afford a tetracyclic compound of Formula 16-7.

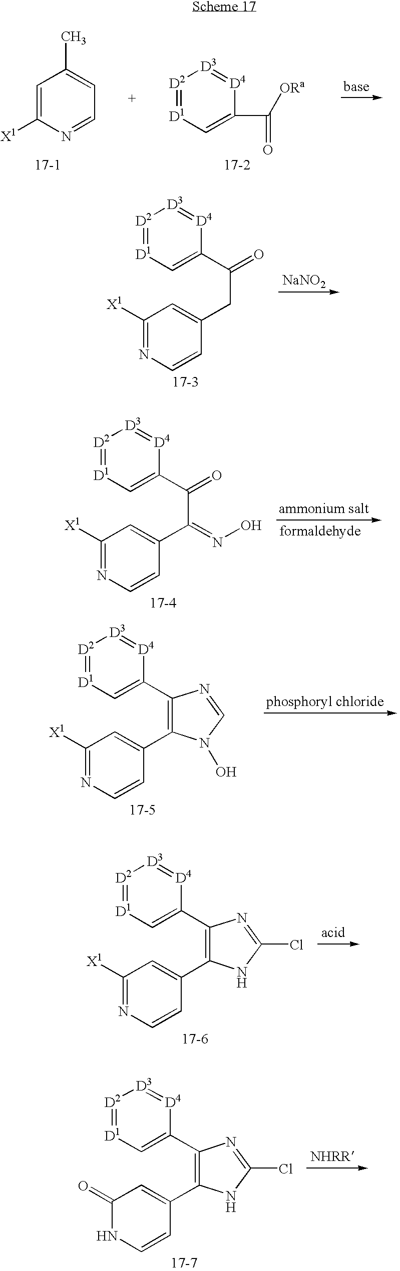

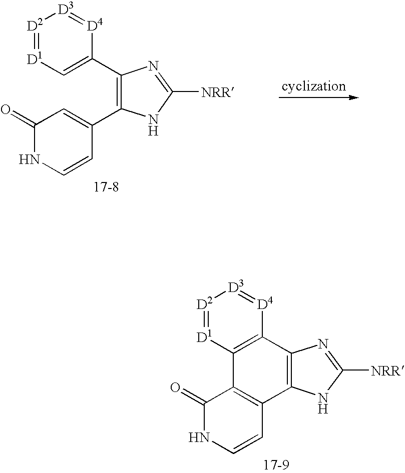

Scheme 17 provides a synthetic route for compounds of Formula 17-9. A compound of Formula 17-1 wherein X¹ is a leaving group such as fluoro can be coupled to a compound of Formula 17-2 under basic condition to afford a compound of Formula 17-3. The compound of Formula 17-3 can be treated with sodium nitrite under acidic condition to afford a keto-oxime compound of Formula 17-4. The compound of Formula 17-4 can be treated with an ammonium salt in the presence of formaldehyde to afford a hydroxyl-imidazole compound of Formula 17-5. The compound of Formula 17-5 can be treated with phosphoryl chloride to afford a 2-chloro-imidazole compound of Formula 17-6. The compound of Formula 17-6 can be treated with an acid to undergo hydrolysis and rearrangement to afford a pyridinone compound of Formula 17-7. Coupling of the compound of Formula 17-7 with an amine compound (NHRR′, can be a cyclic amine) can afford a compound of Formula 17-8. The compound of Formula 17-8 can be irradiated to afford a tetracyclic compound of Formula 17-9.

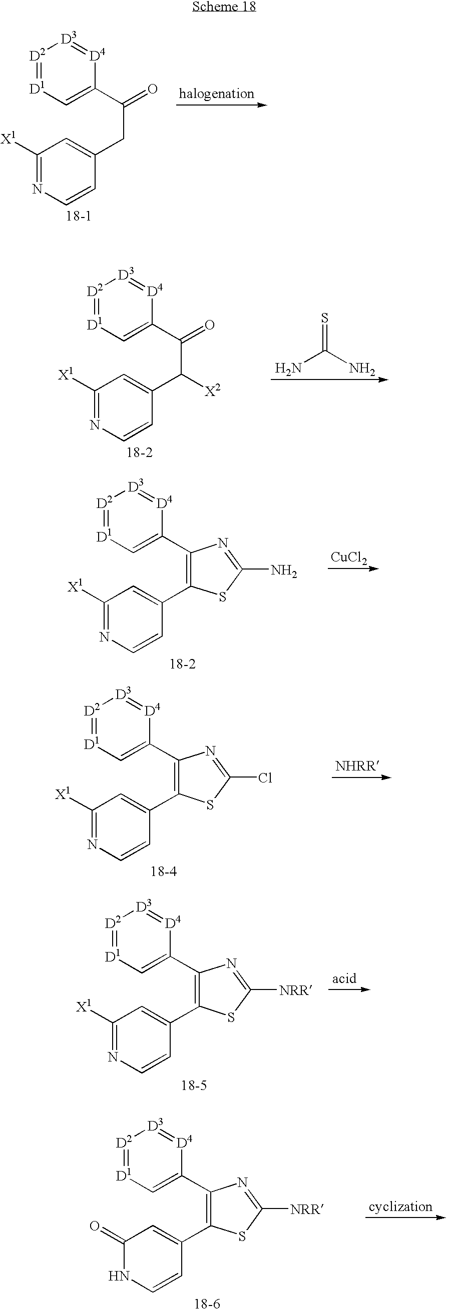

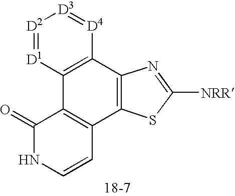

Scheme 18 provides a synthetic route for compounds of Formula 18-7. A compound of Formula 18-1 wherein X¹ is a leaving group such as fluoro can be halogenated by a reagent such as bromine to afford an α-halo keto compound of Formula 18-2. The α-halo keto compound of Formula 18-2 can be treated with thiourea to afford an amino-thiazole compound of Formula 18-3. The amino-thiazole compound of Formula 18-3 can be treated with treated with copper (II) chloride to afford a chloro-thiazole compound of Formula 18-4. The chloro-thiazole compound of Formula 18-4 can be coupled with an amine compound (NHRR′, can be, e.g., a cyclic amine) to afford a compound of Formula 18-5. The compound of Formula 18-5 can be subjected to an acidic condition to afford a compound of Formula 18-6, converting the halo-pyridine moiety to pyridinone. The compound of Formula 18-6 can be irradiated to afford a tetracyclic compound of Formula 18-7.

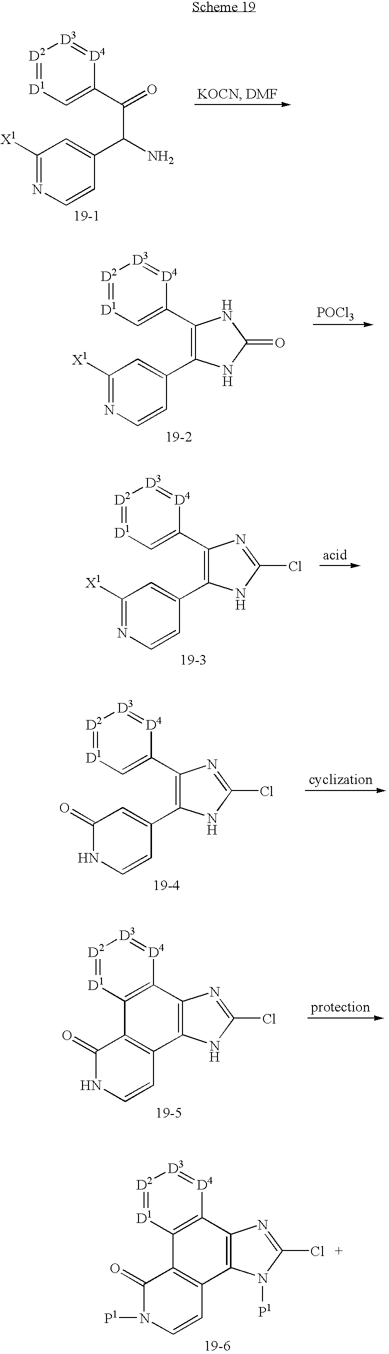

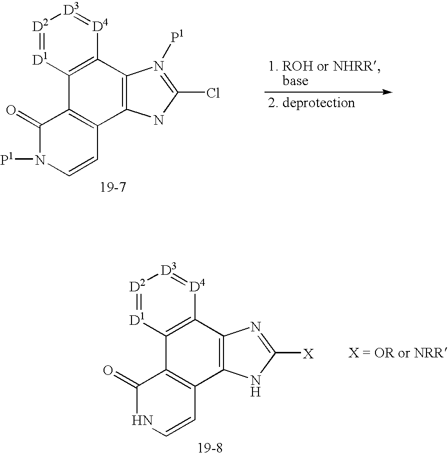

Scheme 19 provides a synthetic route for compounds of Formula 19-8. A compound of Formula 19-1 wherein X¹ is a leaving group such as fluoro can be treated with potassium cyanate at an elevated temperature to afford a dihydro-imidazol-one compound of Formula 19-2. The dihydro-imidazol-one compound of Formula 19-2 can be treated with phosphoryl chloride to afford a chloro-imidazole compound of Formula 19-3. The compound of Formula 19-3 can be subjected to acid conditions to afford a compound of Formula 19-4, converting the halo-pyridine moiety to pyridinone.

The compound of Formula 19-4 can be irradiated to afford a tetracyclic compound of Formula 19-5. The amide groups in the compound of Formula 19-5 can be protected by a suitable protecting group such as SEM to afford a mixture of compounds of Formula 19-6 and 19-7. The mixture of compounds of Formula 19-6 and 19-7 can be treated with an alcohol (ROH) or an amine (NHRR′) under basic conditions, followed by deprotection of the amide groups to afford a compound of Formula 19-8.

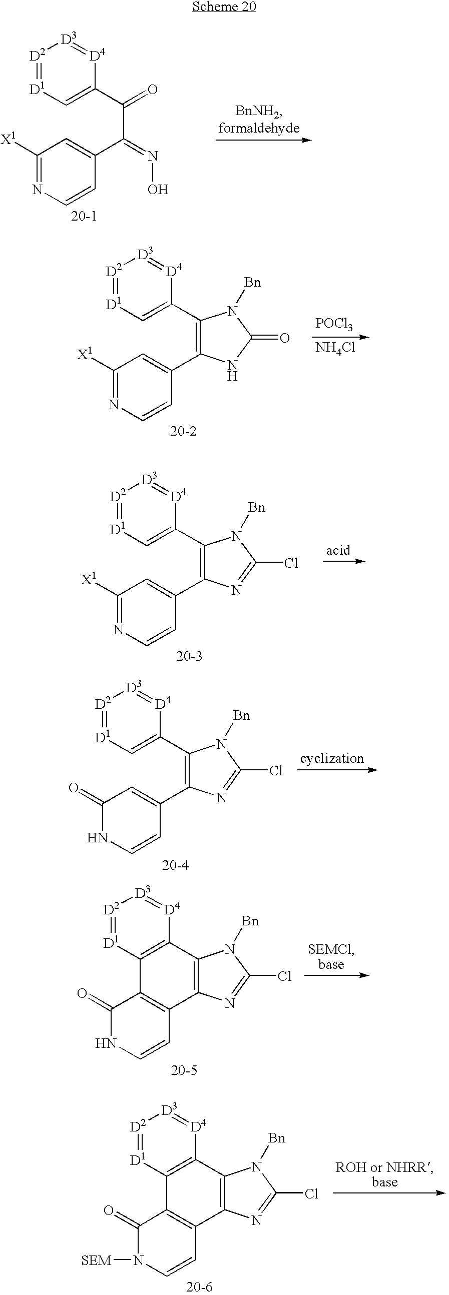

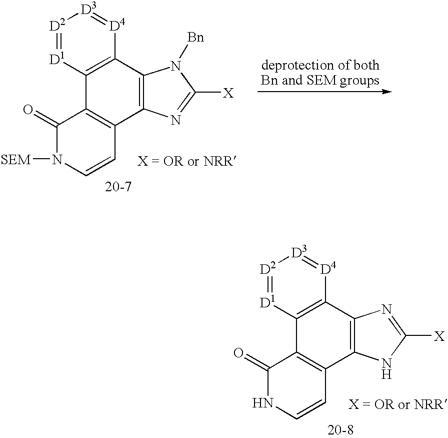

Scheme 20 provides a synthetic route for compounds of Formula 20-8. A compound of Formula 20-1 wherein X¹ is a leaving group such as fluoro can be treated with benzylamine and formaldehyde at an elevated temperature to afford an N-benzyl-dihydro-imidazol-one compound of Formula 20-2. The N-benzyl-dihydro-imidazol-one compound of Formula 20-2 can be treated with phosphoryl chloride and ammonium chloride to afford a chloro-imidazole compound of Formula 20-3. The compound of Formula 20-3 can be subjected to an acid condition to afford a compound of Formula 20-4, converting the halo-pyridine moiety to pyridinone. The compound of Formula 20-4 can be irradiated to afford a tetracyclic compound of Formula 20-5. The unprotected amide group in the compound of Formula 20-5 can be protected by a suitable protecting group such as SEM to afford a compound of Formula 20-6. The compound of Formula 20-6 can be treated with an alcohol (ROH) or an amine (NHRR′) under basic conditions to afford a compound of Formula 20-7. It will be understood by an ordinary person in the art that while the amide groups are protected, the compound of Formula 20-7 can be subjected to various conditions to allow modification on R or R′ groups if so desired. The compound of Formula 20-7 can be subjected to suitable conditions to remove both Bn and SEM groups to afford a compound of Formula 20-7.

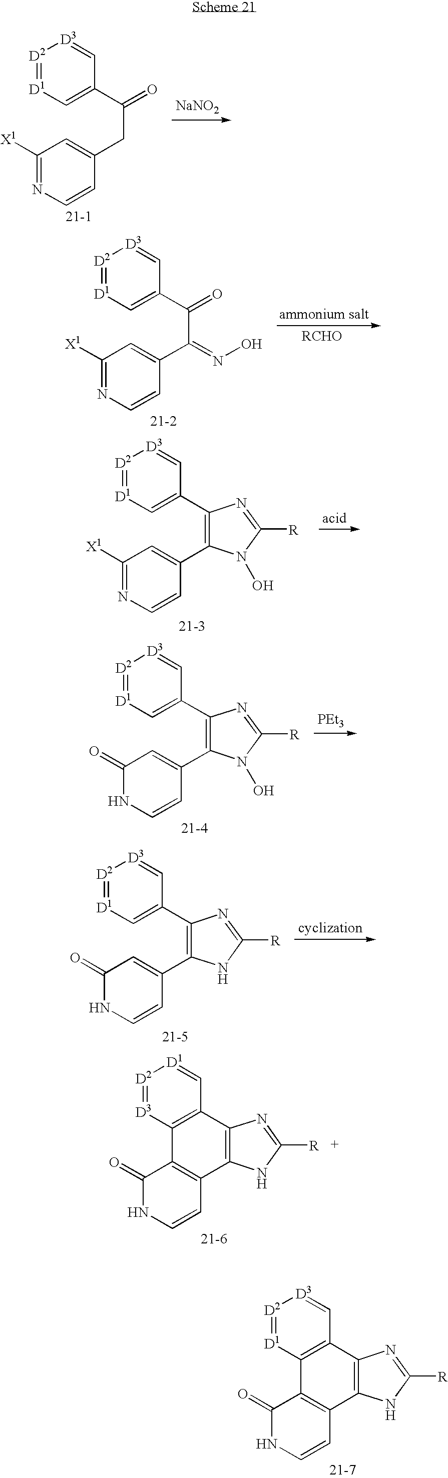

Scheme 21 provides a synthetic route for compounds of Formula 21-6 and 21-7. A compound of Formula 21-1 wherein X¹ is a leaving group such as fluoro can be treated with sodium nitrite under acidic condition to afford a keto-oxime compound of Formula 21-2. The compound of Formula 21-2 can be treated with an aldehyde (RCHO) in the presence of an ammonium salt to afford a hydroxyl-imidazole compound of Formula 21-3. The compound of Formula 21-3 can be subjected to an acid condition to afford a compound of Formula 21-4, converting the halo-pyridine moiety to pyridinone. The compound of Formula 21-4 can be treated with a trialkyl phosphine to remove the hydroxyl group resulting in a imidazole compound of Formula 21-5. The compound of Formula 21-5 can be irradiated to afford a mixture of tetracyclic compounds of Formula 21-6 and 21-7.

Methods

Compounds of the invention can modulate activity of one or more Janus kinases (JAKs). The term “modulate” is meant to refer to an ability to increase or decrease the activity of one or more members of the JAK family of kinases. Accordingly, compounds of the invention can be used in methods of modulating a JAK by contacting the enzyme/kinase with any one or more of the compounds or compositions described herein. In some embodiments, compounds of the present invention can act as inhibitors of one or more JAKs. In some embodiments, compounds of the present invention can act to stimulate the activity of one or more JAKs. In further embodiments, the compounds of the invention can be used to modulate activity of a JAK in an individual in need of modulation of the receptor by administering a modulating amount of a compound of Formula I.

JAKs to which the present compounds bind and/or modulate include any member of the JAK family. In some embodiments, the JAK is JAK1, JAK2, JAK3 or TYK2. In some embodiments, the JAK is JAK1 or JAK2. JAKs further include both wild-type sequences and those natural or unnatural mutations that may arise by genetic translocation of some or all of the gene encoding for a JAK, or by mutation in the JAK kinase domain, or any mutation within the gene encoding for JAK that results in dysregulated kinase activity. In some embodiments, the JAK is a variant of JAK1, JAK2, JAK3 or TYK2, such as a natural variant. In some embodiments, the variant is JAK2V617F, believed to be a constitutively active tyrosine kinase (Levine, et al. Cancer Cell., 2005, 7, 387).

The compounds of the invention can be selective. By “selective” is meant that the compound binds to or inhibits a JAK with greater affinity or potency, respectively, compared to at least one other JAK. In some embodiments, the compounds of the invention are selective inhibitors of JAK1 or JAK2 over JAK3 and/or TYK2. In some embodiments, the compounds of the invention are selective inhibitors of JAK2 (e.g., over JAK2, JAK3 and TYK2). Without wishing to be bound by theory, because inhibitors of JAK3 lead to immunosuppressive effects, a compound which is selective for JAK2 over JAK3 and which is useful in the treatment of cancer (such as multiple myeloma, for example) may offer the additional advantage of having fewer immunosuppressive side effects. Selectivity can be at least about 5-fold, 10-fold, at least about 20-fold, at least about 50-fold, at least about 100-fold, at least about 200-fold, at least about 500-fold or at least about 1000-fold. Selectivity can be measured by methods routine in the art. Selectivity can be tested at the Km ATP concentration of each enzyme. In some embodiments, selectivity of compounds of the invention for JAK2 over JAK3 may be determined by the cellular ATP concentration.

Another aspect of the present invention pertains to methods of treating a JAK-associated disease or disorder in an individual (e.g., patient) by administering to the individual in need of such treatment a therapeutically effective amount or dose of a compound of the present invention or a pharmaceutical composition thereof. A JAK-associated disease can include any disease, disorder or condition that is directly or indirectly linked to expression or activity of the JAK, including overexpression and/or abnormal activity levels. A JAK-associated disease can also include any disease, disorder or condition that can be prevented, ameliorated, or cured by modulating JAK activity.

Examples of JAK-associated diseases include diseases involving the immune system including, for example, organ transplant rejection (e.g., allograft rejection and graft versus host disease). Further examples of JAK-associated diseases include autoimmune diseases such as multiple sclerosis, rheumatoid arthritis, juvenile arthritis, type I diabetes, lupus, psoriasis, inflammatory bowel disease, ulcerative colitis, Crohn's disease, or autoimmune thyroid disorders. Further examples of JAK-associated diseases include allergic conditions such as asthma, food allergies, atopic dermatitis and rhinitis. Further examples of JAK-assoicated diseases include viral diseases such as Epstein Barr Virus (EBV), Hepatitis B, Hepatitis C, HIV, HTLV 1, Varicella-Zoster Virus (VZV) and Human Papilloma Virus (HPV). In further embodiments, the JAK-associated disease is cancer such as, for example, prostate, renal, hepatocellular, pancreatic, gastric, breast, lung, cancers of the head and neck, glioblastoma, leukemia, lymphoma or multiple myeloma.

Examples of further JAK-associated diseases include IL-6 mediated diseases. Examples of IL-6 mediated diseases include cancers (e.g., multiple myeloma, Castleman's disease, and Kaposi's sarcoma) as well as rheumatoid arthritis.

Examples of further JAK-associated diseases include myeloproliferative disorders including polycythemia vera (PV), essential thrombocythemia (ET), myeloidcmetaplasia with meylofibrosis (MMM), and the like.

The present invention further provides methods of treating psoriasis or other skin disorders by administration of a topical formulation containing a compound of the invention.

The present invention further provides a method of treating dermatological side effects of other pharmaceuticals by administration of a compound of the invention. For example, numerous pharmaceutical agents result in unwanted allergic reactions which can manifest as acneiform rash or related dermatitis. Example pharmaceutical agents that have such undesirable side effects include anti-cancer drugs such as gefitinib, cetuximab, erlotinib, and the like. The compounds of the invention can be administered systemically or topically (e.g., localized to the vicinity of the dermatitis) in combination with (e.g., simultaneously or sequentially) the pharmaceutical agent having the undesirable dermatological side effect. In some embodiments, one or more compounds of the invention can be administered topically together with one or more other pharmaceuticals, where the other pharmaceuticals when topically applied in the absence of a compound of the invention cause contact dermatitis, allergic contact sensitization, or similar skin disorder. Accordingly, compositions of the invention include topical formulations containing at least one compound of the invention and a further pharmaceutical agent which can cause dermatitis, skin disorders, or related side effects.

As used herein, the term “contacting” refers to the bringing together of indicated moieties in an in vitro system or an in vivo system. For example, “contacting” a JAK with a compound of the invention includes the administration of a compound of the present invention to an individual or patient, such as a human, having a JAK, as well as, for example, introducing a compound of the invention into a sample containing a cellular or purified preparation containing the JAK.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:
(1) preventing the disease; for example, preventing a disease, condition or disorder in an individual who may be predisposed to the disease, condition or disorder but does not yet experience or display the pathology or symptomatology of the disease (non-limiting examples are preventing graft versus host disease and/or allograft rejection after transplantation, and preventing allergic reactions such as atopic dermatitis or rhinitis);
(2) inhibiting the disease; for example, inhibiting a disease, condition or disorder in an individual who is experiencing or displaying the pathology or symptomatology of the disease, condition or disorder (i.e., arresting further development of the pathology and/or symptomatology) such as inhibiting the autoimmune response in rheumatoid arthritis, lupus or psoriasis, inhibiting tumor growth or stabilizing viral load in the case of a viral infection; and
(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the autoimmune response in rheumatoid arthritis, lupus or psoriasis, shrinking a tumor associated with cancer or lowering viral load in the case of a viral infection.

One or more additional pharmaceutical agents such as, for example, chemotherapeutics, anti-inflammatory agents, and/or immunosuppressants can be used in combination with the compounds of the present invention for treatment of JAK-associated diseases, disorders or conditions. For example, a JAK inhibitor used in combination with a chemotherapeutic in the treatment of multiple myeloma may improve the treatment response as compared to the response to the chemotherapeutic agent alone, without clinically acceptable exacerbation of its toxic effects. Examples of additional pharmaceutical agents used in the treatment of multiple myeloma, for example, can include, without limitation, melphalan, melphalan plus prednisone [MP], doxorubicin, dexamethasone, and velcade. Additive or synergistic effects are desirable outcomes of combining a JAK inhibitor of the present invention with an additional agent. Furthermore, resistance of multiple myeloma cells to agents such as dexamethasome may be reversible upon treatment with a JAK inhibitor of the present invention. The agents can be combined with the present compounds in a single or continuous dosage form, or the agents can be administered simultaneously or sequentially as separate dosage forms.

Pharmaceutical Formulations and Dosage Forms

When employed as pharmaceuticals, the compounds of Formula I can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal, intramuscular or injection or infusion; or intracranial, e.g., intrathecal or intraventricular, administration. Parenteral administration can be in the form of a single or repeated bolus dosing, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of Formula I above in combination with one or more pharmaceutically acceptable carriers (excipients). In some embodiments, the composition is suitable for topical administration. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compounds or compositions of the invention contain from about 5 to about 50 mg of the active ingredient. One having ordinary skill in the art will appreciate that this embodies compounds or compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compounds or compositions of the invention contain from about 50 to about 500 mg of the active ingredient. One having ordinary skill in the art will appreciate that this embodies compounds or compositions containing about 50 to about 100, about 100 to about 150, about 150 to about 200, about 200 to about 250, about 250 to about 300, about 350 to about 400, or about 450 to about 500 mg of the active ingredient.

In some embodiments, the compounds or compositions of the invention contain from about 500 to about 1000 mg of the active ingredient. One having ordinary skill in the art will appreciate that this embodies compounds or compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of a compound of the invention. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgement of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

Labeled Compounds and Assay Methods

Another aspect of the present invention relates to radio-labeled compounds of Formula I that would be useful not only in radio-imaging but also in assays, both in vitro and in vivo, for localizing and quantitating a JAK in tissue samples, including human, and for identifying JAK ligands by inhibition binding of a radio-labeled compound. Accordingly, the present invention includes JAK assays that contain such radio-labeled compounds.

The present invention further includes isotopically-labeled compounds of Formula I. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro metalloprotease labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled” or “labeled compound” is a compound that has incorporated at least one radionuclide. In some embodiments the radionuclide is selected from the group consisting of ³H, ¹⁴C, ¹²⁵I, ³⁵S and ⁸²Br.

Synthetic methods for incorporating radio-isotopes into organic compounds are applicable to compounds of the invention and are well known in the art.

A radio-labeled compound of the invention can be used in a screening assay to identify/evaluate compounds. In general terms, a newly synthesized or identified compound (i.e., test compound) can be evaluated for its ability to reduce binding of the radio-labeled compound of the invention to a metalloprotease. Accordingly, the ability of a test compound to compete with the radio-labeled compound for binding to the metalloprotease directly correlates to its binding affinity.

Kits

The present invention also includes pharmaceutical kits useful, for example, in the treatment or prevention of JAK-associated diseases or disorders, such as cancer, which include one or more containers containing a pharmaceutical composition comprising a therapeutically effective amount of a compound of Formula I. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

The following compounds are examples of JAK inhibitors according to the present invention.

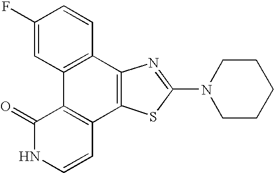

### Example 1

### 9-Fluoro-2-piperidin-1-ylbenzo[h][1,3]thiazolo[5,4-f]isoquinolin-7(6H)-one

Step A

### 2-Bromo-1-(4-fluorophenyl)-2-(2-fluoropyridin-4-yl)ethanone

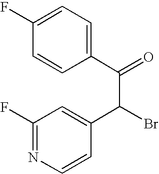

1-(4-Fluorophenyl)-2-(2-fluoropyridin-4-yl)ethanone (6.80 g, 29.2 mmol, Bioorganic & Medicinal Chemistry Letters, 2002, 12, 1219-1223), was dissolved in DMF (60 mL). N-bromosuccinimide (5.19 g, 0.0292 mol) was added and the mixture was stirred at room temperature. After 5 hours, the mixture was poured into half-saturated aqueous NaHCO₃ (400 mL), and extracted with tert-butylmethyl ether (400 mL). The organic phase was washed with half-saturated aqueous NaHCO₃ (2×250 mL), then with brine. The extracts were dried over Na₂SO₄, filtered and concentrated to give the title compound as a light brown oil (9.40 g; HPLC 95 area % pure, 3% dibromo; 97% yield). LC/MS: 312.0, 313.0, (M+H)⁺. ¹H NMR (CDCl₃) δ 8.26 (d, J=5.2, 1H), 8.06 (m, 2H), 7.33 (dt, J=5.2 and 1.5, 1H), 7.20 (m, 2H), 7.13 (s, 1H), 6.16 (s, 1H, BrCH). ¹⁹F NMR (CDCl₃) δ −67.0 (s, F-pyridyl), −102.4 (m, F-phenyl). 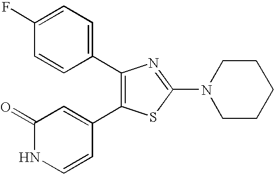
Step B

### 4-[4-(4-Fluorophenyl)-2-piperidin-1-yl-1,3-thiazol-5-yl]pyridine-2(1H)-one

A solution of 2-bromo-1-(4-fluorophenyl)-2-(2-fluoropyridin-4-yl)ethanone (1.00 g, 3.20 mmol) and piperidine-1-carbothioamide (462 mg, 3.20 mmol) in acetic acid (20 mL) was stirred at room temperature. After 1.5 hours, LC/MS showed complete conversion to the desired thiazole (LC/MS: 358.0, (M+H)⁺). To the mixture was added 1.0 mL of water and the resulting mixture was heated to 90° C. After 18 hours, LC/MS showed complete hydrolysis to the desired compound. The mixture was cooled to room temperature, poured into 300 mL 10% aqueous KHCO₃ and extracted with dichloromethane (3×75 mL). The crude product was purified by flash chromatography on silica gel eluting with a gradient of dichloromethane to 7% isopropanol/dichloromethane. Pure fractions were combined and concentrated to give the title compound as a yellow solid (0.70 g, 61%). LC/MS: 356.0 (M+H)⁺. ¹H NMR (DMSO-d₆) δ 11.43 (bs, 1H, NH), 7.49 (dd, J=8.8 and 5.8, 2H), 7.22 (d, J=6.9, 1H), 7.22 (t, J=8.8, 2H), 6.09 (d, J=1.8, 1H), 5.79 (dd, J=6.9 and 1.8, 1H), 3.48 (bs, 4H, NCH₂), 1.61 (bs, 6H, CH₂). ¹⁹F NMR (DMSO-d₆) δ −113.55 (m). 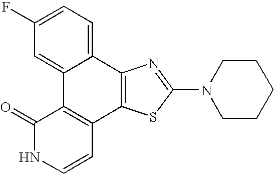
Step C

### 9-Fluoro-2-piperidin-1-ylbenzo[h][1,3]thiazolo[5,4-f]isoquinolin-7(6H)-one

A solution of 4-[4-(4-fluorophenyl)-2-piperidin-1-yl-1,3-thiazol-5-yl]pyridin-2(1H)-one (0.70 g, 1.97 mmol) in THF (1.5 L) in an open crystallizing dish was stirred and exposed to UV light at a distance of 5 cm (Mineralight UVL-56, 365 nm). After 20 hours, LC/MS showed greater than 70% conversion to desired product along with unreacted starting material. Silica gel (8 g) was added and the mixture was concentrated on the rotovap to a dry powder and then loaded onto a silica gel column. The product was eluted with a gradient of dichloromethane to 5% isopropanoudichloromethane. Fractions containing pure product were combined and concentrated, leaving a mixture of solid product suspended in about 35 mL isopropanol. The precipitated product was filtered and dried to give the title compound as a pale purple powder (0.26 g, 37%). LC/MS: 354.0 (M+H)⁺. ¹H NMR (DMSO-d₆) δ 11.66 (bs, 1H, NH), 9.93 (dd, J=14.1 and 2.8, 1H), 8.58 (dd, J=8.9 and 6.8, 1H); 7.55 (m, 2H); 6.49 (d, J=6.8, 1H); 3.75 (bs, 4H, NCH₂); 1.68 (bs, 6H, CH₂).

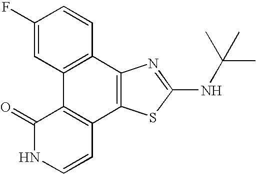

### Example 2

### 2-(tert-Butylamino)-9-fluorobenzo[h][1,3]thiazolo[5,4-f]isoquinolin-7(6H)-one

The title compound was prepared following the procedures described for Example 1 using N-(tert-butyl)thiourea. LC/MS: 342.0, (M+H)⁺. ¹H NMR (DMSO-d₆) 611.65 (m, 1H, pyridone NH), 9.96 (dd, J=14.0 and 2.8, 1H), 8.59 (dd, J=9.0 and 6.8, 1H), 8.43 (s, 1H, NH), 7.56 (m, 1H), 7.50 (t, J=6.7, 1H), 6.49 (d, J=6.7, 1H), 1.52 (s, 9H). ¹⁹F NMR (DMSO-d₆) δ 112.6 (m).

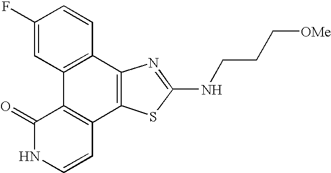

### Example 3

### 9-Fluoro-2-[(3-methoxypropyl)amino]benzo[h][1,3]thiazolo[5,4-f]isoquinolin-7(6H)-one

The title compound was prepared following the procedures described for Example 1 using N-(3-methoxypropyl)thiourea. LC/MS: 358.0, (M+H)⁺. ¹H NMR (DMSO-d₆) δ 11.62 (d, J=5.7, 1H, pyridone NH), 9.95 (dd, J=14.0 and 2.7, 1H), 8.71 (t, J=5.2, 1H, NH), 8.59 (dd, J=9.0 and 6.7, 1H), 7.53 (m, 1H), 7.50 (t, J=6.5, 1H), 6.50 (dd, J=6.8 and 1.0, 1H), 3.54 (q, J=5.9, 2H, NCH₂), 3.47 (t, J=6.0, 2H, OCH₂), 3.28 (s, 3H, OCH₃), 1.92 (m, 2H, CH₂). ¹⁹F NMR (DMSO-d₆) δ 112.5 (m).

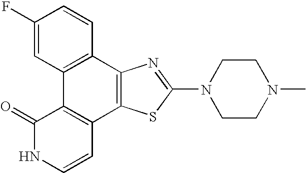

### Example 4

### 9-Fluoro-2-(4-methylpiperazin-1-yl)benzo[h][1,3]thiazolo[5,4-f]isoquinolin-7(6H)-one

The title compound was prepared following the procedures described for Example 1 using 4-methylpiperazine-1-carbothioamide. LC/MS: 369.0, (M+H)⁺. ¹H NMR (DMSO-d₆) δ 11.71 (bs, 1H, pyridone NH), 9.93 (dd, J=13.9 and 2.8, 1H), 8.60 (dd, J=9.0 and 6.6, 1H), 7.54 (m, 2H), 6.52 (dd, J=6.9 and 1.3, 1H), 3.30 (bs, 11H, under water peak).

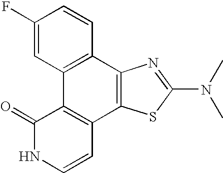

### Example 5

### 2-(Dimethylamino)-9-fluorobenzo[h][1,3]thiazolo[5,4-f]isoquinolin-7(6H)-one

The title compound was prepared following the procedures described for Example 1 using N,N-dimethylthiourea. LC/MS: 314.0, (M+H)⁺. ¹H NMR (DMSO-d₆) δ 11.65 (bs, 1H, pyridone NH), 9.93 (dd, J=14.0 and 2.7, 1H), 8.61 (dd, J=9.0 and 6.8, 1H), 7.52 (m, 2H), 6.53 (d, J=6.8, 1H), 3.28 (s, 6H, NCH₃).

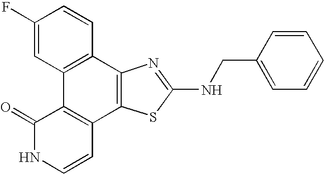

### Example 6

### 2-(Benzylamino)-9-fluorobenzo[h][1,3]thiazolo[5,4-f]isoquinolin-7(6H)-one

The title compound was prepared following the procedures described for Example 1 using N-benzylthiourea. LC/MS: 376.1, (M+H)⁺. ¹H NMR (DMSO-d₆) δ 11.66 (d, J=5.8, 1H, pyridone NH), 9.94 (dd, J=14.1 and 2.9, 1H), 9.17 (bs, 1H), 8.59 (dd, J=8.8 and 6.8, 1H), 7.55 (m, 1H), 7.48 (m, 3H), 7.38 (m, 2H), 7.29 (m, 1H), 6.49 (dd, J=6.8 and 1.5, 1H), 4.73 (m, 2H).

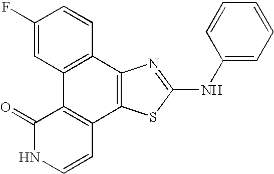

### Example 7

### 2-Anilino-9-fluorobenzo[h][1,3]thiazolo[5,4-f]isoquinolin-7(6H)-one

The title compound was prepared following the procedures described for Example 1 using N-phenylthiourea. LC/MS: 362.0, (M+H)⁺. ¹H NMR (DMSO-d₆) δ 1.79 (d, J=5.1, 1H, pyridone NH), 11.03 (bs, 1H), 9.99 (dd, J=13.8 and 2.7, 111), 8.72 (dd, J=9.0 and 6.6, 1H), 7.92 (d, J=7.6, 2H), 7.64 (m, 1H), 7.56 (t, J=6.4, 1H), 7.46 (m, 2H), 7.12 (t, J=7.3, 1H), 6.63 (d, J=6.8, 1H).

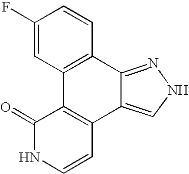

### Example 8

### 9-Fluoro-2,6-dihydro-7H-benzo[h]pyrazolo[4,3-f]isoquinolin-7-one

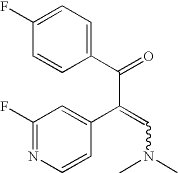 Step A

### 3-(Dimethylamino)-1-(4-fluorophenyl)-2-(2-fluoropyridin-4-yl)prop-2-en-1-one

To a solution of 1-(4-fluorophenyl)-2-(2-fluoropyridin-4-yl)ethanone (4.00 g, 17.2 mmoles) in tetrahydrofuran (50 mL) was added N,N-dimethylformamide dimethyl acetal (12.5 mL, 94.1 mmol) and the solution stirred at room temperature. After 16 hours, the mixture was concentrated on the rotovap, azeotroped once with toluene, and the residue dried under vacuum to afford the crude product as an orange oil (5.00 g) which was used directly without further purification. LC/MS: 289.0 (M+H)⁺. 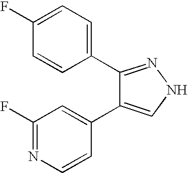
Step B

### 2-Fluoro-4-[3-(4-fluorophenyl)-1H-pyrazol-4-yl]pyridine

The crude product from Step A, 3-(dimethylamino)-1-(4-fluorophenyl)-2-(2-fluoropyridin-4-yl)prop-2-en-1-one (5.00 g, 17.2 mmol) was dissolved in ethanol (60 mL). Hydrazine (1.08 mL, 34.3 mmol) was added and the solution stirred at room temperature overnight. TLC (60% EtOAc/hexane) indicated complete conversion. The solution was diluted with water and then extracted twice with ethyl acetate. The organic extracts were washed with brine, dried over MgSO₄, filtered and concentrated. The residue was purified by flash chromatography on silica gel and eluted with 60% ethyl acetate/hexane. Pure fractions were combined and concentrated to provide the title compound as a pale yellow solid (3.46 g, 78%). LC/MS: 258.1 (M+H)⁺. ¹H NMR (CDCl₃) δ 11.02 (bs, 1H), 8.12 (d, J=5.3, 1H), 7.83 (s, 1H), 7.42 (m, 2H), 7.13 (m, 2H), 7.06 (m, 1H), 6.82 (m, 1H). ¹⁹F NMR (CDCl₃) δ −68.26 (s), −111.86 (m).

Step C

### 4-[3-(4-Fluorophenyl)-1H-pyrazol-4-yl]pyridine-2(1H)-one

The product from Step B, 2-fluoro-4-[3-(4-fluorophenyl)-1H-pyrazol-4-yl]pyridine (1.50 g, 5.83 mmoles) was dissolved in a mixture of THF (30 mL) and 4.0 M HCl (30 mL). The solution was heated at reflux for 6 hours and then cooled to room temperature. The mixture was slowly poured into NaHCO₃/water and extracted three times with ethyl acetate. The organic extracts were dried over MgSO₄, filtered, and concentrated. The crude product was adsorbed onto silica gel, loaded onto a silica gel column and eluted with 10% methanol/dichloromethane. The pure fractions were combined and concentrated to furnish the title compound as a white solid (1.14 g, 77%). LC/MS: 256.1 (M+H)⁺. ¹H NMR (CD₃OD) δ 8.01 (bs, 1H), 7.48 (m, 2H), 7.33 (d, J=6.8, 1H), 7.19 (m, 2H), 6.44 (d, J=1.4, 1H), 6.34 (dd, J=6.8 and 1.4, 1H). ¹⁹F NMR (MeOD) δ −114.14 (m), −115.98 (m). 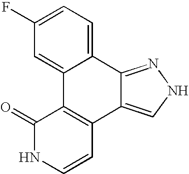
Step D

### 9-Fluoro-2,6-dihydro-7H-benzo[h]pyrazolo[4,3-f]isoquinolin-7-one

The product from Step C, 4-[3-(4-fluorophenyl)-1H-pyrazol-4-yl]pyridin-2(1H)-one, (100 mg, 0.392 mmoles), was dissolved in THF (100 mL) in an open crystallizing dish and exposed to UV light at a distance of 5 cm (Mineralight UVL-56, 365 nm). After 16 hours, the solution was concentrated and the residue was adsorbed onto silica gel and loaded onto a silica gel column. The product was eluted with 10% methanol/dichloromethane. Pure fractions were combined and concentrated to furnish the title compound as a white solid (35 mg, 35%) along with recovered starting material (30 mg). LC/MS: 254.0 (M+H)⁺. ¹H NMR (DMSO-d₆) δ 14.24 (bs, 1H), 11.80 (m, 1H), 10.06 (dd, J=14.1 and 2.7, 11H), 8.67 (s, 1H), 8.52 (m, 1H), 7.64 (m, 2H), 7.19 (d, J=6.8, 1H). ⁹F NMR (DMSO-d₆) δ −111.77 (m).

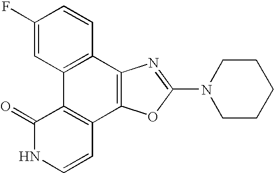

### Example 9

### 9-Fluoro-2-piperidin-1-ylbenzo[h][1,3]oxazolo[5,4-f]isoquinolin-7(6H)-one

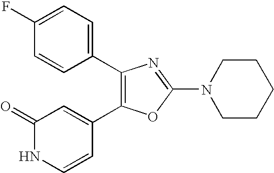 Step A

### 4-[4-(4-Fluorophenyl)-2-piperidin-1-yl-J, 3-oxazol-5-yl]pyridine-2(1H)-one

A solution of 2-bromo-1-(4-fluorophenyl)-2-(2-fluoropyridin-4-yl)ethanone (0.700 g, 2.24 mmol) and piperidine-1-carboxamide (287 mg, 2.24 mmol) in DMF (15 mL) was stirred at 90° C. until complete by LC/MS; desired oxazole: 342.0, (M+H)⁺. The DMF was removed on the rotovap and the crude residue was subjected to acid hydrolysis directly. Acetic acid (20 mL) and water (1.0 mL) were added and the solution was heated to 90° C. After 18 hours, LC/MS showed 92% conversion to the pyridone product. The mixture was cooled to room temperature, poured into 10% aqueous KHCO₃ (300 mL) and extracted with dichloromethane (3×75 mL). The organic extracts were concentrated and the crude product was purified by flash chromatography on silica gel eluting with a gradient of dichloromethane to 8% isopropanol/dichloromethane. Pure fractions were combined and concentrated to give the title compound as a yellow solid (0.32 g, 42%). LC/MS: 340.1, (M+H)⁺. ¹H NMR (DMSO-d₆) δ 11.42 (bs, 1H, pyridone NH), 7.56 (dd, J=8.7 and 5.8, 2H), 7.28 (t, J=8.7, 2H), 7.23 (d, J=7.0, 1H), 6.27 (s, 1H), 5.99 (d, J=7.0, 1H), 3.50 (bs, 4H, NCH₂), 1.58 (bs, 6H, CH₂). ¹⁹F NMR (DMSO-d₆) δ −112.79 (m).

Step B

### 9-Fluoro-2-piperidin-1-ylbenzo[h][1,3]oxazolo[5,4-f]isoquinolin-7(6H)-one

The title compound was prepared following the procedure described for Example 1, Step C. LC/MS: 338.0, (M+H)⁺. ¹H NMR (DMSO-d₆) δ 11.63 (m, 1H, pyridine NH), 9.97 (dd, J=14.1 and 2.6, 1H), 8.33 (dd, J=9.0 and 6.5, 1H), 7.55 (m, 2H), 6.83 (d, J=6.9, 1H), 3.76 (bs, 4H, NCH₂), 1.67 (bs, 6H, CH₂).

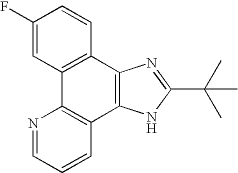

### Example 10

### 2-tert-Butyl-9-fluoro-3H-benzo[h]imidazo[4,5-f]quinoline

Step A

### N,N-diethyl-4-fluoro-2-(3-methylpyridin-2-yl)benzamide

To a solution of sec-butyllithium (1.4 M in cyclohexane, 17.6 mL, 24.6 mmol) and N,N,N′,N′-tetramethylethylenediamine (3.38 mL, 22.4 mmol) in THF (26 mL) at −78° C. was added a solution of N,N-diethyl-4-fluorobenzamide (4.37 g, 22.4 mmol) in THF (25 mL) over 5 minutes. After 30 minutes, ZnCl₂ (0.5 M in THF, 89.6 mL, 44.8 mmol) was added. The reaction was held at −78° C. for 1 hour and then was allowed to warm to room temperature. Upon reaching room temperature, the mixture was then added to a solution of 2-bromo-3-methylpyridine (2.63 mL, 22.4 mmol) and tetrakis(triphenylphosphine)palladium(0) (1.29 g, 1.12 mmol) in THF (25 mL) and the resulting mixture was heated to reflux for 16 hours. The reaction was cooled to ambient temperature and poured into saturated NaHCO₃. The aqueous portion was extracted with three portions of diethyl ether. The combined extracts were washed with brine, dried over Na₂SO₄, filtered and concentrated. The residue was chromatographed (30-50% ethyl acetate/hexanes) to afford N,N-diethyl-4-fluoro-2-(3-methylpyridin-2-yl)benzamide (2.97 g, 46%). ¹H NMR (CDCl₃, 400 MHz): δ 8.40 (d, J=4.7 Hz, 1H); 7.55 (d, J=7.6 Hz, 1H); 7.35 (dd, J=8.6, 5.7 Hz, 1H); 7.18 (dd, J=7.8, 4.7 Hz, 1H); 7.13 (dt, J=8.4, 2.5 Hz, 1H); 7.06 (dd, J=9.2, 2.5 Hz, 1H); 3.50-2.80 (br, 4H); 2.25 (s, 3H); 0.99 (t, J=7.1 Hz, 3H); 0.74 (t, J=7.1 Hz, 3H). MS(ES) 287 (M+1).

Step B

### 9-Fluorobenzo[h]quinol-6(5H)-one

To a solution of N,N-diisopropylamine (4.12 mL, 29.4 mmol) in THF (36 mL) at −78° C. was added n-butyllithium (1.6 M in Hexane, 18.0 mL, 28.8 mmol). The solution was raised to 0° C. and stirred for 20 minutes. To this solution was added dropwise a solution of N,N-diethyl-4-fluoro-2-(3-methylpyridin-2-yl)benzamide (1.65 g, 5.76 mmol) from Step A in THF (26 mL). After 0.5 hour, the reaction was quenched at 0° C. by the addition of pH 7 buffer. The layers were separated and the aqueous was extracted with three portions of diethyl ether. The combined organic extracts were washed with 1.0 N HCl, then brine, and were dried over Na₂SO₄, filtered and concentrated to afford 9-fluorobenzo[h]quinol-6(5H)-one (1.22 g, 99%) used crude in the oxidation step. ¹H NMR ((CD₃)₂SO, 400 MHz): δ 10.78 (s, 1H); 8.76 (dd, J=4.3, 1.7 Hz, 1H); 8.73 (dd, J=10.8, 2.9 Hz, 1H); 8.31 (dd, J=8.9, 5.9 Hz, 1H); 8.21 (dd, J=8.0, 1.6 Hz, 1H); 7.62 (dt, J=8.6, 2.8 Hz, 1H); 7.57 (dd, J=8.1, 4.4 Hz, 1H); 7.03 (s, 1H). MS(ES) 214 (M+1).

Step C

### 9-Fluorobenzo[h]quinoline-5,6-dione

A solution of 9-fluorobenzo[h]quinol-6(5H)-one (0.281 g, 1.32 mmol) from Step B in THF (21 mL) was added to a suspension of chromium (VI) oxide adsorbed on silica gel (5 g, 9% w/w CrO₃) in diethyl ether (15 mL). The reaction was stirred for 2 hours at room temperature. The silica gel was filtered off and rinsed with diethyl ether. The filtrate was concentrated and the residue was chromatographed (1% MeOH/CHCl₃) to afford 9-fluorobenzo[h]quinoline-5,6-dione (132 mg, 44%). ¹H NMR (CDCl₃, 400 MHz): δ 8.70 (dd, J=4.9, 2.0 Hz, 1H); 8.43 (dd, J=8.0, 2.0 Hz, 1H); 8.37 (dd, J=9.8, 2.5 Hz, 1H); 8.25 (dd, J=8.8, 5.7 Hz, 1H); 7.48 (dd, J=7.8, 4.7 Hz, 1H); 7.26 (dd, J=8.4, 2.5 Hz, 1H). MS(ES) 228 (M+1).

Step D

### 2-tert-Butyl-9-fluoro-3H-benzo[h]imidazo[4,5-f]quinoline

To a solution of 9-fluorobenzo[h]quinoline-5,6-dione (412 mg, 1.81 mmol) from Step C in acetic acid (25 mL) was added pivaldehyde (0.30 mL, 2.8 mmol) and ammonium acetate (0.84 g, 11 mmol). The mixture was heated to 100° C. for 16 hours. The reaction was cooled to ambient temperature and neutralized by slow addition to NaHCO₃ solution. The product was extracted with three portions of methylene chloride, and the combined extracts were washed with brine, dried over Na₂SO₄, filtered and concentrated. The reaction mixture was chromatographed (2:1 hexanes/ethyl acetate) to afford 2-tert-butyl-9-fluoro-3H-benzo[h]imidazo[4,5-f]quinoline (465 mg, 87%). ¹H NMR ((CD₃)₂SO, 400 MHz): δ 9.00 (d, J=2.9 Hz, 1H); 8.95 (d, J=7.6 Hz, 1H); 8.86 (dd, J=10.9, 2.7 Hz, 1H); 8.67 (m, 1H); 7.83 (dd, J=8.0, 4.3 Hz, 1H); 7.78 (m, 1H); 5.20-3.40 (br s, 1H); 1.57 (s, 9H). MS(ES) 294 (M+1).

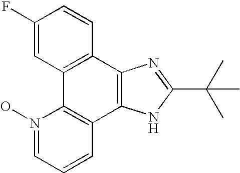

### Example 11

### 2-tert-Butyl-9-fluoro-3H-benzo[h]imidazo[4,5-f]quinoline 7-oxide

To a solution of 2-tert-butyl-9-fluoro-3H-benzo[h]imidazo[4,5-f)quinoline (106 mg, 0.36 mmol) from Example 10 in acetic acid (10 mL) held at 65° C. was added sodium perborate monohydrate (476 mg, 4.8 mmol) portionwise over 1 hour. The reaction was held at this temperature for 16 hours. Upon cooling to room temperature, the reaction was neutralized by careful addition to a solution of NaHCO₃. The product was extracted with three portions of 10% iPrOH/DCM. The combined extracts were dried over Na₂SO₄, filtered and concentrated. Purification by column chromatography (5% MeOH/DCM) afforded 2-tert-butyl-9-fluoro-3H-benzo[h]imidazo[4,5-f]quinoline 7-oxide (18 mg, 16%). ¹H NMR (CD₃OD, 400 MHz): δ 10.50 (br d, J=12.5 Hz, 1H); 8.90-8.40 (m, 3H); 7.70 (br t, J=7.0 Hz, 1H); 7.61 (m, 1H); 1.60 (s, 9H). MS(ES) 310 (M+1).

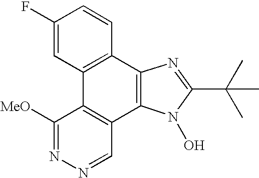

### Example 12

### 2-tert-Butyl-9-fluoro-7-methoxy-3H-benzo[f]imidazo[4,5-h]phthalazin-3-ol trifluoroacetic acid

Step A

### 3-Chloro-5-iodo-6-methoxy-4-methylpyridazine

To a solution of 2,2,6,6-tetramethylpiperidine (1.34 mL, 7.94 mmol) in THF (15 mL) at room temperature was added n-butyllithium (2.5 M in hexane, 3.2 mL, 7.90 mmol). The resulting solution was stirred for 20 minutes, followed by cooling to −78° C. To this was added rapidly a precooled (−78° C.) solution of 3-chloro-6-methoxy-4-methylpyridazine (0.360 g, 2.27 mmol) in THF (15 mL). After 5 minutes, a precooled (−78° C.) solution of iodine (0.96 g, 3.78 mmol) in THF (15 mL) was rapidly introduced. After 15 minutes, the reaction was quenched at −78° C. by the addition of saturated NH₄Cl solution. Upon warming to room temperature, the layers were separated and the aqueous was extracted with diethyl ether. The combined extracts were washed successively with saturated NaHCO₃ and brine, and were dried over Na₂SO₄, filtered and concentrated. Column chromatography (10% Ethyl acetate/Hexanes) afforded 3-chloro-5-iodo-6-methoxy-4-methylpyridazine (0.44 g, 68%). MS(ES) 284.9 (M+1).

Step B

### {2-[(Diethylamino)carbonyl]-5-fluorophenyl}boronic acid

To a solution of N,N,N′,N′-tetramethylethylenediamine (6.96 mL, 46.1 mmol) in THF (100 mL) at −78° C. was added sec-butyllithium (1.4 M in cyclohexane, 32.9 mL, 46.1 mmol), followed by rapid addition over 4 minutes of a solution of N,N-diethyl-4-fluorobenzamide (6.00 g, 30.7 mmol) in THF (25 mL). The mixture was stirred at −78° C. for 10 minutes and was then quenched by the addition of trimethyl borate (10.5 mL, 92.2 mmol). The solution was stirred at this temperature for 15 minutes, followed by removal of the cooling bath. When the reaction mixture reached about 0° C., saturated ammonium chloride solution was added. The reaction was then acidified by the addition of aqueous HCl solution. After stirring for 30 minutes, the layers were separated and the aqueous was extracted with three portions of methylene chloride. The combined organic extracts were dried over Na₂SO₄, filtered and concentrated to afford {2-[(diethylamino)carbonyl]-5-fluorophenyl}boronic acid (6.80 g, 93%). ¹H NMR (CD₃OD, 400 MHz): δ 7.97 (dd, J=8.8, 4.2 Hz, 1H); 7.28 (dd, J=7.8, 2.5 Hz, 1H); 7.18 (dt, J=8.8, 2.7 Hz, 1H); 4.01 (q, J=7.3 Hz, 2H); 3.78 (q, J=7.3 Hz, 2H); 1.47 (t, J=7.1 Hz, 3H); 1.37 (t, J=7.1 Hz, 3H). MS(ES) 222 (M−H₂O+1).

Step C

### 2-(6-Chloro-3-methoxy-5-methylpyridazin-4-yl)-N,N-diethyl-4-fluorobenzamide

A solution of 3-chloro-5-iodo-6-methoxy-4-methylpyridazine (0.350 g, 1.23 mmol) of Step A and {2-[(diethylamino)carbonyl]-5-fluorophenyl}boronic acid (0.392 g, 1.48 mmol) of Step B in toluene (30 mL) and ethanol (0.6 mL) were combined with a solution of K₂CO₃ (2.0 M in water, 0.12 mL) and the resulting solution was deoxygenated by purging with a stream of dry nitrogen for 1 hour. Tetrakis(triphenylphosphine)palladium(0) (0.50 g, 0.43 mmol) was introduced and the reaction was heated to 110° C. for 24 hours. The reaction was partitioned between saturated ammonium chloride and ethyl acetate, and the aqueous portion was extracted with ethyl acetate. The combined organic extracts were dried over Na₂SO₄, filtered and concentrated. The product was purified by column chromatography (40% ethyl acetate/hexanes), and the solid so obtained was washed with 10 mL of methanol to afford 2-(6-chloro-3-methoxy-5-methylpyridazin-4-yl)-N,N-diethyl-4-fluorobenzamide (0.25 g, 58%). ¹H NMR (CDCl₃, 400 MHz): δ 7.38 (dd, J=8.6, 5.6 Hz, 1H); 7.18 (dt, J=8.3, 2.6 Hz, 1H); 6.89 (dd, J=8.9, 2.7 Hz, 1H); 3.99 (s, 3H); 3.59 (dq, J=14.2, 6.8 Hz, 1H); 3.30 (dq, J=14.7, 7.0 Hz, 1H); 3.02 (dq, J=13.9, 7.0 Hz, 1H); 2.96 (dq, J=14.1, 7.0 Hz, 1H); 2.18 (s, 3H); 1.09 (t, J=7.1 Hz, 3H); 0.85 (t, J=7.2 Hz, 3H). MS(ES) 352 (M+1).

Step D

### 4-Chloro-9-fluoro-1-methoxybenzo[f]phthalazin-6-ol

To a solution of N,N-diisopropylamine (0.179 mL, 1.28 mmol) in THF (10 mL) at −78° C. was added n-butyllithium (2.5 M in hexane, 0.51 mL, 1.3 mmol). After stirring for 20 minutes, a solution of 2-(6-chloro-3-methoxy-5-methylpyridazin-4-yl)-N,N-diethyl-4-fluorobenzamide (0.150 g, 0.426 mmol) of Step C in THF (5 mL) was added. The reaction was allowed to warm to −10° C., and was quenched by the addition of saturated ammonium chloride solution. The aqueous layer was separated and extracted with ethyl acetate. The combined organic extracts were dried over Na₂SO₄, filtered and concentrated. The crude mixture was chromatographed (1:1 ethyl acetate/hexanes) to afford 4-chloro-9-fluoro-1-methoxybenzo[f]phthalazin-6-ol (100 mg, 84%). MS(ES) 279 (M+1).

Step E

### 9-Fluoro-1-methoxybenzo[f]phthalazin-6-ol

4-Chloro-9-fluoro-1-methoxybenzo[f]phthalazin-6-ol (220 mg, 0.789 mmol) of Step D and 10% palladium on carbon (25 mg, 0.024 mmol) was stirred in a mixture of ethanol (5.0 mL) and 2.0 M K₂CO₃ (0.9 mL) under an atmosphere of hydrogen for 16 hours. The reaction mixture was filtered and the filtrate was evaporated under reduced pressure. The residue was partitioned between saturated ammonium chloride solution and ethyl acetate. The organic phase was dried over Na₂SO₄, filtered and concentrated to afford 9-fluoro-1-methoxybenzo[f]phthalazin-6-ol (178 mg, 92%). ¹H NMR ((CD₃)₂SO, 400 MHz): δ 11.70 (s, 1H); 9.31 (s, 1H); 9.07 (dd, J=12.6, 2.8 Hz, 1H); 8.50 (dd, J=9.1, 6.4 Hz, 1H); 7.78-7.72 (m, 1H); 7.18 (s, 1H); 4.32 (s, 3H). MS(ES) 245 (M+1).

Step F

### 9-Fluoro-1-methoxybenzo[f]phthalazine-5,6-dione 5-oxime

To a −10° C. solution of 9-fluoro-1-methoxybenzo[f]phthalazin-6-ol (100 mg, 0.41 mmol) of Step E in DMF (10 mL) was added tert-butyl nitrite (65 μL, 0.49 mmol) and 4.0 M hydrogen chloride in 1,4-dioxane (100 μL, 0.4 mmol). The reaction was stirred at this temperature for 45 minutes. The pH was adjusted to 6 using NaHCO₃ solution. All solvent was removed under reduced pressure. The solid obtained was washed with water (3 mL) and ethyl acetate (2 mL) to afford 9-fluoro-1-methoxybenzo[f]phthalazine-5,6-dione 5-oxime (70 mg, 62%). MS(ES) 274 (M+1).

Step G

### 2-tert-Butyl-9-fluoro-7-methoxy-3H-benzo[f]imidazo[4,5-h]phthalazin-3-ol TFA salt

A mixture of 9-fluoro-1-methoxybenzo[f]phthalazine-5,6-dione 5-oxime (50 mg, 0.183 mmol) of Step F, pivaldehyde (60 μL, 0.55 mmol), and ammonium acetate (85 mg, 1.1 mmol) in acetic acid (3 mL) was heated to 80° C. for 3 hours. The reaction mixture was cooled and the pH was adjusted to 6 by the addition of NaHCO₃ solution. The product was extracted using ethyl acetate, and the solvent was removed. The product was purified by prep-HPLC to afford 2-tert-butyl-9-fluoro-7-methoxy-3H-benzo[f]imidazo[4,5-h]phthalazin-3-ol as the trifluoroacetic acid (TFA) salt, (55 mg, 66%). ¹H NMR (CDCl₃, 400 MHz): δ 10.00 (s, 1H); 9.05 (dd, J=12.8, 2.4 Hz, 1H); 8.75 (dd, J=9.2, 6.2 Hz, 1H); 7.71-7.68 (m, 1H); 7.62-7.57 (m, 1H); 4.26 (s, 3H); 1.70 (s, 9H). MS(ES) 341 (M+1).

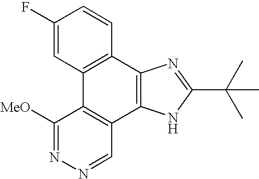

### Example 13

### 2-tert-Butyl-9-fluoro-7-methoxy-3H-1,3,5,6-tetraaza-cyclopenta[l]phenanthrene TFA salt

A solution of 2-tert-butyl-9-fluoro-7-methoxy-3H-benzo[f]imidazo[4,5-h]phthalazin-3-ol TFA salt (36 mg, 0.079 mmol) of Example 12 and triethyl phosphine (150 μL, 0.87 mmol) in N,N-dimethylacetamide (0.50 mL) was heated in a 160° C. oil bath for 45 minutes. The product was purified by HPLC to afford 2-tert-butyl-9-fluoro-7-methoxy-3H-1,3,5,6-tetraaza-cyclopenta[l]phenanthrene TFA salt (14 mg, 40%). MS(ES) 325 (M+1).

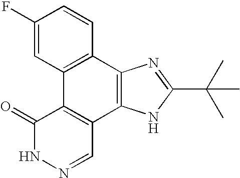

### Example 14

### 2-tert-Butyl-9-fluoro-3,6-dihydro-1,3,5,6-tetraaza-cyclopenta[l]phenanthren-7-one

To 2-tert-butyl-9-fluoro-7-methoxy-3H-1,3,5,6-tetraaza-cyclopenta[l]phenanthrene TFA salt (10 mg, 0.023 mmol) of Example 13 in ethanol (0.2 mL) was added conc. HCl (0.4 mL) and the resulting solution was heated to 80° C. for 80 minutes. The reaction mixture was cooled and the pH was adjusted to 10 by the addition of NaOH solution. The product was extracted with Ethyl acetate. The extracts were dried over Na₂SO₄, filtered and concentrated. The residue was triturated with methylene chloride to afford 2-tert-butyl-9-fluoro-3,6-dihydro-1,3,5,6-tetraaza-cyclopenta[l]phenanthren-7-one (8 mg, 87%). ¹H NMR ((CD₃)₂SO, 500 MHz) (tautomeric mixture): δ 13.58 (br s, 1H); 13.10 (s, major tautomer) and 13.09 (s, minor tautomer) (together 1H); 9.95 (dd, J=13.2, 2.7 Hz, major tautomer) and 9.90 (dd, J=13.8, 2.9 Hz, minor tautomer) (together 1H); 9.33-9.27 (m, 0.5H); 8.96-8.88 (m, 1H); 8.67 (dd, J=8.8, 6.3 Hz, 0.5H); 7.80 (t, J=8.4 Hz, major tautomer) and 7.72 (dt, J=8.9, 2.7 Hz, minor tautomer) (together 1H); 1.55 (s, minor tautomer) and 1.54 (s, major tautomer) (together 9H). MS(ES) 311 (M+1).

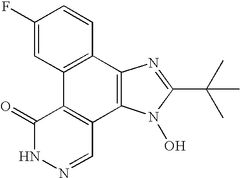

### Example 15

### 2-tert-Butyl-9-fluoro-3H-benzo[f]imidazo[4,5-h]phthalazine-3,7-diol

A solution of 2-tert-butyl-9-fluoro-7-methoxy-3H-benzo[f]imidazo[4,5-h]phthalazin-3-ol (10.5 mg, 0.031 mmol) of Example 12 in ethanol (0.2 mL) and conc. HCl (0.4 mL) was heated to 80° C. for 80 minutes. The product was purified by prep-LCMS to afford 2-tert-butyl-9-fluoro-3H-benzo[f]imidazo[4,5-h]phthalazine-3,7-diol. MS(ES) 327 (M+1).

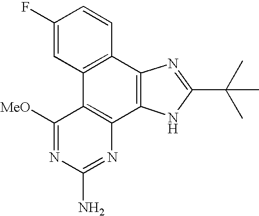

### Example 16

### 2-tert-Butyl-9-fluoro-7-methoxy-3H-benzo[f]imidazo[4,5-h]quinazolin-5-amine

Step A

### tert-Butyl(5-iodo-4-methoxy-6-methylpyrimidin-2-yl)carbamate

To 5-iodo-4-methoxy-6-methylpyrimidin-2-amine (2.47 g, 9.32 mmol) in THF (50 mL) was added di-tert-Butyldicarbonate (4.39 mL, 19.1 mmol) and 4-dimethylaminopyridine (200 mg, 2 mmol). The reaction was stirred for 16 hours. The mixture was partitioned between saturated NaHCO₃ solution and diethyl ether, and the aqueous layer was extracted with two further portions of diethyl ether. The combined organic extracts were dried over Na₂SO₄, filtered and concentrated to afford tert-butyl(5-iodo-4-methoxy-6-methylpyrimidin-2-yl)carbamate (4.27 g, 98%). ¹H NMR (CDCl₃, 400 MHz): δ 4.00 (s, 3H); 2.66 (s, 3H); 1.47 (s, 18H). MS(ES) 466 (M+1).

Step B

### di-tert-Butyl(5-(2-[(diethylamino)carbonyl]-5-fluorophenyl)-4-methoxy-6-methylpyrimidin-2-yl)imidodicarbonate

To a solution of N,N,N′,N′-tetramethylethylenediamine (2.26 mL, 15.0 mmol) in THF (26 mL) at −78° C. was added sec-butyllithium (1.4 M in cyclohexane, 10.7 mL, 15.0 mmol), followed by rapid addition of a solution of N,N-diethyl-4-fluorobenzamide (2.74 g, 14.0 mmol) in THF (8.8 mL). The mixture was stirred at −78° C. for 5 minutes followed by the addition of zinc dichloride (0.5 M in THF, 28.1 mL, 14.0 mmol). The reaction was stirred at −78° C. for 15 minutes, and the cooling bath was then removed. Upon reaching room temperature, this mixture was then added in three portions at 1 hour intervals to a solution of tert-butyl(5-iodo-4-methoxy-6-methylpyrimidin-2-yl)carbamate (2.11 g, 4.53 mmol) of Step A and tetrakis(triphenylphosphine)palladium(0) (0.52 g, 0.45 mmol) in THF (18 mL) at reflux. One hour following the last addition, the aryl iodide was completely consumed. The reaction was cooled to room temperature and the mixture was partitioned between saturated NaHCO₃ and diethyl ether. The aqueous layer was extracted with three further portions of ether. The combined extracts were washed with brine, dried over Na₂SO₄, filtered and concentrated. Silica gel chromatography (10-25-50% ethyl acetate/hexanes) afforded a mixture of product, starting amide, and self-condensed amide. The product was separated by prep-HPLC, followed by neutralization of the resulting TFA salt and extraction into ethyl acetate to afford di-tert-butyl(5-{2-[(diethylamino)carbonyl]-5-fluorophenyl}-4-methoxy-6-methylpyrimidin-2-yl)imidodicarbonate (0.80 g, 33%). ¹H NMR (CDCl₃, 400 MHz): δ 7.36 (dd, J=8.4, 5.6 Hz, 1H); 7.12 (dt, J=8.4, 2.5 Hz, 1H); 6.90 (dd, J=9.2, 2.7 Hz, 1H); 3.84 (s, 3H); 3.58-2.89 (br, 4H); 2.29 (s, 3H); 1.47 (s, 18H); 1.05 (t, J=7.0 Hz, 3H); 0.90 (t, J=7.0 Hz, 3H). MS(ES) 533 (M+1).

Step C

### tert-Butyl(9-fluoro-6-hydroxy-1-methoxybenzo[f]quinazolin-3-yl)carbamate

To a solution of N,N-diisopropylamine (0.82 mL, 5.84 mmol) in THF (7.2 mL) at −78° C. was added n-butyllithium (1.6 M in hexane, 3.58 mL, 5.73 mmol). The solution was warmed to 0° C. and stirred for 15 minutes, followed by cooling to −78° C. To this solution was added dropwise di-tert-butyl(5-{2-[(diethylamino)carbonyl]-5-fluorophenyl}-4-methoxy-6-methylpyrimidin-2-yl)imidodicarbonate (0.61 g, 1.14 mmol) of Step B in THF (5.1 mL). The reaction was stirred for 30 minutes at this temperature, and was then warmed to 0° C. and was stirred for an additional 2 hours. The reaction was quenched at 0° C. by the addition of pH 7 buffer. The layers were separated and the aqueous layer was extracted three times with ethyl acetate. The combined organic extracts were washed with brine, dried over Na₂SO₄, filtered and concentrated. Silica gel chromatography (20% acetone/hexane) afforded tert-butyl(9-fluoro-6-hydroxy-1-methoxybenzo[f]quinazolin-3-yl)carbamate (153 mg, 37%). ¹H NMR (CDCl₃, 400 MHz): δ 12.00-11.20 (br s, 1H); 8.65 (dd, J=12.7, 2.4 Hz, 1H); 8.30 (dd, J=9.0, 6.6 Hz, 1H); 7.26 (m, 1H); 7.15 (s, 1H); 6.65 (br s, 1H); 4.17 (s, 3H); 1.43 (s, 9H). MS(ES) 360 (M+1).

Step D

### tert-Butyl[9-fluoro-5-(hydroxyimino)-1-methoxy-6-oxo-5,6-dihydrobenzo[f]quinazolin-3-yl]carbamate

Tert-butyl nitrite (74 μL, 0.62 mmol) and 4.0 M of HCl in dioxane (0.12 mL, 0.4 mmol) were added to a solution of tert-butyl(9-fluoro-6-hydroxy-1-methoxybenzo[f]quinazolin-3-yl)carbamate (0.103 g, 0.287 mmol) of Step C in DMF (2.1 mL) at 0° C. The reaction was allowed to warm to room temperature. On completion of the reaction, water was introduced and the acidic aqueous medium was extracted with copious quantities of ethyl acetate. The combined organic extracts were dried over Na₂SO₄, filtered and concentrated to afford tert-butyl[9-fluoro-5-(hydroxyimino)-1-methoxy-6-oxo-5,6-dihydrobenzo[f]quinazolin-3-yl]carbamate (110 mg, 99%). ¹H NMR ((CD₃)₂SO, 400 MHz): δ 11.16 (s, 1H); 8.39 (dd, J=11.7, 2.4 Hz, 1H); 8.30 (dd, J=8.4, 6.4 Hz, 1H); 7.42 (dt, J=8.4, 2.5 Hz, 1H); 4.20 (s, 3H); 1.52 (s, 9H). MS(ES) 389 (M+1).

Step E

### 2-tert-Butyl-9-fluoro-7-methoxy-3H-benzo[f]imidazo[4,5-h]quinazolin-5-amine

A solution of tert-butyl[9-fluoro-5-(hydroxyimino)-1-methoxy-6-oxo-5,6-dihydrobenzo[f]quinazolin-3-yl]carbamate (165 mg, 0.425 mmol) of Step D, pivaldehyde (0.143 mL, 1.32 mmol) and ammonium acetate (0.19 g, 2.5 mmol) in acetic acid (5 mL) was heated to reflux for 5 hours. The reaction was cooled and the solvent was removed under reduced pressure. The residue was slurried in water, and 1.0 N NaOH was added to adjust the pH to 9-10. The aqueous mixture was extracted with copious quantities of ethyl acetate, and the volatiles were removed under reduced pressure. The crude product was chromatographed (4% MeOH/DCM) to result in a mixture of 5-amino-2-tert-butyl-9-fluoro-7-methoxy-3H-benzo[f]imidazo[4,5-h]quinazolin-3-ol (¹H NMR (CD₃OD, 500 MHz): δ 8.70 (dd, J=13.4, 2.7 Hz, 1H); 8.51 (dd, J=8.7, 6.3 Hz, 1H); 7.28 (dt, J=8.5, 2.6 Hz, 1H); 4.22 (s, 3H); 1.62 (s, 9H). MS(ES) 356 (M+1)) and 2-tert-butyl-9-fluoro-7-methoxy-3H-benzo[f]imidazo[4,5-h]quinazolin-5-amine (see below for characterization) (56 mg). This mixture of two products was dissolved in acetic acid (3.6 mL). To this was added Zinc powder (414 mg, 6.34 mmol), and the suspension was heated to reflux for 8 hours. A fresh portion of zinc powder (180 mg, 2.75 mmol) was added and the reaction was heated for 30 minutes to complete the reduction of 5-amino-2-tert-butyl-9-fluoro-7-methoxy-3H-benzo[f]imidazo[4,5-h]quinazolin-3-ol to form 2-tert-butyl-9-fluoro-7-methoxy-3H-benzo[f]imidazo[4,5-h]quinazolin-5-amine. The reaction was cooled to room temperature, the solids were filtered off, and the filter cake was washed with acetic acid. The acetic acid was removed under reduced pressure. The resulting residue was slurried in water, and the pH was adjusted to 9-10 by the addition of 1.0 N NaOH. The product was extracted with copious quantities of ethyl acetate. The combined organic extracts were dried over Na₂SO₄, filtered and concentrated to afford 2-tert-butyl-9-fluoro-7-methoxy-3H-benzo[f]imidazo[4,5-h]quinazolin-5-amine (53 mg, 37% over the two steps). ¹H NMR (CDCl₃, 500 MHz): δ 11.30-10.90 (br s, 1H); 8.79 (dd, J=13.3, 2.6 Hz, 1H); 8.65 (br s, 1H); 7.33 (t, J=7.3 Hz, 1H); 6.14 (br s, 1H); 4.22 (s, 3H); 1.56 (s, 9H). MS(ES) 340 (M+1).

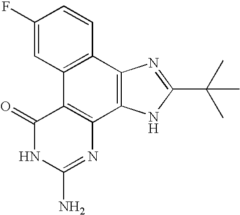

### Example 17

### 5-Amino-2-tert-butyl-9-fluoro-3H-benzo[f]imidazo[4,5-h]quinazolin-7-ol

A solution of 2-tert-butyl-9-fluoro-7-methoxy-3H-benzo[f]imidazo[4,5-h]quinazolin-5-amine (25 mg, 0.074 mmol) of Example 16 in ethanol (3 mL) and conc. HCl (1.5 mL) was heated to 100° C. for 2 hours. The reaction was then cooled to 0° C. and was neutralized by the addition of solid NaOH. The aqueous mixture was extracted with ethyl acetate to afford 5-amino-2-tert-butyl-9-fluoro-3H-benzo[f]imidazo[4,5-h]quinazolin-7-ol (15 mg, 63%). The product was purified by prep-LCMS to afford 5-amino-2-tert-butyl-9-fluoro-3H-benzo[f]imidazo[4,5-h]quinazolin-7-ol as the bis-TFA salt (18 mg). ¹H NMR ((CD₃)₂SO, 400 MHz): δ 9.42 (dd, J=13.1, 2.7 Hz, 1H); 8.63 (m, 1H); 7.80-7.50 (br s, 1H); 7.60 (m, 1H); 4.50 (br s, 5H), 1.52 (s, 9H). MS(ES) 326 (M+1).

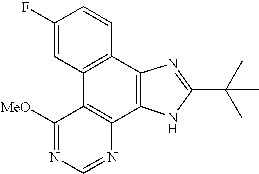

### Example 18

### 2-tert-Butyl-9-fluoro-7-methoxy-3H-benzo[f]imidazo[4,5-h]quinazoline

Step A

### 5-bromo-4-methoxy-6-methylpyrimidine

Sodium methoxide (25 wt % solution in methanol, 1.83 mL, 16.0 mmol) was added to a solution of 5-bromo-4-chloro-6-methylpyrimidine (1.85 g, 8.92 mmol) in methanol (50 mL) and the reaction was stirred at ambient temperature for 1 hour. The reaction was quenched by the addition of pH 7 buffer. The majority of the methanol was removed under reduced pressure. The aqueous portion was diluted with water and was extracted with diethyl ether three times. The combined organic extracts were washed with brine, dried over Na₂SO₄, filtered and concentrated to afford 5-bromo-4-methoxy-6-methylpyrimidine (1.43 g, 79%). ¹H NMR (CDCl₃, 400 MHz): δ 8.48 (s, 1H); 4.00 (s, 3H); 2.54 (s, 3H). MS(ES) 204 (M+1).

Step B

### N,N-diethyl-4-fluoro-2-(4-methoxy-6-methylpyrimidin-5-yl)benzamide

A microwavable vial was charged with {2-[(diethylamino)carbonyl]-5-fluorophenyl}boronic acid (0.424 g, 1.77 mmol), 5-bromo-4-methoxy-6-methylpyrimidine (0.200 g, 0.985 mmol) of Step A, sodium carbonate (0.313 g, 2.96 mmol), toluene (1.5 mL) and water (0.5 mL). The solution was degassed by purging with nitrogen for 10 minutes. Afterwards, tetrakis(triphenylphosphine)palladium(0) (0.110 g, 0.098 mmol) was added. The vial was sealed and microwaved at 160° C. for 10 minutes. The reaction mixture was partitioned between water and ethyl acetate. The aqueous layer was extracted with two further portions of ethyl acetate. The combined organic extracts were washed with brine, dried over Na₂SO₄, filtered and concentrated. The mixture was chromatographed (50-75% ethyl acetate/hexane) to afford N,N-diethyl-4-fluoro-2-(4-methoxy-6-methylpyrimidin-5-yl)benzamide (250 mg, 80%). ¹H NMR (CDCl₃, 500 MHz): δ 8.65 (s, 1H); 7.36 (dd, J=9.2, 6.0 Hz, 1H); 7.13 (dt, J=8.4, 2.6 Hz, 1H); 6.93 (dd, J=9.1, 2.4 Hz, 1H); 3.88 (s, 3H), 3.70-2.85 (br, 4H); 2.31 (s, 3H); 1.05 (t, J=7.1 Hz, 3H); 0.80 (t, J=7.2 Hz, 3H). MS(ES) 318 (M+1).

Step C

### 9-Fluoro-1-methoxybenzo[f]quinazolin-6-ol

To a solution of N,N-diisopropylamine (0.51 mL, 3.6 mmol) in THF (20 mL) at −78° C. was added n-butyllithium (1.6 M in hexanes, 2.17 mL, 3.47 mmol) dropwise. The solution was stirred at this temperature for 15 minutes, at 0° C. for 10 minutes, and then was cooled again to −78° C. This solution was transferred via cannula to a solution of N,N-diethyl-4-fluoro-2-(4-methoxy-6-methylpyrimidin-5-yl)benzamide (462 mg, 1.45 mmol) of Step B in THF (47 mL) held at −15° C. The reaction was stirred at this temperature for 15 minutes and was then quenched by the addition of pH 7 buffer. The product was extracted from the aqueous phase with ethyl acetate. The combined organic extracts were washed with brine, dried over Na₂SO₄, filtered and concentrated to afford 9-fluoro-1-methoxybenzo[f]quinazolin-6-ol (350 mg, 99%). ¹H NMR ((CD₃)₂SO, 500 MHz): δ 11.58 (br s, 1H); 8.93 (dd, J=12.6, 2.5 Hz, 1H); 8.77 (s, 1H); 8.42 (dd, J=9.6, 6.7 Hz, 1H); 7.56 (m, 1H); 7.10 (s, 1H); 4.25 (s, 3H). MS(ES) 245 (M+1).

Step D

### 9-Fluoro-1-methoxybenzo[f]quinazoline-5,6-dione 5-oxime

Tert-butyl nitrite (0.32 mL, 2.7 mmol) and 4.0 M of hydrogen chloride in 1,4-dioxane (0.62 mL, 2.5 mmol) were added to a solution of 9-fluoro-1-methoxybenzo[f]quinazolin-6-ol (186 g, 0.76 mmol) of Step C in DMF (15 mL) at room temperature. After stirring for 20 minutes, the reaction was diluted with water, and extracted with ethyl acetate three times. The combined organic extracts were dried over Na₂SO₄, filtered and concentrated to provide 9-fluoro-1-methoxybenzo[f]quinazoline-5,6-dione 5-oxime (187 mg, 90%) as a mixture of isomers, used crude in the imidazole formation. MS(ES) 274 (M+1).

Step E

### 2-tert-Butyl-9-fluoro-7-methoxy-3H-benzofimidazo[4,5-h]quinazoline

A mixture of 9-fluoro-1-methoxybenzo[f]quinazoline-5,6-dione 5-oxime (176 mg, 0.64 mmol) of Step D, pivaldehyde (0.43 mL, 3.9 mmol), and ammonium acetate (0.57 g, 7.4 mmol) in acetic acid (15 mL) was heated to 100° C. for 16 hours. The reaction was cooled to room temperature and the solvent was removed under reduced pressure. The residue was slurried in water, and 1.0 N NaOH was added to adjust the pH to 9-10. The aqueous mixture was extracted with copious quantities of ethyl acetate, and the volatiles were removed under reduced pressure to afford 2-tert-butyl-9-fluoro-7-methoxy-3H-benzo[f]imidazo[4,5-h]quinazoline (99 mg, 47%). ¹H NMR ((CD₃)₂SO, 500 MHz), major tautomer: δ 13.47 (s, 1H); 9.04 (dd, J=13.0, 2.6 Hz, 1H); 8.99 (s, 1H); 8.63 (dd, J=8.9, 6.6 Hz, 1H); 7.65 (ddd, J=8.4, 8.4, 2.7 Hz, 1H); 4.31 (s, 3H); 1.51 (s, 9H). MS(ES) 325 (M+1).

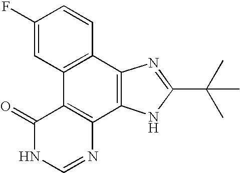

### Example 19

### 2-tert-Butyl-9-fluoro-3H-benzo[f]imidazo[4,5-h]quinazolin-7-ol

2-tert-Butyl-9-fluoro-7-methoxy-3H-benzo[f]imidazo[4,5-h]quinazoline (70 mg, 0.216 mmol) of Example 18 in ethanol (6 mL) and conc. HCl (3 mL) was heated to 100° C. for 1 hour. The reaction was cooled to 0° C. and neutralized by the addition of solid NaOH. The aqueous mixture was extracted with three portions of Ethyl acetate. The combined organic extracts were dried over Na₂SO₄, filtered and concentrated to provide crude 2-tert-butyl-9-fluoro-3H-benzo[f]imidazo[4,5-h]quinazolin-7-ol (43 mg, 64%). The product was purified by silica gel chromatography (4% MeOH/DCM). A portion was purified by prep-LCMS to afford the TFA salt. ¹H NMR (CD₃OD, 500 MHz): δ 9.74 (dd, J=13.1, 3.1 Hz, 1H); 8.68 (dd, J=8.9, 5.8 Hz, 1H); 8.43 (s, 1H); 7.63 (m, 1H), 1.70 (s, 9H). MS(ES) 311 (M+1).

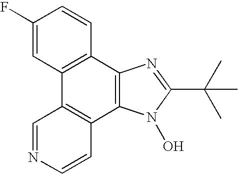

### Example 20

### 2-tert-Butyl-9-fluoro-3H-benzo[h]imidazo[4,5-f]isoquinolin-3-ol

Step A

### N,N-diethyl-4-fluoro-2-(4-methylpyridin-3-yl)benzamide

To a −78° C. solution of 1.4 M of sec-butyllithium in tetrahydrofuran (18 mL) and N,N,N′,N′-tetramethylethylenediamine (3.4 mL, 0.022 mol) in tetrahydrofuran (25 mL, 0.31 mol) was added a solution of 4-fluoro-N,N-diisopropylbenzamide (5.0 g, 0.022 mol) in tetrahydrofuran (25 mL, 0.31 mol) over 5 min. An orange precipitate formed. After 0.5 h, a solution of 0.5 M of zinc dichloride in tetrahydrofuran (40 mL) was added. The reaction was allowed to warm to −60° C. for 2 h. The CO₂ bath was removed for 1 h and the reaction mixture became an orange solution. 3-Bromo-4-methylpyridine (2.5 mL, 0.022 mol) and tetrakis(triphenylphosphine)palladium(0) (1 g, 0.001 mol) were added and the resulting mixture was heated at reflux overnight. The reaction was partitioned between EtOAc and 0.1 N HCl, washed by 1 N NaOH×2, sat. NaCl. The organic phase was dried over sodium sulfate and rotovapped to give 8.75 g of an orange oil. The product was chromatographed with 50% EtOAc/hexanes and sampled in DCM. White solid was collected which was dried at 60° C. under high vacuum overnight to give 4.18 g of product (60% yield). LCMS: 349.1 (M+1), 1.17 min. ¹H NMR (CDCl₃): δ 8.45 (d, 1H); 8.4 (brd, 1H); 7.25 (m, 2H); 7.17 (m, 1H); 6.98 (dd, 1H); 3.7 (brd, 1H); 3.24 (brd, 1H); 2.3 (s, 3H); 1.4 (br s, 3H); 1.0 (brd, 9H).

Step B

### 9-Fluorobenzo[h]isoquinolin-6-ol

To a −78° C. solution of N,N-diisopropylamine (4.5 mL, 0.032 mol) in tetrahydrofuran (40 mL, 0.5 mol) was added 1.6 M of n-butyllithium in hexane sane (18 mL). The CO₂ bath was changed to ice bath for 20 min. The mixture was cooled back to −78° C., then N,N-diethyl-4-fluoro-2-(4-methylpyridin-3-yl)benzamide (4.05 g, 0.0129 mol) of Step A was added turning the solution reddish orange. The reaction mixture was placed in an ice bath for 15 min, and the color changed to orange with precipitation. The reaction was quenched with 1 N HCl (18 mL) at −40° C. Then additional 1 N HCl (60 mL) was added. The reaction mixture was filtered and was washed with EtOAc (10 mL), water (10 mL×3), EtOAc (5 mL×3) to give 3.6 g of a wet yellow solid. The solid was dried under air overnight to give 3.05 g of an off-white solid (91% yield). LCMS: 214.1 (M+1), 0.88 min. ¹H NMR (DMSO-d₆): δ 9.95 (s, 1H); 8.89 (d, 1H); 8.5 (d, 1H); 8.38 (m, 1H); 7.65 (d, 1H); 7.6 (m, 1H); 7.0 (s, 1H).

Step C

### (5E)-9-Fluorobenzo[h]isoquinoline-5,6-dione 5-oxime

To a 0° C. solution of 9-fluorobenzo[h]isoquinolin-6-ol (500.0 mg, 0.002345 mol) of Step B in N,N-dimethylformamide (10 mL, 0.1 mol) was added tert-butyl nitrite (340 μL, 0.0026 mol) and 4.0 M of hydrogen chloride in 1,4-dioxane (590 μL). After 15 min, a brown precipitates formed and the reaction was stirred overnight. EtOAc and water were added, and additional precipitate formed. The solid was filtered and washed with water×4, EtOAc×2 to give 515 mg of brown solid. The wet solid was dried under air overnight to give 390 mg solid (69% yield). LCMS: 243 (M+1), 1.13 min. ¹H NMR (DMSO-d₆) of the major isomer: δ 9.62 (s, 1H); 8.88 (d, 1H); 8.65 (brd, 1H); 8.4 (brd, 1H); 8.2 (brd, 1H); 7.42 (m, 1H).

Step D

### 2-tert-Butyl-9-fluoro-3H-benzo[h]imidazo[4,5-f]isoquinolin-3-ol

A mixture of (5E)-9-fluorobenzo[h]isoquinoline-5,6-dione 5-oxime (185.0 mg, 0.0007638 mol) of Step C, pivaldehyde (260 μL, 0.0024 mol) and ammonium acetate (350 mg, 0.0045 mol) in acetic acid (10 mL, 0.2 mol) was heated to reflux for 2.5 h. Acetic acid was rotovapped. Ethyl acetate and 1 N NaOH were added and the mixture was stirred for 15 min. The resulting mixture was filtered and washed with EtOAc, water×4 and EtOAc to give 170 mg of brown solid. The product was dried at 60° C. overnight to give 99 mg of a brown solid (46% yield). LCMS: 310.1 (M+1), 1.32 min. ¹H NMR (DMSO-d₆): δ 12.39 (brd, 1H); 10.18 (br s, 1H); 8.9 (brd, 1H); 8.8 (brd, 1H); 8.5 (brd, 2H); 7.6 (brd, 1H).

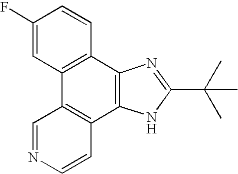

### Example 21

### 2-tert-Butyl-9-fluoro-3H-benzo[h]imidazo[4,5-f]isoquinoline

A suspension of 2-tert-butyl-9-fluoro-3H-benzo[h]imidazo[4,5-f]isoquinolin-3-ol (45.5 mg, 0.000132 mol) of Example 20 and zinc (342.0 mg, 0.005230 mol) in acetic acid (3 mL, 0.05 mol) was heated to reflux overnight. The reaction mixture was filtered and was washed with ethyl acetate and was rotovapped to give 100 mg of an orange oil. The oil was partitioned between ethyl acetate/THF and sat. sodium bicarbonate, washed with sat. NaCl. The organic phase was dried and rotovapped to give 60 mg of orange glass. The product was chromatographed (5% MeOH/CH₂Cl₂, 0.5% NH₄OH) to give 40 mg of off-white solid/glass (89% yield). LCMS: 294.1 (M+1), 1.18 min. ¹H NMR (CD₃OD): δ 10.11 (s, 1H); 8.99 (d, 1H); 8.81 (m, 1H); 8.78 (m, 2H); 7.79 (m, 1H).

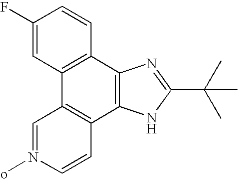

### Example 22

### 2-tert-Butyl-9-fluoro-3H-benzo[h]imidazo[4,5-f]isoquinoline 6-oxide

A solution of 2-tert-butyl-9-fluoro-3H-benzo[h]imidazo[4,5-f]isoquinoline (9.5 mg, 0.000032 mol) of Example 21 and m-chloroperbenzoic acid (20.0 mg, 0.0000892 mol) in methylene chloride (1 mL) and methanol (1 mL,) for 2 h. The mixture was partitioned between EtOAc (40 mL)/THF(40 mL) and sat. sodium bicarbonate×2, washed with sat. NaCl. The organic phase was dried and rotovapped to give a pale orange solid. The crude product was triturated with 1 mL of boiling DCE (1 mL) and EtOH (0.1 mL), and was washed with 110% EtOH/DCE (0.5 mL) to give 9.8 mg of off-white fine powder. 6.8 mg of final product was obtained after drying (72% yield). LCMS: 310.1 (M+1), 1.14 min. ¹H NMR (DMSO-d₆): δ 9.85 (s, 1H); 8.98 (m, 2H); 8.5 (br m, 2H); 7.75 (m, 1H).

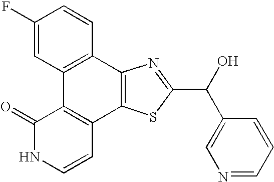

### Example 23

### 9-Fluoro-2-[pyridine(pyridine-3-yl)methyl]benzo[h][1,3]thiazolo[5,4-f]isoquinolin-7(6H)-one

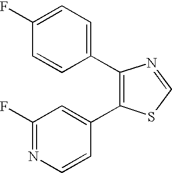 Step A

### 2-Fluoro-4-[4-(4-fluorophenyl)-1,3-thiazol-5-yl]pyridine

To a solution of thioformamide (3.00 g, 49.1 mmoles, Organic Preparations & Procedures International, 1999, 31 (6), 693-694) in THF (100 mL) was added a solution of 2-bromo-1-(4-fluorophenyl)-2-(2-fluoropyridin-4-yl)ethanone (7.00 g, 22.4 mmoles) in THF (10 mL). The mixture was stirred at room temperature for 2 hours, then diluted with aqueous NaHCO₃ and extracted with ethyl acetate. The organic extracts were washed with brine, dried over MgSO₄, filtered and concentrated. The crude product was purified on silica gel eluting with 30% ethyl acetate/hexane. The isolated product, contaminated with 1-(4-fluorophenyl)-2-(2-fluoropyridin-4-yl)ethanone, was then dissolved in ethanol and treated with sodium borohydride (0.51 g, 13 mmoles). After 20 minutes, the desired product was unchanged while the impurity had been reduced to 1-(4-fluorophenyl)-2-(2-fluoropyridin-4-yl)ethanol. The solution was concentrated on the rotovap and the residue was diluted with aqueous NaHCO₃ and extracted with ethyl acetate. The organic extracts were washed with brine, dried over MgSO₄, filtered, concentrated and then purified on silica gel eluting with 20% to 25% ethyl acetate/hexane. Pure fractions were combined and concentrated to provide the title compound as a pale yellow solid (3.00 g, 49%). LC/MS: 275.1 (M+H)⁺. ¹H NMR (CDCl₃) δ 8.92 (s, 1H), 8.18 (d, 1H), 7.50 (m, 2H), 7.12 (dt, 1H), 7.07 (m, 2H), 6.91 (t, 1H). 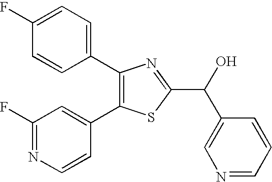
Step B

### [4-(4-Fluorophenyl)-5-(2-fluoropyridin-4-yl)-1,3-thiazol-2-yl](pyridin-3-yl)methanol

The product of Step A, 2-fluoro-4-[4-(4-fluorophenyl)-1,3-thiazol-5-yl]pyridine (0.150 g, 0.547 mmoles), was dissolved in THF (15 mL) and cooled to −78° C. under an atmosphere of nitrogen. n-Butyllithium (0.37 mL, 0.60 mmoles, 1.6 M solution in THF) was added dropwise upon which the solution turned dark orange. After 5 minutes, a solution of 3-pyridinecarboxaldehyde (0.064 g, 0.60 mmoles) in THF (1 mL) was added and the mixture was allowed to slowly warm to room temperature. The resulting dark green solution was quenched by addition of silica gel. The mixture was concentrated to a dry powder, loaded onto a silica gel column and eluted with ethyl acetate. Pure fractions were combined and concentrated to provide the title compound as a white solid (0.126 g, 60%). LC/MS: 382.0 (M+H)⁺. ¹H NMR (CDCl₃) δ 8.79 (d, 1H), 8.60 (dd, 1H), 8.15 (d, 1H), 7.91 (dt, 1H), 7.44 (m, 2H), 7.36 (m, 1H), 7.05 (m, 3H), 6.84 (s, 1H), 6.16 (s, 1H), 4.11 (m, 1H). ¹⁹F NMR (CDCl₃) δ −66.92 (s), −111.99 (m). 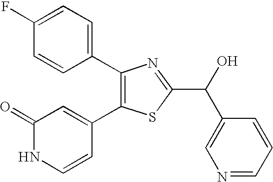
Step C

### 4-(4-(4-Fluorophenyl)-2-[hydroxy(pyridin-3-yl)methyl]-1,3-thiazol-5-yl]pyridin-2(1H)-one

The title compound was prepared following the procedure described for Example 8, Step C. LC/MS: 380.0 (M+H)⁺. ¹H NMR (DMSO-d₆) δ 11.67 (bs, 1H), 8.71 (d, 1H), 8.50 (dd, 1H), 7.86 (dt, 1H), 7.46 (m, 2H), 7.39 (dd, 1H), 7.33 (d, 1H), 7.20 (m, 3H), 6.28 (d, 1H), 6.06 (d, 1H), 5.92 (dd, 1H). ¹⁹F NMR (DMSO-d₆) δ −113.43 (m).

Step D

### 9-Fluoro-2-[hydroxy(pyridin-3-yl)methyl]benzo[h][1,3]thiazolo[5,4-f]isoquinolin-7(6H)-one

The title compound was prepared following the procedure described for Example 8, Step D. LC/MS: 378.1 (M+H)⁺. ¹H NMR (DMSO-d₆) δ 12.00 (bs, 1H), 9.97 (dd, 1H), 8.82 (s, 1H), 8.67 (dd, 1H), 8.52 (d, 1H), 7.93 (d, 1H), 7.61 (m, 2H), 7.43 (m, 2H), 6.86 (d, 1H), 6.33 (d, 1H). ¹⁹F NMR (DMSO-d₆) δ −111.52 (m).

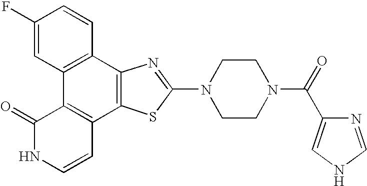

### Example 24

### 9-Fluoro-2-[4-(1H-imidazol-4-ylcarbonyl)piperazin-1-yl]benzo[h][1,3]thiazolo[5,4-f]isoquinolin-7(6H)-one

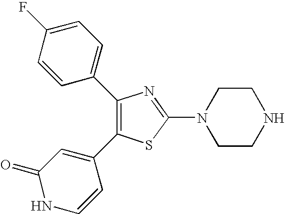 Step A

### 4-[4-(4-Fluorophenyl)-2-piperazin-1-yl-1,3-thiazol-5-yl]pyridin-2(1H)-one

A solution of 2-bromo-1-(4-fluorophenyl)-2-(2-fluoropyridin-4-yl)ethanone (1.01 g, 3.24 mmol) and piperazine-1-carbothioamide (520 mg, 3.6 mmol) in DMF (7.5 mL) was stirred at room temperature. After 115 hours, LC/MS showed complete conversion to the desired thiazole (LC/MS: 359, (M+H)⁺). The DMF was removed by rotary evaporation. THF (10 mL) and 4 M HCl (4 mL) were added to the residue and the resulting mixture was heated to 70° C. After 17 hours, LC/MS showed complete hydrolysis to the desired compound (LC/MS: 357, (M+H)⁺). The product was isolated by preparative HPLC/MS (0.75 g TFA salt, 49%). 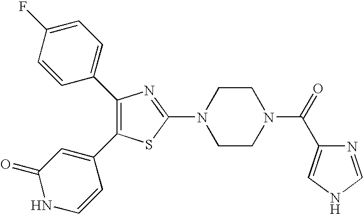
Step B

### 4-{4-(4-Fluorophenyl)-2-[4-(1H-imidazol-4-ylcarbonyl)piperazin-1-yl]-1,3-thiazol-5-yl}pyridin-2(1H)-one

The product of Step A (110 mg, TFA salt, 0.233 mmol), and 1H-imidazole-4-carboxylic acid (29 mg, 0.26 mmol), were stirred in DMF (1.5 mL) under nitrogen. N,N-Diisopropylethylamine (81 μL, 0.47 mmol) was added, then N-(3-dimethylaminopropyl)-N′-ethylcarbodiimide hydrochloride (47 mg, 0.24 mmol) was added and the mixture was stirred at room temperature. After 15 hours, LC/MS showed the desired product, (M+H)⁺ 451 as the main component. The product was isolated by preparative HPLC/MS to provide the title compound (108 mg, TFA salt, 82%). 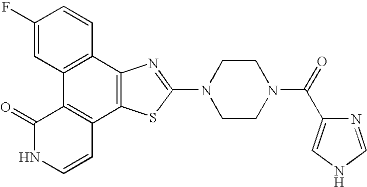
Step C

### 9-Fluoro-2-[4-(]H-imidazol-4-ylcarbonyl)piperazin-1-yl]benzo[h][1,3]thiazolo[5,4-f]isoquinolin-7(6H)-one

A solution of 4-{4-(4-fluorophenyl)-2-[4-(1H-imidazol-4-ylcarbonyl)piperazin-1-yl]-1,3-thiazol-5-yl}pyridin-2(1H)-one (108 mg, TFA salt, 0.20 mmol) in THF (90 mL) was stirred and exposed to UV light at a distance of 6 cm (Mineralight UVGL-25, 365 nm). After 16 hours, LC/MS showed the reaction was complete. The solution was concentrated on the rotovap to dryness. The residue was stirred in isopropanol (15 mL). The product was collected by filtration and dried to give the title compound as an off white powder (26 mg, 29%). LC/MS: 449 (M+H)⁺. ¹H NMR (DMSO-d₆) δ 11.71 (d, 1H, pyridone NH), 9.93 (dd, 1H), 8.60 (dd, 1H); 8.31 (bs, 1H); 7.93 (bs, 1H); 7.55 (m, 1H); 7.52 (t, 1H); 6.52 (dd, 1H); 3.62 (bs, 8H, NCH₂).

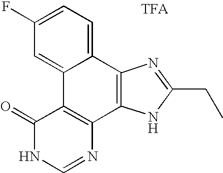

### Example 25

### 2-Ethyl-9-fluoro-3,6-dihydro-7H-benzo[f]imidazo[4,5-h]quinazolin-7-one, trifluoroacetate salt

Method A

To a solution of 9-fluoro-1-methoxybenzo[f]quinazolin-6-ol prepared according to the procedure for Example 18 through Step C (500 mg, 2.05 mmol) in acetic acid (8.5 mL) was added a solution of sodium nitrite (223 mg, 3.23 mmol) in water (2.5 mL). When the reaction was complete as determined by LCMS, the yellow solid was isolated by filtration, and was washed with water and air dried to provide crude 25-1 (550 mg, 98%). MS(ES) 274 (M+1).

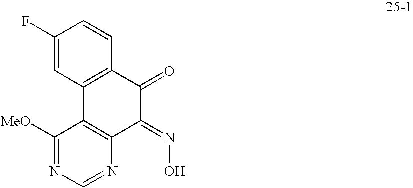

To 25-1 (500 mg, 2.0 mmol) in 1,4-dioxane (20 mL) was added a solution of sodium dithionite (2.80 g, 16.0 mmol) in water (20 mL) and ammonium hydroxide (0.7 mL). The reaction was stirred at room temperature for 1 hour. The mixture was partitioned between water and ethyl acetate, and the aqueous portion was extracted with two further volumes of ethyl acetate. The combined organic extracts were washed with water, brine, dried over sodium sulfate, filtered and concentrated to afford crude 25-2 (420 mg, 79%). ¹H NMR (CD₃OD, 400 MHz): δ 8.89 (dd, 1H); 8.81 (s, 1H); 8.38 (dd, 1H); 7.43 (dt, 1H); 4.33 (s, 3H). MS(ES) 261 (M+1).

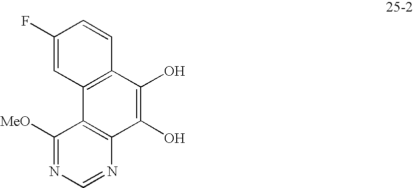

To a solution of 25-2 (420 mg, 1.6 mmol) in water (22 mL) and acetonitrile (9 mL) was added ceric ammonium nitrate (1.045 g, 1.9 mmol) and the reaction was stirred at room temperature for 1.5 hours. The acetonitrile was removed en vacuo and the product was extracted with ethyl acetate. The combined organic extracts dried over sodium sulfate, filtered and concentrated to afford crude 25-3 (393 mg, 93%). ¹H NMR (CD₃OD, 400 MHz): δ 8.77 (s, 1H); 8.28 (dd, 1H); 8.06 (dd, 1H); 7.26 (dt, 1H); 4.22 (s, 3H). MS(ES) 259 (M+1), 277 (M+H₂O+1).

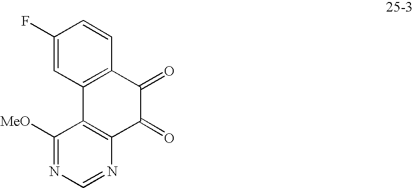

A mixture of 25-3 (106 mg, 0.41 mmol) in ethanol (6.1 mL) and c.HCl (2.8 mL) was heated to 55 degrees C. for 2.5 hours. The reaction was cooled to ambient temperature and the orange solid was isolated by filtration, was washed with water and dried to afford 25-4 (89 mg, 89%). ¹H NMR ((CD₃)₂SO, 400 MHz): δ 9.14 (dd, 1H); 8.38 (s, 1H); 8.12 (dd, 1H); 7.38 (dt, 1H). MS(ES) 245 (M+1), 263 (M+H₂O+1).

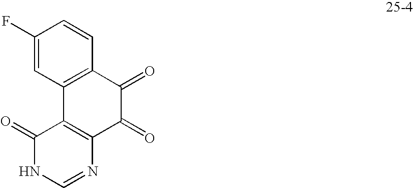

To 25-4 (7 mg, 0.03 mmol) and propionaldehyde (3 uL, 0.03 mmol) in methanol (0.1 mL) was added ammonium hydroxide (16 uL, 0.23 mmol). The reaction was stirred at room temperature for 1 hour. The mixture was concentrated to dryness, reconstituted in a mixture of DMSO and methanol, and purified by prep-LCMS to afford the title compound, as the TFA salt (5 mg, 45%). ¹H NMR (CD₃OD, 400 MHz): δ 9.74 (dd, 1H); 8.44 (dd, 1H); 8.41 (s, 1H); 7.64 (dt, 1H); 3.29 (m, 2H), 1.58 (t, 3H). MS(ES) 283 (M+1).

Method B

Imidazole formation for some analogous compounds was performed prior to hydrolysis.

Further compounds of the invention listed in Table 1 were prepared in a manner analogous to the procedure of Example 25.

TABLE 1

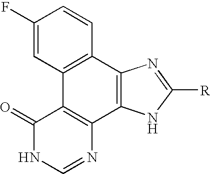

| Ex. | | | | |
|---|---|---|---|---|
| No. | Name | R | MS (ES)(M + 1) | Preparation |
| 26 | trans-2-(9-fluoro-7-oxo- 6,7-dihydro-3H- benzo[f]imidazo[4,5- h]quinazolin-2-yl) cyclopropanecarboxamide · 2TFA | 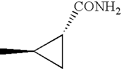 | 338 | Ex. 25 Method B |
| 27 | 1-(9-fluoro-7-oxo-6,7- dihydro-3H- benzo[f]imidazo[4,5- h]quinazolin-2-yl) cyclopropanecarboxylic acid · TFA | 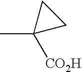 | 339 | Ex. 25 Method B |
| 28 | 2-[2-(dimethylamino)-1,1- dimethylethyl]-9-fluoro- 3,6-dihydro-7H- benzo[f]imidazo[4,5- h]quinazolin-7-one · 2TFA | 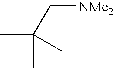 | 354 | Ex. 25 Method A |
| 29 | 4-ethyl-4-(9-fluoro-7-oxo- 6,7-dihydro-3H- benzo[f]imidazo[4,5- h]quinazolin-2- yl)hexanenitrile | 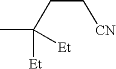 | 378 | Ex. 25 Method A |
| 30 | 4-ethyl-4-(9-fluoro-7-oxo- 6,7-dihydro-3H- benzo[f]imidazo[4,5- h]quinazolin-2- yl)hexanamide | 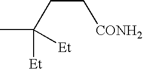 | 396 | Ex. 25 Method A |
| 31 | 2-(4-amino-1,1- dimethylbutyl)-9-fluoro- 3,6-dihydro-7H- benzo[f]imidazo[4,5- h]quinazolin-7-one · 2TFA | 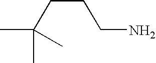 | 354 | Ex. 25 Method A |
| 32 | benzyl [1-(9-fluoro-7-oxo- 6,7-dihydro-3H- benzo[f]imidazo[4,5- h]quinazolin-2-yl)-1- methylethyl]carbamate ·TFA | 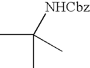 | 446 | Ex. 25 Method A |
| 33 | benzyl [2-(9-fluoro-7-oxo- 6,7-dihydro-3H- benzo[f]imidazo[4,5- h]quinazolin-2-yl)-2- methylpropyl]carbamate ·TFA | 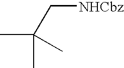 | 460 | Ex. 25 Method A |
| 34 | [2-(9-fluoro-7-oxo-6,7- dihydro-3H- benzo[f]imidazo[4,5- h]quinazolin-2-yl)-2- methylpropoxy]acetonitrile · TFA | 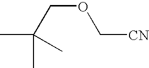 | 366 | Ex. 25 Method A |
| 35 | 2-(1-amino-1-methylethyl)- 9-fluoro-3,6-dihydro-7H- benzo[f]imidazo[4,5- h]quinazolin-7-one · 2TFA | 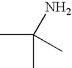 | 312 | Ex. 25 Method A |
| 36 | 4-(9-fluoro-7-oxo-6,7- dihydro-3H- benzo[f]imidazo[4,5- h]quinazolin-2- yl)butanenitrile · TFA | 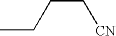 | 322 | Ex. 25 Method A |
| 37 | N-[1-(9-fluoro-7-oxo-6,7- dihydro-3H- benzo[f]imidazo[4,5- h]quinazolin-2-yl)-1- methylethyl]acetamide ·TFA | 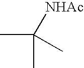 | 354 | Ex. 25 Method A |
| 38 | benzyl 4-(9-fluoro-7-oxo- 6,7-dihydro-3H- benzo[f]imidazo[4,5- h]quinazolin-2- yl)piperidine-1-carboxylate · TFA | 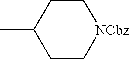 | 472 | Ex. 25 Method A |
| 39 | 3-(9-fluoro-7-oxo-6,7- dihydro-3H- benzo[f]imidazo[4,5- h]quinazolin-2- yl)propanenitrile | 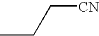 | 308 | Ex. 25 Method A |
| 40 | N-[2-(9-fluoro-7-oxo-6,7- dihydro-3H- benzo[f]imidazo[4,5- h]quinazolin-2-yl)-2- methylpropyl]urea · 2TFA | 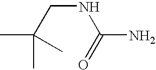 | 369 | Ex. 25 Method A |
| 41 | 4-(9-fluoro-7-oxo-6,7- dihydro-3H- benzo[f]imidazo[4,5- h]quinazolin-2-yl)-4- methylpentanenitrile | 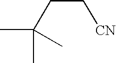 | 350 | Ex. 25 Method A |
| 42 | 2-(1-acetylpiperidin-4-yl)- 9-fluoro-3,6-dihydro-7H- benzo[f]imidazo[4,5- h]quinazolin-7-one · TFA | 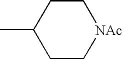 | 380 | Ex. 25 Method A |
| 43 | 3-(9-fluoro-7-oxo-6,7- dihydro-3H- benzo[f]imidazo[4,5- h]quinazolin-2-yl)-3- methylbutanenitrile | 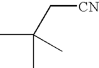 | 336 | Ex. 25 Method A |
| 43a | 9-fluoro-2-(trans-4- hydroxycyclohexyl)-3,6- dihydro-7H- benzo[f]imidazo[4,5- h]quinazolin-7-one | 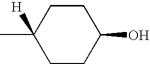 | 353 | Ex. 25 Method A |
| 43b | 9-fluoro-2-(cis-4- hydroxycyclohexyl)-3,6- dihydro-7H- benzo[f]imidazo[4,5- h]quinazolin-7-one | 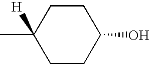 | 353 | Ex. 25 Method A |

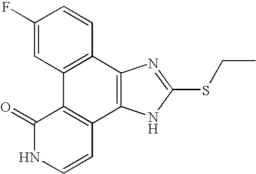

### Example 44

### 2-(Ethylthio)-9-fluoro-3,6-dihydro-7H-benzo [h]imidazo[4,5-f]isoquinolin-7-one

Step A

### 4-(4-Fluorophenyl)-5-(2-hydroxypyridin-4-yl)-1,3-dihydro-2H-imidazole-2-thione

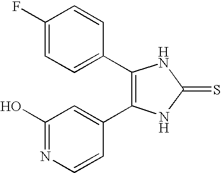

4-(4-Fluorophenyl)-5-(2-fluoropyridin-4-yl)-1,3-dihydro-2H-imidazole-2-thione (prepared as described in J. Med. Chem. 2003, 46, 3230-3244) (1.8 g, 0.0062 mol) was dissolved in acetic acid (50.0 mL, 0.879 mol) and water (10.0 mL, 0.555 mol) and stirred at 100° C. overnight. The mixture was cooled to room temperature to give a voluminous precipitate. The solids were collected. The acetic acid solution was concentrated to half the volume and the resulting solids were collected. The combined solid was dried in vacuo to give 4-(4-fluorophenyl)-5-(2-hydroxypyridin-4-yl)-1,3-dihydro-2H-imidazole-2-thione as a yellow orange colored solid (1.5 gm, 84%). LC/MS: 288 (M+H)⁺. ¹H NMR (DMSO-d₆) δ 12.75 (s, 1H), 12.60 (s, 1H), 7.45 (m, 2H), 7.25 (m, 3H), 6.40 (s, 1H), 5.82 (m, 1H).

Step B

### 4-[2-(Ethylthio)-4-(4-fluorophenyl)-1H-imidazol-5-yl]pyridin-2-ol

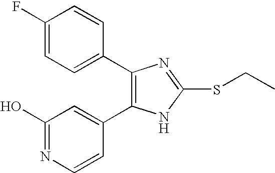

4-(4-Fluorophenyl)-5-(2-hydroxypyridin-4-yl)-1,3-dihydro-2H-imidazole-2-thione (0.125 g, 0.000435 mol) was dissolved in N,N-dimethylformamide (5.0 mL, 0.064 mol) and tetrahydrofuran (5.0 mL, 0.062 mol), then the potassium carbonate (0.18 g, 0.0013 mol) was added. To this stirring suspension the iodoethane (0.035 mL, 0.00044 mol) was added. The reaction was stirred for 4 h and was complete. This was diluted with THF and filtered to remove the solids. The filtrate was concentrated to remove the THF and DMF to give 4-[2-(ethylthio)-4-(4-fluorophenyl)-1H-imidazol-5-yl]pyridin-2-ol as an oil. LC/MS: 316 (M+H)⁺.

Step C

### 2-(Ethylthio)-9-fluoro-3,6-dihydro-7H-benzo[h]imidazo[4,5-f]isoquinolin-7-one

The crude oil 4-[2-(ethylthio)-4-(4-fluorophenyl)-1H-imidazol-5-yl]pyridin-2-ol (0.137 g, 0.000435 mol) was dissolved in methanol (75.0 mL, 1.85 mol) and the iodine (0.015 g, 0.000059 mol) was added. The reaction was irradiated in an open crystallizing dish with stirring to UV light (Mineralight UVL-56, 365 nm) and the reaction was monitored by HPLC. This was complete after stirring for 1.5 hs. The reaction was concentrated to give a semisolid residue. The crude product was purified by HPLC on C-18 column eluting acetonitrile:water gradient with 0.1% TFA to give the title compound as a white amorphous solid (26.0 mg, 19%). LC/MS: 314 (M+H)⁺. ¹H NMR (DMSO-d₆) δ 11.65 (bs, 1H), 10.03 (d, 1H), 8.42 (m, 1H), 7.55 (m, 2H), 7.18 (m, 1H), 3.35 (q, 2H), 1.20 (t, 3H).

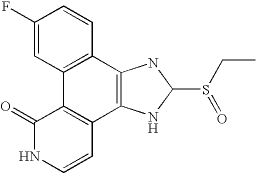

### Example 45

### 2-(Ethylsulfinyl)-9-fluoro-3,6-dihydro-7H-benzo[h]imidazo[4,5-f]isoquinolin-7-one

2-(Ethylthio)-9-fluoro-3,6-dihydro-7H-benzo[h]imidazo[4,5-f]isoquinolin-7-one (0.055 g, 0.00018 mol) was dissolved in tetrahydrofuran (5.0 mL, 0.062 mol) and the m-chloroperbenzoic acid (0.091 g, 0.00053 mol) was added. The reaction became a cloudy suspension after a few minutes. This was monitored by HPLC. After stirring for 2 h the reaction was incomplete. Additional MCPBA was added slowly over several hs until all the starting material was consumed giving the sulfone and sulfoxide products. The reaction was concentrated to give a semisolid residue. Product appears to be mostly the sulfoxide based on the LC/MS. The crude product was purified by HPLC on C-18 column eluting acetonitrile:water gradient with 0.1% TFA to give the title compound as a white amorphous solid (22.0 mg, 37%). LC/MS: 330 (M+H)⁺. ¹H NMR (DMSO-d₆) δ 11.85 (bs, 1H), 10.05 (d, 1H), 8.65 (bm, 1H), 7.60 (m, 2H), 7.3 (bm, 1H), 3.45 (m, 1H), 3.30 (m, 1H), 1.19 (dt, 3H).

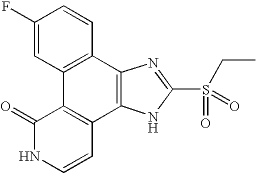

### Example 46

### 2-(Ethylsulfonyl)-9-fluoro-3,6-dihydro-7H-benzo[h]imidazo[4,5-f]isoquinolin-7-one

2-(Ethylthio)-9-fluoro-3,6-dihydro-7H-benzo[h]imidazo[4,5-f]isoquinolin-7-one (0.055 g, 0.00018 mol) was dissolved in tetrahydrofuran (5.0 mL, 0.062 mol) and the m-chloroperbenzoic acid (0.091 g, 0.00053 mol) was added. The reaction became a cloudy suspension after a few minutes. This was monitored by HPLC. After stirring for 2 h the reaction was incomplete. Additional MCPBA was added slowly over several hs until all the starting material was consumed giving the sulfone and sulfoxide products. The reaction was concentrated to give a semisolid residue. Product appears to be mostly the sulfone based on the LC/MS. The crude product was purified by HPLC on C-18 column eluting acetonitrile:water gradient with 0.1% TFA to give the title compound as a white amorphous solid (14.0 mg, 22%). LC/MS: 346 (M+H)⁺. ¹H NMR (DMSO-d₆) δ 11.9 (bs, 1H), 10.05 (d, 1H), 8.7 (bs, 1H), 7.65 (m, 2H), 7.23 (m, 1H), 3.62 (q, 2H), 1.13 (t, 3H).

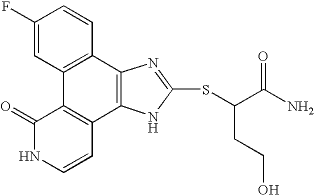

### Example 47

### 2-[(9-Fluoro-7-oxo-6,7-dihydro-3H-benzo[h]imidazo[4,5-f]isoquinolin-2-yl)thiol]-4-hydroxybutanamide

9-Fluoro-2-[(2-oxotetrahydrofuran-3-yl)thio]-3,6-dihydro-7H-benzo[h]imidazo[4,5-f]isoquinolin-7-one (Ex. 52), (0.025 g, 0.000068 mol) was dissolved in 2.0 M of ammonia in tetrahydrofuran (2.0 mL). The reaction was stirred for 1.5 h at room temperature. The reaction was concentrated in vacuo to give the crude product. The product was purified by HPLC on a C-18 column eluting acetonitrile; water gradient containing 0.1% TFA to give the title compound as a white amorphous solid (0.009 g, 30%). LC/MS: 387 (M+H)⁺. ¹H NMR (DMSO-d₆) δ 11.70 (bs, 1H), 9.95 (d, 1H), 8.38 (m, 1H), 7.78 (s, 1H), 7.52 (m, 2H), 7.25 (s, 1H), 7.10 (m, 1H), 4.43 (m, 1H), 3.53 (m, 1H), 3.47 (m, 1H), 2.07 (m, 1H), 1.95 (m, 1H).

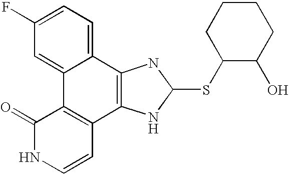

### Example 48

### 9-Fluoro-2-[(2-hydroxycyclohexyl)thiol]-3,6-dihydro-7H-benzo [h]imidazo[4,5-f]isoquinolin-7-one

Using a procedure analogous to Example 44 but using cyclohexene oxide in Step B, 4-{4-(4-fluorophenyl)-2-[(2-hydroxycyclohexyl)thio]-1H-imidazol-5-yl}pyridine-2(1H)-one was prepared and converted to the title compound as an amorphous solid (0.007 g, 36%). LC/MS: 384 (M+H)⁺. ¹H NMR (DMSO-d₆) δ 11.78 (bs, 1H), 10.02 (d, 1H), 8.42 (m, 1H), 7.58 (m, 2H), 7.15 (m, 1H), 3.70 (m, 1H), 3.53 (m, 1H), 2.17 (m, 1H), 1.93 (m, 1H), 1.7-1.2 (m, 6H).

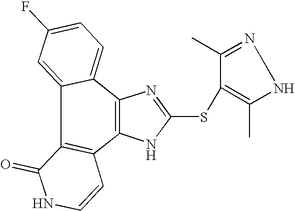

### Example 49

### 2-[(3,5-Dimethyl-1H-pyrazol-4-yl)thiol]-9-fluoro-3,6-dihydro-7H-benzolimidazo[4,5-f]isoquinolin-7-one

Step A

Using a procedure analogous to Example 44 but using 3-chloropentane-2,4-dione in Step B, 3-{[4-(4-fluorophenyl)-5-(2-hydroxypyridin-4-yl)-1H-imidazol-2-yl]thio}pentane-2,4-dione was prepared as a crude solid residue (0.067 gm, 100%). LC/MS: 386 (M+H)⁺.

Step B

Hydrazine hydrate (0.024 mL, 0.00048 mol) was added to a solution of 3-{[4-(4-fluorophenyl)-5-(2-hydroxypyridin-4-yl)-1H-imidazol-2-yl]thio}pentane-2,4-dione (0.08 g, 0.0002 mol) and potassium carbonate (0.072 gm, 0.052 mol) in DMF 3.0 ml at room temperature for 1 h. The reaction was diluted THF and filtered to remove the solids and concentrated in vacuo to give 4-[2-[(3,5-dimethyl-4H-pyrazol-4-yl)thio]-4-(4-fluorophenyl)-1H-imidazol-5-yl]pyridine-2-ol a semisolid. LC/MS: 382 (M+H)⁺.

Step C

Using a procedure analogous to Example 1 but using 4-[2-[(3,5-dimethyl-4H-pyrazol-4-yl)thio]-4-(4-fluorophenyl)-1H-imidazol-5-yl]pyridine-2-ol in Step C, the title compound was prepared as an off white amorphous solid (0.012 gm, 15%), LC/MS: 380 (M+H)⁺. ¹H NMR (DMSO-d₆) δ 11.70 (bs, 1H), 10.00 (d, 1H), 8.42 (m, 1H), 7.52 (m, 2H), 7.19 (m, 1H), 2.23 (s, 6H).

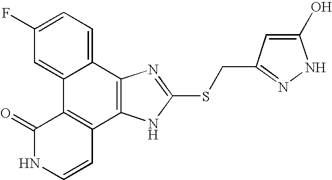

### Example 50

### 9-Fluoro-2-{[(5-hydroxy-1H-pyrazol-3-yl)methyl]thio}-3,6-dihydro-7H-benzo[h]imidazo[4,5-f]isoquinolin-7-one

Step A

Using a procedure analogous to Example 44 but using ethyl 4-bromo-3-oxobutanoate in Step B, ethyl 4-{[4-(4-fluorophenyl)-5-(2-oxo-1,2-dihydropyridin-4-yl)-1H-imidazol-2-yl]thio}-3-oxobutanoate was prepared as a crude solid residue (0.12 gm, 90%). LC/MS: 416 (M+H)⁺.

Step B

Using a procedure analogous to Example 49 but using ethyl 4-{[4-(4-fluorophenyl)-5-(2-oxo-1,2-dihydropyridin-4-yl)-1H-imidazol-2-yl]thio}-3-oxobutanoate in Step B, 4-(4-(4-fluorophenyl)-2-{[(5-oxo-4,5-dihydro-1H-pyrazol-3-yl)methyl]thio}-1H-imidazol-5-yl)pyridine-2(1H)-one was prepared as a crude solid residue (0.12 gm, 90%). LC/MS: 416 (M+H)⁺.

Step C

Using a procedure analogous to Example 1, Step C, but using 4-(4-(4-fluorophenyl)-2-{[(5-oxo-4,5-dihydro-1H-pyrazol-3-yl)methyl]thio}-1H-imidazol-5-yl)pyridine-2(1H)-one, the title compound was prepared as a crude solid residue (0.12 gm, 24%). LC/MS: 382 (M+H)⁺, ¹H NMR (DMSO-d₆) δ 11.68 (bs, 1H), 10.05 (d, 1H), 8.45 (m, 1H), 7.58 (m, 2H), 7.19 (m, 114), 5.28 (s, 1H), 4.52 (s, 2H).

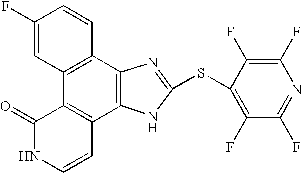

### Example 51

### 9-Fluoro-2-[(2,3,5,6-tetrafuoropyridin-4-yl)thiol]-3,6-dihydro-7H-benzolhlimidazo[4,5-f]isoquinolin-7-one

Using a procedure analogous to Example 44 but using 4-bromo-2,3,5,6-tetrafluoropyridine in Step B, the title compound was prepared as an amorphous solid residue (0.015 gm, 24%). LC/MS: 435 (M+H)⁺. ¹H NMR (DMSO-d₆) δ 11.82 (bs, 1H), 10.02 (d, 1H), 8.40 (m, 1H), 7.60 (m, 2H), 7.12 (m, 1H).

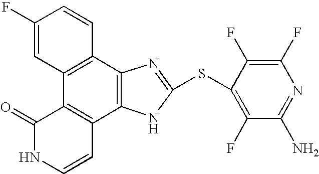

### Example 52

### 2-[(2,6-Diamino-3,5-difluoropyridin-4-yl)thio]-9-fluoro-3,6-dihydro-7H-benzo[h]imidazo[4,5-f]isoquinolin-7-one

9-Fluoro-2-[(2,3,5,6-tetrafluoropyridin-4-yl)thio]-3,6-dihydro-7H-benzo[h]imidazo[4,5-f]isoquinolin-7-one (Ex. 51) (0.02 gm, 0.00046 mol) was taken up in aqueous ammonia hydroxide (2 ml) in a sealed tube and heated in the microwave to 150° C. for 3 hs. The reaction was concentrated, taken up in DMF and made acidic with TFA. The product was purified by HPLC on a C-18 column eluting acetonitrile; water gradient containing 0.1% TFA to give the title compound as a white amorphous solid (0.006 g, 30%). LC/MS: 429 (M+H)⁺. ¹H NMR (DMSO-d₆) δ 11.75 (bs, 1H), 10.05 (d, 1H), 8.42 (m, 1H), 7.59 (m, 2H), 7.19 (m, 1H).

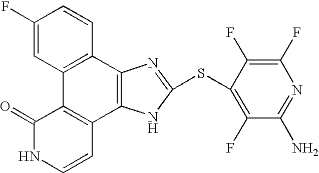

### Example 53

### 2-[(2-Amino-3,5,6-trifluoropyridin-4-yl)thio]-9-fluoro-3,6-dihydro-7H-benzo[h]imidazo[4,5-f]isoquinolin-7-one

9-Fluoro-2-[(2,3,5,6-tetrafluoropyridin-4-yl)thio]-3,6-dihydro-7H-benzo[h]imidazo[4,5-f]isoquinolin-7-one (Ex. 51) (0.02 gm, 0.00046 mol) was taken up in aqueous ammonia hydroxide (2 ml) in a sealed tube and heated in the microwave to 150° C. for 70 minutes. The reaction was concentrated, taken up in DMF and made acidic with TFA. The product was purified by HPLC on a C-18 column eluting acetonitrile; water gradient containing 0.1% TFA to give the title compound as a white amorphous solid (0.006 g, 30%). LC/MS: 432 (M+H)⁺. ¹H NMR (DMSO-d₆) δ 11.79 (bs, 1H), 10.05 (d, 1H), 8.42 (m, 1H), 7.50 (m, 2H), 7.15 (m, 1 h), 6.7 (bs, 2H).

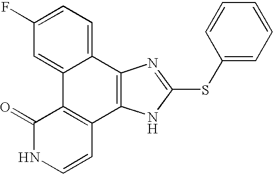

### Example 53a

### 9-Fluoro-2-(phenylthio)-3,6-dihydro-7H-benzo[h]imidazo[4,5-f]isoquinolin-7-one

Step A

The 4-[5-(4-fluorophenyl)-2-thioxo-2,3-dihydro-1H-imidazol-4-yl]pyridin-2(1H)-one (55.0 mg, 0.000191 mol) from Example 44 step A was combined with bromobenzene (45 mg, 0.00029 mol) in toluene (3.0 mL, 0.028 mol), ethanol (0.5 mL, 0.008 mol), DMF (1 mL), and sodium carbonate (59 mg, 0.00056 mol) in water (0.5 mL, 0.03 mol). The mixture was degassed with N₂ and tetrakis(triphenylphosphine)palladium(0) (39 mg, 0.000033 mol) was added. The mixture was heated in microwave at 150° C. for 30 min, twice. The reaction mixture was concentrated in vacuo to remove the organic solvents and the remaining residue was purified by HPLC on a C-18 column eluting with an acetonitirle: water gradient containing 0.1% TFA to give 4-[4-(4-fluorophenyl)-2-(phenylthio)-1H-imidazol-5-yl]pyridin-2(1H)-one as a white amorphous solid (43%, MS 364 (M+H)⁺, ¹H NMR (DMSO-d₆) δ 7.5 (m, 2H), 7.44-7.2 (m, 8H), 6.39 (s, 1H), 6.20 (d, 1H).

Step B

Using a procedure analogous to Example 44, Step C, but using, 4-[4-(4-fluorophenyl)-2-(phenylthio)-1H-imidazol-5-yl]pyridin-2(1H)-one, the title compound was prepared as a crude solid residue (0.12 gm, 24%). LC/MS: 362 (M+H)⁺, ¹H NMR (DMSO-d₆) δ 11.8 (d, 1H), 10.05 (d, 1H), 8.44 (m, 1H), 7.6 (m, 2H), 7.43-7.19 (m, 6H).

Table 2 below contains further examples of the present invention.

TABLE 2

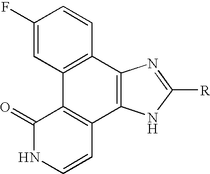

| Ex. No. | R | Mass Spec | Compound Name | Preparation |
|---|---|---|---|---|
| 54 | —S—CH₂—C₆H₅ | 376 (M + H)⁺ | 2-(benzylthio)-9-fluoro-3,6-dihydro- | Ex. 44 |
| | | | 7H-benzo[h]imidazo[4,5-f]isoquinolin- | |
| | | | 7-one | |
| 55 | —S(O)—CH₂—C₆H₅ | 392 (M + H)³⁰ | 2-(benzylsulfinyl)-9-fluoro-3,6- | Ex. 44 |
| | | | dihydro-7H-benzo[h]imidazo[4,5- | |
| | | | f]isoquinolin-7-one | |
| 56 | —S(O)₂—CH₂—C₆H₅ | 408 (M + H)⁺ | 2-(benzylsulfonyl)-9-fluoro-3,6- | Ex. 44 |
| | | | dihydro-7H-benzo[h]imidazo[4,5- | |
| | | | f]isoguinolin-7-one | |
| 57 | —S—CH₂—C₆H₄(3-CN) | 401 (M + H)⁺ | 3-{[(9-fluoro-7-oxo-6,7-dihydro-3H- | Ex. 44 |
| | | 801 | benzo[h]imidazo[4,5-f]isoquinolin-2- | |
| | | (2M + H)⁺ | yl)thio]methyl}benzonitrile | |
| 58 | —S—(CH₂)₂—OCH₃ | 344 (M + H)⁺ | 9-fluoro-2-[(2-methoxyethyl)thio]-3,6- | Ex. 44 |
| | | | dihydro-7H-benzo[h]imidazo[4,5- | |
| | | | f]isoquinolin-7-one | |
| 59 | 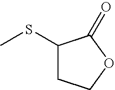 | 370 (M + H)⁺ | 9-fluoro-2-[(2-oxotetrahydrofuran-3- yl)thio]-3,6-dihydro-7H- benzo[h]imidazo[4,5-f]isoquinolin-7- one | Ex. 44 |
| 60 | —S—(CH₂)₂-Pthalimide | 459 (M + H)⁺ | 2-{2-[(9-fluoro-7-oxo-6,7-dihydro-3H- | Ex. 44 |
| | | | benzo[h]imidazo[4,5-f]isoquinolin-2- | |
| | | | yl)thio]ethyl}-1H-isoindole-1,3(2H)- | |
| | | | dione | |
| 61 | —S—CH₂—(3-C₅H₄N) | 377 (M + H)⁺ | 9-fluoro-2-[(pyridin-3-ylmethyl)thio]- | Ex. 44 |
| | | | 3,6-dihydro-7H-benzo[h]imidazo[4,5- | |
| | | | f]isoguinolin-7-one | |
| 62 | —S—CH-(C₂H₅)₂ | 356 (M + H)⁺ | 2-[(1-ethylpropyl)thio]-9-fluoro-3,6- | Ex. 44 |
| | | | dihydro-7H-benzo[h]imidazo[4,5- | |
| | | | f]isoquinolin-7-one | |
| 63 | —S(O)—CH—(C₂H₅)₂ | 372 (M + H)⁺ | 2-[(1-ethylpropyl)sulfinyl]-9-fluoro- | Ex. 44 |
| | | | 3,6-dihydro-7H-benzo[h]imidazo[4,5- | |
| | | | f]isoquinolin-7-one | |
| 64 | —S(O)₂—CH—(C₂H₅)₂ | 388 (M + H)⁺ | 2-[(1-ethylpropyl)sulfonyl]-9-fluoro- | Ex. 44 |
| | | | 3,6-dihydro-7H-benzo[h]imidazo[4,5- | |
| | | | f]isoquinolin-7-one | |
| 65 | —S—CH₂—C₆H₃(3,5-(OCH₃)₂) | 436 (M + H)⁺ | 2-[(3,5-dimethoxybenzyl)thio]-9- | Ex. 44 |
| | | | fluoro-3,6-dihydro-7H- | |
| | | | benzo[h]imidazo[4,5-f]isoquinolin-7- | |
| | | | one | |
| 66 | —S—CH₂—CO₂—C₂H₅ | 372 (M + H)⁺ | ethyl [(9-fluoro-7-oxo-6,7-dihydro-3H- | Ex. 44 |
| | | | benzo[h]imidazo[4,5-f]isoquinolin-2- | |
| | | | yl)thio]acetate | |
| 67 | —S—CH—(CH₃)₂ | 328 (M + H)⁺ | 9-fluoro-2-(isopropylthio)-3,6-dihydro- | Ex. 44 |
| | | | 7H-benzo[h]imidazo[4,5-f]isoquinolin- | |
| | | | 7-one | |
| 68 | —S—CH₂—CO₂H | 344 (M + H)⁺ | [(9-fluoro-7-oxo-6,7-dihydro-3H- | Ex. 44 |
| | | | benzo[h]imidazo[4,5-f]isoquinolin-2- | |
| | | | yl)thio]acetic acid | |
| 69 | —S—CH(CH₃)—C₆H₅ | 390 (M + H)⁺ | 9-fluoro-2-[(1-phenylethyl)thio]-3,6- | Ex. 44 |
| | | | dihydro-7H-benzo[h]imidazo[4,5- | |
| | | | f]isoquinolin-7-one | |
| 70 | —S—CH₂—CN | 325 (M + H)⁺ | [(9-fluoro-7-oxo-6,7-dihydro-3H- | Ex. 44 |
| | | 649 | benzo[h]imidazo[4,5-f]isoquinolin-2- | |
| | | (2M + H)⁺ | yl)thio]acetonitrile | |
| 71 | —S(O)—CH₂-CN | 341 (M + H)⁺ | [(9-fluoro-7-oxo-6,7-dihydro-3H- | Ex. 44 |
| | | | benzo[h]imidazo[4,5-f]isoquinolin-2- | |
| | | | yl)sulfinyl]acetonitrile | |
| 72 | —SCH(CH₃)—CONH—C₆H₅ | 433 (M + H)⁺ | 2-[(9-fluoro-7-oxo-6,7-dihydro-3H- | Ex. 44 |
| | | | benzo[h]imidazo[4,5-f]isoquinolin-2- | |
| | | | yl)thio]-N-phenylpropanamide | |
| 73 | —S—(CH₂)₃—OH | 344 (M + H)⁺ | 9-fluoro-2-[(3-hydroxypropyl)thio]- | Ex. 44 |
| | | | 3,6-dihydro-7H-benzo[h]imidazo[4,5- | |
| | | | f]isoquinolin-7-one | |
| 74 | —S—CH₂—CO—CF₃ | 396 (M + H)⁺ | 9-fluoro-2-[(3,3,3-trifluoro-2- | Ex. 44 |
| | | 791 | oxopropyl)thio]-3,6-dihydro-7H- | |
| | | (2M + H)⁺ | benzo[h]imidazo[4,5-f]isoquinolin-7- | |
| | | | one | |
| 75 | —S—(CH₂)₃—CO₂—C₂H₅ | 400 (M + H)⁺ | ethyl 4-[(9-fluoro-7-oxo-6,7-dihydro- | Ex. 44 |
| | | | 3H-benzo[h]imidazo[4,5-f]isoquinolin- | |
| | | | 2-yl)thio]butanoate | |
| 76 | —S—(CH₂)₂—NH₂ | 329 (M + H)⁺ | 2-[(2-aminoethyl)thio]-9-fluoro-3,6- | Ex. 44 |
| | | | dihydro-7H-benzo[h]imidazo[4,5- | |
| | | | f]isoquinolin-7-one | |
| 77 | —S—CH₂—C₆H₁₁ | 382 (M + H)⁺ | 2-[(cyclohexylmethyl)thio]-9-fluoro- | Ex. 44 |
| | | | 3,6-dihydro-7H-benzo[h]imidazo[4,5- | |
| | | | f]isoquinolin-7-one | |
| 78 | —S—(CH₂)—CO—NH₂ | 343 (M + H)⁺ | 2-[(9-fluoro-7-oxo-6,7-dihydro-3H- | Ex. 44 |
| | | | benzo[b]imidazo[4,5-f]isoquinolin-2- | |
| | | | yl)thio]acetamide | |
| 79 | —S—C₆H₁₁ | 368 (M + H)⁺ | 2-(cyclohexylthio)-9-fluoro-3,6- | Ex. 44 |
| | | | dihydro-7H-benzo[h]imidazo[4,5- | |
| | | | f]isoquinolin-7-one | |
| 80 | —SCH₂CH—(OCH₃)₂ | 374 (M + H)⁺ | 2-[(2,2-dimethoxyethyl)thio]-9-fluoro- | Ex. 44 |
| | | | 3,6-dihydro-7H-benzo[h]imidazo[4,5- | |
| | | | f]isoquinolin-7-one | |
| 81 | —SCH₂CO—C(CH₃)₃ | 384 (M + H)⁺ | 2-[(3,3-dimethyl-2-oxobutyl)thio]-9- | Ex. 44 |
| | | | fluoro-3,6-dihydro-7H- | |
| | | | benzo[h]imidazo[4,5-f]isoquinolin-7- | |
| | | | one | |
| 82 | —SCH(CH₃)—CO₂C₂H₅ | 386 (M + H)⁺ | ethyl 2-[(9-fluoro-7-oxo-6,7-dihydro- | Ex. 44 |
| | | | 3H-benzo[h]imidazo[4,5-f]isoquinolin- | |
| | | | 2-yl)thio]propanoate | |
| 83 | —S—(CH₂)₃—CN | 353 (M + H)⁺ | 4-[(9-fluoro-7-oxo-6,7-dihydro-3H- | Ex. 44 |
| | | | benzo[h]imidazo[4,5-f]isoquinolin-2- | |
| | | | yl)thio]butanenitrile | |
| 84 | —SCH(C₂H₅)—CO₂C₂H₅ | 400 (M + H)⁺ | ethyl 2-[(9-fluoro-7-oxo-6,7-dihydro- | Ex. 44 |
| | | | 3H-benzo[h]imidazo[4,5-f]isoquinolin- | |
| | | | 2-yl)thio]butanoate | |
| 85 | —SCH((CH₂)₂OH)— | 415 (M + H)⁺ | 2-[(9-fluoro-7-oxo-6,7-dihydro-3H- | Ex. 44 |
| | CON(CH₃)₂ | | benzo[h]imidazo[4,5-f]isoquinolin-2- | |
| | | | yl)thio]-4-hydroxy-N,N- | |
| | | | dimethylbutanamide | |
| 86 | —S—CH₂—C₆H₄(3-CO₂CH₃) | 434 (M + H)⁺ | methyl 3-{[(9-fluoro-7-oxo-6,7- | Ex. 44 |
| | | | dihydro-3H-benzo[h]imidazo[4,5- | |
| | | | f]isoquinolin-2- | |
| | | | yl)thio]methyl}benzoate | |
| 87 | 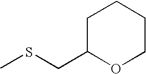 | 384 (M + H)⁺ | 9-fluoro-2-[(tetrahydro-2H-pyran-2- ylmethyl)thio]-3,6-dihydro-7H- benzo[h]imidazo[4,5-f]isoquinolin-7- one | Ex. 44 |
| 88 | —S—(CH₂)₃—CO₂H | 372 (M + H)⁺ | 4-[(9-fluoro-7-oxo-6,7-dihydro-3H- | Ex. 44 |
| | | | benzo[h]imidazo[4,5-f]isoquinolin-2- | |
| | | | yl)thio]butanoic acid | |
| 89 | —S—(CH₂)₃—CONH₂ | 371 (M + H)⁺ | 4-[(9-fluoro-7-oxo-6,7-dihydro-3H- | Ex. 44 |
| | | | benzo[h]imidazo[4,5-f]isoquinolin-2- | |
| | | | yl)thio]butanamide | |
| 90 | —SCN | 311 (M + H)⁺ | 9-fluoro-7-oxo-6,7-dihydro-3H- | Ex. 44 |
| | | | benzo[h]imidazo[4,5-f]isoquinolin-2-yl | |
| | | | thiocyanate | |
| 91 | —SCH(CH₃)—CN | 339 (M + H)⁺ | 2-[(9-fluoro-7-oxo-6,7-dihydro-3H- | Ex. 44 |
| | | | benzo[h]imidazo[4,5-f]isoquinolin-2- | |
| | | | yl)thio]propanenitrile | |
| 92 | —SCH₂C—(CH₃)₂—(OH) | 358 (M + H)⁺ | 9-fluoro-2-[(2-hydroxy-2- | Ex. 44 |
| | | | methylpropyl)thio]-3,6-dihydro-7H- | |
| | | | benzo[h]imidazo[4,5-f]isoquinolin-7- | |
| | | | one | |
| 93 | —SCH₂C—(CH₃)₂—(CH₂OH) | 372 (M + H)⁺ | 9-fluoro-2-[(3-hydroxy-2,2- | Ex. 44 |
| | | | dimethylpropyl)thio]-3,6-dihydro-7H- | |
| | | | benzo[h]imidazo[4,5-f]isoquinolin-7- | |
| | | | one | |
| 94 | 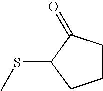 | 368 (M + H)⁺ | 9-fluoro-2-[(2-oxocyclopentyl)thio]- 3,6-dihydro-7H-benzo[h]imidazo[4,5- f]isoquinolin-7-one | Ex. 44 |
| 95 | 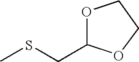 | 372 (M + H)⁺ | 2-[(1,3-dioxolan-2-ylmethyl)thio]-9- fluoro-3,6-dihydro-7H- benzo[h]imidazo[4,5-f]isoquinolin-7- one | Ex. 44 |
| 96 | —SCH((CH₂)₂OH)- | 415 (M + H)⁺ | N-ethyl-2-[(9-fluoro-7-oxo-6,7- | Ex. 44 |
| | CONHC₂H₅ | | dihydro-3H-benzo[h]imidazo[4,5- | |
| | | | f]isoquinolin-2-yl)thio]-4- | |
| | | | hydroxybutanamide | |
| 97 | —S—(CH₂)₂—OH | 330 (M + H)⁺ | 9-fluoro-2-[(2-hydroxyethyl)thio]-3,6- | Ex. 44 |
| | | | dihydro-7H-benzo[h]imidazo[4,5- | |
| | | | f]isoquinolin-7-one | |
| 98 | 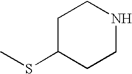 | 369 (M + H)⁺ | 9-fluoro-2-(piperidin-4-ylthio)-3,6- dihydro-7H-benzo[h]imidazo[4,5- f]isoquinolin-7-one | Ex. 44 |
| 99 | —S—(CH₂)₂—CN | 339 (M + H)⁺ | 3-[(9-fluoro-7-oxo-6,7-dihydro-3H- | Ex. 44 |
| | | | benzo[h]imidazo[4,5-f]isoquinolin-2- | |
| | | | yl)thio]propanenitrile | |
| 100 | 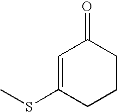 | 380 (M + H)⁺ | 9-fluoro-2-[(3-oxocyclohex-1-en-1- yl)thio]-3,6-dihydro-7H- phenanthro[9,10-d]imidazol-7-one | Ex. 44 |
| 101 | —S—(4-C₅H₄N) | 363 (M + H)⁺ | 9-fluoro-2-(pyridin-4-ylthio)-3,6- | Ex. 44 |
| | | | dihydro-7H-benzo[h]imidazo[4,5- | |
| | | | f]isoquinolin-7-one | |
| 102 | 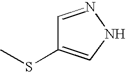 | 352 (M + H)⁺ | 9-fluoro-2-(1H-pyrazol-4-ylthio)-3,6- dihydro-7H-benzo[h]imidazo[4,5- f]isoquinolin-7-one | Ex. 49 |
| 103 | —S(O)—CH₂—C(CH₃)₂—(OH) | 374 (M + H)⁺ | 9-fluoro-2-[(2-hydroxy-2- | Ex. 44 |
| | | | methylpropyl)sulfinyl]-3,6-dihydro- | |
| | | | 7H-benzo[h]imidazo[4,5-f]isoquinolin- | |
| | | | 7-one | |
| 104 | —S(O)—(CH₂)₂CN | 355 (M + H)⁺ | 3-[(9-fluoro-7-oxo-6,7-dihydro-3H- | Ex. 44 |
| | | | benzo[h]imidazo[4,5-f]isoquinolin-2- | |
| | | | yl)sulfinyl]propanenitrile | |
| 105 | —SCH₂CH(OH)—C(CH₃)₃ | 386 (M + H)⁺ | 9-fluoro-2-[(2-hydroxy-3,3- | Ex. 44 |
| | | | dimethylbutyl)thio]-3,6-dihydro-7H- | |
| | | | benzo[h]imidazo[4,5-f]isoquinolin-7- | |
| | | | one | |
| 106 | —SCH₂C(O)—CH₃ | 342 (M + H)⁺ | 9-fluoro-2-[(2-oxopropyl)thio]-3,6- | Ex. 44 |
| | | | dihydro-7H-benzo[h]imidazo[4,5- | |
| | | | f]isoquinolin-7-one | |
| 107 | —S(O)—(4-C₅H₄N) | 379 (M + H)⁺ | 9-fluoro-2-(pyridin-4-ylsulfinyl)-3,6- | Ex. 44 |
| | | | dihydro-7H-benzo[h]imidazo[4,5- | |
| | | | f]isoquinolin-7-one | |
| 108 | —S(O)—(CH₂)₃CN | 369 (M + H)⁺ | 4-[(9-fluoro-7-oxo-6,7-dihydro-3H- | Ex. 44 |
| | | | benzo[h]imidazo[4,5-f]isoquinolin-2- | |
| | | | yl)sulfinyl]butanenitrile | |
| 109 | 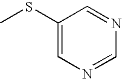 | 364 (M + H)⁺ | 9-fluoro-2-(pyrimidin-5-ylthio)-3,6- dihydro-7H-benzo[b]imidazo[4,5- f]isoquinolin-7-one | Ex. 49 |
| 110 | —SCH₂C(O)CH₂CO₂C₂H₅ | 414 (M + H)⁺ | ethyl 4-[(9-fluoro-7-oxo-6,7-dihydro- | Ex. 44 |
| | | | 3H-benzo[h]imidazo[4,5-f]isoquinolin- | |
| | | | 2-yl)thio]-3-oxobutanoate | |
| 111 | —SCH₂CH(CH₃)CN | 353 (M + H)⁺ | 3-[(9-fluoro-7-oxo-6,7-dihydro-3H- | Ex. 44 |
| | | | benzo[h]imidazo[4,5-f]isoquinolin-2- | |
| | | | yl)thio]-2-methylpropanenitrile | |
| 112 | —S(O)CH₂CH(CH₃)CN | 369 (M + H)⁺ | 3-[(9-fluoro-7-oxo-6,7-dihydro-3H- | Ex. 44 |
| | | | benzo[h]imidazo[4,5-f]isoquinolin-2- | |
| | | | yl)sulfinyl]-2-methylpropanenitrile | |
| 113 | —SCH₂CH(OH)CH₃ | 344 (M + H)⁺ | 9-fluoro-2-[(2-hydroxypropyl)thio]- | Ex. 44 |
| | | | 3,3a,6,11b-tetrahydro-7H- | |
| | | | benzo[h]imidazo[4,5-f]isoquinolin-7- | |
| | | | one | |
| 114 | 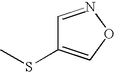 | 353 (M + H)⁺ | 9-fluoro-2-(isoxazol-4-ylthio)-3,6- dihydro-7H-benzo[h]imidazo[4,5- f]isoquinolin-7-one | Ex. 49 |
| 115 | —S—(CH₂)₂—CONH₂ | 357 (M + H)⁺ | 3-[(9-fluoro-7-oxo-6,7-dihydro-3H- | Ex. 44 |
| | | | benzo[h]imidazo[4,5-f]isoquinolin-2- | |
| | | | yl)thio]propanamide | |
| 116 | 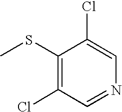 | 438, 435 (M + H)⁺ | 2-[(3,5-dichloropyridin-4-yl)thio]-9- fluoro-3,6-dihydro-7H- benzo[h]imidazo[4,5-f]isoquinolin-7- one | Ex. 44 |
| 117 | 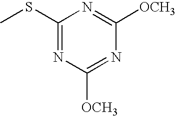 | 425 (M + H)⁺ | 2-[(4,6-dimethoxy-1,3,5-triazin-2- yl)thio]-9-fluoro-3,6-dihydro-7H- benzo[h]imidazo[4,5-f]isoquinolin-7- one | Ex. 44 |
| 118 | 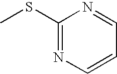 | 364 (M + H)⁺ | 9-fluoro-2-(pyrimidin-2-ylthio)-3,6- dihydro-7H-benzo[h]imidazo[4,5- f]isoquinolin-7-one | Ex. 44 |
| 119 | —SCH₂C(CH₃)₃ | 356 (M + H)⁺ | 2-[(2,2-dimethylpropyl)thio]-9-fluoro- | Ex. 44 |
| | | | 3,6-dihydro-7H-benzo[h]imidazo[4,5- | |
| | | | f]isoquinolin-7-one | |
| 120 | —SCH₂CH(CH₃)₂ | 342 (M + H)⁺ | 9-fluoro-2-(isobutylthio)-3,6-dihydro- | Ex. 44 |
| | | | 7H-benzo[h]imidazo[4,5-f]isoquinolin- | |
| | | | 7-one | |
| 121 | —S(O)CH₂C(CH₃)₃ | 372 (M + H)⁺ | 2-[(2,2-dimethylpropyl)sulfinyl]-9- | Ex. 44 |
| | | | fluoro-3,6-dihydro-7H- | |
| | | | benzo[h]imidazo[4,5-f]isoquinolin-7- | |
| | | | one | |
| 122 | —S(O)C₆H₁₁ | 384 (M + H)⁺ | 2-(cyclohexylsulfinyl)-9-fluoro-3,6- | Ex. 44 |
| | | | dihydro-7H-benzo[h]imidazo[4,5- | |
| | | | f]isoquinolin-7-one | |
| 123 | 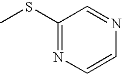 | 364 (M + H)⁺ | 9-fluoro-2-(pyrazin-2-ylthio)-3,6- dihydro-7H-benzo[h]imidazo[4,5- f]isoquinolin-7-one | Ex. 44 |
| 124 | 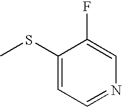 | 381 (M + H)⁺ | 9-fluoro-2-[(3-fluoropyridin-4-yl)thio]- 3,6-dihydro-7H-benzo[h]imidazo[4,5- f]isoquinolin-7-one | Ex. 44 |
| 125 | 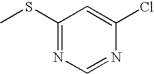 | 398 (M + H)⁺ | 2-[(6-chloropyrimidin-4-yl)thio]-9- fluoro-3,6-dihydro-7H- benzo[h]imidazo[4,5-f]isoquinolin-7- one | Ex. 44 |
| 126 | 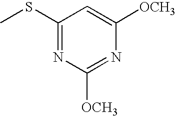 | 424 (M + H)⁺ | 2-[(2,6-dimethoxypyrimidin-4-yl)thio]- 9-fluoro-3,6-dihydro-7H- benzo[h]imidazo[4,5-f]isoquinolin-7- one | Ex. 44 |
| 127 | 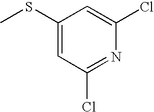 | 433, 435 (M + H)⁺ | 2-[(2,6-dichloropyridin-4-yl)thio]-9- fluoro-3,6-dihydro-7H- benzo[h]imidazo[4,5-f]isoquinolin-7- one | Ex. 44 |
| 128 | 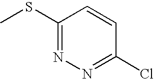 | 398 (M + H)⁺ | 2-[(6-chloropyridazin-3-yl)thio]-9- fluoro-3,6-dihydro-7H- benzo[h]imidazo[4,5-f]isoquinolin-7- one | Ex. 44 |
| 129 | 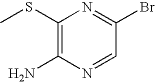 | 459 (M + H)⁺ | 2-[(3-amino-6-bromopyrazin-2- yl)thio]-9-fluoro-3,6-dihydro-7H- benzo[h]imidazo[4,5-f]isoquinolin-7- one | Ex. 44 |
| 130 | 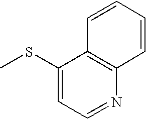 | 413 (M + H)⁺ | 9-fluoro-2-(quinolin-4-ylthio)-3,6- dihydro-7H-benzo[h]imidazo[4,5- f]isoquinolin-7-one | Ex. 44 |
| 131 | 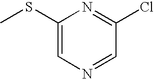 | 398 (M + H)⁺ | 2-[(6-chloropyrazin-2-yl)thio]-9- fluoro-3,6-dihydro-7H- benzo[h]imidazo[4,5-f]isoquinolin-7- one | Ex. 44 |
| 132 | 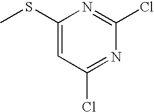 | 432, 434 (M + H)⁺ | 2-[(2,6-dichloropyrimidin-4-yl)thio]-9- fluoro-3,6-dihydro-7H- benzo[h]imidazo[4,5-f]isoquinolin-7- one | Ex. 44 |
| 133 | 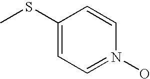 | 379 (M + H)⁺ | 9-fluoro-2-[(1-oxidopyridin-4-yl)thio]- 3,6-dihydro-7H-benzo[b]imidazo[4,5- f]isoquinolin-7-one | Ex. 44 |
| 134 | 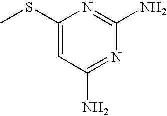 | 394 (M + H)⁺ | 2-[(2,6-diaminopyrimidin-4-yl)thio]-9- fluoro-3,6-dihydro-7H- benzo[h]imidazo[4,5-f]isoquinolin-7- one | Ex. 44 |
| 135 | 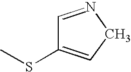 | 366 (M + H)⁺ | 9-fluoro-2-[(1-methyl-1H-pyrazol-4- yl)thio]-3,6-dihydro-7H- benzo[h]imidazo[4,5-f]isoquinolin-7- one | Ex. 49 |
| 136 | 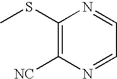 | 389 (M + H)⁺ | 3-[(9-fluoro-7-oxo-6,7-dihydro-3H- benzo[h]imidazo[4,5-f]isoquinolin-2- yl)thio]pyrazine-2-carbonitrile | Ex. 44 |
| 137 | 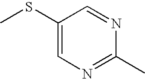 | 378 (M + H)⁺ | 9-fluoro-2-[(2-methylpyrimidin-5- yl)thio]-3,6-dihydro-7H- benzo[h]imidazo[4,5-f]isoquinolin-7- one | Ex. 49 |
| 138 | 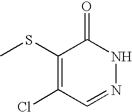 | 414 (M + H)⁺ | 2-[(5-chloro-3-hydroxypyridazin-4- yl)thio]-9-fluoro-3,6-dihydro-7H- benzo[h]imidazo[4,5-f]isoquinolin-7- one | Ex. 44 |
| 139 | 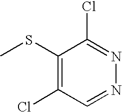 | 432, 434 (M + H)⁺ | 2-[(3,5-dichloropyridazin-4-yl)thio]-9- fluoro-3,6-dihydro-7H- benzo[h]imidazo[4,5-f]isoquinolin-7- one | Ex. 44 |
| 140 | 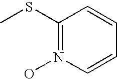 | 379 (M + H)⁺ | 9-fluoro-2-[(1-oxidopyridin-2-yl)thio]- 3,6-dihydro-7H-benzo[h]imidazo[4,5- f]isoquinolin-7-one | Ex. 44 |
| 140a | 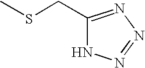 | 368 (M + H)⁺ | 9-fluoro-2-[(1H-tetrazol-5- ylmethyl)thio]-3,6-dihydro-7H- benzo[h]imidazo[4,5-f]isoquinolin-7- one trifluoroacetate | Ex. 44 |
| 140b | 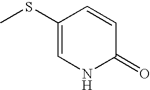 | 379 (M + H)⁺ | 9-fluoro-2-[(6-hydroxypyridin-3- yl)thio]-3,6-dihydro-7H- benzo[h]imidazo[4,5-f]isoquinolin-7- one | Ex. 44 |
| 140c | 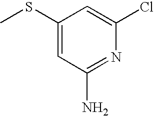 | 412, 414 (M + H)⁺ | 2-[(2-amino-6-chloropyridin-4- yl)thio]-9-fluoro-3,6-dihydro-7H- benzo[h]imidazo[4,5-f]isoquinolin-7- one trifluoroacetate | Ex. 44 |
| 140d | 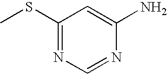 | 379 (M + H)⁺ | 2-[(6-aminopyrimidin-4-yl)thio]-9- fluoro-3,6-dihydro-7H- benzo[h]imidazo[4,5-f]isoquinolin-7- one | Ex. 44 |
| 140e | 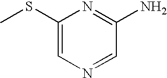 | 379 (M + H)⁺ | 2-[(6-aminopyrimidin-4-yl)thio]-9- fluoro-3,6-dihydro-7H- benzo[h]imidazo[4,5-f]isoquinolin-7- one | Ex. 44 |
| 140f | 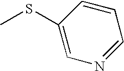 | 363 (M + H)⁺ | 9-fluoro-2-(pyridin-3-ylthio)-3,6- dihydro-7H-benzo[h]imidazo[4,5- f]isoquinolin-7-one trifluoroacetate | Ex. 44 |
| 140h | 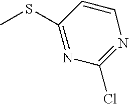 | 398, 400 (M + H)⁺ | 2-[(2-chloropyrimidin-4-yl)thio]-9- fluoro-3,6-dihydro-7H- benzo[h]imidazo[4,5-f]isoquinolin-7- one | Ex. 44 |
| 140i | 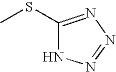 | 354 (M + H)⁺ | 9-fluoro-2-(1H-tetrazol-5-ylthio)-3,6- dihydro-7H-benzo[h]imidazo[4,5- f]isoquinolin-7-one | Ex. 44 |
| 140j | 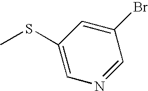 | 441, 443 (M + H)⁺ | 2-[(5-bromopyridin-3-yl)thio]-9- fluoro-3,6-dihydro-7H- benzo[h]imidazo[4,5-f]isoquinolin-7- one trifluoroacetate | Ex. 44 |
| 140k | 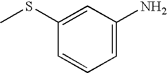 | 377 (M + H)⁺ | 2-[(3-aminophenyl)thio]-9-fluoro-3,6- dihydro-7H-benzo[h]imidazo[4,5- f]isoquinolin-7-one trifluoroacetate | Ex. 53a |
| 140l | 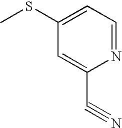 | 388 (M + H)⁺ | 4-[(9-fluoro-7-oxo-6,7-dihydro-3H- benzo[h]imidazo[4,5-f]isoquinolin-2- yl)thio]pyridine-2-carbonitrile | Ex. 53a |
| 140m | 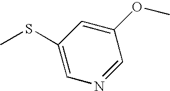 | 393 (M + H)⁺ | 9-fluoro-2-[(5-methoxypyridin-3- yl)thio]-3,6-dihydro-7H- benzo[h]imidazo[4,5-f]isoquinolin-7- one trifluoroacetate | Ex. 53a |
| 140n | 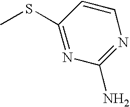 | 379 (M + H)⁺ | 2-[(2-aminopyrimidin-4-yl)thio]-9- fluoro-3,6-dihydro-7H- benzo[h]imidazo[4,5-f]isoquinolin-7- one | Ex. 44 |
| 140o | 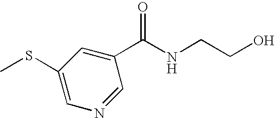 | 450 (M + H)⁺ | 5-[(9-fluoro-7-oxo-6,7-dihydro-3H- benzo[h]imidazo [4,5-f]isoquinolin-2- yl)thio]-N-(2- hydroxyethyl)nicotinamide trifluoroacetate (salt) | Ex. 44 |
| 140p | 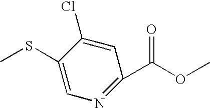 | 455, 457 (M + H)⁺ | methyl 4-chloro-5-[(9-fluoro-7-oxo- 6,7-dihydro-3H-benzo[h]imidazo[4,5- f]isoquinolin-2-yl)thio]pyridine-2- carboxylate | Ex. 44 |
| 140q | 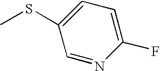 | 381 (M + H)⁺ | 9-fluoro-2-[(6-fluoropyridin-3- yl)thio]-3,6-dihydro-7H- benzo[h]imidazo[4,5-f]isoquinolin-7- one trifluoroacetate | Ex. 44 |
| 140r | 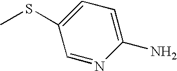 | 378 (M + H)⁺ | 2-[(6-aminopyridin-3-yl)thio]-9- fluoro-3,6-dihydro-7H- benzo[h]imidazo[4,5-f]isoquinolin-7- one trifluoroacetate | Ex. 44 |
| 140s | 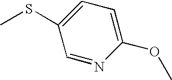 | 393 (M + H)⁺ | 9-fluoro-2-[(6-methoxypyridin-3- yl)thio]-3,6-dihydro-7H- benzo[h]imidazo[4,5-f]isoquinolin-7- one trifluoroacetate | Ex. 44 |
| 140t | 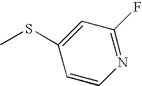 | 381 (M + H)⁺ | 9-fluoro-2-[(2-fluoropyridin-4- yl)thio]-3,6-dihydro-7H- benzo[h]imidazo[4,5-f]isoquinolin-7- one | Ex. 44 |
| 140u | 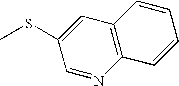 | 413 (M + H)⁺ | 9-fluoro-2-(quinolin-3-ylthio)-3,6- dihydro-7H-benzo[h]imidazo[4,5- f]isoquinolin-7-one trifluoroacetate | Ex. 44 |
| 140v | 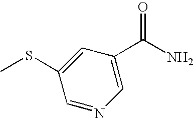 | 406 (M + H)⁺ | 5-[(9-fluoro-7-oxo-6,7-dihydro-3H- benzo[h]imidazo[4,5-t]isoquinolin-2- yl)thio]nicotinamide trifluoroacetate | Ex. 44 |
| 140w | 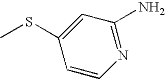 | 378 (M + H)⁺ | 2-[(2-aminopyridin-4-yl)thio]-9- fluoro-3,6-dihydro-7H- benzo[h]imidazo[4,5-f]isoquinolin-7- one | Ex. 44 |
| 140x | 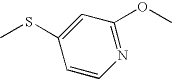 | 393 (M + H)⁺ | 9-fluoro-2-[(2-methoxypyridin-4- yl)thio]-3,6-dihydro-7H- benzo[h]imidazo[4,5-f]isoquinolin-7- one | Ex. 44 |
| 140y | 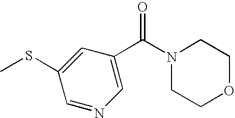 | 476 (M + H)⁺ | 9-fluoro-2-{[5-(morpholin-4- ylcarbonyl)pyridin-3-yl]thio}-3,6- dihydro-7H-benzo[h]imidazo[4,5- f]isoquinolin-7-one trifluoroacetate | Ex. 44 |
| 140z | 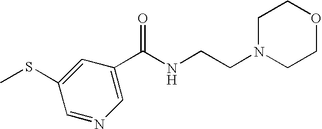 | 519 (M + H)⁺ | 5-[(9-fluoro-7-oxo-6,7-dihydro-3H- benzo[h]imidazo[4,5-f]isoquinolin-2- yl)thio]-N-[2-(tetrahydro-2H-pyran-4- yl)ethyl]nicotinamide | Ex. 44 |
| 140aa | 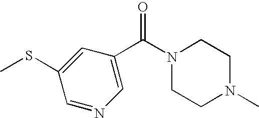 | 489 (M + H)⁺ | 9-fluoro-2-({5-[(4-methylpiperazin-1- yl)carbonyl]pyridin-3-yl}thio)-3,6- dihydro-7H-benzo[h]imidazo[4,5- f]isoquinolin-7-one | Ex. 44 |
| 140ab | 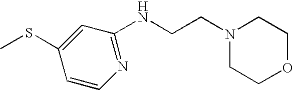 | 491 (M + H)⁺ | 9-fluoro-2-({2-[(2-morpholin-4- ylethyl)amino]pyridin-4-yl}thio)-3,6- dihydro-7H-benzo[h]imidazo[4,5- f]isoquinolin-7-one | Ex. 44 |
| 140ac | 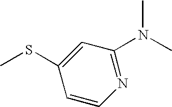 | 406 (M + H)⁺ | 2-{[2-(dimethylamino)pyridin-4- yl]thio}-9-fluoro-3,6-dihydro-7H- benzo[h]imidazo[4,5-f]isoquinolin-7- one | Ex. 44 |
| 140ad | 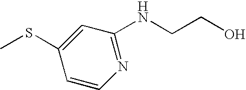 | 422 (M + H)⁺ | 9-fluoro-2-({2-[(2- hydroxyethyl)amino]pyridin-4- yl}thio)-3,6-dihydro-7H- benzo[h]imidazo[4,5-f]isoquinolin-7- one | Ex. 44 |
| 140ae | 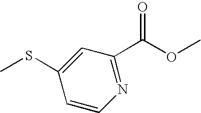 | 421 (M + H)⁺ | methyl 4-[(9-fluoro-7-oxo-6,7- dihydro-3H-benzo[h]imidazo[4,5- f]isoquinolin-2-yl)thio]pyridin-2- carboxylate | Ex. 44 |
| 140af | 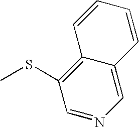 | 413 (M + H)⁺ | 9-fluoro-2-(isoquinolin-4-ylthio)-3,6- dihydro-7H-benzo[b]imidazo[4,5- f]isoquinolin-7-one trifluoroacetate | Ex. 44 |
| 140ag | 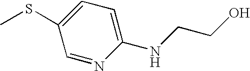 | 422 (M + H)⁺ | 9-fluoro-2-({6-[(2- hydroxyetbyl)amino]pyridin-3- yl}thio)-3,6-dihydro-7H- benzo[h]imidazo[4,5-f]isoquinolin-7- one trifluoroacetate (salt) | Ex. 44 |
| 140ah | 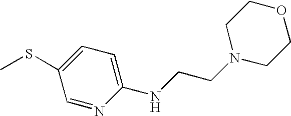 | 491 (M + H)⁺ | 9-fluoro-2-({6-[(2-morpholin-4- ylethyl)amino]pyridin-3-yl}thio)-3,6- dihydro-7H-benzo[h]imidazo[4,5- f]isoquinolin-7-one trifluoroacetate | Ex. 44 |
| 140ai | 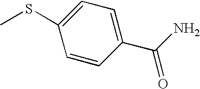 | 405 (M + H)⁺ | 4-[(9-fluoro-7-oxo-6,7-dihydro-3H- benzo[h]imidazo[4,5-f]isoquinolin-2- yl)thio]benzamide | Ex. 53a |
| 140aj | 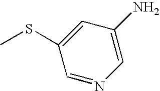 | 378 (M + H)⁺ | 2-[(5-aminopyridin-3-yl)thio]-9- fluoro-3,6-dihydro-7H- benzo[h]imidazo[4,5-f]isoquinolin-7- one | Ex. 53a |
| 140ak | 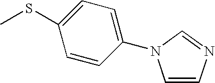 | 428 (M + H)⁺ | 9-fluoro-2-{[4-(1H-imidazol-1- yl)phenyl]thio}-3,6-dihydro-7H- benzo[h]imidazo[4,5-f]isoquinolin-7- one trifluoroacetate | Ex. 53a |
| 140al | 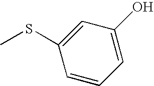 | 378 (M + H)⁺ | 9-fluoro-2-[(3-hydroxyphenyl)thio]- 3,6-dihydro-7H-benzo[h]imidazo[4,5- f]isoquinolin-7-one trifluoroacetate (salt) | Ex. 53a |
| 140am | 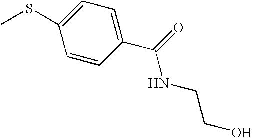 | 449 (M + H)⁺ | 4-[(9-fluoro-7-oxo-6,7-dihydro-3H- benzo[h]imidazo[4,5-f]isoquinolin-2- yl)thio]-N-(2- hydroxyethyl)benzamide trifluoroacetate (salt) | Ex. 53a |
| 140an | 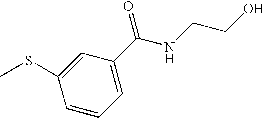 | 449 (M + H)⁺ | 3-[(9-fluoro-7-oxo-6,7-dihydro-3H- benzo[h]imidazo[4,5-f]isoquinolin-2- yl)thio]-N-(2- hydroxyethyl)benzamide trifluoroacetate (salt) | Ex. 53a |
| 140ao | 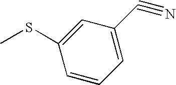 | 387 (M + H)⁺ | 3-[(9-fluoro-7-oxo-6,7-dihydro-3H- benzo[h]imidazo[4,5-f]isoquinolin-2- yl)thio]benzonitrile trifluoroacetate | Ex. 53a |
| 140ap | 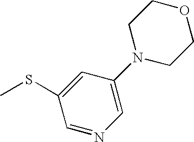 | 448 (M + H)⁺ | 9-fluoro-2-[(5-morpholin-4-ylpyridin- 3-yl)thio]-3,6-dihydro-7H- benzo[h]imidazo[4,5-f]isoquinolin-7- one trifluoroacetate | Ex. 53a |
| 140aq | 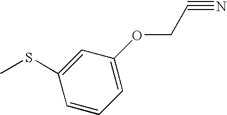 | 417 (M + H)⁺ | {3-[(9-fluoro-7-oxo-6,7-dihydro-3H- benzo[h]imidazo[4,5-f]isoquinolin-2- yl)thio]phenoxy}acetonitrile trifluoroacetate | Ex. 53a |
| 140ar | 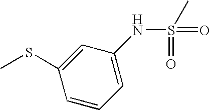 | 455 (M + H)⁺ | N-{3-[(9-fluoro-7-oxo-6,7-dihydro- 3H-benzo[h]imidazo[4,5- f]isoquinolin-2- yl)thio]phenyl}methanesulfonamide trifluoroacetate | Ex. 53a |
| 140as | 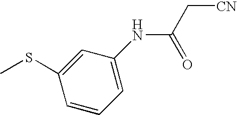 | 443 (M + H)⁺ | 2-cyano-N-{3-[(9-fluoro-7-oxo-6,7- dihydro-3H-benzo[h]imidazo[4,5- f]isoquinolin-2- yl)thio]phenyl}acetamide trifluoroacetate | Ex. 53a |
| 140at | 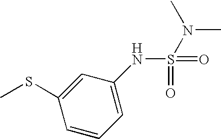 | 484 (M + H)⁺ | N′-{3-[(9-fluoro-7-oxo-6,7-dihydro- 3H-benzo[h]imidazo[4,5- f]isoquinolin-2-yl)thio]phenyl}-N,N- dimethylsulfamide | Ex. 53a |
| 140au | 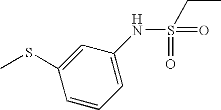 | 469 (M + H)⁺ | N-{3-[(9-fluoro-7-oxo-6,7-dihydro- 3H-benzo[h]imidazo[4,5- f]isoquinolin-2- yl)thio]phenyl}ethanesulfonamide | Ex. 53a |
| 140av | 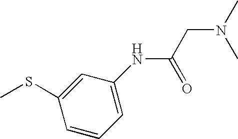 | 462 (M + H)⁺ | 2-(dimethylamino)-N-{3-[(9-fluoro-7- oxo-6,7-dihydro-3H- benzo[h]imidazo[4,5-f]isoquinolin-2- yl)thio]phenyl}acetamide trifluoroacetate | Ex. 53a |
| 140aw | 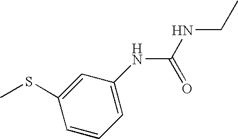 | 448 (M + H)⁺ | N-ethyl-N′-{3-[(9-fluoro-7-oxo-6,7- dihydro-3H-benzo[h]imidazo[4,5- f]isoquinolin-2-yl)thio]phenyl}urea trifluoroacetate | Ex. 53a |

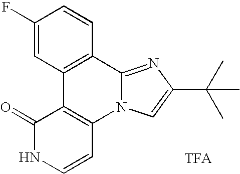

### Example 141

### 2-tert-Butyl-10-fluorobenzo[c]imidazo[1,2-a]-1,6-naphthyridin-8(7H)-one trifluoroacetate salt

Step 1

### 3-Iodopyridin-4-amine and 3,5-diiodopyridin-4-amine

Into a 3-neck round bottom flask 4-pyridinamine (9.21 g, 0.0978 mol) in water (35.00 mL) was heated to reflux with sodium carbonate (6.12 g, 0.0577 mol). Into the reaction was added dropwise a solution of potassium iodide (19.48 g, 0.1173 mol) and iodine (18.37 g, 0.07238 mol) in water (77.00 mL). After the addition was over the reaction was continued for 2 hours and extracted with ethyl acetate and washed with water and saturated NaCl, dried (MgSO₄) and stripped in vacuo. The reaction was chromatographed on silica gel using 40% EtOAc/haxanes, followed by EtOAc to give 6.6 g product. ¹H NMR(CDCl₃): 8.72 (s, 1H), 8.2 (d, 1H), 6.62 (d, 1H), 4.61 (br s, 2H).

Step 2

### 4-Fluoro-N,N-diisopropylbenzamide

Into a 1-Neck round-bottom flask N,N-diisopropylamine (13.0 mL, 0.0931 mol) was dissolved in dichloromethane (100.00 mL) and was cooled at 0° Celsius. Into the reaction was added 4-fluorobenzoyl chloride (5.00 mL, 0.0423 mol) dropwise and the reaction was stirred at 0° Celsius for 2 hours and at 25 Celsius for 16 hours. Extracted with dichloromethane, and the organic extract was washed with water, saturated solution of NaCl, dried (MgSO₄) and stripped in vacuo. The product (9.4 grams) was used in the next reaction without further purification.

Step 3

### 2-[(Diisopropylamino)carbonyl]-5-fluorophenylboronic acid

Into a 1-Neck round-bottom flask N,N,N′,N′-tetramethylethylenediamine (2.271 mL, 0.01505 mol) was dissolved in tetrahydrofuran (32.55 mL) and was cooled at −78° Celsius. Into the reaction was added 1.300 M of sec-butyllithium in cyclohexane (11.58 mL) and 4-fluoro-N,N-diisopropylbenzamide (2.24 g, 0.0100 mol) in tetrahydrofuran (10 mL) was added over for 5 minutes. The reaction was stirred for 15 minutes and boric acid, trimethyl ester (3.42 mL, 0.0301 mol) was added and was stirred at −78° Celsius for 30 minutes, allowed to warm at 0° Celsius and quenched with sat. NH₄Cl and 40 mL 1 N HCl was added. The reaction was stirred at 25° Celsius for 16 hours and was extracted with dichloromethane (80 mL). The dichloromethane extract was extracted with 1 N NaOH (2×70 mL) and the combined NaOH extracts were washed with dichloromethane, acidified with concHCl and extracted with dichloromethane (2×70 mL). The combined extract was washed with brine, dried and was rotovaped to give the product (7.2 g). ¹H NMR(CDCl₃): δ 7.61 (m, 1H), 7.28 (m, 1H), 7.01 (m, 1H), 4.15 (m, 1H), 3.44 (m, 1H), 1.32 (m, 6H), 1.13 (m, 6H).

Step 4

### 2-(4-Aminopyridin-3-yl)-4-fluoro-N,N-diisopropylbenzamide

Into a 1-Neck round-bottom flask 2-[(diisopropylamino)carbonyl]-5-fluorophenylboronic acid (2.49 g, 0.00932 mol) was mixed with 3-iodopyridin-4-amine (1.9 g, 0.0085 mol), and potassium carbonate (2.30 g, 0.0167 mol), in toluene (83.00 mL), ethanol (11 mL) and water (8.30 mL) and was degassed. Into the reaction was added tetrakis(triphenyl-phosphine)palladium(0) (367 mg, 0.000318 mol) and was heated at 80 Celsius for 24 hours. Extracted with ethyl acetate and washed with water and saturated NaCl, dried (MgSO₄) and stripped in vacuo. The residue was triturated with ether to give the product (2.0 g), >95% purity by HPLC. ¹H NMR(CDCl₃): δ 8.15 (d, 1H), 8.03 (m, 1H), 7.26 (m, 1H), 7.15 (m, 1H), 7.02 (m, 1H); 6.56 (d, 1H), 4.53 (br s, 2H), 3.58 (m, 1H), 3.31 (m, 1H), 1.48 (m, 3H), 1.14 (m, 3H), 1.01 (m, 3H), 0.82 (m, 3H). MS(ES) 316 (M+1).

Step 5

### 9-Fluorobenzo[c]-1,6-naphthyridin-6(5H)-one

Into a 1-Neck round-bottom flask 2-(4-aminopyridin-3-yl)-4-fluoro-N,N-diisopropylbenzamide (0.200 g, 0.000634 mol) was dissolved in tetrahydrofuran (4.00 mL) and was cooled at 0° Celsius. To that 1.00 M of sodium hexamethyldisilazane in tetrahydrofuran was added and the reaction was stirred at 0° Celsius for 3 hours and at 250 Celsius for 16 hours at which time a white solid was formed (it started forming after the NaHMDS addition). HPLC analysis showed no starting material. The reaction was quenched with water (10 mL) and partitioned between that and EtOAc. The precipitated solid was filtered and dried to give the product (1.0 g). ¹H NMR(DMSO d₆): δ 12 (br s, 1H), 9.55 (s, 1H), 8.50 (m, 1H), 8.49 (m, 1H), 8.36 (m, 1H), 7.55 (m, 1H), 7.24 (d, 1H). MS(ES) 215 (M+1).

Step 6

### 5-(3,3-Dimethyl-2-oxobutyl)-9-fluorobenzo[c]-1,6-naphthyridin-6(5H)-one

Into a 1-neck round-bottom flask 9-fluorobenzo[c]-1,6-naphthyridin-6(5H)-one (250.00 mg, 0.0011672 mol) was dissolved in N,N-dimethylformamide (5.556 mL) and 1.00 M of potassium tert-butoxide in tetrahydrofuran (1.17 mL) was added at which time the reaction became homogeneous. Into the reaction was added 1-bromo-3,3-dimethyl-2-butanone (0.17 mL, 0.0012 mol) and the reaction was stirred at 25 Celsius for 2 hours. HPLC and mass specral analysis showed mainly product present. Extracted with ethyl acetate and washed with water and saturated NaCl, dried (MgSO4), and stripped in vacuo. The reaction was chromatographed on silica gel using 1:1 EtOAc/hexanes to give the product (224 mg). ¹H NMR(CDCl₃): δ 9.38 (s, 1H), 8.58 (d, 1H), 8.50 (m, 1H), 7.99 (m, 1H), 7.35 (m, 1H), 6.77 (d, 1H), 5.35 (s, 2H), 1.39 (s, 9H). MS(ES) 313 (M+1).

Step 7

### 5-(3,3-Dimethyl-2-oxobutyl)-9-fluorobenzo[c]-1,6-naphthyridin-6(5H)-one 2-oxide

Into a 1-neck round-bottom flask 5-(3,3-dimethyl-2-oxobutyl)-9-fluorobenzo[c]-1,6-naphthyridin-6(5H)-one (242.00 mg, 0.775 mmol) was dissolved in dichloromethane (5.15 mL) and m-chloroperbenzoic acid (545.79 mg, 0.0018977 mol) was added The reaction was stirred at 25° Celsius for 2.5 hours at which time HPLC analysis showed no starting material. Then it was quenched with 10% Na₂S₂O₄ and extracted with dichloromethane, and the organic extract was washed with NaHCO₃ (2×), saturated solution of NaCl, dried (MgSO₄) and stripped in vacuo to give the product (223 mg). ¹H NMR(CDCl₃): δ 9.00 (d, 1H), 8.55 (m, 1H), 8.25 (m, 1H), 7.73 (m, 1H), 7.42 (m, 1H), 6.77 (d, 1H), 5.33 (s, 2H), 1.38 (s, 914). MS(ES) 329 (M+1).

Step 8

### 5-(3,3-Dimethyl-2-oxobutyl)-9-fluoro-1-hydroxybenzo[c]-1,6-naphthyridin-6(5H)-one

Into a 1-Neck round-bottom flask 5-(3,3-dimethyl-2-oxobutyl)-9-fluorobenzo[c]-1,6-naphthyridin-6(5H)-one 2-oxide (220.0 mg, 0.0006700 mol) in acetic anhydride (5.00 mL, 0.0530 mol) was heated at 147° Celsius for 2 hours. Then it was allowed to cool and was mixed with sat. NaHCO₃ solution. After the acetic anhydride had reacted, the product was extracted with ethyl acetate and washed with saturated solution of NaHCO₃, brine, dried and stripped in vacuo. The reaction was chromatographed on silica gel using 1:1 EtOAc/hexanes as eluent. The acetate was cleaved during chromatography and only a small amount of it was recovered (15 mg), using 1:1 EtOAc/hexanes as eluent. Then the column was eluted with THF, EtOAc and 10% MeOH/EtOAc to give some of the corresponding pyridone (67 mg). ¹H NMR(DMSO d6): δ 11.98 (br d, 1H), 9.52 (dd, 1H), 8.32 (m, 1H), 7.57 (m, 1H), 7.42 (m, 1H), 6.27 (d, 1H), 5.45 (s, 2H), 1.24 (s, 9H). MS(ES) 329 (M+1).

Step 9

### 2-tert-butyl-10-fluorobenzo[c]imidazo[1,2-a]-1,6-naphthyridin-8(7H)-one trifluoroacetate salt

In a 3 mL microwave vial 5-(3,3-dimethyl-2-oxobutyl)-9-fluoro-1-hydroxybenzo[c]-1,6-naphthyridin-6(5H)-one (34.00 mg, 0.0001036 mol) with ammonium acetate (34.00 mg, 0.0004411 mol) was dissolved in acetic acid (0.30 mL) and N,N-dimethylformamide (0.30 mL) was heated at 200 Celsius for 2 hours (four 30 min experiments) in a microwave reactor. LCMS analysis showed 3:1 product starting material. The solvent was stripped off and the residue was dissolved in 1:1 DMSO/THF and purified by preparative LCMS to give the product (13 mg). ¹H NMR(DMSO d6): δ 12.33 (br s, 1H), 9.67 (dd, 1H), 8.65 (m, 1H), 8.42 (s, 1H), 7.85 (m, 1H), 7.62 (m, 1H), 7.35 (m, 1H), 1.41 (s, 9H). MS(ES) 310 (M+1).

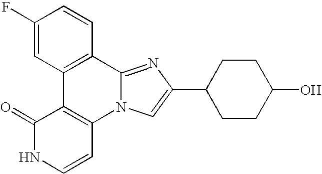

### Example 142

### 10-Fluoro-2-(4-hydroxycyclohexyl)benzo[c]imidazo[1,2-a]-1,6-naphthyridin-8(7H)-one

Step 1

### 4-(Acetyloxy)cyclohexanecarboxylic acid

4-Hydroxycyclohexanecarboxylic acid (10.0 g, 0.069 mol, mixture of cis and trans isomers) was added to acetic anhydride (48.7 mL, 0.310 mol), followed by sulfuric acid (10 uL, 0.0002 mol). The reaction was heated to 100° C. until TLC (stained with phosphomolybdic acid stain) indicated completion of reaction (1-2 hours). The reaction was cooled to room temperature and excess acetic anhydride was removed by rotary evaporation. Water (10 mL) was added to the residue and was warmed to 50° C. until TLC indicated complete hydrolysis of the resultant anhydride to the carboxylic acid. The reaction was again evaporated under reduced pressure to leave the crude product which crystallizes to a brown solid. The crude product was then dissolved in saturated NaHCO₃ solution and transferred to a separatory funnel, and washed with EtOAc. The aqueous phase was acidified with concentrated HCl to pH of 2, then extracted with EtOAc. The EtOAc phase was washed with water, saturated NaCl, dried over MgSO₄ and evaporated to dryness in vacuo to leave the product (11.9 g, 92%). ¹H NMR (400 MHz, CDCl₃): δ 4.95 (m, 0.5H), 4.71 (m, 0.5H), 2.45 (m, 0.5H), 2.33 (m, 0.5H), 2.07 (m, 2H), 2.06 (s, 1.5H), 2.04 (s, 1.5H), 1.84 (m, 3H), 1.61 (m, 2H), 1.40 (m, 1H).

Step 2

### 4-(2-diazoacetyl)cyclohexyl acetate

4-(Acetyloxy)cyclohexanecarboxylic acid (630 mg, 0.0034 mol) was dissolved in dichloromethane (5.0 mL) and the solution was cooled to 0° C., then N,N-dimethylformamide (30 μL) was added, followed by dropwise addition of oxalyl chloride (430 μL, 0.0051 mol). The reaction was held at 0° C. for 30 min, then warmed to room temperature for 30 min. The reaction was reduced in vacuo to leave the crude acid chloride as an orange oil.

A solution of 2.0 M of trimethylsilyldiazomethane in hexane (6.7 mL) was added to tetrahydrofuran (4.8 mL,) and this resulting solution was cooled to 0° C. The crude acid chloride prepared above was dissolved in tetrahydrofuran (4.8 mL), and this solution was added dropwise via syringe to the trimethylsilyidiazomethane solution. The reaction was held at 0° C. for 18 h, then reduced to dryness in vacuo to leave the crude diazoketone (711 mg, 100%). ¹H NMR (400 MHz, CDCl₃): δ 5.00 (m, 0.5H), 4.69 (m, 0.5H), 2.67 (m, 0.5H), 2.55 (m, 0.5H), 2.08 (m, 1H), 2.04 (s, 3H), 1.80 (m, 4H), 1.60 (m, 3H), 1.39 (m, 1H).

Step 3

### 4-(2-bromoacetyl)cyclohexyl acetate

Acetic acid (9.6 mL) was added to 4-(2-diazoacetyl)cyclohexyl acetate (711 mg, 0.0034 mol) and this solution was cooled to 0° C., then 6 M HBr (6 mL) was added quickly, causing vigorous gas evolution. The solution was stirred at 0° C. for 15 min, then the reaction was transferred to a separatory funnel and partitioned between water and DCM. The phases were separated and the aqueous phase was washed with additional DCM. The combined organic phase was washed with sat'd NaHCO₃, then saturated NaCl, and dried over MgSO₄. The solvent was removed in vacuo to leave the crude product (895 mg, 54%). ¹H NMR (400 MHz, CDCl₃): δ 5.00 (m, 0.5H), 4.69 (m, 0.5H), 3.97 (s, 1H), 3.95 (s, 1H), 2.75 (m, 1H), 2.08 (m, 1H), 2.05 (s, 1.5H), 2.04 (s, 1.5H), 1.98 (m, 3H), 1.78 (m, 2H), 1.50 (m, 2H).

Step 4

### 4-[2-(9-fluoro-6-oxobenzo[c]-1,6-naphthyridin-5(6H)-yl)acetyl]cyclohexyl acetate

N,N-Dimethylformamide (3.731 mL) was added to 9-fluorobenzo[c]-1,6-naphthyridin-6(5H)-one (167.9 mg, 0.0007839 mol) and the mixture was cooled to 0° C. A 1.00 M solution of potassium tert-butoxide in tetrahydrofuran (0.862 mL) was added dropwise, then the cooling bath was removed and the reaction was allowed to warm to room temperature. The reaction was held at room temperature for 30 min, then cooled back to 0° C. and a solution of 4-(2-bromoacetyl)cyclohexyl acetate (243 mg, 0.000784 mol) in N,N-dimethylformamide (0.56 mL) was added. The reaction was held at 0° C. for 1 h, then warmed to room temperature until HPLC indicated complete reaction (1-2 h). Water and ether were added and the resulting precipitate was isolated by filtration. HPLC indicated that this solid is enriched in the trans isomer. The resulting filtrate was extracted with CHCl₃ and the organic phase was washed with water, saturated NaCl, and dried over MgSO₄ to provide crude material enriched in the cis isomer. The isomers were separated by column chromatography (1% MeOH/EtOAc) to give pure material of each isomer as well as recovering some mixed isomers, (total yield of both isomers 236 mg, 76%). Trans isomer ¹H NMR (400 MHz, CDCl₃): δ 9.40 (bs, 1H), 8.60 (bs, 1H), 8.51 (m, 1H), 7.99 (dd, 1H), 7.35 (m, 1H), 6.80 (d, 1H), 5.23 (s, 2H), 4.73 (m, 1H), 2.66 (m, 1H), 2.14 (m, 4H), 2.06 (s, 3H), 1.66 (m, 2H), 1.47 (m, 2H). Cis isomer ¹H NMR (400 MHz, CDCl₃): □ 9.40 (s, 1H), 8.59 (d, 1H), 8.52 (m, 1H), 9.00 (m, 1H), 7.35 (m, 1H), 6.82 (d, 1H), 5.25 (s, 2H), 5.04 (m, 1H), 2.73 (m, 1H), 2.07 (s, 3H), 1.98 (m, 3H), 1.911 (m, 3H), 1.64 (m, 2H). MS(ES) 397 (M+1).

Step 5

trans-4-[2-(9-fluoro-2-oxido-6-oxobenzo[c]-1,6-naphthyridin-5(6H)-yl)acetyl]cyclohexyl acetate; trans-4-[2-(9-Fluoro-6-oxobenzo[c]-1,6-naphthyridin-5(6H)-yl)acetyl]cyclohexyl acetate (104.0 mg, 0.0002624 mol) was suspended in dichloromethane (3.36 mL), then m-chloroperbenzoic acid (185 mg, 0.000643 mol) was added. The reaction was stirred at room temperature until HPLC indicated complete reaction (1-2 h). Reaction was treated with 10% Na₂S₂O₄, then after a few minutes of stirring added saturated NaHCO₃ to neutral pH. The phases were separated, and the organic phase was washed with saturated NaHCO₃, water, saturated NaCl, dried over MgSO₄ and evaporated in vacuo to leave the crude product (108 mg, 99%). ¹H NMR (400 MHz, CDCl₃): δ 8.99 (s, 1H), 8.52 (m, 1H), 8.23 (m, 1H), 7.71 (m, 1H), 7.42 (m, 1H), 6.80 (d, 1H), 5.21 (s, 2H), 4.73 (m, 1H), 2.66 (m, 1H), 2.14 (m, 4H), 2.06 (s, 3H), 1.65 (m, 2H), 1.49 (m, 2H). MS(ES) 413 (M+1).

Step 6

### 4-[2-(9-fluoro-1,6-dioxo-2,6-dihydrobenzo[c]-1,6-naphthyridin-5(1H)-yl)acetyl]cyclohexyl acetate

Acetic anhydride (2.7 mL, 0.029 mol) was added to 4-[2-(9-fluoro-2-oxido-6-oxobenzo[c]-1,6-naphthyridin-5(6H)-yl)acetyl]cyclohexyl acetate (108 mg, 0.000262 mol) and the mixture was heated to 145° C. until HPLC indicated complete reaction (1-2 h). The reaction was cooled to room temperature and water (0.57 mL, 0.031 mol) was added and the reaction was stirred at room temperature for 16 h. The reaction was evaporated to dryness in vacuo and the residue was washed with ether to leave the crude product (82 mg, 76%). MS(ES) 413 (M+1).

Step 7

### 10-fluoro-2-(4-hydroxycyclohexyl)benzo[c]imidazo[1,2-a]-1,6-naphthyridin-8(7H)-one

4-[2-(9-fluoro-1,6-dioxo-2,6-dihydrobenzo[c]-1,6-naphthyridin-5(1H)-yl)acetyl]cyclohexyl acetate (135.0 mg, 0.000194 mol) was suspended in N,N-dimethylformamide (0.68 mL) and acetic acid (0.68 mL), and ammonium acetate (378 mg, 0.00491 mol) was added. This mixture was heated to 210° C. in the microwave for 1.5 h to give a mixture of cyclized products both with and without the acetate group present. The solvent was removed in vacuo and to the residue was added tetrahydrofuran (1.0 mL) and 1.0 M of sodium hydroxide (2.0 mL), the reaction was heated to 50° C. until HPLC indicated complete cleavage of the acetate group (1-2 h). The reaction was cooled to room temperature and the solvent was removed in vacuo. The crude reaction product was purified by column chromatography (1:1 EtOAc/Hex to 100% EtOAc) to recover product as a mixture of the cis- and trans-isomers (75.1 mg, 65%). The isomers were separated by preparative HPLC to recover the pure isomers. (trans isomer) ¹H NMR (400 MHz, DMSO-d₆): δ 9.64 (dd, 1H), 8.50 (m, 1H), 8.21 (m, 1H), 7.78 (d, 1H), 7.49 (m, 1H), 7.15 (d, 1H), 4.54 (d, 1H), 3.47 (bs, 1H), 2.64 (m, 1H), 2.09 (m, 2H), 1.94 (m, 2H), 1.52 (m, 2H), 1.34 (m, 2H). MS(ES) 352 (M+1). (cis isomer) ¹H NMR (400 MHz, DMSO-d₆): δ 9.65 (d, 1H), 8.52 (m, 1H), 8.24 (s, 1H), 7.77 (d, 1H), 7.50 (m, 1H), 7.22 (d, 1H), 4.31 (d, 1H), 3.83 (bs, 1H), 2.77 (m, 1H), 1.99 (m, 2H), 1.80 (m, 2H), 1.68 (m, 2H), 1.60 (m, 2H). MS(ES) 352 (M+1).

### Example 143

### trans-4-(10-Fluoro-8-oxo-7,8-dihydrobenzo[c]imidazo[1,2-a]-1,6-naphthyridin-2-yl)cyclohexyl (dimethylamino)acetate

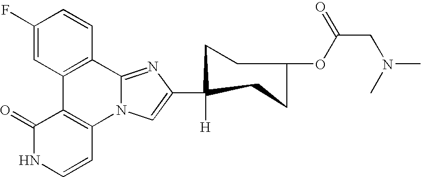

trans-10-Fluoro-2-(4-hydroxycyclohexyl)benzo[c]imidazo[1,2-a]-1,6-naphthyridin-8(7H)-one (25.0 mg, 0.0711 mmol) of Example 142 and (dimethylamino)acetic acid (7.70 mg, 0.0747 mmol) were combined in chloroform (1.252 mL) and N,N′-dicyclohexylcarbodiimide (44.0 mg, 0.213 mmol) and 4-dimethylaminopyridine (8.69 mg, 0.0711 mmol) were added. The reaction was stirred at room temperature until LCMS indicated complete reaction (5-16 h). The reaction was diluted with chloroform and the suspended solids were removed by filtration, The filtrate was reduced to dryness in vacuo; the residue was washed with DMSO (3 mL) and methanol (3 mL) to yield upon filtration a solid consisting primarily of the product with slight contamination of DCU and DMAP. This solid was dissolved in DMSO (3 mL) and methanol (1 mL) with heating and this solution was purified by reverse phase prep LC/MS (acetonitrile/water/TFA) to give the product as a bis-TFA salt (6.0 mg, 13%). ¹H NMR (500 MHz, DMSO-d₆): δ (d, 1H), 9.97 (bs, 1H), 9.65 (dd, 1H), 8.52 (m, 1H), 8.34 (s, 1H), 7.83 (m, 1H), 7.55 (m, 1H), 7.22 (d, 1H), 4.89 (m, 1H), 4.22 (s, 2H), 2.84 (s, 6H), 2.81 (m, 1H), 2.22 (m, 2H), 2.10 (m, 2H), 1.64 (m, 4H); MF=C₂₄H₂₅FN₄O₃; LCMS calculated for C₂₄H₂₆FN₄O₃(M+H)⁺: m/z=437.20, found 437.30.

Further compounds of the invention are provided in Tables 3, 4 and 5 below.

TABLE 3

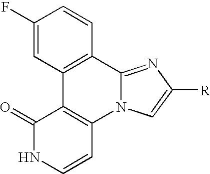

| Ex. | | | | MS |
|---|---|---|---|---|
| No. | Name | R | Method | (M + 1) |
| 144 | 10-fluoro-2-(1- | c-Pr(Me) | Ex. 141 | 308 |
| | methylcyclopropyl)benzo[c]imidazo[1,2-a]-1,6- | | | |
| | naphthyridin-8(7H)-one | | | |
| 145 | 2-cyclopropyl-10-fluorobenzo[c]imidazo[1,2-a]- | c-Pr | Ex. 141 | 294 |
| | 1,6-naphthyridin-8(7H)-one | | | |
| 146 | 2-cyclobutyl-10-fluorobenzo[c]imidazo[1,2-a]- | c-Bu | Ex. 141 | 308 |
| | 1,6-naphthyridin-8(7H)-one | | | |
| 147 | 2-ethyl-10-fluorobenzo[c]imidazo[1,2-a]-1,6- | Et | Ex. 141 | 282 |
| | naphthyridin-8(7H)-one | | | |
| 148 | ethyl 2-ethyl-2-(10-fluoro-8-oxo-7,8- | C(Et)₂CO₂Et | Ex. 141 | 396 |
| | dihydrobenzo[c]imidazo[1,2-a]-1,6- | | | |
| | naphthyridin-2-yl)butanoate | | | |
| 149 | 2-[1-ethyl-1-(hydroxymethyl)propyl]-10- | C(Et)₂CH₂OH | Ex. 141 | 354 |
| | fluorobenzo[c]imidazo[1,2-a]-1,6-naphthyridin- | | | |
| | 8(7H)-one | | | |
| 150 | 2-(1-ethylpropyl)-10-fluorobenzo[c]imidazo[1,2- | CH(Et)₂ | Ex. 141 | 324 |
| | a]-1,6-naphthyridin-8(7H)-one | | | |
| 151 | 10-fluoro-2-(4- | 4-oxocyclohexyl | Ex. 142 | 350 |
| | oxocyclohexyl)benzo[c]imidazo[1,2-a]-1,6- | | | |
| | naphthyridin-8(7H)-one | | | |
| 152 | 10-fluoro-2-methylbenzo[c]imidazo[1,2-a]-1,6- | Me | Ex. 141 | 268 |
| | naphthyridin-8(7H)-one | | | |
| 152a | 10-fluoro-2-(cis-4- | cis-4-methoxycyclohexyl | Ex. 142 | 366 |
| | methoxycyclohexyl)benzo[c]imidazo[1,2-a]-1,6- | | | |
| | naphthyridin-8(7H)-one | | | |
| 152b | 10-fluoro-2-(trans-4- | trans-4- | Ex. 142 | 366 |
| | methoxycyclohexyl)benzo[c]imidazo[1,2-a]-1,6- | methoxycyclohexyl | | |
| | naphthyridin-8(7H)-one | | | |
| 152c | 10-fluoro-2-[4- | 4- | Ex. 142 | 365 |
| | (hydroxyimino)cyclohexyl]benzo[c]imidazo[1,2- | (hydroxyimino)cyclohexyl | | |
| | a]-1,6-naphthyridin-8(7H)-one | | | |
| 152d | 2-(1-acetylpiperidin-4-yl)-10- | 1-acetylpiperidin-4-yl | Ex. 142 | 379 |
| | fluorobenzo[c]imidazo[1,2-a]-1,6-naphthyridin- | | | |
| | 8(7H)-one | | | |

TABLE 4

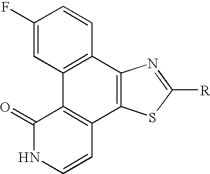

| | | | Mass | |
|---|---|---|---|---|
| Ex. | | | Spec | Method of |
| No. | Name | R | (M + H) | Preparation |
| 153 | 2-amino-9- | NH₂ | 286 | Ex. 1 |
| | fluorobenzo[h][1,3]thiazolo[5,4- | | | |
| | f]isoquinolin-7(6H)-one | | | |
| 154 | 9-fluoro-2-(4-hydroxypiperidin-1- yl)benzo[h][1,3]thiazolo[5,4- f]isoquinolin-7(6H)-one | 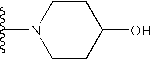 | 370 | Ex. 1 |
| 155 | 9-fluoro-2-{[(1S)-1-(hydroxymethyl)-3- methylbutyl]amino}benzo[h][1,3]thia zolo[5,4-f]isoquinolin-7(6H)-one | 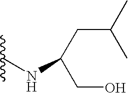 | 386 | Ex. 1 |
| 156 | 9-fluoro-2-{[(1R)-2-hydroxy-1- phenylethyl]amino}benzo[h][1,3]thia zolo[5,4-f]isoquinolin-7(6H)-one | 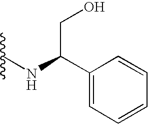 | 406 | Ex. 1 |
| 157 | (2S)-2-[(9-fluoro-7-oxo-6,7- dihydrobenzo[h][1,3]thiazolo[5,4- f]isoquinolin-2-yl)amino]-4-methoxy- N-methylbutanamide | 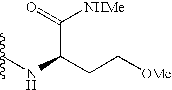 | 415 | Ex. 1 |
| 158 | 9-fluoro-2-(4-oxopiperidin-1- yl)benzo[h][1,3]thiazolo[5,4- f]isoquinolin-7(6H)-one | 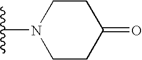 | 368 | Ex. 1 |
| 159 | 9-fluoro-2-{[(1R)-1-(hydroxymethyl)-3- methylbutyl]amino}benzo[h][1,3]thia zolo[5,4-f]isoquinolin-7(6H)-one | 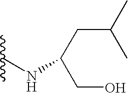 | 386 | Ex. 1 |
| 160 | 9-fluoro-2-(3-hydroxypiperidin-1- yl)benzo[h][1,3]thiazolo[5,4- f]isoquinolin-7(6H)-one | 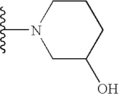 | 370 | Ex. 1 |
| 161 | 9-fluoro-2-{[(1S)-2-hydroxy-1- phenylethyl]amino}benzo[h][1,3]thia zolo[5,4-f]isoquinolin-7(6H)-one | 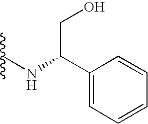 | 406 | Ex. 1 |
| 162 | 9-fluoro-2-(3-hydroxypyrrolidin-1- yl)benzo[h][1,3]thiazolo[5,4- f]isoquinolin-7(6H)-one | 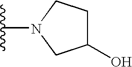 | 356 | Ex. 1 |
| 163 | 9-fluorobenzo[h][1,3]thiazolo[5,4- | H | 271 | Ex. 23, 1 |
| | f]isoquinolin-7(6H)-one | | | |
| 164 | 9-fluoro-2-(1-hydroxy-4- oxocyclohexyl)benzo[h][1,3]thiazolo [5,4-f]isoquinolin-7(6H)-one | 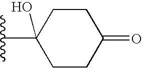 | 383 | Ex. 23 |
| 165 | 2-[4-(ethoxyimino)piperidin-1-yl]-9- fluorobenzo[h][1,3]thiazolo[5,4- f]isoquinolin-7(6H)-one | 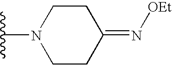 | 411 | Ex. 1 |
| 166 | 9-fluoro-2-[4- (hydroxyimino)piperidin-1- yl]benzo[h][1,3]thiazolo[5,4- f]isoquinolin-7(6H)-one | 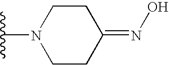 | 383 | Ex. 1 |
| 167 | 4-(9-fluoro-7-oxo-6,7- | —(CH₂)₃CN | 338 | Ex.23 |
| | dihydrobenzo[h][1,3]thiazolo[5,4- | | | |
| | f]isoquinolin-2-yl)butanenitrile | | | |
| 168 | 2-(1,4-dihydroxycyclohexyl)-9- fluorobenzo[h][1,3]thiazolo[5,4- f]isoquinolin-7(6H)-one | 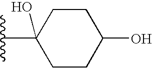 | 385 | Ex. 23 |
| 169 | 2-(1-acetyl-4-hydroxypiperidin-4-yl)- 9-fluorobenzo[h][1,3]thiazolo[5,4- f]isoquinolin-7(6H)-one | 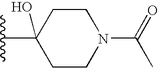 | 412 | Ex. 23 |
| 170 | 9-fluoro-2-(4-hydroxy-1- isobutyrylpiperidin-4- yl)benzo[h][1,3]thiazolo[5,4- f]isoquinolin-7(6H)-one | 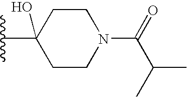 | 440 | Ex. 23 |
| 171 | 9-fluoro-2- isonicotinoylbenzo[h][1,3]thiazolo[5, 4-f]isoquinolin-7(6H)-one | 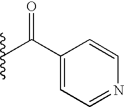 | 377 | Ex. 23 |
| 172 | 9-fluoro-2-(1- oxidoisonicotinoyl)benzo[h][1,3]thiazolo[5,4-f]isoquinolin-7(6H)-one | 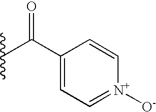 | 392 | Ex. 23 |
| 173 | 9-fluoro-2- | Ph | 347 | Ex. 1 |
| | phenylbenzo[h][1,3]thiazolo[5,4- | | | |
| | f]isoquinolin-7(6H)-one | | | |
| 174 | 2-ethyl-9- | Et | 299 | Ex. 1 |
| | fluorobenzo[h][1,3]thiazolo[5,4- | | | |
| | f]isoquinolin-7(6H)-one | | | |
| 175 | 9-fluoro-2-morpholin-4- ylbenzo[h][1,3]thiazolo[5,4- f]isoquinolin-7(6H)-one | 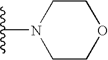 | 356 | Ex. 1 |
| 176 | 9-fluoro-2-[(pyridin-3- ylmethyl)amino]benzo[h][1,3]thiazolo [5,4-f]isoquinolin-7(6H)-one | 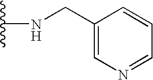 | 377 | Ex. 1 |
| 177 | 9-fluoro-2-[(3-morpholin-4- ylpropyl)amino]benzo[h][1,3]thiazolo [5,4-f]isoquinolin-7(6H)-one | 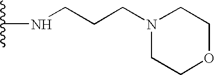 | 413 | Ex. 1 |
| 178 | 9-fluoro-2-[(3- methoxybenzyl)amino]benzo[h][1,3]thiazolo[5,4-f]isoquinolin-7(6H)-one | 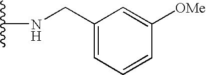 | 406 | Ex. 1 |
| 179 | 9-fluoro-2-{[(1R)-1-(3- methoxyphenyl)ethyl]amino}benzo [h][1,3]thiazolo[5,4-f]isoquinolin- 7(6H)-one | 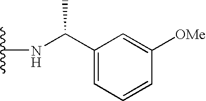 | 420 | Ex. 1 |
| 180 | 9-fluoro-2-{[(1S)-1-(3- methoxyphenyl)ethyl]amino}benzo [h][1,3]thiazolo[5,4-f]isoquinolin- 7(6H)-one | 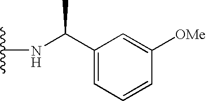 | 420 | Ex. 1 |
| 181 | 9-fluoro-2-[(3- | —NH(CH₂)₃OH | 344 | Ex. 1 |
| | hydroxypropyl)amino]benzo[h][1,3] | | | |
| | thiazolo[5,4-f]isoquinolin-7(6H)-one | | | |
| 182 | 9-fluoro-2-[(2- hydroxyethyl)(methyl)amino]benzo [h][1,3]thiazolo[5,4-f]isoquinolin- 7(6H)-one | 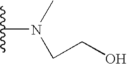 | 344 | Ex. 1 |
| 183 | 9-fluoro-2-{[2-(4- hydroxyphenyl)ethyl]amino}benzo[h][1,3]thiazolo[5,4-f]isoquinolin-7(6H)- one | 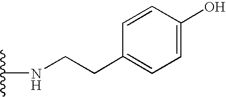 | 406 | Ex. 1 |
| 184 | 9-fluoro-2-[(2-methoxy-1- methylethyl)amino]benzo[h][1,3]thiazolo[5,4-f]isoquinolin-7(6H)-one | 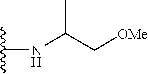 | 358 | Ex. 1 |
| 185 | N,N-diethyl-1-(9-fluoro-7-oxo-6,7- dihydrobenzo[h][1,3]thiazolo[5,4- f]isoquinolin-2-yl)piperidine-3- carboxamide | 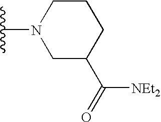 | 453 | Ex. 1 |
| 186 | 9-fluoro-2-[(3- phenylpropyl)amino]benzo[h][1,3]thiazolo[5,4-f]isoquinolin-7(6H)-one | 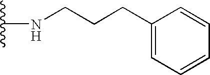 | 404 | Ex. 1 |
| 187 | 9-fluoro-2-{[(1R)-1-(4- methoxyphenyl)ethyl]amino}benzo [h][1,3]thiazolo[5,4-f]isoquinolin- 7(6H)-one | 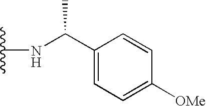 | 420 | Ex. 1 |
| 188 | 9-fluoro-2-{[(1S)-1-(4- methoxyphenyl)ethyl]amino}benzo [h][1,3]thiazolo[5,4-f]isoquinolin- 7(6H)-one | 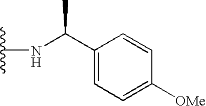 | 420 | Ex. 1 |
| 189 | 9-fluoro-2-(pyridin-3- ylamino)benzo[h][1,3]thiazolo[5,4- f]isoquinolin-7(6H)-one | 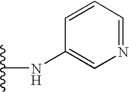 | 363 | Ex. 1 |
| 190 | 9-fluoro-2-(4-pyridin-2-ylpiperazin-1- yl)benzo[h][1,3]thiazolo[5,4- f]isoquinolin-7(6H)-one | 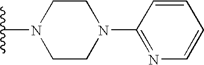 | 432 | Ex. 1 |
| 191 | 9-fluoro-2-{[3-(1H-imidazol-1- yl)propyl]amino}benzo[h][1,3]thiazolo [5,4-f]isoquinolin-7(6H)-one | 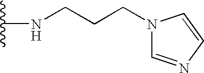 | 394 | Ex. 1 |
| 192 | 9-fluoro-2-[(2-morpholin-4- ylethyl)amino]benzo[h][1,3]thiazolo [5,4-f]isoquinolin-7(6H)-one | 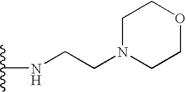 | 399 | Ex. 1 |
| 193 | 9-fluoro-2-[(2- | —NH(CH₂)₂OMe | 344 | Ex. 1 |
| | methoxyethyl)amino]benzo[h][1,3] | | | |
| | thiazolo[5,4-f]isoquinolin-7(6H)-one | | | |
| 194 | 9-fluoro-2-[(tetrahydrofuran-2- ylmethyl)amino]benzo[h][1,3]thiazolo [5,4-f]isoquinolin-7(6H)-one | 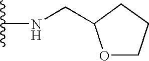 | 370 | Ex. 1 |
| 195 | 9-fluoro-2-[(2-pyridin-2- ylethyl)amino]benzo[h][1,3]thiazolo [5,4-f]isoquinolin-7(6H)-one | 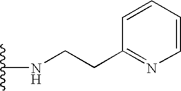 | 391 | Ex. 1 |
| 196 | 9-fluoro-2-{[2-(1H-imidazol-4- yl)ethyl]amino}benzo[h][1,3]thiazolo [5,4-f]isoquinolin-7(6H)-one | 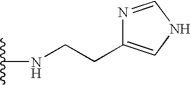 | 380 | Ex. 1 |
| 197 | 9-fluoro-2-[4-(2- hydroxyethyl)piperazin-1- yl]benzo[h][1,3]thiazolo[5,4- f]isoquinolin-7(6H)-one | 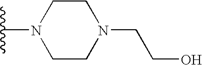 | 399 | Ex. 1 |
| 198 | 2-{[3- (dimethylamino)propyl]amino}-9- fluorobenzo[h][1,3]thiazolo[5,4- f]isoquinolin-7(6H)-one | 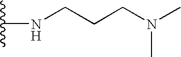 | 371 | Ex. 1 |
| 199 | 9-fluoro-2-(4-hydroxy-4-pyridin-2- ylpiperidin-1- yl)benzo[h][1,3]thiazolo[5,4- f]isoquinolin-7(6H)-one | 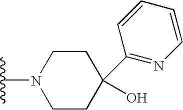 | 447 | Ex. 1 |
| 200 | 2-[bis(2-methoxyethyl)amino]-9- fluorobenzo[h][1,3]thiazolo[5,4- f]isoquinolin-7(6H)-one | 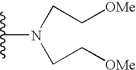 | 402 | Ex. 1 |
| 201 | (9-fluoro-7-oxo-6,7- | —CH₂CN | 310 | Ex. 1 |
| | dihydrobenzo[h][1,3]thiazolo[5,4- | | | |
| | f]isoquinolin-2-yl)acetonitrile | | | |
| 202 | 2-{benzyl[2- (dimethylamino)ethyl]amino}-9- fluorobenzo[h][1,3]thiazolo[5,4- f]isoquinolin-7(6H)-one | 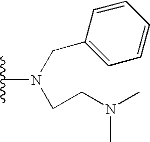 | 447 | Ex. 1 |
| 203 | 9-fluoro-2-{[3- (trifluoromethyl)phenyl]amino}benzo [h][1,3]thiazolo[5,4-f]isoquinolin- 7(6H)-one | 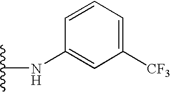 | 430 | Ex. 1 |
| 204 | 9-fluoro-2-[(2- methoxyphenyl)amino]benzo[h][1,3]thiazolo[5,4-f]isoquinolin-7(6H)-one | 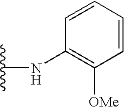 | 392 | Ex. 1 |
| 205 | 9-fluoro-2- | —NHCH₂CH₂CH₃ | 328 | Ex. 1 |
| | (propylamino)benzo[h][1,3]thiazolo | | | |
| | [5,4-f]isoquinolin-7(6H)-one | | | |
| 206 | 9-fluoro-2- (isopropylamino)benzo[h][1,3]thiazolo [5,4-f]isoquinolin-7(6H)-one | 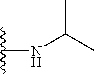 | 328 | Ex. 1 |
| 207 | 3-[(9-fluoro-7-oxo-6,7- dihydrobenzo[h][1,3]thiazolo[5,4- f]isoquinolin-2-yl)amino]benzonitrile | 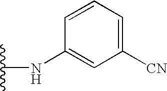 | 387 | Ex. 1 |
| 208 | 9-fluoro-2-[(3- hydroxyphenyl)amino]benzo[h][1,3]thiazolo[5,4-f]isoquinolin-7(6H)-one | 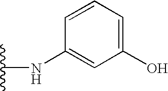 | 378 | Ex. 1 |
| 209 | 9-fluoro-2-[(trans-4-hydroxy-4- pyridin-2- ylcyclohexyl)amino]benzo[h][1,3]thiazolo[5,4-f]isoquinolin-7(6H)-one | 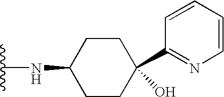 | 461 | Ex. 1 |
| 210 | 9-fluoro-2-[cis-4-hydroxy-3- (hydroxymethyl)piperidin-1- yl]benzo[h][1,3]thiazolo[5,4- f]isoquinolin-7(6H)-one | 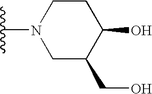 | 400 | Ex. 1 |
| 211 | 9-fluoro-2-[trans-4-hydroxy-3- (hydroxymethyl)piperidin-1- yl]benzo[h][1,3]thiazolo[5,4- f]isoquinolin-7(6H)-one | 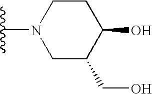 | 400 | Ex. 1 |
| 212 | 9-fluoro-2-[(2- | —NH(CH₂)₂OH | 330 | Ex. 1 |
| | hydroxyethyl)amino]benzo[h][1,3] | | | |
| | thiazolo[5,4-f]isoquinolin-7(6H)-one | | | |
| 213 | 9-fluoro-2-[(4- hydroxybenzyl)amino]benzo[h][1,3]thiazolo[5,4-f]isoquinolin-7(6H)-one | 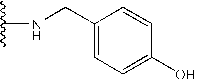 | 392 | Ex. 1 |
| 214 | 9-fluoro-2-[4- (hydroxymethyl)piperidin-1- yl]benzo[h][1,3]thiazolo[5,4- f]isoquinolin-7(6H)-one | 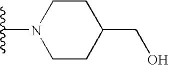 | 384 | Ex. 1 |
| 215 | 9-fluoro-2-[3- (hydroxymethyl)piperidin-1- yl]benzo[h][1,3]thiazolo[5,4- f]isoquinolin-7(6H)-one | 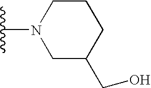 | 384 | Ex. 1 |
| 216 | 9-fluoro-2-[(2-hydroxy-1- methylethyl)amino]benzo[h][1,3]thiazolo[5,4-f]isoquinolin-7(6H)-one | 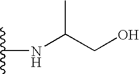 | 344 | Ex. 1 |
| 217 | 9-fluoro-2-[(2S)-2- (hydroxymethyl)pyrrolidin-1- yl]benzo[h][1,3]thiazolo[5,4- f]isoquinolin-7(6H)-one | 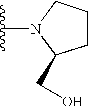 | 370 | Ex. 1 |
| 218 | 9-fluoro-2-[(2R)-2- (hydroxymethyl)pyrrolidin-1- yl]benzo[h][1,3]thiazolo[5,4- f]isoquinolin-7(6H)-one | 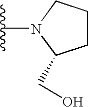 | 370 | Ex. 1 |
| 219 | 2-(4-acetylpiperazin-1-yl)-9- fluorobenzo[h][1,3]thiazolo[5,4- f]isoquinolin-7(6H)-one | 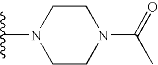 | 397 | Ex. 24 |
| 220 | 9-fluoro-2-piperazin-1- ylbenzo[h][1,3]thiazolo[5,4- f]isoquinolin-7(6H)-one | 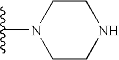 | 355 | Ex. 24 |
| 221 | 2-{4- [(dimethylamino)acetyl]piperazin-1- yl}-9- fluorobenzo[h][1,3]thiazolo[5,4- f]isoquinolin-7(6H)-one | 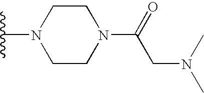 | 440 | Ex. 24 |
| 222 | 9-fluoro-2-[4-(2- hydroxyethyl)piperidin-1- yl]benzo[h][1,3]thiazolo[5,4- f]isoquinolin-7(6H)-one | 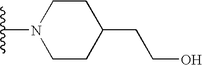 | 398 | Ex. 1 |
| 223 | 9-fluoro-2-{4-[(2R)-2- hydroxypropyl]piperazin-1- yl}benzo[h][1,3]thiazolo[5,4- f]isoquinolin-7(6H)-one | 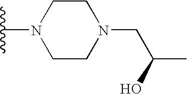 | 413 | Ex. 24 |
| 224 | 2-[(2,3-dihydroxypropyl)amino]-9- fluorobenzo[h][1,3]thiazolo[5,4- f]isoquinolin-7(6H)-one | 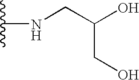 | 360 | Ex. 1 |
| 225 | 4-(9-fluoro-7-oxo-6,7- dihydrobenzo[h][1,3]thiazolo[5,4- f]isoquinolin-2-yl)piperazine-1- carboxamide | 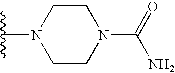 | 398 | Ex. 24 |
| 226 | methyl 4-(9-fluoro-7-oxo-6,7- dihydrobenzo[b][1,3]thiazolo[5,4- f]isoquinolin-2-yl)piperazine-1- carboxylate | 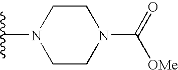 | 413 | Ex. 24 |
| 227 | ethyl 4-(9-fluoro-7-oxo-6,7- dihydrobenzo[h][1,3]thiazolo[5,4- f]isoquinolin-2-yl)piperazine-1- carboxylate | 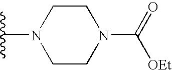 | 427 | Ex. 24 |
| 228 | 4-(9-fluoro-7-oxo-6,7- dihydrobenzo[h][1,3]thiazolo[5,4- f]isoquinolin-2-yl)piperazine-1- carbaldehyde | 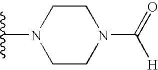 | 383 | Ex. 24 |
| 229 | 9-fluoro-2-[4-(1H-tetrazol-5- ylacetyl)piperazin-1- yl]benzo[h][1,3]thiazolo[5,4- f]isoquinolin-7(6H)-one | 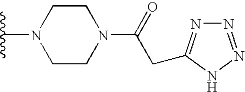 | 465 | Ex. 24 |
| 230 | 9-fluoro-2-(pyridin-2- ylamino)benzo[h][1,3]thiazolo[5,4- f]isoquinolin-7(6H)-one | 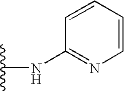 | 363 | Ex. 1 |
| 231 | 9-fluoro-2-(pyridin-4- ylamino)benzo[h][1,3]thiazolo[5,4- f]isoquinolin-7(6H)-one | 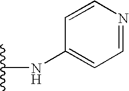 | 363 | Ex. 1 |
| 232 | 9-fluoro-2-[(3R,4R)-3-methoxy-4- (methylamino)pyrrolidin-1- yl]benzo[h][1,3]thiazolo[5,4- f]isoquinolin-7(6H)-one | 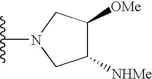 | 399 | Ex. 1 |
| 233 | 9-fluoro-2-[(3S,4R)-3-methoxy-4- (methylamino)pyrrolidin-1- yl]benzo[h][1,3]thiazolo[5,4- f]isoquinolin-7(6H)-one | 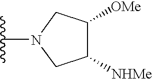 | 399 | Ex. 1 |
| 234 | benzyl (3R,4R)-3-[(9-fluoro-7-oxo- 6,7-dihydrobenzo[h][1,3]thiazolo[5,4- f]isoquinolin-2-yl)(methyl)amino]-4- methoxypyrrolidine-1-carboxylate | 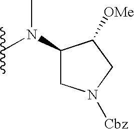 | 533 | Ex. 1 |
| 235 | 2-(1-ethyl-1-hydroxypropyl)-9- fluorobenzo[h][1,3]thiazolo[5,4- f]isoquinolin-7(6H)-one | 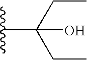 | 357 | Ex. 23 |
| 236 | 9-fluoro-2- | —CH₂OH | 301 | Ex. 23 |
| | (hydroxymethyl)benzo[h][1,3]thiazolo | | | |
| | [5,4-f]isoquinolin-7(6H)-one | | | |
| 237 | 2-(ethoxymethyl)-9- | —CH₂OEt | 329 | Ex. 23 |
| | fluorobenzo[h][1,3]thiazolo[5,4- | | | |
| | f]isoquinolin-7(6H)-one | | | |
| 238 | 3-(9-fluoro-7-oxo-6,7- | —CH₂CH₂CN | 324 | Ex. 23 |
| | dihydrobenzo[h][1,3]thiazolo[5,4- | | | |
| | f]isoquinolin-2-yl)propanenitrile | | | |
| 239 | [1-(9-fluoro-7-oxo-6,7- dihydrobenzo[h][1,3]thiazolo[5,4- f]isoquinolin-2-yl)piperidin-4- yl]acetonitrile | 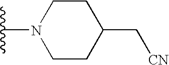 | 393 | Ex. 1 |
| 240 | 1-(9-fluoro-7-oxo-6,7- dihydrobenzo[h][1,3]thiazolo[5,4- f]isoquinolin-2-yl)piperidine-4- carbonitrile | 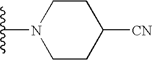 | 379 | Ex. 1 |
| 241 | 3-[(9-fluoro-7-oxo-6,7- dihydrobenzo[h][1,3]thiazolo[5,4- f]isoquinolin-2- yl)methoxy]propanenitrile | 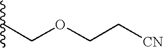 | 354 | Ex. 23 |
| 242 | 9-fluoro-2-{4-[2-(1H-1,2,4-triazol-1- yl)ethyl]piperidin-1- yl}benzo[h][1,3]thiazolo[5,4- f]isoquinolin-7(6H)-one | 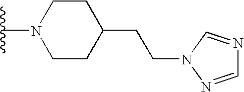 | 449 | Ex. 1 |
| 243 | 9-fluoro-2-[4-(1H-1,2,4-triazol-1- ylmethyl)piperidin-1- yl]benzo[h][1,3]thiazolo[5,4- f]isoquinolin-7(6H)-one | 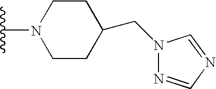 | 435 | Ex. 1 |
| 244 | 9-fluoro-2-[4-(1H-imidazol-1- ylmethyl)piperidin-1- yl]benzo[h][1,3]thiazolo[5,4- f]isoquinolin-7(6H)-one | 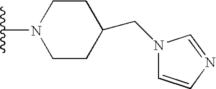 | 434 | Ex. 1 |
| 245 | 3-[1-(9-fluoro-7-oxo-6,7- dihydrobenzo[h][1,3]thiazolo[5,4- f]isoquinolin-2-yl)piperidin-4- yl]propanenitrile | 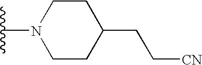 | 407 | Ex. 1 |
| 246 | 9-fluoro-2-{4-[2-(1H-imidazol-1- yl)ethyl]piperidin-1- yl}benzo[h][1,3]thiazolo[5,4- f]isoquinolin-7(6H)-one | 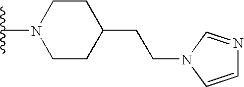 | 448 | Ex. 1 |
| 247 | 2-{4- [(diethylamino)methyl]piperidin-1- yl}-9- fluorobenzo[h][1,3]thiazolo[5,4- f]isoquinolin-7(6H)-one | 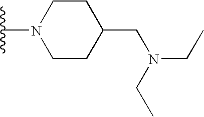 | 439 | Ex. 1 |
| 247a | 9-fluoro-2-(pyridin-3- ylmethyl)benzo[h][1,3]thiazolo[5,4- f]isoquinolin-7(6H)-one | 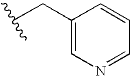 | 362 | Ex. 1 |
| 247b | 9-fluoro-2-(pyridin-2- ylmethyl)benzo[h][1,3]thiazolo[5,4- f]isoquinolin-7(6H)-one | 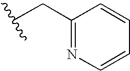 | 362 | Ex. 1 |
| 247c | 9-fluoro-2-[(1-oxidopyridin-3- yl)methyl]benzo[h][1,3]thiazolo[5,4- f]isoquinolin-7(6H)-one | 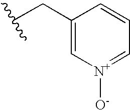 | 378 | Ex. 1 |
| 247d | 9-fluoro-2-(pyridin-4- ylmethyl)benzo[h][1,3]thiazolo[5,4- f]isoquinolin-7(6H)-one | 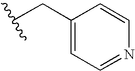 | 362 | Ex. 1 |
| 247e | 9-fluoro-2-[pyridin(6- methoxypyridin-3- yl)methyl]benzo[h][1,3]thiazolo[5,4- f]isoquinolin-7(6H)-one | 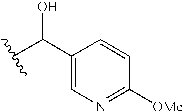 | 408 | Ex. 23 |
| 247f | 9-fluoro-2-[3-hydroxy-1-(pyridin-4- ylmethyl)piperidin-3- yl]benzo[h][1,3]thiazolo[5,4- f]isoquinolin-7(6H)-one | 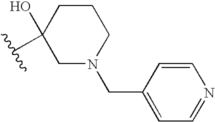 | 461 | Ex. 23 |
| 247g | 2-(1-acetyl-3-hydroxypiperidin-3-yl)- 9-fluorobenzo[b][1,3]thiazolo[5,4- f]isoquinolin-7(6H)-one | 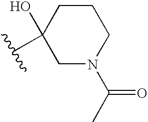 | 412 | Ex. 23 |
| 247h | 9-fluoro-2-[(4-hydroxypiperidin-1- yl)methyl]benzo[h][1,3]thiazolo[5,4- f]isoquinolin-7(6H)-one | 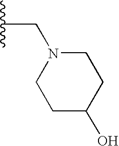 | 384 | Ex. 23 |
| 247i | 2-[(4-acetylpiperazin-1-yl)carbonyl]- 9-fluorobenzo[h][1,3]thiazolo[5,4- f]isoquinolin-7(6H)-one | 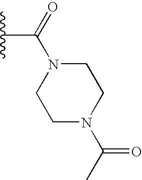 | 425 | Ex. 23 |
| 247j | 9-fluoro-2-[3-hydroxy-1- (methylsulfonyl)piperidin-3- yl]benzo[h][1,3]thiazolo[5,4- f]isoquinolin-7(6H)-one | 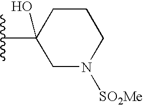 | 448 | Ex. 23 |
| 247k | 9-fluoro-2-(1-hydroxy-1-pyridin-3- ylethyl)benzo[h][1,3]thiazolo[5,4- f]isoquinolin-7(6H)-one | 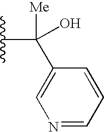 | 392 | Ex. 23 |
| 247l | 9-fluoro-2-(1-hydroxy-1-pyridin-4- ylethyl)benzo[h][1,3]thiazolo[5,4- f]isoquinolin-7(6H)-one | 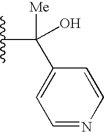 | 392 | Ex. 23 |
| 247m | 9-fluoro-2-[1-hydroxy-1-(1- oxidopyridin-3- yl)ethyl]benzo[h][1,3]thiazolo[5,4- f]isoquinolin-7(6H)-one | 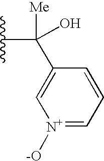 | 408 | Ex. 23 |
| 247n | 9-fluoro-2-[1-hydroxy-1-(1- oxidopyridin-4- yl)ethyl]benzo[h][1,3]thiazolo[5,4- f]isoquinolin-7(6H)-one | 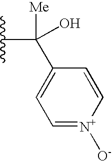 | 408 | Ex. 23 |
| 247o | 2-{4- [(dimethylamino)methyl]piperidin-1- yl}-9- fluorobenzo[h][1,3]thiazolo[5,4- f]isoquinolin-7(6H)-one | 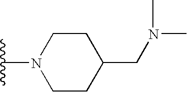 | 411 | Ex. 1 |
| 247p | 9-fluoro-2-(pyridine-3- yloxy)benzo[b][1,3]thiazolo[5,4- f]isoquinolin-7(6H)-one | 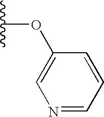 | 364 | Ex. 23 |
| 247q | 9-fluoro-2-(pyridin-4- ylthio)benzo[h][1,3]thiazolo[5,4- f]isoquinolin-7(6H)-one | 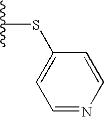 | 380 | Ex. 23 |
| 247r | 9-fluoro-2-[(trans-4- hydroxycyclohexyl)amino]benzo[h][1,3]thiazolo[5,4-f]isoquinolin-7(6H)- one | 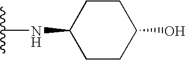 | 384 | Ex. 1 |
| 247s | 9-fluoro-2-(4-pyrazin-2-ylpiperazin- 1-yl)benzo[h][1,3]thiazolo[5,4- f]isoquinolin-7(6H)-one | 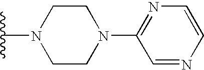 | 433 | Ex. 24 |
| 247t | (9-fluoro-7-oxo-6,7- dihydrobenzo[h][1,3]thiazolo[5,4- f]isoquinolin-2-yl)-N- methylpiperazine-1-carboxamide | 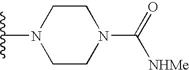 | 412 | Ex. 24 |
| 247u | 9-fluoro-2-[4-(1H-pyrazol-3- ylcarbonyl)piperazin-1- yl]benzo[h][1,3]thiazolo[5,4- f]isoquinolin-7(6H)-one | 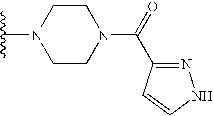 | 449 | Ex. 24 |
| 247v | 9-fluoro-2-[4-(1H-pyrazol-4- ylcarbonyl)piperazin-1- yl]benzo[h][1,3]thiazolo[5,4- f]isoquinolin-7(6H)-one | 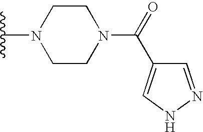 | 449 | Ex. 24 |
| 247w | 9-fluoro-2-[4-(1H-imidazol-2- ylcarbonyl)piperazin-1- yl]benzo[h][1,3]thiazolo[5,4- f]isoquinolin-7(6H)-one | 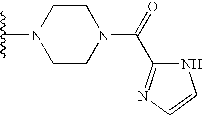 | 449 | Ex. 24 |
| 247x | 2-[4-(aminoacetyl)piperazin-1-yl]-9- fluorobenzo[h][1,3]thiazolo[5,4- f]isoquinolin-7(6H)-one | 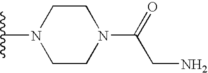 | 412 | Ex. 24 |
| 247y | 2-[4-(azetidin-3-ylcarbonyl)piperazin- 1-yl]-9- fluorobenzo[h][1,3]thiazolo[5,4- f]isoquinolin-7(6H)-one | 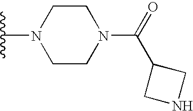 | 438 | Ex. 24 |
| 247z | N-(tert-butyl)-4-(9-fluoro-7-oxo-6,7- dihydrobenzo[h][1,3]thiazolo[5,4- f]isoquinolin-2-yl)piperazine-1- carboxamide | 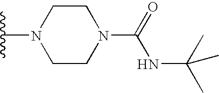 | 454 | Ex. 24 |
| 247aa | 9-fluoro-2-{4-[(2S)-2- hydroxypropyl]piperazin-1- yl}benzo[h][1,3]thiazolo[5,4- f]isoquinolin-7(6H)-one | 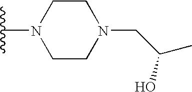 | 413 | Ex. 24 |
| 247ab | 3-[4-(9-fluoro-7-oxo-6,7- dihydrobenzo[h][1,3]thiazolo[5,4- f]isoquinolin-2-yl)piperazin-1-yl]-3- oxopropanenitrile | 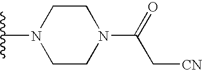 | 422 | Ex. 24 |
| 247ac | 9-fluoro-2-[4- (methylsulfonyl)piperazin-1- yl]benzo[h][1,3]thiazolo[5,4- f]isoquinolin-7(6H)-one | 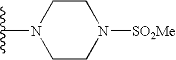 | 433 | Ex. 24 |
| 247ad | 4-(9-fluoro-7-oxo-6,7- dihydrobenzo[h][1,3]thiazolo[5,4- f]isoquinolin-2-yl)-N,N- dimethylpiperazine-1-carboxamide | 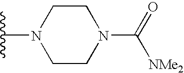 | 426 | Ex. 24 |
| 247ae | 1-(9-fluoro-7-oxo-6,7- dihydrobenzo[h][1,3]thiazolo[5,4- f]isoquinolin-2-yl)piperidine-4- carboxylic acid | 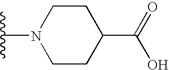 | 398 | Ex. 1 |
| 247af | 9-fluoro-2-{4-hydroxy-4- [(methylamino)methyl]piperidin-1- yl}benzo[h][1,3]thiazolo[5,4- f]isoquinolin-7(6H)-one | 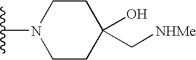 | 413 | Ex. 1 |
| 247ag | 9-fluoro-2-[4-hydroxy-4-(1H-1,2,4- triazol-1-ylmethyl)piperidin-1- yl]benzo[h][1,3]thiazolo[5,4- f]isoquinolin-7(6H)-one | 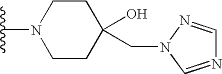 | 451 | Ex. 1 |
| 247ah | 9-fluoro-2-[4-hydroxy-4- (hydroxymethyl)piperidin-1- yl]benzo[h][1,3]thiazolo[5,4- f]isoquinolin-7(6H)-one | 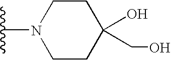 | 400 | Ex. 1 |
| 247ai | 9-fluoro-2-piperidin-3- ylbenzo[h][1,3]thiazolo[5,4- f]isoquinolin-7(6H)-one | 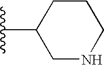 | 354 | Ex. 1 |
| 247aj | 2-(1-acetylpiperidin-3-yl)-9- fluorobenzo[h][1,3]thiazolo[5,4- f]isoquinolin-7(6H)-one | 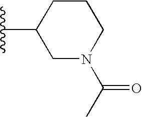 | 396 | Ex. 1 |
| 247al | 9-fluoro-2-[1- (methylsulfonyl)piperidin-3- yl]benzo[h][1,3]thiazolo[5,4- f]isoquinolin-7(6H)-one | 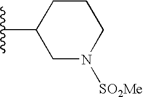 | 432 | Ex. 1 |
| 247am | 9-fluoro-2-[1-(pyridin-4- ylmethyl)piperidin-3- yl]benzo[h][1,3]thiazolo[5,4- f]isoquinolin-7(6H)-one | 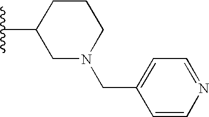 | 445 | Ex. 1 |
| 247an | 3-[3-(9-fluoro-7-oxo-6,7- dihydrobenzo[b][1,3]thiazolo[5,4- f]isoquinolin-2-yl)piperidin-1-yl]-3- oxopropanenitrile | 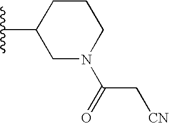 | 421 | Ex. 1 |
| 247ao | 9-fluoro-2-{1-[(2S)-2- hydroxypropyl]piperidin-3- yl}benzo[h][1,3]thiazolo[5,4- f]isoquinolin-7(6H)-one | 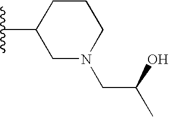 | 412 | Ex. 1 |
| 247ap | 2-{1- [(dimethylamino)acetyl]piperidin-3- yl}-9- fluorobenzo[h][1,3]thiazolo[5,4- f]isoquinolin-7(6H)-one | 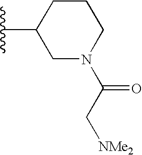 | 441 | Ex. 1 |
| 247aq | 9-fluoro-2-[4-(morpholin-4- ylmethyl)piperidin-1- yl]benzo[h][1,3]thiazolo[5,4- f]isoquinolin-7(6H)-one | 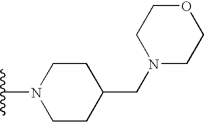 | 453 | Ex. 1 |
| 247ar | 9-fluoro-2-[4-(2-morpholin-4- ylethyl)piperidin-1- yl]benzo[h][1,3]thiazolo[5,4- f]isoquinolin-7(6H)-one | 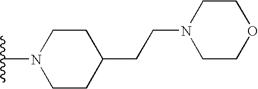 | 467 | Ex. 1 |

TABLE 5

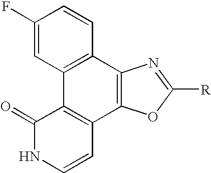

| | | | Mass | |
|---|---|---|---|---|
| Ex. | | | Spec | Method of |
| No. | Name | R | (M + H) | Preparation |
| 248 | 4-(9-fluoro-7-oxo-6,7- dihydrobenzo[h][1,3]oxazolo[5,4- f]isoquinolin-2-yl)butanenitrile | —(CH₂)₃CN | 322 | Ex. 23 |
| 249 | 9-fluoro-2-[3-(1H-1,2,4-triazol-1- yl)propyl]benzo[h][1,3]oxazolo[5,4- f]isoquinolin-7(6H)-one | 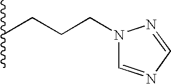 | 364 | Ex. 23 |
| 250 | 9-fluoro-2-{[(1R)-1-phenylethyl]amino}benzo[h][1,3]oxazolo[5,4- f]isoquinolin-7(6H)-one | 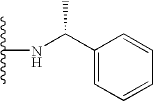 | 374 | Ex. 9 |
| 251 | 9-fluoro-2-{[(1S)-1- phenylethyl]amino}benzo[h][1,3]oxazolo[5,4-f]isoquinolin-7(6H)-one | 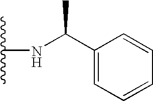 | 374 | Ex. 9 |
| 252 | 9-fluoro-2-[(2- methoxybenzyl)amino]benzo[h][1,3]oxazolo[5,4-f]isoquinolin-7(6H)-one | 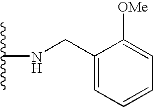 | 390 | Ex. 9 |
| 253 | 9-fluoro-2-[(4- methoxybenzyl)amino]benzo[h][1,3]oxazolo[5,4-f]isoquinolin-7(6H)-one | 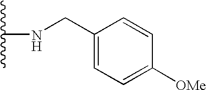 | 390 | Ex. 9 |
| 254 | 9-fluoro-2-[(3- methoxybenzyl)amino]benzo[h][1,3]oxazolo[5,4-f]isoquinolin-7(6H)-one | 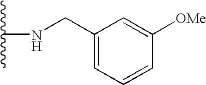 | 390 | Ex. 9 |
| 255 | 9-fluoro-2-(4-oxopiperidin-1- yl)benzo[h][1,3]oxazolo[5,4- f]isoquinolin-7(6H)-one | 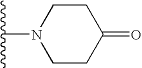 | 352 | Ex. 9 |
| 256 | 9-fluoro-2-(4-hydroxypiperidin-1- yl)benzo[h][1,3]oxazolo[5,4- f]isoquinolin-7(6H)-one | 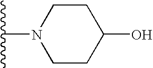 | 354 | Ex. 9 |
| 256a | 9-fluoro-2-{[1- (methylsulfonyl)piperidin-4- yl]amino}benzo[h][1,3]oxazolo[5,4- f]isoquinolin-7(6H)-one | 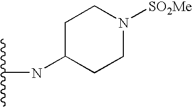 | 431 | Ex. 9 |
| 256b | 2-[(1-acetylpiperidin-4-yl)amino]-9- fluorobenzo[h][1,3]oxazolo[5,4- f]isoquinolin-7(6H)-one | 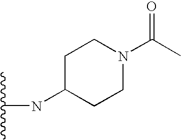 | 395 | Ex. 9 |
| 256c | 9-fluoro-2-[hydroxy(pyridin-3- yl)methyl]benzo[h][1,3]oxazolo[5,4- f]isoquinolin-7(6H)-one | 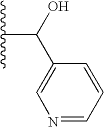 | 362 | Ex. 23 |
| 256d | 2-(4-acetylpiperazin-1-yl)-9- fluorobenzo[h][1,3]oxazolo[5,4- f]isoquinolin-7(6H)-one | 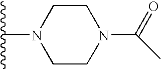 | 381 | Ex. 9 |
| 256e | 4-(9-fluoro-7-oxo-6,7- dihydrobenzo[h][1,3]oxazolo[5,4- f]isoquinolin-2-yl)piperazine-1- carboxamide | 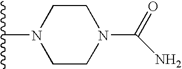 | 382 | Ex. 9 |
| 256f | 2-[(4-acetylpiperazin-1-yl)methyl]-9- fluorobenzo[h][1,3]oxazolo[5,4- f]isoquinolin-7(6H)-one | 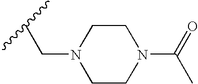 | 395 | Ex. 23 |
| 256g | 9-fluoro-2-[(trans-4- hydroxycyclohexyl)amino]benzo[h][1,3]oxazolo[5,4-f]isoquinolin-7(6H)- one | 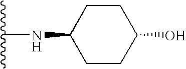 | 368 | Ex. 9 |
| 256h | 9-fluoro-2-(pyridin-3- yloxy)benzo[h][1,3]oxazolo[5,4- f]isoquinolin-7(6H)-one | 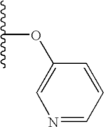 | 348 | Ex. 23 |
| 256i | 2-[(1-acetylpiperidin-3-yl)amino]-9- fluorobenzo[h][1,3]oxazolo[5,4- f]isoquinolin-7(6H)-one | 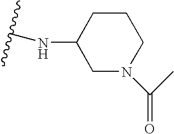 | 395 | Ex. 9 |
| 256j | 9-fluoro-2-{[1- (methylsulfonyl)piperidin-3- yl]amino}benzo[h][1,3]oxazolo[5,4- f]isoquinolin-7(6H)-one | 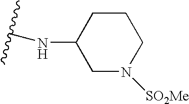 | 431 | Ex. 9 |
| 256k | 9-fluoro-2-[4-(1,3-thiazol-2- yl)piperazin-1- yl]benzo[h][1,3]oxazolo[5,4- f]isoquinolin-7(6H)-one | 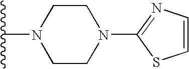 | 422 | Ex. 9 |

### Example 257

### 2-(trans-4-Hydroxycyclohexyl)imidazo[1,2-a]pyrido[4,3-c]-1,6-naphthyridin-8(7H)-one bis(trifluoroacetate) (salt)

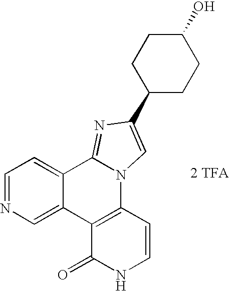 Step A: 2-Butoxypyridin-4-amine

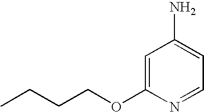

2-Chloropyridin-4-amine (20.0 g, 0.156 mol) and sodium hydroxide (25 g, 0.63 mol) were dissolved in 1-butanol (160 mL, 1.8 mol). The solution was heated at reflux for 48 h. The solution was cooled to rt, diluted with water (500 mL), and then extracted with ethyl acetate (2×250 mL). The combined organic layer was washed with brine (250 mL), dried over sodium sulfate, and concentrated in vacuo to afford product as a yellow oil (28.2 g, 98%). LCMS for C₉H₁₅N₂O (M+H)⁺: m/z=167; ¹H NMR (500 MHz, CD₃OD): δ 7.57 (d, J=5.9 Hz, 1H), 6.21 (dd, J=5.9, 1.9 Hz, 1H), 5.93 (d, J=1.9 Hz, 1H), 4.06 (t, J=6.4H, 2H), 1.72-1.68 (m, 2H), 1.48-1.41 (m, 2H), 0.99 (t, J=5.8 Hz, 3H).
Step B: 2-Butoxy-3-iodopyridin-4-amine

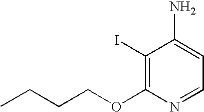

2-Butoxypyridin-4-amine (21.2 g, 0.115 mol) was suspended in acetic acid (250 mL, 4.4 mol) and N-iodosuccinimide (27.1 g, 0.120 mol) was added in portions (ca. 5% per portion) over 60 minutes. The reaction mixture was stirred vigorously at rt for an additional 30 minutes. The solution was concentrated under vacuum to remove most of the acetic acid. The residue was diluted with ethyl acetate (400 mL), neutralized with saturated aqueous sodium bicarbonate solution (400 mL), and the organic layer was separated. The organic layer was washed with aqueous. sodium thiosulfate sol. (1 M, 150 mL), water (400 mL), and then brine (400 mL). The organic layer was dried over sodium sulfate and then concentrated in vacuo. Silica gel chromatography (5-35% ethyl acetate:hexanes) afforded product as a colorless oil (13.8 g, 41%). LCMS for C₉H₁₄IN₂O (M+H)⁺: m/z=293.; ¹H NMR (400 MHz, CD₃OD): δ 7.53 (d, J=5.9 Hz, 1H), 6.29 (d, J=5.9 Hz, 1H), 4.20 (t, J=6.3 Hz, 2H), 1.76-1.72 (m, 2H), 1.54-1.49 (m, 2H), 0.98 (t, J=6.4 Hz, 3H).
Step C: 4-[(Diisopropylamino)carbonyl]pyridin-3-ylboronic acid

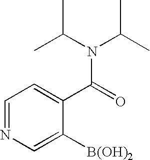

n-Butyllithium in hexane (46.8 mL, 1.6 M) in THF (120 mL) was chilled to −78° C. N,N-diisopropylisonicotinamide (10.3 g, 0.0499 mol) in tetrahydrofuran (120 mL) was added and the solution was stirred 1 h at −78° C. Triisopropyl borate (17.3 mL, 0.0749 mol) was added and the solution was warmed to 0° C. and stirred 0.5 h at 0° C. Aqueous hydrochloric acid solution (1 M, 250 mL) was added and the solution was washed with ethyl acetate (250 mL), adjusted to be basic (pH 10) with saturated sodium bicarbonate solution (250 mL), washed with ethyl acetate (250 mL), diluted with brine (50 mL), and then extracted with THF (500 mL). The organic layer was dried over sodium sulfate, filtered, and concentrated. The residue was diluted with water (100 mL) and frozen under vacuum. to afford product as a fluffy white solid (4.1 g, 33%). LCMS for C₁₂H₂₀BN₂O₃ (M+H)⁺: m/z=251; ¹H NMR (300 MHz, CD₃OD): δ 8.75 (s, 1H), 8.59 (d, J=4.98 Hz, 1H), 7.39 (bs, 1H), 3.92 (m, 1H), 3.71 (bs, 1H), 1.63 (m, 6H), 1.22 (m, 6H)
Step D: 1-Butoxypyrido[4,3-c]-1,6-naphthyridin-6(5H)-one

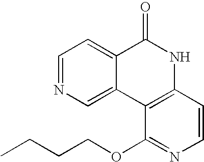

4-[(Diisopropylamino)carbonyl]pyridin-3-ylboronic acid (13.0 g, 0.0520 mol), 2-butoxy-3-iodopyridin-4-amine (12 g, 0.032 mol), potassium carbonate (22.3 g, 0.162 mol), and tetrakis(triphenylphosphine)palladium(0) (2.82 g, 2.44 mmol) were dissolved in N,N-dimethylformamide (200 mL) and water (50.0 mL) and the solution was degassed. The solution was heated under nitrogen atmosphere at bath temp =135° C. for 30 minutes. The solution was diluted aqueous hydrochloric acid (1M, 300 mL), and washed with ethyl acetate (2×300 mL). The aqueous layer was changed to pH 10 with saturated sodium bicarbonate solution (500 mL) and the product was extracted with ethyl acetate (2×300 mL). The organic layer was dried over sodium sulfate, filtered, and concentrated in vacuo. The material (yellow oil, 13.4 g, ca. 80% pure, 89%) was used crude with the impurities in the subsequent cyclization reaction. LCMS for C₂₁H₃₁N₄O₂(M+H)⁺: m/z=371; 4′-Amino-2′-butoxy-N,N-diisopropyl-3,3′-bipyridine-4-carboxamide (13.4 g, 0.0289 mol) in THF (200 mL) was treated with a solution sodium hexamethyldisilazane in THF (1.0 M, 57.9 mL) and stirred at rt for 30 minutes. The solution was quenched with methanol (100 mL) and concentrated in vacuo. The residue was triterated with diethyl ether (700 mL), and allowed to stir with agitation for 60 minutes. The white precipitate was collected by suction filtration to afford product as a white solid (8.1 g, 93% pure, 97% yield). LCMS calculated for C₁₅H₁₆N₃O₂ (M+H)⁺: m/z=270; ¹H NMR (400 MHz, CD₃OD): δ 10.4 (s, 1H), 8.63 (d, J=5.3 Hz, 1H), 8.54 (s, 1H), 8.30 (d, J=5.3 Hz, 1H), 7.92 (d, J=5.9 Hz, 1H), 7.00 (d, J=5.9 Hz, 1H), 4.56 (t, J=6.5 Hz, 2H), 2.03-1.95 (m, 2H), 1.69-1.63 (m, 2H), 0.98 (t, J=7.3 Hz, 3H).
Step E: 5-[2-(4-Hydroxycyclohexyl)-2-oxoethyljpyrido[4,3-c]-1,6-naphthyridine-1,6(2H, 5H)-dione hydrochloride

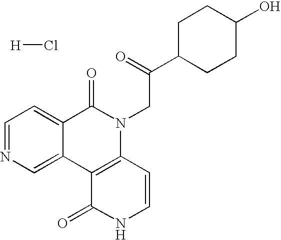

1-Butoxypyrido[4,3-c]-1,6-naphthyridin-6(5H)-one (8.25 g, 27.6 mmol), 4-(2-bromoacetyl)cyclohexyl acetate (8.46 g, 29.0 mmol), and potassium carbonate (12.4 g, 89.7 mmol) in N,N-dimethylformamide (90 mL) were stirred at rt for 1 h. The solution was diluted with ethyl acetate (200 mL) and washed with water (200 mL) and brine (200 mL). The organic phase was dried over sodium sulfate, filtered, and concentrated in vacuo to afford product as a yellow solid. (15.1 g, 85% pure, 97% yield). LCMS calculated for C₂₅H₃₀N₃O₅(M+H)⁺: m/z=452; ¹H NMR (400 MHz, CDCl₃); trans-4-[2-(1-Butoxy-6-oxopyrido[4,3-c]-1,6-naphthyridin-5(6H)-yl)acetyl]cyclohexyl acetate (15.1 g, 28.4 mmol) in aqueous hydrochloric acid (4.0 M, 200 mL) was heated at reflux for one hour. The solution was concentrated in vacuo and the residue diluted with 100 mL DMF to form a green suspension green. This solution was treated with 400 mL diethylether to precipitate a green solid that was collected by suction filtration to afford product as a yellow-green solid (12.8 g). LCMS calculated for C₁₉H₂₀N₃O₄ (free baseM+H)⁺: m/z=354; ¹H NMR (300 MHz, d₆-DMSO): δ 12.42 (brs, 1H), 10.89 (s, 1H), 8.73 (d, J=5.6 Hz, 1H), 8.52 (s, 1H), 8.19 (d, J=5.7 Hz, 1H), 7.83 (t, J=7.0 Hz, 1H), 6.44 (d, J=7.0 Hz, 1H), 3.41-3.37 (m, 1H), 2.71-2.66 (m, 1H), 2.09-2.05 (m, 2H), 1.93-1.90 (m, 2H), 1.85-1.75 (m, 2H), 1.45-1.32 (m, 2H).
Step F: 2-(trans-4-Hydroxycyclohexyl)imidazo[1,2-a]pyrido[4,3-c]-1,6-naphthyridin-8(7H)-one bis(trifluoroacetate) (salt)

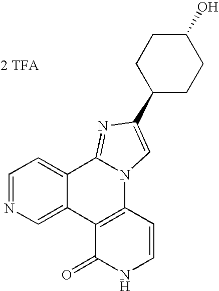

5-[2-(4-Hydroxycyclohexyl)-2-oxoethyl]pyrido[4,3-c]-1,6-naphthyridine-1,6(2H,5H)-dione hydrochloride (10.2 g, 22.2 mol) and ammonium acetate (20.6 g, 0.267 mol) in N,N-dimethylformamide (60.0 mL, 0.775 mol) was divided into 12 batches and each batch was heated in a sealed tube in the microwave at 200° C. for 40 minutes during which the pressure rose to 10 bar These batches were cooled, diluted with DMF (15 mL per batch to 25 mL per batch total volume), and purified after filtration by reverse phase preparative HPLC to afford the desired trans adduct as a fluffy white powder (1.7 g, 13%) and the cis adduct (Example 258) as a fluffy yellow powder (1.8 g, 14%). LCMS for C₁₉H₁₉N₄O₂ (free base M+H)⁺: m/z=335; ¹H NMR (400 MHz, d₆-DMSO): δ 12.42 (brs, 1H), 10.91 (s, 1H), 8.77 (d, J=5.6 Hz, 1H), 8.53 (s, 1H), 8.49 (d, J=5.7 Hz, 1H), 7.88 (t, J=7.0 Hz, 1H), 7.26 (d, J=7.1 Hz, 1H), 3.45-3.40 (m, 1H), 2.71-2.66 (m, 1H), 2.09-2.05 (m, 2H), 1.93-1.90 (m, 2H), 1.56-1.50 (m, 2H), 1.35-1.27 (m, 2H).

### Example 258

### 2-(cis-4-Hydroxycyclohexyl)imidazo[1,2-a]pyrido[4,3-c]-1,6-naphthyridin-8(7H)-one bis(trifluoroacetate) (salt)

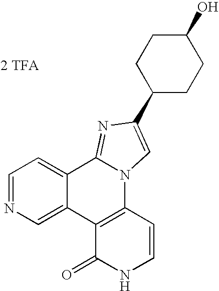

Example 258 was separated from the trans-isomer prepared in Step F of Example 257, by reverse phase preparative chromatography. LCMS for C₁₉H₁₉N₄O₂ (free base M+H)⁺: m/z=335;

### Example 259

### 4-(8-Oxo-7,8-dihydroimidazo[1,2-a]pyrido[4,3-c]-1,6-naphthyridin-2-yl)piperidine-1-carbaldehyde trifluoroacetate

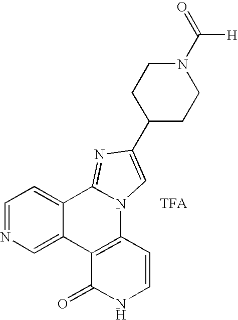

A mixture of 1-Butoxypyrido[4,3-c]-1,6-naphthyridin-6(5H)-one (658 mg, 2.20 mmol), tert-butyl 4-(bromoacetyl)piperidine-1-carboxylate (707 mg, 2.31 mmol), and potassium carbonate (912 mg, 6.60 mmol) in N,N-dimethylformamide (20.0 mL) was stirred at rt for 30 minutes. The solid precipitate was removed by filtration and the solution diluted with ethyl acetate (100 mL). The organic layer was washed with water (100 mL) and brine (100 mL), dried over sodium sulfate, and then concentrated in vacuo to afford a yellow solid which was used crude in the next step (1.12 g, 90% pure, 93% yield). LCMS for C₂₇H₃₅N₄O₅ (M+H)⁺: m/z=495;

tert-Butyl 4-[(1-butoxy-6-oxopyrido[4,3-c]-1,6-naphthyridin-5(6H)-yl)acetyl]-piperidine-1-carboxylate (1.10 g, 2.11 mmol) in aqueous hydrochloric acid (4.0 M, 12 mL) was heated at reflux for 1.5 h. The solution was concentrated to afford a yellow solid (1.02 g, ca. 85% pure, 100% yield), used crude in the next step. LCMS for C₁₈H₁₉N₄O₃ (free base M+H)⁺: m/z=339;

5-(2-oxo-2-piperidin-4-ylethyl)pyrido[4,3-c]-1,6-naphthyridine-1,6(2H,5H)-dione dihydrochloride (60.0 mg, 0.131 mmol) and ammonium acetate (506 mg, 6.56 mmol) in N,N-dimethylformamide (2.00 mL) were heated at 200° C. for 15 minutes. The solution was diluted with methanol (3 mL) and purified by preparative HPLC to afford product as a white solid (31 mg, 51%). LCMS for C₁₉H₁₈N₅O₂ (free base M+H)⁺: m/z=348. ¹H NMR (500 MHz, CD₃OD): δ 11.11 (s, 1H), 8.81 (m, 2H), 8.48 (s, 1H), 8.09 (s, 1H), 7.88 (d, J=7.32 Hz, 1H), 7.29 (d, J=7.32 Hz, 1H), 4.46 (d, J=13.18 Hz, 1H), 3.90 (d, J=13.47 Hz, 1H), 3.38 (m, 1H), 3.07-2.93 (m, 1H), 2.32-2.22 (m, 2H), 1.86-1.74 (m, 2H).

### Example 260

### 2-Piperidin-4-ylimidazo[1,2-a]pyrido[4,3-c]-1,6-naphthyridin-8(7H)-one bis(trifluoroacetate)

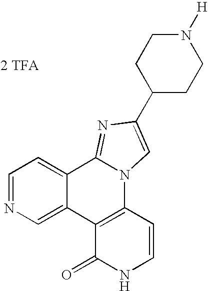

4-(8-Oxo-7,8-dihydroimidazo[1,2-a]pyrido[4,3-c]-1,6-naphthyridin-2-yl)piperidine-1-carbaldehyde trifluoroacetate (480 mg, 0.83 mmol) in aqueous hydrochloric acid solution (4.0 M, 5.00 mL) was heated at reflux for 1 h. The solution was concentrated in vacuo and diluted with water (10 mL) and methanol (5 mL). The solution was purified by preparative HPLC and lyophilized to afford a fluffy tan solid (238 mg, 52%). LCMS calculated for C₁₈H₁₈N₅O (free base M+H)⁺: m/z=320. ¹H NMR (400 MHz, d₆-DMSO): δ 12.63 (brs, 1H), 10.97 (s, 1H), 9.11 (brs, 1H), 8.99 (brs, 1H), 8.87 (d, J=6.1 Hz, 1H), 8.75 (s, 1H), 8.61 (d, J=6.0 Hz, 1H) 7.98 (t, J=6.9 Hz, 1H), 7.38 (d, J=7.0 Hz, 1H), 3.37-3.34 (m, 2H), 3.21-3.04 (m, 3H), 2.28-2.24 (m, 2H), 2.04-1.99 (m, 2H).

### Example 261

### 2-[1-(Cyclopropylmethyl)piperidin-4-yl]imidazo[1,2-a]pyrido[4,3-c]-1,6-naphthyridin-8(7H)-one bis(trifluoroacetate)

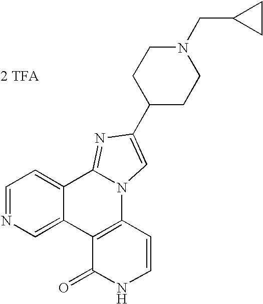

2-Piperidin-4-ylimidazo[1,2-a]pyrido[4,3-c]-1,6-naphthyridin-8(7H)-one bis(trifluoroacetate) (20.0 mg, 0.0365 mmol) and cyclopropanecarboxaldehyde (10.2 mg, 0.146 mmol) were dissolved in methanol (2.0 mL) and stirred 5 minutes. Triethylamine (38.2 μL, 0.274 mmol) was added followed by sodium triacetoxyborohydride (46.5 mg, 0.219 mmol). The solution stirred 1 h at rt. The solution was filtered and purified by preparative HPLC to afford product as a fluffy white solid (17.2 mg, 78%).

LCMS calculated for C₂₁H₂₄N₅O (free base M+H)⁺: m/z=374. ¹H NMR (400 MHz, d₆-DMSO): δ 12.39 (brs, 1H), 10.92 (s, 1H), 9.34 (brs, 1H), 8.75 (d, J=5.8 Hz, 1H), 8.57 (s, 1H), 8.37 (d, J=5.8 Hz, 1H) 7.89 (t, J=6.9 Hz, 1H), 7.26 (d, J=6.9 Hz, 1H), 3.66-3.60 (m, 2H), 3.18-2.95 (m, 5H), 2.37-2.30 (m, 2H), 2.01-1.95 (m, 2H), 1.13-1.07 (m, 1H), 0.65-0.60 (m, 2H), 0.35-0.31 (m, 2H).

### Example 262

### 2-[1-(Propyl)piperidin-4-yl]imidazo[1,2-a]pyrido[4,3-c]-1,6-naphthyridin-8(7H)-one bis(trifluoroacetate)

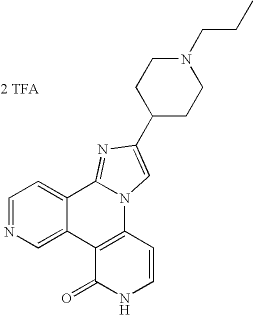

Example 262 was prepared according to a procedure similar to that used in Example 261, except using propionaldehyde instead of cyclopropanecarboxaldehyde as starting material. LCMS calculated for C₂₁H₂₄N₅O (free base M+H)⁺: m/z=362.

### Example 263

### 2-tert-Butylimidazo[1,2-a]pyrido[4,3-c]-1,6-naphthyridin-8(7H)-one bis(trifluoroacetate)

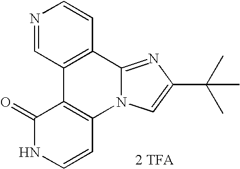

This compound was prepared by a procedure similar to that in Steps E and F of Example 257, except starting with 1-butoxypyrido[4,3-c]-1,6-naphthyridin-6(5H)-one and bromopinancolone. The product then was isolated with preparative chromatography as a fluffy white powder. LCMS for C₁₇H₁₆N₄O (free base M+H)⁺: m/z=293; ¹H NMR (400 MHz, d₆-DMSO): 612.39 (brs, 1H), 11.05 (brs, 1H), 8.95 (brs, 1H), 8.65 (brs, 1H), 8.59 (s, 1H), 7.93 (t, J=6.7 Hz, 1H), 7.38 (d, J=6.0 Hz, 1H), 1.42 (s, 9H).

### Example 264

### 2-(4-Hydroxypiperidin-1-yl)-1,9-dihydro-8H-imidazo[4,5-q-2,8-phenanthrolin-8-one bis(trifluoroacetate) (salt)

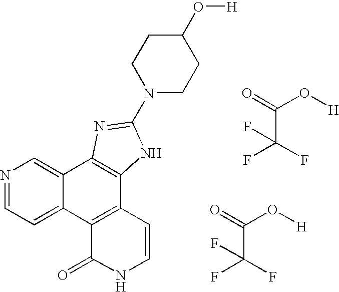

### Step 1: 2-(2-Fluoropyridin-4-yl)-1-pyridin-3-ylethanone

A solution of sodium bis(trimethylsilyl)amide in tetrahydrofuran (1.0 M, 110 mL) was added in tetrahydrofuran (50 mL) and cooled in an ice bath to 0° C. 2-Fluoro-4-methylpyridine (5.00 g, 0.0450 mol) was added slowly and the mixture was stirred for 45 minutes at 0° C. Methyl nicotinate (6.79 g, 0.0495 mol) was added slowly at 0° C. to the mixture and the resulting mixture was stirred at rt overnight. The mixture was poured into 2 M HCl aqueous solution (30 mL) and then made basic with 5 M NaOH (to pH 12). The mixture then was extracted with EtOAc 3×, and the combined organic layer was washed with brine, dried over Na₂SO₄, filtered and concentrated on roto-vap to give a yellow/orange solid residue. Recrystallization in EtOAc gave the desired product (6.0 g, 61%). LCMS calculated for C₁₂H₁₀FN₂O (M+H)⁺: m/z=217.1.

### Step 2: (1E)-1-(2-fluoropyridin-4-yl)-2-pyridin-3-ylethane-1,2-dione 1-oxime

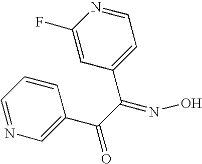

To an acetic acid (30 mL) solution of 2-(2-fluoropyridin-4-yl)-1-pyridin-3-ylethanone (2.5 g, 12 mmol) was added a solution of sodium nitrite (0.96 g, 14 mmol) in water (8 mL) dropwise at 5° C. The solution was stirred in ice bath for 2 hrs, then 60 mL of water was added and a white solid formed. The mixture was filtered and the solid was washed with water 3× to give the desired product (2.30 g, 81%).

LCMS calculated for C₁₂H₉FN₃O₂ (M+H)⁺: m/z=246.1.

### Step 3: 5-(2-Fluoropyridin-4-yl)-4-pyridin-3-yl-1H-imidazol-1-ol

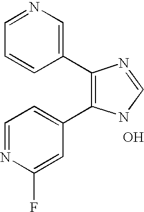

A solution of (1E)-1-(2-fluoropyridin-4-yl)-2-pyridin-3-ylethane-1,2-dione 1-oxime (500 mg, 2.04 mmol), formaldehyde (61.2 mg, 2.04 mmol) and ammonium acetate (650 mg, 8.43 mmol) in acetic acid (20 mL) was heated at 100° C. for 3 min in the microwave. The solvent was evaportated and the crude product was purified by Prep-LCMS to give the desired product (245 mg, 46%).

¹H NMR (400 MHz, d₆-DMSO): 68.15 (m, 3H), 8.10 (d, 2H), 7.80 (s, 1H), 7.40 (d, 1H), 7.25 (s, 1H); LCMS calculated for C₁₃H₁₀FN₄O (M+H)⁺: m/z=257.2.

### Step 4: 4-(2-Chloro-4-pyridin-3-yl-1h-imidazol-5-yl)-2-fluoropyridine

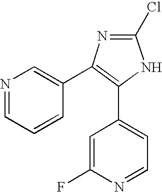

A solution of 5-(2-fluoropyridin-4-yl)-4-pyridin-3-yl-1h-imidazol-1-ol (75 mg, 293 μmol) and phosphoryl chloride (3.39 mL, 36.4 mmol) in chloroform (3 mL) was heated to 70° C. for 5 hours. Purification by Prep-LCMS gave the desired product (42 mg, 52%).

¹H NMR (400 MHz, d₆-DMSO): δ 8.05 (m, 3H), 7.24 (m, 2H), 7.05 (m, 2H); LCMS calculated for C₁₃H₉ClFN₄ (M+H)⁺: m/z=275.2.

### Step 5: 4-(2-Chloro-4-pyridin-3-yl-1H-imidazol-5-yl)pyridine-2(1H)-one bis(trifluoroacetate)

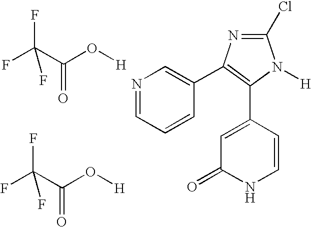

A solution of 4-[2-chloro-4-(4-fluorophenyl)-1h-imidazol-5-yl]-2-fluoropyridine (20 mg, 68.6 μmol), hydrogen chloride in water (4.00 M, 310 μL, 1.24 mmol) and tetrahydrofuran (310 μL, 3.83 mmol) was was heated to 70° C. for 16 hours. Evaporation of the THF and purification by Prep-LCMS gave the desired product (14 mg, 70%).

LCMS calculated for C₁₇H₁₁ClF₆N₄O₅ (M+H)⁺: m/z=273.2.

### Step 6: 4-[2-(4-Hydroxypiperidin-1-yl)-4-pyridin-3-yl-1H-imidazol-5-yl]pyridin-2(1H)-one bis(trifluoroacetate) (salt)

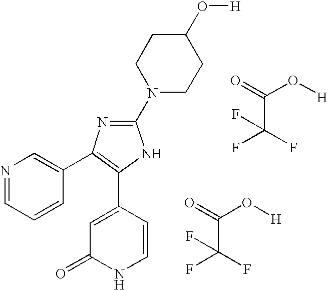

A solution of 4-(2-chloro-4-pyridin-3-yl-1h-imidazol-5-yl)pyridin-2(1h)-one bis(trifluoroacetate) (115 mg, 230 μmol) and 4-hydroxypiperidine (500 mg, 4.94 mmol) in n-methylpyrrolidinone (1 mL) was heated in a microwave at 220° C. for 30 min. The mixture was dissolved in methanol and the desired product was isolated and purified by PrepLCMS (107 mg, 82%).

LCMS calculated for C₂₂H₂₂F₆N₅O₆ (M+H)⁺: m/z=338.2.

### Step 7: 2-(4-Hydroxypiperidin-1-yl)-1,9-dihydro-8H-imidazo[4,5-f]-2,8-phenanthrolin-8-one bis(trifluoroacetate) (salt)

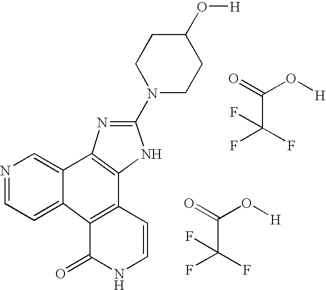

A solution of 4-[2-(4-hydroxypiperidin-1-yl)-4-pyridin-3-yl-1h-imidazol-5-yl]pyridin-2(1h)-one bis(trifluoroacetate) (salt) (54 mg, 95.5 μmol) in tetrahydrofuran (50 mL, 616 mmol) was irradiated using a handheld UV (long wave) lamp for 1.5 hours. Evaporation of solvent and purification by Prep-LCMS gave the desired regioisomer (3 mg, 5%).

¹H NMR (400 MHz, CD₃OD): δ 10.5 (d, 1H), 9.9 (s, 1H), 8.6 (d, 1H), 7.7 (d, 1H), 7.3 (d, 1H), 4.0 (m, 2H), 3.5 (m, 3H), 2.2 (m, 1H), 2.0 (m, 2H), 1.7 (m, 1H); LCMS calculated for C₂₂H₂₀F₆N₅O₆ (M+H)⁺: m/z=336.2.

### Example 265

### 2-(4-Hydroxypiperidin-1-yl)[1,3]thiazolo[4,5-f]-2,9-phenanthrolin-8(9H)-one

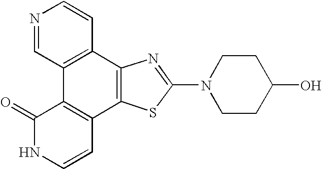 Step A

### 2-(2-Fluoropyridin-4-yl)-1-pyridin-4-ylethanone

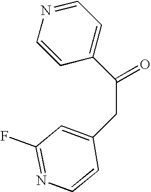

Sodium bis(trimethylsilyl)amide (280 mL, 1.0 M solution in THF) was added to a 3-neck 1 L flask under an atmosphere of nitrogen. The solution was diluted with 100 mL more THF and cooled to 0° C. 2-Fluoro-4-methylpyridine (15.0 g, 0.135 mol) in THF (50 mL) was added slowly via addition funnel and the resulting dark brown solution was stirred for one hour. The mixture was then cooled to −10° C. (ice/salt bath), and 4-pyridinecarboxylic acid ethyl ester (22.2 mL, 0.148 mol) in THF (50 mL) was added slowly maintaining the temperature below 0° C. during the addition. The mixture was warmed to room temperature, stirred for 90 minutes, then slowly poured into 4M HCl (150 mL) and stirred for 15 minutes. The aqueous layer (pH 4) was made basic (pH 12) by addition of 5 M NaOH. The solution was extracted three times with ethyl acetate and the combined extracts were washed with brine, dried over MgSO₄, filtered and concentrated to give a bright yellow solid. The residue was dissolved in dichloromethane (150 mL), filtered and then diluted with hexanes (150 mL). The dichloromethane was removed on the rotovap resulting in precipitation of desired product as a thick sludge. The solids were filtered, washed with cold hexane, and dried under vacuum to give a yellow solid (17 g). The precipitated product was further purified by chromatography on silica gel eluting with ethyl acetate. Pure fractions were combined and concentrated to provide the title compound as a pale yellow solid (16.5 g, 56%). LC/MS: 217 (M+H)⁺. ¹H NMR (CDCl₃) δ 8.87 (d, 2H), 8.21 (d, 1H), 7.77 (d, 2H), 7.08 (m, 1H), 6.86 (s, 1H), 4.34 (s, 2H).

Step B

### 2-Bromo-2-(2-fluoropyridin-4-yl)-1-pyridin-4-ylethanone hydrobromide

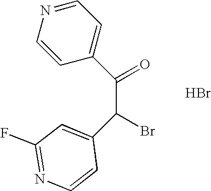

2-(2-Fluoropyridin-4-yl)-1-pyridin-4-ylethanone (14.00 g, 0.0648 mol) was dissolved in acetic acid (140 mL) and then a solution of bromine (10.3 g, 0.0648 mol) dissolved in acetic acid (10 mL) was added dropwise with stirring. The mixture was stirred at room temperature for ten minutes after the addition and a thick precipitate had slowly formed. The mixture was slowly diluted with ethyl acetate (150 mL) with stirring and scraping to further precipitate the product. The solids were filtered and washed with ethyl acetate and diethyl ether. The solid was dried under vacuum to provide the desired product as a light yellow powder (24.2 g, 100%). ¹H NMR (DMSO-d₆) δ 8.96 (d, 2H), 8.32 (d, 1H), 8.11 (d, 2H), 7.54 (m, 1H), 7.39 (s, 1H), 7.17 (s, 1H), 1.91 (s, residual AcOH).

Step C

### 5-(2-Fluoropyridin-4-yl)-4-pyridin-4-yl-1,3-thiazol-2-amine

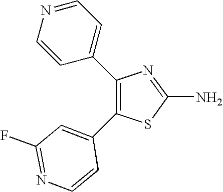

2-Bromo-2-(2-fluoropyridin-4-yl)-1-pyridin-4-ylethanone hydrobromide (14.0 g, 0.0372 mol) was dissolved in DMF (80 mL). Thiourea (4.25 g, 0.0558 mol) was added and the solution was heated to 60° C. for two hours. A solid precipitate formed and LC/MS analysis showed reaction to be complete. The mixture was cooled to room temperature, then poured slowly into saturated aqueous NaHCO₃ (400 mL) producing an immediate precipitate. The mixture was diluted with more water (450 mL) and stirred for 40 minutes. The solids were collected by suction filtration, washed twice with water, air dried, then dried further in a vacuum oven overnight at 50° C. to provide the desired product as a yellow solid (8.86 g, 87%). LC/MS: 273 (M+H)⁺. ¹H NMR (DMSO-d₆) δ 8.56 (d, 2H), 8.12 (d, 1H), 7.65 (bs, 2H, NH₂), 7.38 (d, 2H), 7.08 (dt, 1H), 6.93 (s, 1H).

Step D

### 4-(2-Chloro-4-pyridin-4-yl-1,3-thiazol-5-yl)-2-fluoropyridine

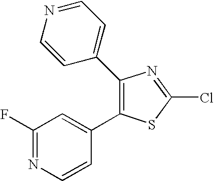

To a solution of 5-(2-fluoropyridin-4-yl)-4-pyridin-4-yl-1,3-thiazol-2-amine (8.80 g, 0.0323 mol) and copper (II) chloride (5.21 g, 0.0388 mol) in acetonitrile (880 mL) at 60° C. was added tert-butyl nitrite (5.76 mL, 0.0485 mol) dropwise over 15 minutes. After the addition was complete, the mixture was stirred at 60° C. for one hour, cooled to room temperature, and then filtered through Celite and the solids washed with ethyl acetate. The filtrate was concentrated by rotovap and the residue (˜11.7 g) was mixed with dichloromethane and saturated NaHCO₃. The mixture was again filtered through Celite, and transferred to a separatory funnel. The layers were separated and the aqueous layer was extracted twice with dichloromethane. The combined organic extracts were dried over Na₂SO₄, filtered and concentrated to give 5.9 g crude product (TLC: Rf 0.38, ethyl acetate). The crude product was purified by flash chromatography on silica gel using a hexane-ethyl acetate gradient. The pure fractions were combined and concentrated to give the desired product as a light yellow solid (4.20 g, 44%). LC/MS: 292, 294 (M+H)⁺. ¹H NMR (DMSO-d₆) δ 8.60 (d, 2H), 8.32 (d, 1H), 7.41 (d, 2H), 7.14 (dt, 1H), 7.13 (s, 1H).

Step E

### 1-[5-(2-Fluoropyridin-4-yl)-4-pyridin-4-yl-1,3-thiazol-2-yl]piperidin-4-ol

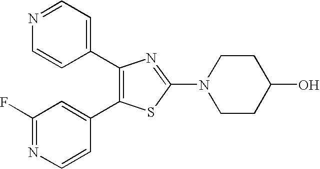

4-Hydroxypiperidine (3.64 g, 0.0360 mol) was added to a solution of 4-(2-chloro-4-pyridin-4-yl-1,3-thiazol-5-yl)-2-fluoropyridine (4.20 g, 0.0144 mol) in DMF (30 mL) under an atmosphere of nitrogen and stirred at room temperature for 16 hours. A precipitate had formed and the yellow mixture was poured into aqueous sodium bicarbonate, diluted with more water and then extracted three times with ethyl acetate. The combined extracts were washed with brine, dried over MgSO₄, filtered, and concentrated. The residue was chromatographed on a silica gel column and eluted with 10% methanol/ethyl acetate. Pure fractions were combined and concentrated to give a light yellow powder (4.60 g, 89%). The product was further purified by recrystallization using 10% methanol/ethyl acetate to provide yellow needles. LC/MS: 357 (M+H)⁺. ¹H NMR (DMSO-d₆). δ 8.58 (d, 2H), 8.14 (d, 1H), 7.41 (d, 2H), 7.10 (dt, 1H), 6.96 (s, 1H), 4.87 (d, 1H), 3.77 (m, 3H), 3.32 (m, 2H), 1.85 (m, 2H), 1.49 (m, 2H).

### Step F—Alternative Synthesis of 1-[5-(2-fluoropyridin-4-yl)-4-pyridin-4-yl-1,3-thiazol-2-yl]piperidin-4-ol

4-Hydroxypiperidine-1-carbothioamide—4-Hydroxypiperidine (1.90 g, 0.0188 mol) was added to a solution of 1,1′-thiocarbonyldiimidazole (3.68 g, 0.0206 mol) in THF (30 mL) and stirred at room temperature for 1.5 hours. Ammonia was then added (7M in methanol, 25 mL) and the mixture was stirred at room temperature for 15 hours. HPLC/MS showed clean conversion to the desired intermediate product (LC/MS: 161 (M+H)⁺). The solution was concentrated and the residue was stirred in 60 mL ether for 20 minutes, however, the product did not solidify. The ether was decanted leaving the crude product as a viscous oil (4.8 g) which was used without further purification (theoretical yield of 4-hydroxypiperidine-1-carbothioamide=3.0 g; contains 1.8 g of imidazole).

To a solution of the crude 4-hydroxypiperidine-1-carbothioamide (prepared above) in DMF (40 mL), was added 2-bromo-2-(2-fluoropyridin-4-yl)-1-pyridin-4-ylethanone hydrobromide (5.60 g, 0.015 mol) and the mixture was stirred at room temperature. After 1 hour, the product began to precipitate. After stirring for 48 hours, the mixture was poured into saturated aqueous NaHCO₃ (400 mL) and extracted several times with ethyl acetate. The combined organic extracts were washed with saturated aqueous NaHCO₃, brine, then dried over Na₂SO₄, filtered and concentrated. The residue was triturated with tert-butyl methyl ether (35 mL), filtered and dried to give the title product of Step E as a light yellow powder (3.20 g). The filtrate was chromatographed as in Step E to provide an additional 1.00 g of product (total yield=4.20 g 79%).

Step G

### 4-[2-(4-Hydroxypiperidin-1-yl)-4-pyridin-4-yl-1,3-thiazol-5-yl]pyridin-2(1H)-one

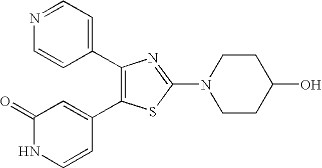

A solution of 1-[5-(2-fluoropyridin-4-yl)-4-pyridin-4-yl-1,3-thiazol-2-yl]piperidin-4-ol (4.00 g, 0.0112 mol) dissolved in THF (35 mL) and aqueous HCl (4.0 M, 35 mL) was heated to 60° C. overnight. LC/MS analysis indicated complete conversion to desired product. The solution was cooled to room temperature, and neutralized by slow addition of 50% NaOH solution (ice bath cooling). Near pH 7, a thick orange precipitate had formed, however, it quickly dissolved at a higher pH. The aqueous layer (pH 10) was repeatedly extracted with ethyl acetate (10 times). The combined organic extracts were concentrated and dried under vacuum to give the desired product as an orange solid (4.40 g, 97%) which was used without further purification. LC/MS: 355 (M+H)⁺. ¹H NMR (DMSO-d₆) δ 11.57 (bs, 1H), 8.57 (d, 2H), 7.44 (d, 2H), 7.29 (d, 1H), 6.16 (m, 1H), 5.86 (dd, 1H), 4.89 (d, 1H), 3.75 (m, 3H), 3.30 (m, 2H), 1.86 (m, 2H), 1.48 (m, 2H).

Step H

### 2-(4-Hydroxypiperidin-1-yl)[1,3]thiazolo[4,5-f]-2,9-phenanthrolin-8(9H)-one

4-[2-(4-Hydroxypiperidin-1-yl)-4-pyridin-4-yl-1,3-thiazol-5-yl]pyridine-2(1H)-one (4.40 g, 0.0124 mol) was dissolved in methanol (8 L) in a pyrex flask and exposed to UV light from a high intensity UV lamp. After 4.5 hours of irradiation, HPLC showed 99% conversion. The solution was concentrated by rotovap and the residue was triturated with 100 mL 2-propanol and heated to 70° C. The mixture was cooled to room temperature, filtered and the solids washed with more 2-propanol. After drying under vacuum, the desired product was obtained as a yellow green powder (2.50 g, 54%).

MSA salt formation: The above product was slurried in 500 mL hot methanol. One equivalent of methanesulfonic acid was added causing the product to dissolve momentarily, then a precipitate formed. The hot mixture was stirred for 30 minutes, concentrated by rotovap to 150 mL, then stirred at room temperature overnight. The solids were filtered, washed with methanol, then dried under vacuum to give a yellow powder 2.20 g. ¹H NMR of the solid was consistent with the half-mesylate salt. LC/MS: 353 (M+H)⁺. ¹H NMR (DMSO-d₆) δ 12.24 (br d, 1H, pyridone NH), 11.37 (s, 1H), 8.86 (d, 1H), 8.74 (d, 1H), 7.74 (t, 1H), 6.69 (d, 1H), 4.00 (m, 2H), 3.86 (m, 1H), 3.58 (m, 2H), 2.29 (s, 1.5H), 1.93 (m, 2H), 1.57 (m, 2H).

TABLE 6

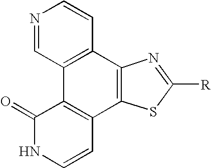

| | | | Mass | Similar |
|---|---|---|---|---|
| Ex. | | | Spec | Method of |
| No. | Name | R | (M + H) | Preparation |
| 266 | 2-(isopropylamino)[1,3]thiazolo[4,5- f]-2,9-phenanthrolin-8(9H)-one | 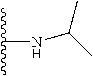 | 311 | Ex. 265 |
| 267 | 2-[(3- methoxypropyl)amino][1,3]thiazolo[4,5- f]-2,9-phenanthrolin-8(9H)-one | 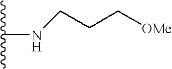 | 341 | Ex. 265 |
| 268 | 2-{[(1R)-1-(hydroxymethyl)-3- methylbutyl]amino}[1,3]thiazolo[4,5- f]-2,9-phenanthrolin-8(9H)-one | 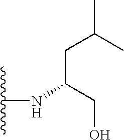 | 369 | Ex. 265 |
| 269 | 2-{[1-(methylsulfonyl)piperidin-4- yl]oxy}[1,3]thiazolo[4,5-f]-2,9- phenanthrolin-8(9H)-one | 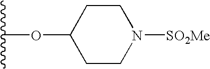 | 431 | Ex. 265 |
| 270 | 2-{[(1R)-1- (hydroxymethyl)butyl]amino}[1,3]thiazolo[4,5-f]-2,9-phenanthrolin-8(9H)- one | 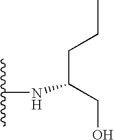 | 355 | Ex. 265 |
| 271 | 2-{[(1R)-1- (hydroxymethyl)propyl]amino}[1,3]thiazolo[4,5-f]-2,9-phenanthrolin- 8(9H)-one | 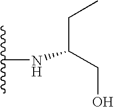 | 341 | Ex. 265 |
| 272 | 2-{[(1R)-1-(hydroxymethyl)-2- methylpropyl]amino}[1,3]thiazolo[4,5- f]-2,9-phenanthrolin-8(9H)-one | 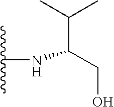 | 355 | Ex. 265 |
| 273 | 2-{[(1R,2R)-2-hydroxycyclohexyl]amino}[1,3]thiazolo[4,5- f]-2,9-phenanthrolin-8(9H)-one | 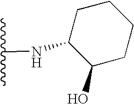 | 367 | Ex. 265 |
| 274 | 2-{[(1R,2R)-2-hydroxycyclopentyl]amino}[1,3]thiazolo[4,5- f]-2,9-phenanthrolin-8(9H)-one | 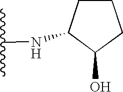 | 353 | Ex. 265 |
| 275 | 2-{[(1R)-1-(hydroxymethyl)-2,2- dimethylpropyl]amino}[1,3]thiazolo[4,5- f]-2,9-phenanthrolin-8(9H)-one | 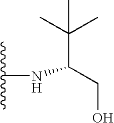 | 369 | Ex. 265 |
| 276 | 2-(4-{[3-(2-oxopyrrolidin-1- yl)propyl]amino}piperidin-1- yl)[1,3]thiazolo[4,5-f]-2,9- phenanthrolin-8(9H)-one | 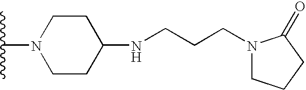 | 477 | Ex. 265 |
| 277 | 2-{[(1S,2S)-1-(hydroxymethyl)-2- methylbutyl]amino}[1,3]thiazolo[4,5- f]-2,9-phenanthrolin-8(9H)-one | 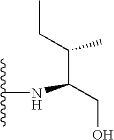 | 369 | Ex. 265 |
| 278 | 2-{4-[(3-morpholin-4- ylpropyl)amino]piperidin-1- yl}[1,3]thiazolo[4,5-f]-2,9- phenanthrolin-8(9H)-one | 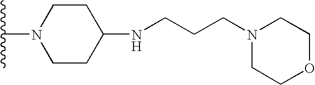 | 479 | Ex. 265 |
| 279 | 2-(dimethylamino)-N-[1-(8-oxo-8,9- dihydro[1,3]thiazolo[4,5-f]-2,9- phenanthrolin-2-yl)piperidin-4- yl]acetamide | 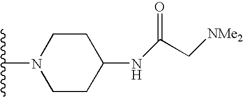 | 437 | Ex. 265 |
| 280 | 2-(4-hydroxypiperidin-1- yl)[1,3]thiazolo[4,5-f]-2,9- phenanthrolin-8(9H)-one | 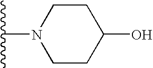 | 353 | Ex. 265 |
| 281 | 2-(propylamino)[1,3]thiazolo[4,5-f]- 2,9-phenanthrolin-8(9H)-one | 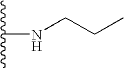 | 311 | Ex. 265 |
| 282 | 2-(4-acetylpiperazin-1- yl)[1,3]thiazolo[4,5-f]-2,9- phenanthrolin-8(9H)-one | 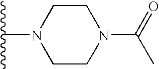 | 380 | Ex. 265 |
| 283 | 2-piperazin-1-yl[1,3]thiazolo[4,5-f]- 2,9-phenanthrolin-8(9H)-one | 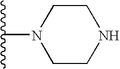 | 338 | Ex. 265 |
| 284 | 2-{4-[(2S)-2- hydroxypropyl]piperazin-1- yl}[1,3]thiazolo[4,5-f]-2,9- phenanthrolin-8(9H)-one | 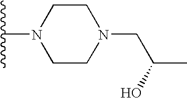 | 396 | Ex. 265 |
| 285 | 2-[4-(1H-imidazol-5- ylacetyl)piperazin-1- yl][1,3]thiazolo[4,5-f]-2,9- phenanthrolin-8(9H)-one | 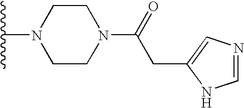 | 446 | Ex. 265 |
| 286 | 2-{4-[(2R)-2- hydroxypropyl]piperazin-1- yl}[1,3]thiazolo[4,5-f]-2,9- phenanthrolin-8(9H)-one | 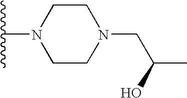 | 396 | Ex. 265 |
| 287 | 2-[4-(2-hydroxyethyl)piperazin-1- yl][1,3]thiazolo[4,5-f]-2,9- phenanthrolin-8(9H)-one | 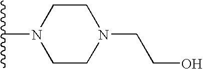 | 382 | Ex. 265 |
| 288 | 2-{[(2R)-2-amino-3,3- dimethylbutyl]oxy}[1,3]thiazolo[4,5- f]-2,9-phenanthrolin-8(9H)-one | 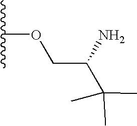 | 369 | Ex. 265 |
| 289 | 2-{[(1S)-1-(hydroxymethyl)-2,2- dimethylpropyl]amino}[1,3]thiazolo[4,5- f]-2,9-phenanthrolin-8(9H)-one | 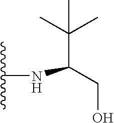 | 369 | Ex. 265 |
| 290 | N-[1-(8-oxo-8,9- dihydro[1,3]thiazolo[4,5-f]-2,9- phenanthrolin-2-yl)piperidin-4-yl]-3- pyridin-3-ylpropanamide | 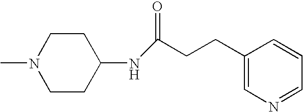 | 485 | Ex. 265 |
| 290a | 2-[4-(2-morpholin-4- ylethoxy)piperidin-1- yl][1,3]thiazolo[4,5-f]-2,9- phenanthrolin-8(9H)-one | 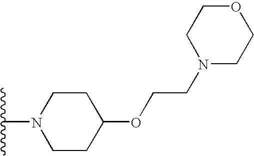 | 466 | Ex. 265 |
| 290b | 2-{4-[(2-morpholin-4- ylethoxy)methyl]piperidin-1- yl}[1,3]thiazolo[4,5-f]-2,9- phenanthrolin-8(9H)-one | 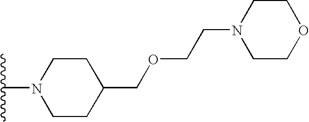 | 480 | Ex. 265 |
| 290c | 2-(4-{methyl[3-(2-oxopyrrolidin-1- yl)propyl]amino}piperidin-1- yl)[1,3]thiazolo[4,5-f]-2,9- phenanthrolin-8(9H)-one | 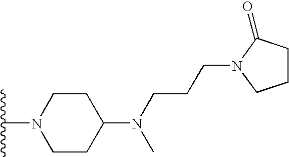 | 491 | Ex. 265 |
| 290d | 2-[4-(morpholin-4- ylmethyl)piperidin-1- yl][1,3]thiazolo[4,5-f]-2,9- phenanthrolin-8(9H)-one | 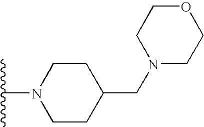 | 436 | Ex. 265 |
| 290e | 2-[4-(2-morpholin-4- ylethyl)piperidin-1- yl][1,3]thiazolo[4,5-f]-2,9- phenanthrolin-8(9H)-one | 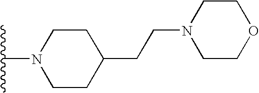 | 450 | Ex. 265 |

TABLE 7

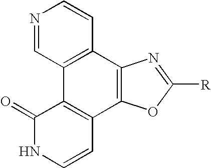

| | | | Mass | Similar |
|---|---|---|---|---|
| Ex. | | | Spec | Method of |
| No. | Name | R | (M + H) | Preparation |
| 291 | 2-(4-hydroxypiperidin-1- yl)[1,3]oxazolo[4,5-f]-2,9- phenanthrolin-8(9H)-one | 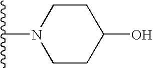 | 337 | Ex. 265 |
| 292 | 2-(4-oxopiperidin-1- yl)[1,3]oxazolo[4,5-f]-2,9- phenanthrolin-8(9H)-one | 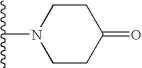 | 335 | Ex. 265 |
| 293 | 2-(4-hydroxy-4-methylpiperidin-1- yl)[1,3]oxazolo[4,5-f]-2,9- phenanthrolin-8(9H)-one | 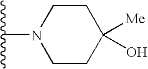 | 351 | Ex. 265 |

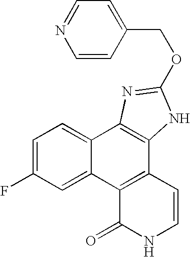

### Example 294

### 9-Fluoro-2-(pyridine-4-ylmethoxy)-3,6-dihydro-7H-benzo[h]imidazo[4,5-f]isoquinolin-7-one

### Step 1: 4-(4-Fluorophenyl)-5-(2-fluoropyridin-4-yl)-1,3-dihydro-2H-imidazol-2-one

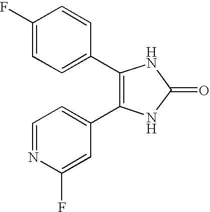

To a solution of 2-amino-1-(4-fluorophenyl)-2-(2-fluoropyridin-4-yl)ethanone hydrochloride (prepared as described in J. Med. Chem. 2003, 46, 3230-3244) (4.34 g, 15.2 mmol) in N,N-dimethylformamide (160 mL) was added potassium cyanate (3.10 g, 38.3 mmol). The mixture was heated to reflux for 1 hour and cooled to room temperature. The reaction mixture was diluted with water and the precipitate was filtered off, washed with water and dried under vacuum. (3.40 g, 81.6%).

¹H NMR (400 MHz, d₆-DMSO): 610.93 (s, 1H), 10.84 (s, 1H), 8.05 (d, 1H), 7.48 (dd, 2H), 7.31 (t, 2H), 7.06 (dt, 1H), 6.96 (s, 1H); MF=C₁₄H₉F₂N₃O; LCMS calculated for C₁₄H₁₀F₂N₃O(M+H)⁺: m/z=274.079.

### Step 2: 4-[2-Chloro-4-(4-fluorophenyl)-]H-imidazol-5-yl]-2-fluoropyridine

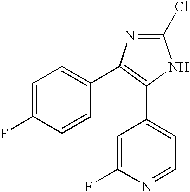

A suspension of 4-(4-fluorophenyl)-5-(2-fluoropyridin-4-yl)-1,3-dihydro-2h-imidazol-2-one (3.40 g, 12.4 mmol) in phosphoryl chloride (100 mL) was heated to reflux for 2 hours. The excess phosphoryl chloride was evaporated and the residue was poured onto crushed ice, neutralized with solid NaOH, and extracted with EtOAc. The extracts were dried over Na₂SO₄, filtered and concentrated to afford 3.22 g crude chloroimidazole, which was used without further purification in the next hydrolysis step.

¹H NMR (400 MHz, CD₃OD): δ 8.06 (d, 1H), 7.49 (dd, 2H), 7.28 (dt, 1H), 7.23 (t, 2H), 7.07 (s, 1H); MF=C₁₄H₈ClF₂N₃; LCMS calculated for C₁₄H₉ClF₂N₃(M+H)⁺: m/z=292.045.

### Step 3: 4-[2-Chloro-4-(4-fluorophenyl)-1H-imidazol-5-yl]pyridin-2(]H)-one

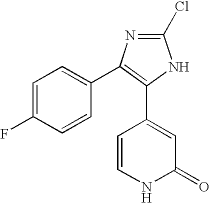

A solution of 4-[2-chloro-4-(4-fluorophenyl)-1H-imidazol-5-yl]-2-fluoropyridine (3.22 g, 0.0110 mol) in 4.00 M of hydrogen chloride in water (50 mL) and tetrahydrofuran (50 mL, 0.6 mol) was heated to 70° C. for 16 hours. The mixture was added to pH 7 buffer and the aqueous portion was extracted with 10% iPrOH/CHCl₃. The combined extracts were dried over sodium sulfate, decanted and concentrated. The product was purified by column chromatography (7% MeOH/0.7% NH₄OH/DCM) (1.80 g, 56%).

¹H NMR (300 MHz, d₆-DMSO): δ 11.57-11.23 (br s, 1H), 7.51 (dd, 2H), 7.36-7.21 (m, 3H), 6.32 (d, 1H), 6.12 (dd, 1H); MF=C₁₄H₉ClFN₃O; LCMS calculated for C₁₄H₁₀ClFN₃O(M+H)⁺: m/z=290.050.

### Step 4: 2-Chloro-9-fluoro-3,6-dihydro-7H-benzo[h]imidazo[4,5-f]isoquinolin-7-one

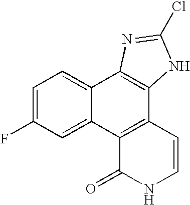

A solution of 4-[2-chloro-4-(4-fluorophenyl)-1H-imidazol-5-yl]pyridin-2(1H)-one (1.8 g, 0.0062 mol) (3629-37) in methanol (60 mL) and tetrahydrofuran (60 mL) was irradiated using 365 nm light for 9 hours. The solvent was evaporated and the orange solid obtained (2.3 g) was triturated with diethyl ether overnight, filtered, rinsed with ether and air dried. (1.74 g, 97%).

¹H NMR (300 MHz, d₆-DMSO): δ 11.87-11.65 (br s, 1H), 10.04 (dd, 1H), 8.45-8.35 (m, 1H), 7.69-7.54 (m, 2H), 7.11 (d, 1H); MF=C₁₄H₇ClFN₃O; LCMS calculated for C₁₄H₈ClFN₃O(M+H)⁺: m/z=288.034.

### Step 5: 2-Chloro-9-fluoro-3,6-bis[2-(trimethylsilyl)ethoxy]methyl-3,6-dihydro-7H-benzo[h]imidazo[4,5-f]isoquinolin-7-one and 2-chloro-9-fluoro-1,6-bis[2-(trimethylsilyl)ethoxy]methyl-3,6-dihydro-7H-benzo[h]imidazo[4,5-f]isoquinolin-7-one

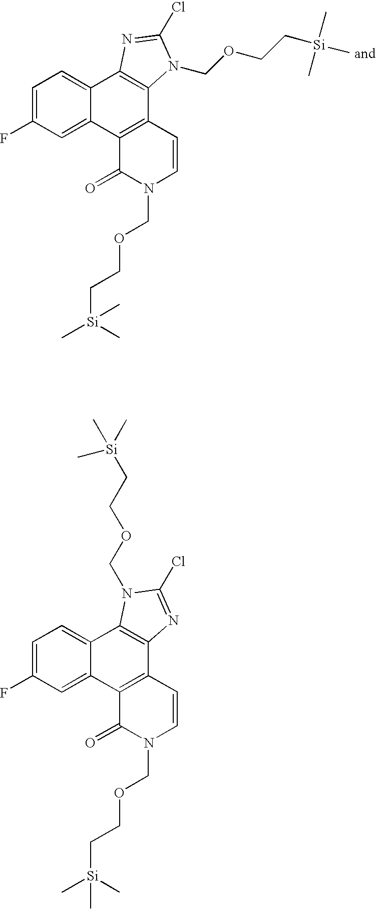

To a suspension of 2-chloro-9-fluoro-3,6-dihydro-7H-benzo[h]imidazo[4,5-f]isoquinolin-7-one (0.400 g, 0.00139 mol) in tetrahydrofuran (8 mL) was added sodium hydride (0.13 g, 0.0056 mol). The mixture was stirred for 3 minutes, then [β-(Trimethylsilyl)ethoxy]methyl chloride (0.52 mL, 0.0029 mol) was added. The reaction mixture was stirred for 15 minutes and then was poured into a mixture of brine and ether, and the aqueous portion was extracted with three volumes of ether. The combined extracts were dried over sodium sulfate, filtered and concentrated. Column chromatography using 15% EA/HX afforded product as a mixture of regioisomers (348 mg, 46%). ¹H NMR (300 MHz, CDCl₃): δ 10.24, (dd, 1H), 10.04 (dd, 1H), 8.64 (dd, 1H), 8.52 (dd, 1H), 7.64 (d, 1H), 7.58 (d, 1H), 7.51-7.41 (m, 3H), 7.35 (d, 1H), 5.92 (s, 2H), 5.86 (s, 2H), 5.58 (s, 4H), 3.82-3.66 (m, 814), 1.05-1.92 (m, 8H), 0.05-−0.08, m, 36H); MF=C₂₆H₃₅ClFN₃O₃Si₂; LCMS calculated for C₂₆H₃₆ClFN₃O₃Si₂(M+H)⁺: m/z=548.197.

### Step 6: 9-Fluoro-2-(pyridine-4-ylmethoxy)-3,6-dihydro-7H-benzo[h]imidazo[4,5-f]isoquinolin-7-one

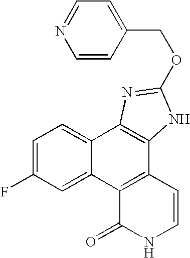

To a solution of 2-chloro-9-fluoro-3,6-bis[2-(trimethylsilyl)ethoxy]methyl-3,6-dihydro-7H-benzo[h]imidazo[4,5-f]isoquinolin-7-one (0.050 g, 0.000091 mol) in dry N,N-dimethylformamide (0.3 mL) was added 4-pyridinemethanol (0.17 g, 0.0016 mol) followed by sodium hydride (0.010 g, 0.00042 mol). The reaction mixture was stirred at room temperature for 1 hour. The reaction mixture was quenched by addition of water, then diluted with a small amount of ethyl acetate and chromatographed directly (50% ethyl Acetate/hexanes) to remove excess pyridinemethanol. The product collected was mixed with methylene chloride (4.0 mL) and trifluoroacetic acid (1.0 mL, 0.013 mol) with stirring for 3 days. The solvents were evaporated and the residue was then mixed with potassium carbonate (0.020 g, 0.00014 mol) in methanol (4.0 mL, 0.099 mol) with stirring for 1 hour.

Some methanol was removed and water was added. The pH was adjusted to 7 with 1.0 N HCl and the solid product was filtered off and washed with a small amount of water. The solid obtained was further purified by prep-LCMS to yield the desired product (11 mg, 33%).

¹H NMR (500 MHz, d₆-DMSO, 90° C.): δ 13.44-12.39 (br s, 1H), 11.29 (s, 1H), 10.02 (d, 1H), 8.63 (d, 2H), 8.36 (br s, 1H), 7.55 (d, 2H), 7.53-7.47 (m, 2H), 7.07 (br s, 1H), 5.72 (s, 2H); ¹⁹F NMR (500 MHz, d₆-DMSO, 90° C.): δ −115.0; MF=C₂₀H₁₃FN₄O₂; LCMS calculated for C₂₀H₁₄FN₄O₂(M+H)⁺: m/z=361.110.

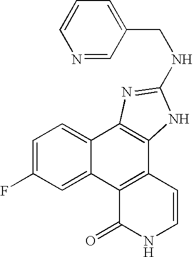

### Example 295

### 9-Fluoro-2-[(pyridin-3-ylmethyl)amino]-3,6-dihydro-7H-benzo[h]imidazo[4,5-f]isoquinolin-7-one

A solution of 2-chloro-9-fluoro-3,6-bis[2-(trimethylsilyl)ethoxy]methyl-3,6-dihydro-7H-benzo[h]imidazo[4,5-f]isoquinolin-7-one prepared according to the method described in Example 294 (0.100 g, 0.182 mmol) in picolamine (0.4 mL, 0.004 mol) in a 0.2-0.5 mL microwavable vessel was microwaved at 180° C. for 1 hour. Water was added and the aqueous mixture was extracted with ethyl acetate. The extracts were washed with water and brine, dried over sodium sulfate, filtered and concentrated. The crude adduct was deprotected by stirring with TFA (1 mL) in DCM (1 mL) for 16 hours. The solvents were evaporated. The residue was stirred in MeOH containing a sufficient quantity of K₂CO₃ to make a basic medium for 2 hours. The methanol was evaporated. The residue was added in water and to the resulting slurry was added aq. HCl (1.0 N) solution to adjust the pH to 7. Then water was removed, and the residue was dissolved in DMSO. The desired product was isolated and purified using prep-LCMS (12 mg, 18%).

¹H NMR (300 MHz, d₆-DMSO): δ 11.42 (s, 1H), 9.98 (dd, 1H), 8.69 (s, 1H), 8.46 (d, 1H), 8.36 (dd, 1H), 7.87 (d, 1H), 7.53-7.41 (m, 3H), 7.37 (dd, 1H), 7.05 (d, 1H), 4.65 (d, 2H); ¹⁹F NMR (300 MHz, d₆-DMSO): δ −115.8; MF=C₂₀H₁₄FN₅O; LCMS calculated for C₂₀H₁₅FN₅O(M+H)⁺: m/z=360.126.

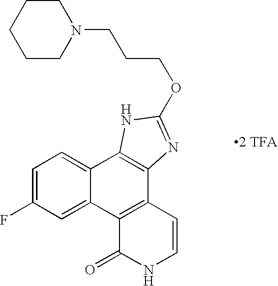

### Example 296

### 9-fluoro-2-(3-piperidin-1-ylpropoxy)-1,6-dihydro-7H-benzo[h]imidazo[4,5-f]isoquinolin-7-one (bis-TFA salt)

### Step 1: 1-Benzyl-5-(4-fluorophenyl)-4-(2-fluoropyridin-4-yl)-1,3-dihydro-2H-imidazol-2-one

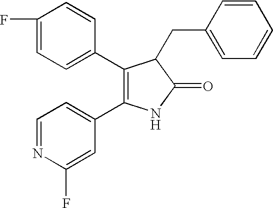

To a solution of aqueous formaldehyde (12 M, 5.68 mL) in acetonitrile (400 mL) was added benzylamine (8.33 mL, 0.0763 mol) and the reaction mixture was stirred without heating for 1 hour. (2E)-1-(4-Fluorophenyl)-2-(2-fluoropyridin-4-yl)ethane-1,2-dione 2-oxime (20.0 g, 0.0763 mol) (prepared as described in J. Med. Chem. 2003, 46, 3230-3244) was then added. The reaction mixture was warmed slowly to gentle reflux for 5 days, with further addition of benzylamine (2.10 mL, 0.020 mol) and aqueous formaldehyde (12 M, 1.4 mL) on the third day. The solvent was removed and the resulting crude solid was triturated with ether to afford the desired product (12.8 g, 46%). MF=C₂₁H₁₅F₂N₃O; LCMS calculated for C₂₁H₁₆F₂N₃O(M+H)⁺: m/z=364.126.

### Step 2: 4-[1-Benzyl-2-chloro-5-(4-fluorophenyl)-1H-imidazol-4-yl]-2-fluoropyridine

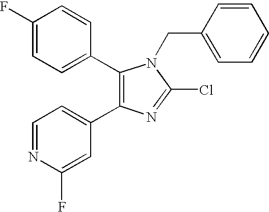

A suspension of 1-benzyl-5-(4-fluorophenyl)-4-(2-fluoropyridin-4-yl)-1,3-dihydro-2H-imidazol-2-one (14.3 g, 0.0394 mol) and ammonium chloride (4.6 g, 0.086 mol) in phosphoryl chloride (150 mL) was heated to reflux for 15 hours. The reaction mixture was cooled and excess POCl₃ evaporated. The resulting residue was poured over ice, neutralized (pH=7) with NaOH and extracted with DCM. The combined organic extracts were washed with brine, dried over sodium sulfate, decanted, and concentrated. The crude solid was triturated with a minimum amount of methanol, filtered and washed with some methanol to afford 5.8 g of the product. Additional product (4.5 g) was obtained from the mother liquor by column chromatography using 20% ethyl acetate/hexanes. (Total: 10.3 g, 62%).

¹H NMR (300 MHz, CDCl₃): δ 7.99 (d, 1H), 7.32-7.27 (m, 3H), 7.17-7.08 (m, 5H), 6.99-6.96 (m, 1H), 6.91-6.84 (m, 2H), 5.00 (s, 2H); MF=C₂₁H₁₄ClF₂N₃; LCMS calculated for C₂₁H₁₅ClF₂N₃(M+H)⁺: m/z=382.092.

### Step 3: 4-[1-Benzyl-2-chloro-5-(4-fluorophenyl)-1H-imidazol-4-yl]pyridin-2(1H)-one

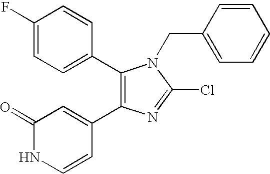

A mixture of 4-[1-benzyl-2-chloro-5-(4-fluorophenyl)-1h-imidazol-4-yl]-2-fluoropyridine (10.9 g, 20.3 mmol), hydrogen chloride in water (4.00 M, 100 mL) and tetrahydrofuran (200 ml) was heated to 70° C. for 50 hours. After cooling, the mixture was neutralized (pH=7) with solid NaOH. The mixture was extracted with 10% iPrOH/CHCl₃ (3 times). The organic extracts were combined, dried over sodium sulfate, filtered and concentrated to an oily solid. Trituration with ether overnight afforded a white precipitate: 8.5 g. NMR indicates 75% purity. (6.38 g, 62%).

MF=C₂₁H₁₅ClFN₃O; LCMS calculated for C₂₁H₁₆ClFN₃O(M+H)⁺: m/z=380.097.

### Step 4: 1-Benzyl-2-chloro-9-fluoro-1,6-dihydro-7H-benzo[h]imidazo[4,5-f]isoquinolin-7-one

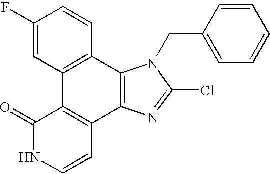

A solution of 4-[1-benzyl-2-chloro-5-(4-fluorophenyl)-1H-imidazol-4-yl]pyridin-2(1H)-one (8.5 g, 0.022 mol) in methanol (2.4 L) was split into 3 batches and each batch was irraditated through Pyrex using a medium-pressure Hg vapor lamp for 4 hours. Then methanol was evaporated and the resulting solid was triturated with ether and dried in a vacuum oven overnight at 60° C. to yield the desired product (5.30 g, 63%).

¹H NMR (300 MHz, d₆-DMSO): δ 11.86 (br d, 1H), 10.21 (dd, 1H), 8.24 (dd, 1H), 7.65 (t, 1H), 7.46 (ddd, 1H), 7.39-7.25 (m, 4H), 7.17-7.11 (m, 2H), 6.04 (s, 2H); MF=C₂₁H₁₃ClFN₃O; LCMS calculated for C₂₁H₁₄ClFN₃O(M+H)⁺: m/z=378.081.

### Step 5: 1-Benzyl-2-chloro-9-fluoro-6-[2-(trimethylsilyl)ethoxy]methyl-1,6-dihydro-7H-benzo[h]imidazo[4,5-f]isoquinolin-7-one

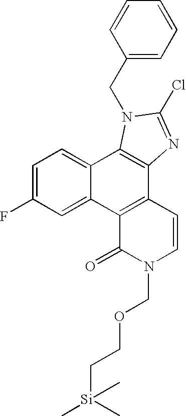

To a suspension of 1-benzyl-2-chloro-9-fluoro-1,6-dihydro-7H-benzo[h]imidazo[4,5-f]isoquinolin-7-one (5.47 g, 0.0145 mol) in tetrahydrofuran (125 mL) at 0° C. was added a solution of potassium tert-butoxide in tetrahydrofuran (1.00 M, 14.9 mL). After 10 minutes, the suspension became a solution and [β-(trimethylsilyl)ethoxy]methyl chloride (2.48 mL, 0.0140 mol) was added. The reaction mixture was stirred for 15 minutes. Water was added and the layers were separated. The aqueous layer was extracted with ethyl acetate. The combined organic layers were washed with brine, dried over sodium sulfate, filtered and concentrated. (7.00 g, 95%).

¹H NMR (300 MHz, CDCl₃): δ 10.23 (dd, 1H), 7.98 (dd, 1H), 7.66 (d, 1H), 7.48 (d, 1H), 7.40-7.31 (m, 3H), 7.22 (ddd, 1H), 7.16-7.11 (m, 2H), 5.89 (s, 2H), 5.58 (s, 2H), 3.71 (dd, 2H), 1.00 (dd, 2H), 0.00 (s, 9H); MF=C₂₇H₂₇ClFN₃O₂Si; LCMS calculated for C₂₇H₂₈ClFN₃O₂Si(M+H)⁺: m/z=508.162.

### Step 6: 1-Benzyl-9-fluoro-2-(3-piperidin-1-ylpropoxy)-6-[2-(trimethylsilyl)ethoxy]methyl-1,6-dihydro-7H-benzo[h]imidazo[4,5-f]isoquinolin-7-one

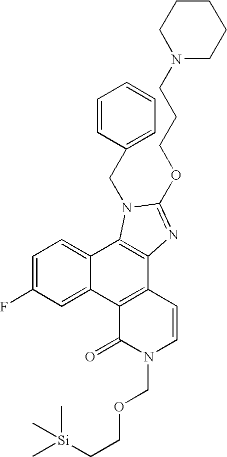

To a mixture of 1-benzyl-2-chloro-9-fluoro-6-[2-(trimethylsilyl)ethoxy]methyl-1,6-dihydro-7H-benzo[h]imidazo[4,5-f]isoquinolin-7-one (0.150 g, 0.295 mmol) in 1-piperidinepropanol (0.76 mL, 0.0050 mol) were added sodium hydride (0.054 g, 0.0013 mol) and N,N-Dimethylformamide (0.8 mL, 0.01 mol). The reaction mixture was stirred at room temperature for 40 min. To the reaction mixture were added ethyl acetate and water, and the layers were separated. The aqueous layer was extracted with ethyl acetate (2 times). The combined organic layers were dried over sodium sulfate, filtered and concentrated. The crude product was directly used in the next deprotection step.

MF=C₃₅H₄₃FN₄O₃Si; LCMS calculated for C₃₅H₄₄FN₄O₃Si(M+H)⁺: m/z=615.317.

### Step 7: 1-Benzyl-9-fluoro-2-(3-piperidin-1-ylpropoxy)-1,6-dihydro-7H-benzo[h]imidazo[4,5-f]isoquinolin-7-one

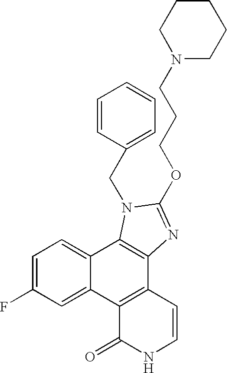

A solution of 1-benzyl-9-fluoro-2-(3-piperidin-1-ylpropoxy)-6-[2-(trimethylsilyl)ethoxy]methyl-1,6-dihydro-7H-benzo[h]imidazo[4,5-f]isoquinolin-7-one (0.182 g, 0.000296 mol) in trifluoroacetic acid (1 mL, 0.01 mol) and methylene chloride (4 mL, 0.06 mol) was stirred at room temperature for 3 days. Additional TFA (5 mL) was added and the reaction was continued for 3 hours to completion. The solvents were evaporated. The residue was then stirred with methanol (4 mL, 0.1 mol) and ammonium hydroxide (4 mL, 0.1 mol) for 1 hour. Methanol and most of the water was evaporated and the solid product was filtered off. The product was further purified using prep-LCMS to afford 71 mg of solid as bis-TFA salt (free base:48 mg, yield: 33%), which was used without further purification in the next debenzylation step.

MF=C₂₉H₂₉FN₄O₂; LCMS calculated for C₂₉H₃₀FN₄O₂(M+H)⁺: m/z=485.235.

### Step 8: 9-Fluoro-2-(3-piperidin-1-ylpropoxy)-1,6-dihydro-7H-benzo[h]imidazo[4,5-f]isoquinolin-7-one (bis-TFA salt)

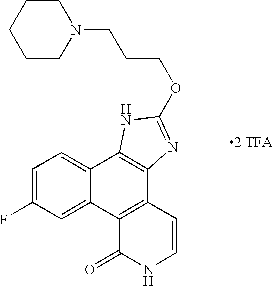

To a degassed mixture of 1-benzyl-9-fluoro-2-(3-piperidin-1-ylpropoxy)-1,6-dihydro-7H-benzo[h]imidazo[4,5-f]isoquinolin-7-one (0.046 g, 0.000095 mol) (as 68 mg of the bis-TFA salt) in ethanol (6 mL) and hydrogen chloride in water (3.0 M, 1.6 mL) was added palladium (10% on carbon, 0.029 g, 0.00026 mol), and the reaction mixture was stirred under a balloon of hydrogen for 21 hours. The catalyst was removed by filtration and the solvent was evaporated. The product was purified by prep-LCMS to yield the desired product as the bis-TFA salt (13 mg, 35%).

¹H NMR (300 MHz, d₆-DMSO): δ 10.03 (dd, 1H), 8.35 (dd, 1H), 7.60-7.51 (m, 2H), 7.06 (d, 1H), δ 4.66 (t, 2H), 3.52 (d, 2H), 3.31-3.21 (m, 2H), 2.99-2.87 (m, 2H), 2.34-2.22 (m, 2H), 1.90-1.53 (m, 6H); ¹⁹F NMR (300 MHz, d₆-DMSO): δ −74.5, −114.8; MF=C₂₂H₂₃FN₄O₂; LCMS calculated for C₂₂H₂₄FN₄O₂(M+H)⁺: m/z=395.188.

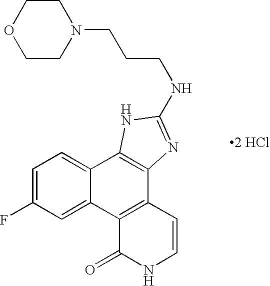

### Example 297

### 9-Fluoro-2-[(3-morpholin-4-ylpropyl)amino]-1,6-dihydro-7H-benzo[h]imidazo[4,5-f]isoquinolin-7-one (bis-HCl salt)

### Step 1: 1-Benzyl-9-fluoro-2-[(3-morpholin-4-ylpropyl)amino]-6-[2-(trimethylsilyl)ethoxy]methyl-1,6-dihydro-7H-benzo[h]imidazo[4,5-f]isoquinolin-7-one

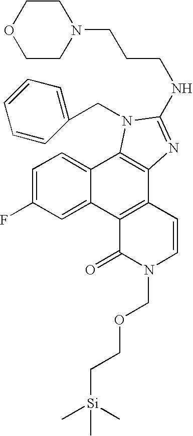

A solution of 1-benzyl-2-chloro-9-fluoro-6-[2-(trimethylsilyl)ethoxy]methyl-1,6-dihydro-7H-benzo[h]imidazo[4,5-f)isoquinolin-7-one (0.150 g, 0.295 mmol, prepared according to Example 296) in 4-morpholinepropanamine (neat, 0.5 mL, 0.003 mol) in a 0.2-0.5 mL microwavable vessel was microwaved to 180° C. for 1 hour. The reaction mixture was added to a mixture of water and ethyl acetate. Layers were separated. The aqueous layer was extracted with two further volumes of ethyl acetate. The combined organic layers were dried over sodium sulfate, filtered and concentrated (191 mg, 94%). The crude product was directly used in the next deprotection step.

MF=C₃₄H₄₂FN₅O₃Si; LCMS calculated for C₃₄H₄₃FN₅O₃Si(M+H)⁺: m/z=616.312.

### Step 2: 1-Benzyl-9-fluoro-2-[(3-morpholin-4-ylpropyl)amino]-1,6-dihydro-7H-benzo[h]imidazo[4,5-f]isoquinolin-7-one

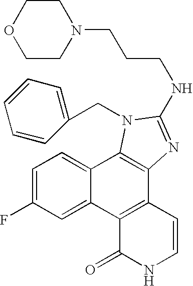

1-Benzyl-9-fluoro-2-[(3-morpholin-4-ylpropyl)amino]-6-[2-(trimethylsilyl)ethoxy]methyl-1,6-dihydro-7H-benzo[h]imidazo[4,5-f]isoquinolin-7-one (0.182 g, 0.000266 mol) was stirred in trifluoroacetic acid (2 mL, 0.03 mol) at room temperature for 1 hour. The TFA was evaporated. The residue was dissolved in methanol (1.3 mL, 0.032 mol) and ammonium hydroxide (1.3 mL, 0.033 mol), and the solution was stirred at room temperature for 1 hour. The methanol and ammonium hydroxide were evaporated. The residue was suspended in water, and the resulting yellow solid was filtered off and washed with a small amount of water. The product was further purified by prep-LCMS to afford 85 mg of product as the bis-TFA salt (58 mg worth of product as free base, yield: 45%). MF=C₂₈H₂₈FN₅O₂; LCMS calculated for C₂₈H₂₉FN₅O₂(M+H)⁺: m/z=486.231.

### Step 3: 9-Fluoro-2-[(3-morpholin-4-ylpropyl)amino]-1,6-dihydro-7H-benzo[h]imidazo[4,5-f]isoquinolin-7-one (bis-HCl salt)

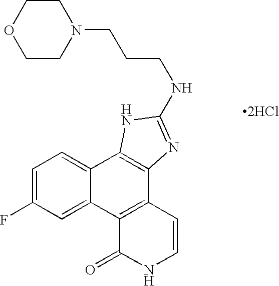

To a degassed mixture of 1-benzyl-9-fluoro-2-[(3-morpholin-4-ylpropyl)amino]-1,6-dihydro-7H-benzo[h]imidazo[4,5-f]isoquinolin-7-one (0.028 g, 0.000058 mol) (as 42 mg of the bis-TFA salt) in ethanol (20 mL) and hydrogen chloride in water (3.0 M, 2 mL) was added palladium (10% on carbon, 0.021 g, 0.00017 mol), and the reaction mixture was stirred under a balloon of hydrogen for 24 h. The catalyst was removed by filtration and the methanol was removed on the rotovap to give an oil. Ethyl acetate and methanol were added to the oil, and a precipitate formed. The solvents were removed to afford a powdery off-white solid product as the bis-HCl salt. (5 mg, 22%).

¹H NMR (300 MHz, d₆-DMSO): δ 11.87 (br s, 1H), 10.05, (dd, 1H), 8.94-8.68 (m, 1H), 7.70-7.60 (m, 2H), 7.45 (br d, 1H), 4.05-3.02 (m, 12H), 2.22-2.09 (m, 2H); ¹⁹F NMR (300 MHz, d₆-DMSO): δ −112.0; MF=C₂₁H₂₂FN₅O₂; LCMS calculated for C₂₁H₂₃FN₅O₂(M+H)⁺: m/z=396.184.

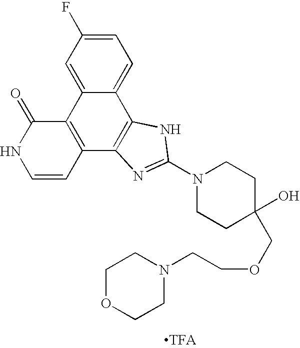

### Example 298

### 9-Fluoro-2-4-hydroxy-4-[(2-morpholin-4-ylethoxy)methyl]piperidin-1-yl-1,6-dihydro-7H-benzo[h]imidazo[4,5-f]isoquinolin-7-one TFA salt

### Step 1: 1-Benzyl-2-(1,4-dioxa-8-azaspiro[4,5]dec-8-yl)-9-fluoro-6-[2-(trimethylsilyl)ethoxy]methyl-1,6-dihydro-7H-benzo[h]imidazo[4,5-f]isoquinolin-7-one

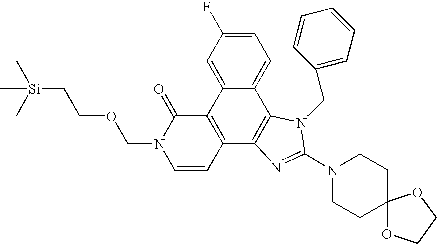

1-Benzyl-2-chloro-9-fluoro-6-[2-(trimethylsilyl)ethoxy]methyl-1,6-dihydro-7H-benzo[h]imidazo[4,5-f]isoquinolin-7-one (0.150 g, 0.000295 mol, prepared according to Example 296) and 1,4-dioxa-8-azaspiro[4.5]decane (0.378 mL, 0.00295 mol) were mixed in a 0.2-0.5 mL microwavable vessel, and the mixture was heated in the microwave at 200° C. for 180 minutes. The brown mixture was then dissolved in CH₂Cl₂, and the product was isolated and purified on a 12 g combiflash column with hexanes/EtOAc=(from 100:0 to 60:40) as an off-white solid (98 mg, 53.99%).

¹H NMR (300 MHz, CDCl₃): δ 10.23 (dd, 1H), 7.89 (dd, 1H), 7.62 (d, 1H), 7.52 (d, 1H), 7.37-7.27 (m, 3H), 7.22-7.17 (m, 2H), 7.13 (ddd, 1H), 5.71 (s, 2H), 5.60 (s, 2H), 3.98 (s, 4H), 3.70 (dd, 2H), 3.35 (dd, 4H), 1.86 (dd, 4H), 1.56 (s, 2H), 0.99 (dd, 2H), 0.00, (s, 9H); MF=C₃₄H₃₉FN₄O₄Si; LCMS calculated for C₃₄H₄₀FN₄O₄Si(M+H)⁺: m/z=615.280.

### Step 2: 1-Benzyl-9-fluoro-2-(4-oxopiperidin-1-yl)-6-[2-(trimethylsilyl)ethoxy]methyl-1,6-dihydro-7H-benzo[h]imidazo[4,5-f]isoquinolin-7-one

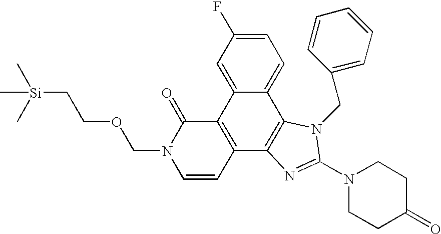

1-Benzyl-2-(1,4-dioxa-8-azaspiro[4,5]dec-8-yl)-9-fluoro-6-[2-(trimethylsilyl)ethoxy]methyl-1,6-dihydro-7H-benzo[h]imidazo[4,5-f]isoquinolin-7-one (0.423 g, 0.000688 mol) was dissolved in 20 mL THF, and 20 mL of 3N HCl was added. The mixture was heated to 45° C. for 3.5 hours. The pH of the solution was adjusted to 11 using an aqueouse solution of NaOH. Then THF was removed by evaporation, and the product was extracted with EtOAc. The organic extract was washed with water twice and brine once, dried over sodium sulfate and concentrated to give a light yellow solid (384 mg, 98%), which was used in the next step without further purification.

¹H NMR (400 MHz, CDCl₃): δ 10.24 (dd, 1H), 7.89 (dd, 1H), 7.63 (d, 1H), 7.51 (d, 1H), 7.40-7.30 (m, 3H), 7.24-7.19 (m, 2H), 7.16 (ddd, 1H), 5.80 (s, 2H), 5.60 (s, 2H), 3.71 (dd, 2H), 3.58 (dd, 4H), 2.67 (dd, 4H), 1.00 (dd, 2H), 0.00 (s, 9H); MF=C₃₂H₃₅FN₄O₃Si; LCMS calculated for C₃₂H₃₆FN₄O₃Si(M+H)⁺: m/z=571.254.

### Step 3: 1-Benzyl-9-fluoro-2-(]-oxa-6-azaspiro[2,5]oct-6-yl)-6-[2-(trimethylsilyl)ethoxy]methyl-1,6-dihydro-7H-benzo[h]imidazo[4,5-f]isoquinolin-7-one

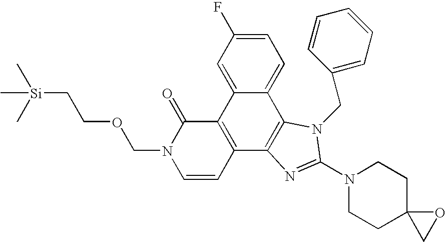

Sodium hydride (83 mg, 0.0020 mol) was added to a solution of trimethylsufoxonium iodide (450 mg, 0.0020 mol) in anhydrous dimethyl sulfoxide (16 mL, 0.23 mol) under an atmosphere of nitrogen. The mixture was stirred at room temperature for 1.2 hours. A solution of 1-benzyl-9-fluoro-2-(4-oxopiperidin-1-yl)-6-[2-(trimethylsilyl)ethoxy]methyl-1,6-dihydro-7H-benzo[h]imidazo[4,5-f]isoquinolin-7-one (581 mg, 0.00102 mol) in 10 ml DMSO was added. The reaction mixture turned pink upon the addition of the ketone. After the addition was complete, the mixture was stirred at room temperature for 1.5 hours. The mixture was poured into water, and the product was extracted with CH₂Cl₂. The organic layer was washed with water twice and with brine once, dried over sodium sulfate and concentrated to give a light yellow solid (651 mg, 99.52%), which was used in the next step without further purification.

MF=C₃₃H₃₇FN₄O₃Si; LCMS calculated for C₃₃H₃₈FN₄O₃Si(M+H)⁺: m/z=585.270.

### Step 4: 1-Benzyl-9-fluoro-2-4-hydroxy-4-[(2-morpholin-4-ylethoxy)methyl]piperidin-1-yl-1,6-dihydro-7H-benzo[h]imidazo[4,5-f]isoquinolin-7-one

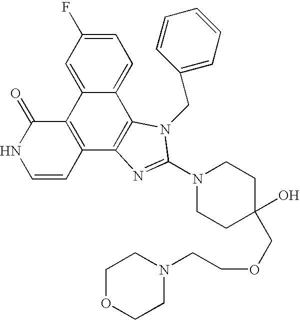

4-Morpholineethanol (154 uL, 0.00126 mol) was dissolved in 4 mL of DMSO, and sodium hydride (38.3 mg, 0.000955 mol) was added. The mixture was stirred for one hour at room temperature. 1-Benzyl-9-fluoro-2-(1-oxa-6-azaspiro[2.5]oct-6-yl)-6-[2-(trimethylsilyl)ethoxy]methyl-1,6-dihydro-7H-benzo[h]imidazo[4,5-f]isoquinolin-7-one (101 mg, 0.000157 mol) in 4 mL of DMSO was then added. The resulting mixture was stirred at 60° C. for 2 hours. The reaction mixture then was quenched with water and concentrated. Purification on prep-LCMS with CAN-TFA method and concentration gave 113 mg of a viscous oil, which was used without further purification in the next deprotection step.

MF=C₃₃H₃₆FN₅O₄; LCMS calculated for C₃₃H₃₇FN₅O₄(M+H)⁺: m/z=586.283.

### Step 5: 9-Fluoro-2-4-hydroxy-4-[(2-morpholin-4-ylethoxy)methyl]piperidin-1-yl-7H-benzo[h]imidazo[4,5-f]isoquinolin-7-one TFA salt

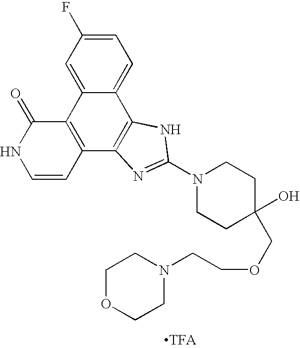

1-Benzyl-9-fluoro-2-4-hydroxy-4-[(2-morpholin-4-ylethoxy)methyl]piperidin-1-yl-1,6-dihydro-7H-benzo[h]imidazo[4,5-f]isoquinolin-7-one bis-TFA salt (113 mg, 0.000156 mol) was dissolved in ethanol (10.0 mL, 0.171 mol). A solution of hydrogen chloride in water (3.0M, 0.60 mL) was added, followed by palladium (10% on carbon, 10.0 mg, 0.0000940 mol) The mixture was degassed and shaken on the parr hydrogenator under 50 psi of hydrogen overnight. The mixture was filtered and the solid was washed with a copious amount of MeOH. The filtrate was concentrated and the crude product was purified using prep-LCMS with TFA/CAN method. Lyophilization of the eluate afforded the product as the TFA salt (12 mg, gray powder).

¹H NMR (400 MHz, CD₃OD): δ 9.94 (dd, 1H), 8.33 (dd, 1H), 7.57 (dd, 1H); 7.53 (dt, 1H), 7.14 (d, 1H), 4.17-3.19 (m, 18H), 2.03-1.86 (m, 4H); MF=C₂₆H₃₀FN₅O₄; LCMS calculated for C₂₆H₃₁FN₅O₄(M+H)⁺: m/z=496.236.

Table 8 contains further examples prepared in a manner analogous to those described above.

TABLE 8

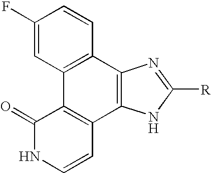

| Ex. | | | | |
|---|---|---|---|---|
| No. | Name | R | MS (ES) (M + 1) | Preparation |
| 299 | 9-fluoro-2-(pyridin-3- yloxy)-3,6-dihydro-7H- benzo[h]imidazo[4,5- f]isoquinolin-7-one · TFA | 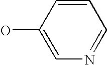 | 347 | Example 294 |
| 300 | 9-fluoro-2-isopropoxy-3,6- dihydro-7H- benzo[h]imidazo[4,5- f]isoquinolin-7-one · TFA | 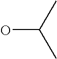 | 312 | Example 294 |
| 301 | 9-fluoro-2-(pyridin-3- ylmethoxy)-3,6-dihydro- 7H-benzo[h]imidazo[4,5- f]isoquinolin-7-one | 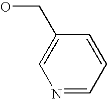 | 361 | Example 294 |
| 302 | 9-fluoro-2-(4-oxopyridin- 1(4H)-yl)-3,6-dihydro-7H- benzo[h]imidazo[4,5- f]isoquinolin-7-one | 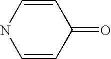 | 347 | Example 294 |
| 303 | 9-fluoro-2-[(cis-4- hydroxycyclohexyl)oxy]- 3,6-dihydro-7H- benzo[h]imidazo[4,5- f]isoquinolin-7-one | 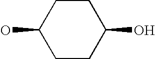 | 368 | Example 294 |
| 304 | 9-fluoro-2-[(trans-4- hydroxycyclohexyl)oxy]- 3,6-dihydro-7H- benzo[h]imidazo[4,5- f]isoquinolin-7-one | 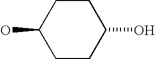 | 368 | Example 294 |
| 305 | 9-fluoro-2-(pyridin-2- ylmethoxy)-3,6-dihydro- 7H-benzo[h]imidazo[4,5- f]isoquinolin-7-one | 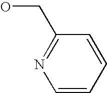 | 361 | Example 294 |
| 306 | 9-fluoro-2-[(trans-4- hydroxycyclohexyl)amino]- 3,6-dihydro-7H- benzo[h]imidazo[4,5- f]isoquinolin-7-one | 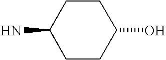 | 367 | Example 295 |
| 307 | 9-fluoro-2-[(pyridin-4- ylmethyl)amino]-3,6- dihydro-7H- benzo[h]imidazo[4,5- f]isoquinolin-7-one · 2TFA | 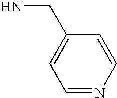 | 360 | Example 295 |
| 308 | 9-fluoro-2-[(1- oxidopyridin-2- yl)methoxy]-3,6-dihydro- 7H-benzo[h]imidazo[4,5- f]isoquinolin-7-one · TFA | 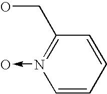 | 377 | Example 294 |
| 309 | 9-fluoro-2-[(1- oxidopyridin-3- yl)methoxy]-3,6-dihydro- 7H-benzo[h]imidazo[4,5- f]isoquinolin-7-one | 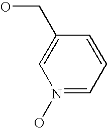 | 377 | Example 294 |
| 310 | 9-fluoro-2-[(1R,3R)-3- hydroxycyclohexyl]oxy- 3,6-dihydro-7H- benzo[h]imidazo[4,5- f]isoquinolin-7-one | 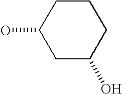 | 368 | Example 294 |
| 311 | cis- and trans-9-fluoro-2- [3-hydroxycyclohexyl]oxy- 3,6-dihydro-7H- benzo[h]imidazo[4,5- f]isoquinolin-7-one | 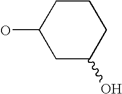 | 368 | Example 294 |
| 312 | 9-fluoro-2-[1- (methylsulfonyl)piperidin- 4-yl]oxy-3,6-dihydro-7H- benzo[h]imidazo[4,5- f]isoquinolin-7-one · TFA | 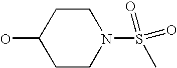 | 431 | Example 294 |
| 312 | 2-[(1-acetylpiperidin-4- yl)oxy]-9-fluoro-3,6- dihydro-7H- benzo[h]imidazo[4,5- f]isoquinolin-7-one · TFA | 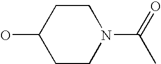 | 395 | Example 294 |
| 314 | 3-4-[(9-fluoro-7-oxo-6,7- dihydro-3H- benzo[h]imidazo[4,5- f]isoquinolin-2- yl)oxy]piperidin-1-yl-3- oxopropanenitrile · TFA | 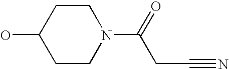 | 420 | Example 294 |
| 315 | 9-fluoro-2-[(trans-4- hydroxycyclohexyl)(methyl) amino]-3,6-dihydro-7H- benzo[h]imidazo[4,5- f]isoquinolin-7-one · TFA | 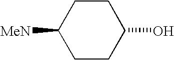 | 381 | Example 295 |
| 316 | 9-fluoro-2-(3-morpholin-4- ylpropoxy)-1,6-dihydro- 7H-benzo[h]imidazo[4,5- f]isoquinolin-7-one · 2TFA | 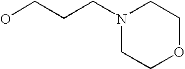 | 397 | Example 296 |
| 317 | 9-fluoro-2-[3-(2- oxopyrrolidin-1- yl)propyl]amino-1,6- dihydro-7H- benzo[h]imidazo[4,5- f]isoquinolin-7-one · TFA | 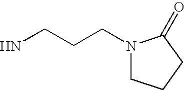 | 394 | Example 297 |
| 318 | 2-(4-{[bis(2- methoxyethyl)amino]methyl}piperidin-1-yl)-9-fluoro- 1,6-dihydro-7H- benzo[h]imidazo[4,5- f]isoquinolin-7-one · 2TFA | 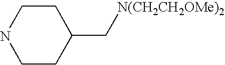 | 482 | Example 297 |
| 319 | 9-fluoro-2-{4-[(2- morpholin-4- ylethoxy)methyl]piperidin- 1-yl}-1,6-dihydro-7H- benzo[h]imidazo[4,5- f]isoquinolin-7-one · 2TFA | 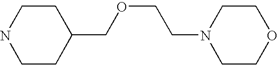 | 480 | Example 297 |
| 320 | 2-[(cyclopropylmethyl)(3- morpholin-4- ylpropyl)amino]-9-fluoro- 1,6-dihydro-7H- benzo[h]imidazo[4,5- f]isoquinolin-7-one · 2TFA | 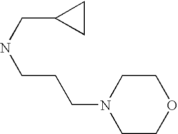 | 450 | Example 297 |
| 321 | 9-fluoro-2-4-hydroxy-4-[(3- piperidin-1- ylpropoxy)methyl]piperidin-1- yl-1,6-dihydro-7H- benzo[h]imidazo[4,5- f]isoquinolin-7-one · 2TFA | 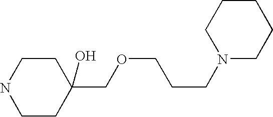 | 508 | Example 298 |
| 322 | 9-fluoro-2-4-hydroxy-4-[(3- morpholin-4- ylpropoxy)methyl]piperidin-1- yl-1,6-dihydro-7H- benzo[h]imidazo[4,5- f]isoquinolin-7-one · 2TFA | 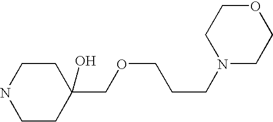 | 510 | Example 298 |

### Example 323

### 10-Fluoro-2-[4-(hydroxyimino)cyclohexyl]benzo[c]imidazo[1,2-a]-1,6-naphthyridin-8(7H)-one

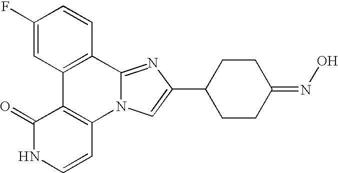

A mixture of 10-fluoro-2-(4-oxocyclohexyl)benzo[c]imidazo[1,2-a]-1,6-naphthyridin-8(7h)-one (115 mg, 329 μmol), methanol (1.55 mL, 38.3 mmol), hydroxylamine hydrochloride (96.0 mg, 1.38 mmol) and potassium bicarbonate (134 mg, 1.34 mmol) was stirred at 23° C. for 16 hours. The reaction mixture was reduced to dryness and the residue was triturated with water to give the desired product (98.7 mg, 82.3%).

¹H NMR (400 MHz, DMSO-d₆): 612.17 (s, 1H), 10.24 (s, 1H), 9.61 (dd, 1H), 8.49 (m, 1H), 8.28 (s, 1H), 7.79 (m, 1H), 7.50 (m, 1H), 7.17 (d, 1H), 3.18 (m, 1H), 2.98 (m, 1H), 2.36 (m, 1H), 2.24 (m, 3H), 1.94 (m, 1H), 1.66 (m, 2H); MF=C₂₀H₁₇FN₄O₂; LCMS calculated for C₂₀H₁₈FN₄O₂(M+H)⁺: m/z 15=365.1, found 365.2.

### Example 324

### 9-Fluoro-2-4-[(2-morpholin-4-ylethoxy)imino]piperidin-1-yl-1,6-dihydro-7H-benzo[h]imidazo[4,5-f]isoquinolin-7-one, bis-TFA salt

### Step 1: 1-Benzyl-9-fluoro-2-[4-(hydroxyimino)piperidin-1-yl]-6-[2-(trimethylsilyl)ethoxy]methyl-1,6dihydro-7H-benzo[h]imidazo[4,5-f]isoquinolin-7-one

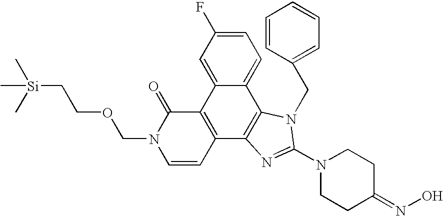

To a solution of 1-benzyl-9-fluoro-2-(4-oxopiperidin-1-yl)-6-[2-(trimethylsilyl)ethoxy]methyl-1,6-dihydro-7H-benzo[h]imidazo[4,5-f]isoquinolin-7-one (prepared as described in Step 2 of Example 298, 421 mg, 738 μmol) in methanol (10 mL) were added potassium bicarbonate (3.00E2 mg, 2.99 mmol) and hydroxylamine hydrochloride (215 mg, 3.10 mmol), and the resultant mixture was stirred at 25° C. overnight. The solvent was removed in vacuo then water was added, and the solid was stirred with water. The solid product was recovered by filtration and dried under vacuum to give a light yellow solid (409 mg, 94.66%).

¹H NMR (400 MHz, CDCl₃): δ 10.24 (dd, 1H), 7.90 (dd, 1H), 7.63 (d, 1H), 7.58 (s, 1H), 7.52 (d, 1H), 7.40-7.28 (m, 3H), 7.23-7.18 (m 2H), 7.15 (ddd, 1H), 5.77 (s, 2H), 5.60 (s, 2H), 3.71 (t, 2H), 3.40 (t, 2H), 3.35 (t, 2H), 2.81 (t, 2H), 2.50 (t, 2h), 0.99 (t, 2H), −0.01 (s, 9H); MF=C₃₂H₃₆FN₅O₃Si; LCMS calculated for C₃₂H₃₇FN₅O₃Si(M+H)⁺: m/z=586.265.

### Step 2: 1-Benzyl-9-fluoro-2-4-[(2-morpholin-4-ylethoxy)imino]piperidin-1-yl-6-[2-(trimethylsilyl)ethoxy]methyl-1,6-dihydro-7H-benzo[h]imidazo[4,5-f]isoquinolin-7-one

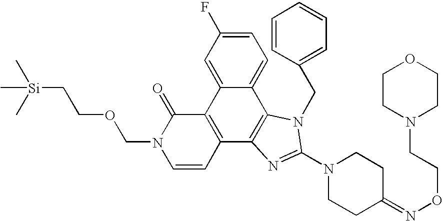

1-Benzyl-9-fluoro-2-[4-(hydroxyimino)piperidin-1-yl]-6-[2-(trimethylsilyl)ethoxy]methyl-1,6-dihydro-7H-benzo[h]imidazo[4,5-f]isoquinolin-7-one (41 mg, 70.0 μmol) was dissolved in 2 mL of DMF, and sodium hydride (11.4 mg, 2.80E2 μmol) was added. After five minutes, 4-(2-chloroethyl)morpholine hydrochloride (19.5 mg, 105 μmol) was added. After 30 minutes, the temperature was raised to 70° C. for one hour. The mixture was cooled and quenched with a few drops of water, and was purified by prep-HPLC (CAN/TFA method) to afford two isomers; major isomer: (33 mg, 68%).

MF=C₃₈H₄₇FN₆O₄Si; LCMS calculated for C₃₈H₄₈FN₆O₄Si(M+H)⁺: m/z=699.349.

### Step 3: 1-Benzyl-9-fluoro-2-4-[(2-morpholin-4-ylethoxy)imino]piperidin-1-yl-1,6-dihydro-7H-benzo[h]imidazo[4,5-f]isoquinolin-7-one

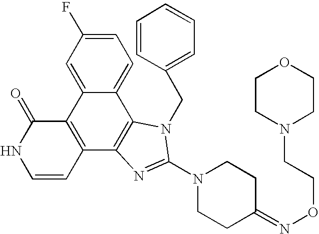

1-Benzyl-9-fluoro-2-4-[(2-morpholin-4-ylethoxy)imino]piperidin-1-yl-6-[2-(trimethylsilyl)ethoxy]methyl-1,6-dihydro-7h-benzo[h]imidazo[4,5-f]isoquinolin-7-one (221 mg, 224 μmol) was dissolved in 100 mL of 20% TFA in DCM and the solution was stirred at 25° C. for 3 hours. The solvent was removed in vacuo. The crude product was subjected to hydrogenation in the subsequent step without further purification.

MF=C₃₂H₃₃FN₆O₃; LCMS calculated for C₃₂H₃₄FN₆O₃(M+H)⁺: m/z=569.268.

### Step 4: 9-Fluoro-2-4-[(2-morpholin-4-ylethoxy)imino]piperidin-1-yl-1,6-dihydro-7H-benzo[h]imidazo[4,5-f]isoquinolin-7-one, bis-TFA salt

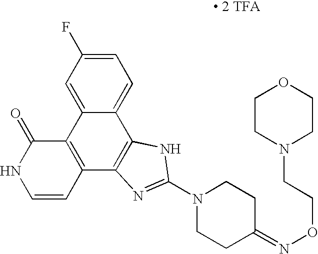

To a solution of 1-benzyl-9-fluoro-2-4-[(2-morpholin-4-ylethoxy)imino]piperidin-1-yl-1,6-dihydro-7H-benzo[h]imidazo[4,5-f]isoquinolin-7-one) (101 mg, 178 μmol) in 25 ml of EtOH and 1.0 mL of hydrogen chloride in water (3.0 M) was added palladium (10 mg, 10% on carbon, 94.0 μmol). The mixture was subjected to 50 psi of hydrogen overnight. The mixture was then filtered, and the catalyst was washed with a copious amount of MeOH. The filtrate was concentrated and the product was isolated and purified on prep-HPLC with CAN/TFA method to afford 20 mg of light yellow powder after lyophilization as the TFA salt (20 mg, 16%).

¹H NMR (300 MHz, CD₃OD): δ 9.69 (d, 1H), 8.04 (dd, 1H), 7.46-7.33 (m, 2H), 6.85 (d, 1H), 4.54-4.44 (br m, 2H), 4.16-3.20 (m, 14H), 3.01 (br t, 2H), 2.82 (br t, 2H); ¹⁹F NMR (300 MHz, CD₃OD): δ−113.2, −77.2; MF=C₂₅H₂₇FN₆O₃; LCMS calculated for C₂₅H₂₈FN₆O₃(M+H)⁺: m/z=479.221.

The following compounds in Table 9 were prepared by procedures substantially as described in Step G of Example 12, in Example 13 and in Example 14.

TABLE 9

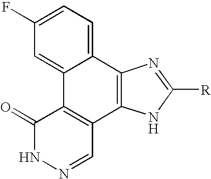

| Ex. | | | MS (ES) | |
|---|---|---|---|---|
| No. | Name | R | (M + 1) | Preparation |
| 325 | 4-ethyl-4-(9-fluoro-7-hydroxy- 3H-benzo[f]imidazo[4,5- h]phthalazin-2-yl)hexanenitrile | 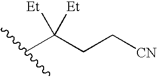 | 378 | Example 12, Step G through Examples 13 & 14. |
| 326 | 4-ethyl-4-(9-fluoro-7-oxo-6,7- dihydro-3H- benzo[f]imidazo[4,5- h]phthalazin-2-yl)hexanoic acid | 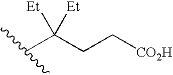 | 397 | Example 12, Step G through Examples 13 & 14. |
| 327 | 4-ethyl-4-(9-fluoro-7-hydroxy- 3H-benzo[f]imidazo[4,5-h]phthalazin-2-yl)hexanamide · 2TFA | 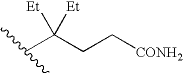 | 396 | Example 12, Step G through Examples 13 & 14. |
| 328 | methyl 2-(9-fluoro-7-hydroxy- 3H-benzo[f]imidazo[4,5- h]phthalazin-2-yl)-2- methylpropanoate · 2TFA | 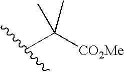 | 355 | Example 12, Step G through Examples 13 & 14. |
| 329 | 2-(9-fluoro-7-hydroxy-3H- benzo[f]imidazo[4,5- h]phthalazin-2-yl)-2- methylpropanoic acid · 2TFA | 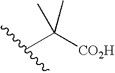 | 341 | Example 12, Step G through Examples 13 & 14. |
| 330 | 9-fluoro-2-(2-hydroxy-1,1- dimethylethyl)-3H- benzo[f]imidazo[4,5- h]phthalazin-7-ol · 2TFA | 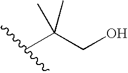 | 327 | Example 12, Step G through Examples 13 & 14. |
| 331 | 4-(9-fluoro-7-oxo-6,7-dihydro- 3H-benzo[f]imidazo[4,5- h]phthalazin-2-yl)-4- methylpentanenitrile · 2TFA | 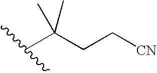 | 350 | Example 12, Step G through Examples 13 & 14. |
| 332 | (2E)-4-(9-fluoro-7-oxo-6,7- dihydro-3H- benzo[f]imidazo[4,5- h]phthalazin-2-yl)-4- methylpent-2-enenitrile · 2TFA | 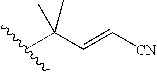 | 348 | Example 12, Step G through Examples 13 & 14. |
| 333 | 9-fluoro-2-[4- (methylthio)phenyl]-3,6- dihydro-7H- benzo[f]imidazo[4,5- h]phthalazin-7-one | 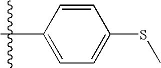 | 377 | Example 12, Step G through Examples 13 & 14. |
| 334 | 9-fluoro-2-[4- (methylsulfinyl)phenyl]-3,6- dihydro-7H- benzo[f]imidazo[4,5- h]phthalazin-7-one | 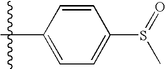 | 393 | Example 12, Step G through Examples 13 & 14. |
| 335 | 4-(9-fluoro-7-hydroxy-3H- benzo[f]imidazo[4,5- h]phthalazin-2-yl)-4- methylpentanamide | 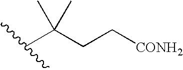 | 368 | Example 12, Step G through Examples 13 & 14. |
| 336 | 3-(9-fluoro-7-hydroxy-3H- benzo[f]imidazo[4,5-h]phthalazin-2-yl)propanenitrile · TFA | 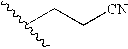 | 308 | Example 12, Step G through Examples 13 & 14. |
| 337 | 9-fluoro-2-(2-pyridin-2- ylethyl)-3H- benzo[f]imidazo[4,5- h]phthalazin-7-ol | 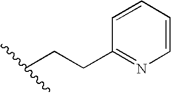 | 360 | Example 12, Step G through Examples 13 & 14. |
| 338 | 9-fluoro-2-(pyridin-4- ylmethyl)-3,6-dihydro-7H- benzo[f]imidazo[4,5- h]phthalazin-7-one | 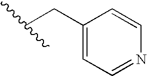 | 346 | Example 12, Step G through Examples 13 & 14. |
| 339 | 9-fluoro-2-(pyridin-3- ylmethyl)-3,6-dihydro-7H- benzo[f]imidazo[4,5- h]phthalazin-7-one · 3TFA | 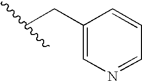 | 346 | Example 12, Step G through Examples 13 & 14. |
| 340 | 2-[4-(benzyloxy)cyclohexyl]-9- fluoro-3,6-dihydro-7H- benzo[f]imidazo[4,5- h]phthalazin-7-one | 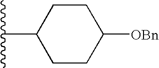 | 443 | Example 12, Step G through Examples 13 & 14. |
| 341 | 9-fluoro-2-(4- hydroxycyclohexyl)-3,6- dihydro-7H- benzo[f]imidazo[4,5- h]phthalazin-7-one | 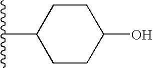 | 353 | Example 12, Step G through Examples 13 & 14. |

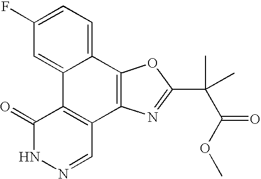

### Example 342

### Methyl 2-(9-fluoro-7-hydroxybenzo[f][1,3]oxazolo[5,4-h]phthalazin-2-yl)-2-methylpropanoate, bis-TFA salt

This compound was prepared by a procedure substantially as described in Step G of Example 12 and in Example 14, except using appropriate starting materials. In Step G of Example 12, a corresponding oxazole compound also formed as a by-product, This by-product was further subjected to the conditions described in Example 14 to undergo the desired rearrangement.

¹H NMR (500 MHz, CD₃OD): δ 9.91 (dd, 1H), 8.95 (s, 1H), 8.39 (dd, 1H), 7.66 (ddd, 1H), 3.73 (s, 3H), 1.87 (s, 6H); MF=C₁₈H₁₄FN₃O₄; LCMS calculated for C₁₈H₁₄FN₃O₄(M+H)⁺: m/z=356.33.

Table 10 below contains further examples of the present invention, which were prepared substantially as described in Example 342 except using appropriate starting materials.

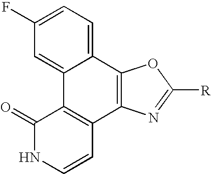

| Ex. | | | | |
|---|---|---|---|---|
| No. | Name | R | MS (ES) (M + 1) | Preparation |
| 343 | benzyl[4-(9-fluoro-7- hydroxybenzo[f][1,3]oxazolo[5,4- h]phthalazin-2-yl)-4- methylpentyl]carbamate · 2TFA | 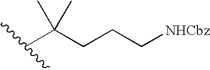 | 489 | Example 12, Step G, then Example 14 |
| 344 | 2-(4-amino-1,1- dimethylbutyl)-9- fluorobenzo[f][1,3]oxazolo[5,4- h]phthalazin-7-ol · 2TFA | 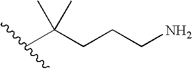 | 355 | Example 12, Step G, then Example 14 |
| 345 | 2-(9-fluoro-7- hydroxybenzo[f][1,3]oxazolo[5,4- h]phthalazin-2-yl)-2- methylpropanoic acid · 2TFA | 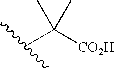 | 342 | Example 12, Step G, then Example 14 |

### Example 346

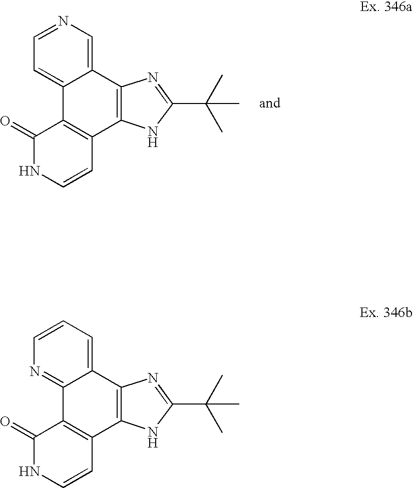

### 2-Tert-butyl-1,9-dihydro-8H-imidazo[4,5-f]-2,8-phenanthrolin-8-one and 2-tert-butyl-3,6-dihydro-7H-imidazo[4,5-f]-1,9-phenanthrolin-7-one

### Step A: 2-(2-Fluoropyridin-4-yl)-1-pyridin-3-ylethanone

A dry flask was charged with sodium bis(trimethylsilyl)amide in tetrahydrofuran (11.0M, 108 mL, 108 mmol) and tetrahydrofuran (50 mL) and the mixture was cooled to 0° C. 2-Fluoro-4-methylpyridine (5 g, 45.0 mmol) was slowly added and the mixture was stirred for 45 minutes. Then 3-pyridinecarboxylic acid, ethyl ester (7.48 g, 49.5 mmol) was added dropwise and the reaction mixture was stirred at 0° C. for 1 hour. The mixture was poured into 2 M HCl (30 mL) and then the aqueous layer was adjusted basic (pH 12) by addition of 5 M NaOH aqueous slution. The mixture was extracted with EtOAc three times, the combined organic extracts were washed with brine, dried over Na₂SO₄, filtered and concentrated to give a yellow/orange solid residue. Recrystalization from ethyl acetate afforded 3.5 g of the product, The mother liquor was chromatographed eluting with 40% ethyl acetate in hexane to afford a further 3.1 g of desired product (6.60 g, 68%).

¹H NMR (300 MHz, CDCl₃): δ9.22 (d, 1H), 8.83 (dd, 1H), 8.26 (dt, 1H), 8.20 (d, 1H), 7.47 (dd, 1H), 7.09 (d, 1H), 6.86 (s, 1H), 4.35 (s, 2H); MF=C₁₂H₉FN₂O; LCMS calculated for C₁₂H₁₀FN₂O(M+H)⁺: m/z=217.078.

### Step B: 1-(2-Fluoropyridin-4-yl)-2-pyridin-3-ylethane-1,2-dione 1-oxime

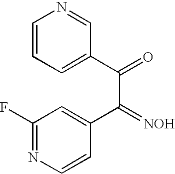

To a solution of (e)-2-(2-fluoropyridin-4-yl)-1-pyridin-3-ylethylenol (3 g, 13.9 mmol) in acetic acid (30 mL) at 5° C. was added slowly dropwise a solution of sodium nitrite (1.15 g, 16.6 mmol) in water (10 mL, 555 mmol). Following the addition, the reaction mixture was stirred for 2 hours. 60 mL of water was added and the resulting precipitate was isolated by filtration and was washed with water and dried under vacuum to afford the product as a white solid (3.20 g, 94%).

MF=C₁₂H₈FN₃O₂; LCMS calculated for C₁₂H₉FN₃O₂(M+H)⁺: m/z=246.068.

### Step C: 2-tert-Butyl-5-(2-fluoropyridin-4-yl)-4-pyridin-3-yl-1H-imidazol-1-ol

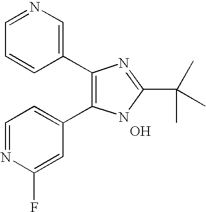

A solution of 1-(2-fluoropyridin-4-yl)-2-pyridin-3-ylethane-1,2-dione 1-oxime (6 g, 24.5 mmol), pivaldehyde (15.2 mL, 97.9 mmol), and ammonium acetate (18.9 g, 245 mmol) in acetic acid (69.6 mL) was stirred at 80° C. for 2 hours. The acetic acid was removed in vacuo and the resulting residue was neutralized by the addition of 5 M NaOH. Following neutralization, the product was extracted with ethyl acetate. The extracts were dried over sodium sulfate and concentrated to afford product, used without further purification in the subsequent step (4.60 g, 60%).

MF=C₁₇H₁₇FN₄O; LCMS calculated for C₁₇H₁₈FN₄O(M+H)⁺: m/z=313.146.

### Step D: 4-(2-tert-Butyl-4-pyridin-3-yl-1H-imidazol-5-yl)-2-fluoropyridine

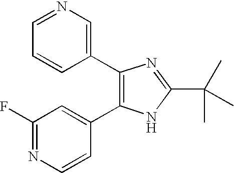

A solution of 2-tert-butyl-5-(2-fluoropyridin-4-yl)-4-pyridin-3-yl-1h-imidazol-1-ol (7 g, 20.2 mmol) and triethyl phosphine (13.8 ml, 80.7 mmol) in N,N-dimethylacetamide (56.3 ml, 605 mmol) was heated to 160° C. for 30 min. The product was purified using prep-HPLC (MeCN/TFA) to afford the desired product (5.10 g).

MF=C₁₇H₁₇FN₄; LCMS calculated for C₁₇H₁₈FN₄(M+H)⁺: m/z=297.152.

### Step E: 4-(2-tert-Butyl-4-pyridin-3-yl-1H-imidazol-5-yl)pyridin-2(1H)-one

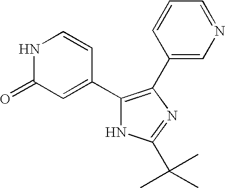

A solution of 4-(2-tert-butyl-4-pyridin-3-yl-1h-imidazol-5-yl)-2-fluoropyridine (5.10 g, 17.2 mmol) in tetrahydrofuran (100 mL) and hydrogen chloride in water (4.00 M, 100 mL) was stirred at 60° C. for 16 hours. The volatile solvent was removed in vacuo and the aqueous portion was neutralized using saturated bicarbonate. The product was extracted with ethyl acetate. The extracts were dried over sodium sulfate, filtered and concentrated. The residue was washed with methanol to afford the desired product (4.50 g).

MF=C₁₇H₁₈N₄O; LCMS calculated for C₁₇H₁₉N₄O(M+H)⁺: m/z=295.156.

### Step F: 2-tert-Butyl-1,9-dihydro-8H-imidazo[4,5-f]-2,8-phenanthrolin-8-one and 2-tert-butyl-3,6-dihydro-7H-imidazo[4,5-f]-1,9-phenanthrolin-7-one

A solution of 4-(2-tert-butyl-4-pyridin-3-yl-1h-imidazol-5-yl)pyridine-2(1h)-one (4.50 g, 15.3 mmol) in methanol (800 mL, 19.7 mol) was irradiated through Pyrex® using a medium pressure Hg vapor lamp for 2 hours. The methanol was removed in vacuo and the mixture was chromatographed on silica gel using 5-10% MeOH in DCM to afford two isomeric products [isomer A (2-tert-butyl-1,9-dihydro-8H-imidazo[4,5-f]-2,8-phenanthrolin-8-one): 1.80 g, 40%, and isomer B (2-tert-butyl-3,6-dihydro-7H-imidazo[4,5-f]-1,9-phenanthrolin-7-one): 1.90 g, 42%].

¹H NMR (500 MHz, d₆-DMSO, 120° C.): (isomer A: 69.88 (d, 1H), 9.83 (s, 1H), 8.61 (d, 1H), 7.57 (d, 1H), 7.32 (d, 1H), 1.57 (s, 9H); isomer B: 69.63 (d, 1H), 9.35 (d, 1H), 8.32 (dd, 1H), 8.08 (d, 1H), 7.62 (d, 1H), 1.56 (s, 9H); MF=C₁₇H₁₆N₄O; LCMS calculated for C₁₇H₁₇N₄O(M+H)⁺: m/z=293.140.

Table 11 contains further examples prepared substantially as described in Example 346, except using an appropriate aldehyde in Step C. Analogs were purified by prep-HPLC using acetonitrile/water containing trifluoroacetic acid to afford products as the trifluoroacetate salts, where indicated.

TABLE 11

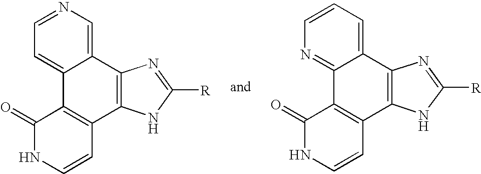

| Ex. | | | |
|---|---|---|---|
| No. | Name | R | MS (ES) (M + 1) |
| 347a | 2-cyclohexyl-1,9-dihydro-8H- imidazo[4,5-f]-2,8-phenanthrolin-8-one | 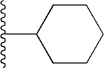 | 319 |
| 347b | 2-cyclohexyl-3,6-dihydro-7H- imidazo[4,5-f]-1,9-phenanthrolin-7-one | 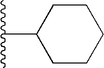 | 319 |
| 348a | 3-(8-oxo-8,9-dihydro-1H-imidazo[4,5-f]- 2,8-phenanthrolin-2-yl)propanenitrile | 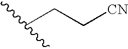 | 290 |
| 348b | 3-(7-oxo-6,7-dihydro-3H-imidazo[4,5-f]- 1,9-phenanthrolin-2-yl)propanenitrile | 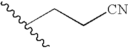 | 290 |
| 349a | 2-(trans-4-hydroxycyclohexyl)-1,9- dihydro-8H-imidazo[4,5-f]-2,8- phenanthrolin-8-one · 2TFA | 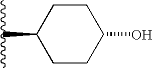 | 335 |
| 349b | 2-(trans-4-hydroxycyclohexyl)-3,6- dihydro-7H-imidazo[4,5-f]-1,9- phenanthrolin-7-one · 2TFA | 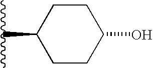 | 335 |
| 350a | 2-[1-(methylsulfonyl)piperidin-4-yl]-1,9- dihydro-8H-imidazo[4,5-f]-2,8- phenanthrolin-8-one | 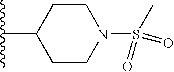 | 398 |
| 350b | 2-[1-(methylsulfonyl)piperidin-4-yl]-3,6- dihydro-7H-imidazo[4,5-f]-1,9- phenanthrolin-7-one | 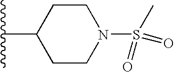 | 398 |
| 351a | 3-oxo-3-[4-(8-oxo-8,9-dihydro-1H- imidazo[4,5-f]-2,8-phenanthrolin-2- yl)piperidin-1-yl]propanenitrile | 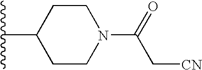 | 387 |
| 352a | 3-methyl-3-(8-oxo-8,9-dihydro-1H- imidazo[4,5-f]-2,8-phenanthrolin-2- yl)butanenitrile · 2TFA | 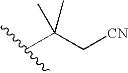 | 318 |
| 352b | 3-methyl-3-(7-oxo-6,7-dihydro-3H- imidazo[4,5-f]-1,9-phenanthrolin-2- yl)butanenitrile · 2TFA | 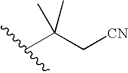 | 318 |

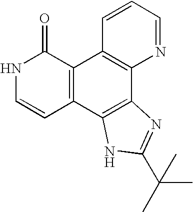

### Example 353

### 2-tert-Butyl-1,9-dihydro-8H-imidazo[4,5-f]-2,7-phenanthrolin-8-one

### Step A: 2-(2-Fluoropyridin-4-yl)-1-pyridin-2-ylethanone

To a mixture of a solution of sodium bis(trimethylsilyl)amide in tetrahydrofuran (1.0 M, 108 mL, 108 mmol) and tetrahydrofuran (50 mL, 616 mmol) at 0° C. was slowly added 2-fluoro-4-methylpyridine (5 g, 45.0 mmol). The mixture was stirred for 45 minutes at this temperature, followed by an addition of 2-pyridinecarboxylic acid, ethyl ester (7.48 g, 49.5 mmol). The reaction mixture was allowed to warm to 25° C. and stirred for additional 2 hours. The mixture was poured into 2 M HCl (30 mL) and the pH was then adjusted to 12 by an addition of 5 M NaOH. The product was extracted with three portions of ethyl acetate. The combined organic extracts were washed with brine, were dried over sodium sulfate, filtered and concentrated in vacuo to afford a yellow/orange residue. By recrystallization from ethyl acetate, 3.0 g of desired product was obtained. The mother liquor was subjected to flash column chromatography (30% ethyl acetate/hexanes) to yield additional 3.7 g of desired product (6.70 g, 69%).

¹H NMR (300 MHz, CDCl₃): δ8.73 (dq, 1H), 8.16 (d, 1H), 8.08 (dt, 1H), 7.87 (dt, 1H), 7.53 (dq, 1H), 7.18-7.14 (m, 1H), 6.95-6.92 (m, 1H), 4.60 (s, 2H); MF=C₁₂H₉FN₂O; LCMS calculated for C₁₂H₁₀FN₂O(M+H)⁺: m/z=217.078.

### Step B: (2-Fluoropyridin-4-yl)-2-pyridin-2-ylethane-1,2-dione 1-oxime

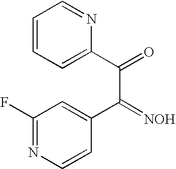

To a solution of (E)-2-(2-fluoropyridin-4-yl)-1-pyridin-2-ylethylenol (3 g, 13.9 mmol) in acetic acid (30 mL) at 5° C. was added dropwise a solution of sodium nitrite (1.15 g, 16.6 mmol) in water (10 mL, 555 mmol). The reaction mixture was stirred at 25° C. for several hours, followed by an addition of water (60 mL). The resulting precipitate was isolated by filtration. The solid was washed with water and was dried under vacuum to afford a white solid (3.10 g, 91%).

MF=C₁₂H₈FN₃O₂; LCMS calculated for C₁₂H₉FN₃O₂(M+H)⁺: m/z=246.068.

### Step C: 2-tert-Butyl-5-(2-fluoropyridin-4-yl)-4-pyridin-2-yl-1H-imidazol-1-ol

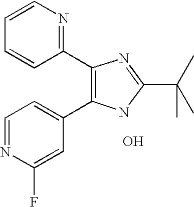

A solution of (2-fluoropyridin-4-yl)-2-pyridin-2-ylethane-1,2-dione 1-oxime (200 mg, 816 μmol), pivaldehyde (1.14 mL, 7.34 mmol), and ammonium acetate (1.13 g, 14.7 mmol) in acetic acid (5 mL, 87.9 mmol) was heated to 80° C. for 2 hours. The reaction mixture was cooled and diluted with water. The pH was adjusted to 10 by an addition of 5 M NaOH and the mixture was extracted with ethyl acetate three times. The combined organic layer was dried with sodium sulfate, filtered, and concentrated in vacuo to yield the desired product (210 mg, 82%).

MF=C₁₇H₁₇FN₄O; LCMS calculated for C₁₇H₁₈FN₄O(M+H)⁺: m/z=313.146.

### Step D: 4-(2-tert-Butyl-4-pyridin-2-yl-1H-imidazol-5-yl)-2-fluoropyridine

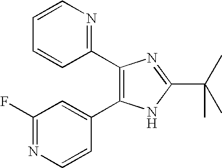

A solution of 2-tert-butyl-5-(2-fluoropyridin-4-yl)-4-pyridin-2-yl-1h-imidazol-1-ol (200 mg, 576 μmol) and triethyl phosphite (889 μL, 5.19 mmol) in N,N-dimethylacetamide (5 mL) was heated to 160° C. for 40 min. The reaction mixture was diluted with water, adjusted to be basic (pH=10) by an addition of 5 M NaOH, and extracted with ethyl acetate three times. The combined organic layer was dried with sodium sulfate, filtered, and concentrated in vacuo. The crude residue was purified by prep-HPLC (MeCN/TFA) to yield the desired product (45 mg).

¹H NMR (300 MHz, CDCl₃): δ 10.77-10.61 (br s, 1H), 8.50 (dq, 1H), 8.17 (d, 1H), 7.61 (dt, 1H), 7.49 (dt, 1H), 7.45 (ddd, 1H), 7.25-7.23 (m, 1H), 7.16 (ddd, 1H), 1.46 (s, 9H); MF=C₁₇H₁₇FN₄; LCMS calculated for C₁₇H₁₈FN₄(M+H)⁺: m/z=297.152.

### Step E: 4-(2-tert-Butyl-4-pyridin-2-yl-1H-imidazol-5-yl)pyridin-2(1H)-one

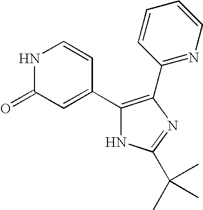

A solution of 4-(2-tert-butyl-4-pyridin-2-yl-1h-imidazol-5-yl)-2-fluoropyridine (45 mg, 152 μmol) in 6.00 M HCl (5 mL, 30 mmol) and tetrahydrofuran (5 mL, 61.6 mmol) was stirred at 70° C. for 5 hours. The reaction mixture was neutralized and then adjusted to be basic (pH 10) by addition of 5 M NaOH, and extracted with ethyl acetate three times. The combined organic layer was dried with sodium sulfate, filtered, and concentrated in vacuo to yield the desired product (35 mg, 78%).

MF=C₁₇H₁₈N₄O; LCMS calculated for C₁₇H₁₉N₄O(M+H)⁺: m/z=295.156.

### Step F: 2-tert-Butyl-1,9-dihydro-8H-imidazo[4,5-f]-2,7-phenanthrolin-8-one

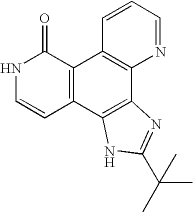

A solution of 4-(2-tert-butyl-4-pyridin-2-yl-1h-imidazol-5-yl)pyridin-2(1h)-one (40 mg, 136 μmol) in methanol (150 ml) was irradiated through Pyrex® with a medium-pressure Hg vapor lamp for 6 hours. The methanol was removed in vacuo and the product was purified by prep-HPLC (16 mg, 40%).

¹H NMR (300 MHz, CD₃OD): δ 10.58 (dd, 1H), 8.81 (dd, 1H), 7.63 (d, 1H), 7.61 (dd, 1H), 7.51 (d, 1H), 1.58 (s, 9H); MF=C₁₇H₁₆N₄O; LCMS calculated for C₁₇H₁₇N₄O(M+H)⁺: m/z=293.140.

Table 12 contains further examples prepared substantially as described in Example 353, except using an appropriate aldehyde in Step C.

TABLE 12

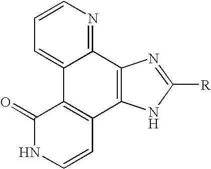

| Ex. | | | |
|---|---|---|---|
| No. | Name | R | MS (ES) (M + 1) |
| 354 | 2-cyclohexyl-1,9-dihydro- 8H-imidazo[4,5-f]-2,7- phenanthrolin-8-one | 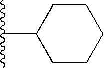 | 319 |
| 355 | 2-cyclopentyl-1,9-dihydro- 8H-imidazo[4,5-f]-2,7- phenanthrolin-8-one | 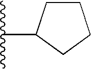 | 305 |

### Example 356

### 2-tert-Butyl-1,9-dihydro-8H-imidazo[4,5-f]-2,8-phenanthrolin-8-one 5-oxide

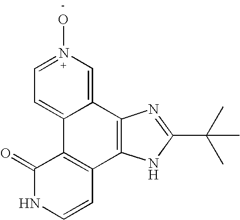

To a chloroform solution containing 10% isopropanol was added m-chloroperbenzoic acid (970 mg, 3.93 mmol). The mixture was stirred for 10 min, followed by an addition of 2-tert-butyl-1,9-dihydro-8H-imidazo[4,5-f]-2,8-phenanthrolin-8-one (prepared in Example 346, Step G, 230 mg, 0.79 mmol). The solution was stirred for 6 hours. Sodium bicarbonate solution (saturated) was added to adjust the pH to 7. Additional chloroform solution containing 10% isopropanol was added and the layers were separated. The organic layer was dried and concentrated. The product was purified by flash column chromatography (8% methanol in DCM) to yield the desired product.

¹H NMR (500 MHz, d₆-DMSO): 610.14 (d, 1H), 9.53 (s, 1H), 8.51-8.46 (m, 1H), 7.73 (d, 1H), 7.36 (d, 1H), 1.53 (s, 9H); MF=C₁₇H₁₆N₄O₂; LCMS calculated for C₁₇H₁₇N₄O₂(M+H)⁺: m/z=309.135.

Table 13 contains a further example prepared substantially as described in Example 356, except using appropriate starting materials.

TABLE 13

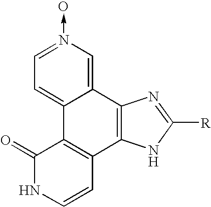

| Ex. | | | MS (ES) |
|---|---|---|---|
| No. | Name | R | (M + 1) |
| 357 | 3-methyl-3-(5-oxido-8-oxo-8,9- dihydro-1H-imidazo[4,5-f]- 2,8-phenanthrolin-2-yl) butanenitrile · TFA | 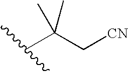 | 334 |

### Example A

In Vitro JAK Assay

Compounds herein were tested for inhibitory activity of Jak targets according to the following in vitro assay described in Park et al., Analytical Biochemistry 1999, 269, 94-104. The catalytic domains of human Jak1 (a.a. 837-1142), Jak2 (a.a. 828-1132) and Jak3 (a.a. 781-1124) with an N-terminal His tag were expressed using baculovirus in insect cells and purified. The catalytic activity of JAK1, JAK2 or JAK3 was assayed by measuring the phosphorylation of a biotinylated peptide. The phosphorylated peptide was detected by homogenous time resolved fluorescence (HTRF). IC₅₀s of compounds were measured for each kinase in the reactions that contain the enzyme, ATP and 500 nM peptide in 50 mM Tris (pH 7.8) buffer with 100 mM NaCl, 5 mM DTT, and 0.1 mg/mL (0.01%) BSA. The ATP concentration in the reactions was 90 μM for Jak1, 30 μM for Jak2 and 3 μM for Jak3. Reactions were carried out at room temperature for 1 hr and then stopped with 20 μL 45 mM EDTA, 300 nM SA-APC, 6 nM Eu-Py20 in assay buffer (Perkin Elmer, Boston, Mass.). Binding to the Europium labeled antibody took place for 40 minutes and HTRF signal was measured on a Fusion plate reader (Perkin Elmer, Boston, Mass.). Certain compounds recited herein showed an IC₅₀ of 10 μM or less for at least one of the above-mentioned Jak targets and were therefore considered active.

### Example B

### Contact Delayed-Type Hypersensitivity Assay

Efficacy of compounds of the invention for treatment of psoriasis can be tested in the T-cell driven murine DTH model. The murine skin contact delayed-type hypersensitivity (DTH) response is considered to be a valid model of clinical contact dermatitis, and other T-lymphocyte mediated immune disorders of the skin, such as psoriasis (Immunol Today. 1998 January; 19 (1):37-44). Murine DTH shares multiple characteristics with psoriasis, including the immune infiltrate, the accompanying increase in inflammatory cytokines, and keratinocyte hyperproliferation. Furthermore, many classes of agents that are efficacious in treating psoriasis in the clinic are also effective inhibitors of the DTH response in mice (Agents Actions. 1993 January; 38 (1-2):116-21;).

In the DTH model, sensitization occurs with the topical application of antigen to the skin on days 0 and 1 resulting in a DTH response upon challenge with the same antigen on day 5. Twenty-four or forty eight hours later, the reactive skin site exhibits a cellular infiltrate resulting in an indurated type inflammation and keratinocyte hyperproliferation. In the initial experiment, a test compound is administered continuously using mini-osmotic pumps to deliver 150 mg/kg/d. In this paradigm, the Jak inhibitor is present throughout both the sensitization and challenge phases of the DTH response. The inflammatory response is monitored by measuring the ear thickness prior to and after immune challenge. Differences in ear thickness are calculated for each mouse and then averaged for the group. Comparisons can then be made between vehicle and treated groups in the context of the negative controls (challenged without sensitization) and therapeutic positive control mice (treated with dexamethasone or other efficacious agent).

### Example C

In Vitro Mutant Jak (mtJAK) Assay

Compounds herein can be tested for inhibitory activity of mutant Jak (mtJak) targets according to the following in vitro assay described in Park et al., Analytical Biochemistry 1999, 269, 94-104 with variations described herein. Activating mutations, residing anywhere within the coding region of the Jak DNA, cDNA, or mRNA, can be introduced to nucleic acid sequences encoding for Jaks using standard molecular biology techniques (e.g. nucleotide mutagenesis) familiar to those schooled in the art. This includes, but is not limited to mutations in the codon for a.a. 617 that results in a substitution of the wild-type valine with a phenylalanine. The kinase domain (a.a. 828-1132), the pseudo-kinase and kinase domains (a.a. 543-827 and 828-1132, respectively), or the entire Jak protein, with an N-terminal His tag, can be expressed using baculovirus in insect cells and purified. Similar strategies can be employed to generate mutant Jak1, Jak3, or Tyk2. The catalytic activity of Jak can then be assayed by measuring the phosphorylation of a biotinylated peptide. The phosphorylated peptide can be detected by homogenous time resolved fluorescence (HTRF) using suitable and optimized buffers and concentrations of ATP, peptide, kinase, etc. Compounds having an IC₅₀ of about 10 μM or less for any of the above-mentioned Jak targets will typically be considered active.

### Example D

Cell-Based mtJAK Assay

As a complement to the in vitro kinase assay, cells expressing the mutated form(s) of Jak may be identified (e.g. HEL cells, ATCC) or constructed (by transfection, infection, or similar technique to introduce the nucleic acid encoding for the Jak) using techniques familiar to those schooled in the art. Cells may then be treated with compounds for various times (usually between 0 and 4 hours) and collected for protein extraction using methods familiar to those schooled in the art. Cellular protein extracts can then be analyzed for both total and phospho-Jak using, for example, the following antibodies: total Jak1 (Cell Signaling, #9138), phospho-Jak1 (Abcam, #ab5493), total Jak2 (Upstate #06-255), phospho-Jak (Cell Signaling, #3771), total Jak3 (Santa Cruz, #sc-513), phospho-Jak3 (Santa Cruz, #sc-16567), total Tyk2 (Santa Cruz #sc-169), phospho-Tyk2 (Cell Signal #9321), and phospho-tyrosine (Upstate, #05-231). Methodologies to perform these analyses include but are not limited to immunoblotting, immunoprecipitation, ELISA, RIA, immunocytochemistry, and FACS.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

What is claimed is:

1. A compound of Formula I: 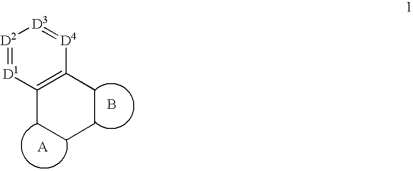
or pharmaceutically acceptable salt or prodrug thereof, wherein:
D¹ is N, NO, or CR^{1a};
D² is N, NO, or CR^{1b};
D³ is N, NO, or CR^{1c};
D⁴ is N, NO or CR^{1d};
Ring A is 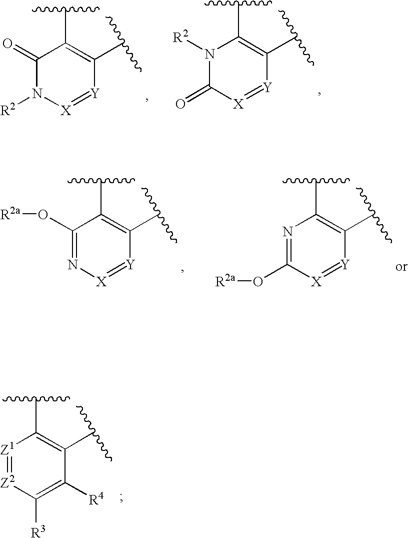
X and Y are each, independently, N or CR⁵;
Z¹ and Z² are each, independently, N, CR⁶, or NO; wherein at least one of Z, and Z² is other than CR⁶;
Ring B is 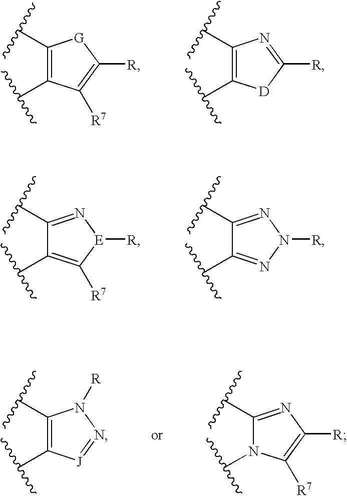
D is O, S, or NR⁸;
E is N or CR⁹;
G is O, S, or NR⁸;
J is N or CR⁷;
R is —W¹—W²—W³—W⁴;
W¹ is absent, C₁₋₆ alkyl, C₂₋₆ alkenyl, C₂₋₆ alkynyl, O, S, NR¹¹, CO, COO, CONR¹¹, SO, SO₂, SONR¹¹, SO₂NR¹¹, or NR¹¹CONR¹², wherein said C₁₋₆ alkyl, C₂₋₆ alkenyl, C₂₋₆ alkynyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
W² is absent, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by one or more halo, CN, NO₂, OH, ═NH, ═NOH, ═NO—(C₁₋₄ alkyl), C₁₋₄ haloalkyl, C₁₋₄ alkoxy, C₁₋₄ haloalkoxy, amino, C₁₋₄ alkylamino or C₂₋₈ dialkylamino;
W³ is absent, C₁₋₆ alkyl, C₂₋₆ alkenyl, C₂₋₆ alkynyl, O, S, NR¹⁰, ═N—, ═N—O—, ═N—O—(C₁₋₄ alkyl), O—(C₁₋₄ alkyl), S—(C₁₋₄ alkyl), NR¹⁰—(C₁₋₄ alkyl), (C₁₋₄ alkyl)-O—(C₁₋₄ alkyl), (C₁₋₄ alkyl)-S—(C₁₋₄ alkyl), (C₁₋₄ alkyl)-NR¹⁰—(C₁₋₄ alkyl), CO, COO, C(O)—(C₁₋₄ alkyl), C(O)O—(C₁₋₄ alkyl), C(O)—(C₁₋₄ alkyl)-C(O), NR¹⁰C(O)—(C₁₋₄ alkyl), C(O)NR¹⁰—(C₁₋₄ alkyl), NR¹⁰C(O)O—(C₁₋₄ alkyl), NR¹⁰C(O)O, CONR¹⁰, SO, SO₂, SONR¹⁰, SO₂NR¹⁰, or NR¹⁰CONR¹¹, wherein said C₁₋₆ alkyl, C₂₋₆ alkenyl, C₂₋₆ alkynyl are each optionally substituted by 1, 2 or 3 halo, OH, CN, C₁₋₄ alkoxy, C₁₋₄ haloalkoxy, amino, C₁₋₄ alkylamino or C₂₋₈ dialkylamino;
W⁴ is H, NR¹⁰R¹¹, CN, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2, 3, 4 or 5 halo, OH, CN, C₁₋₄ alkoxy, ═NH, ═NOH, ═NO—(C₁₋₄ alkyl), C₁₋₄ haloalkyl, C₁₋₄ haloalkoxy, COOH, COO—(C₁₋₄ alkyl), amino, C₁₋₄ alkylamino or C₂₋₈ dialkylamino;
R^{1a}, R^{1b}, R^{1c} and R^{1d} are each, independently, H, halo, C₁₋₄ alkyl, C₂₋₄ alkenyl, C₂₋₄ alkynyl, C₁₋₄ haloalkyl, OH, C₁₋₄ alkoxy, C₁₋₄ haloalkoxy, CN, NO₂, C(O)—(C₁₋₄ alkyl), C(O)OH, C(O)O—(C₁₋₄ alkyl), C(O)NH₂, C(O)NH(C₁₋₄ alkyl), C(O)N(C₁₋₄ alkyl)₂, S(O)₂NH₂, S(O)₂NH(C₁₋₄ alkyl), S(O)₂N(C₁₋₄ alkyl)₂, S(O)₂—(C₁₋₄ alkyl), NH₂, NH(C₁₋₄ alkyl), or N(C₁₋₄ alkyl)₂;
R² is H, OH, C₁₋₆ alkyl, C₂₋₆ alkenyl, C₂₋₆ alkynyl, carbocyclyl, heterocyclyl, carbocyclylalkyl or heterocyclylalkyl;
R^{2a} is C₁₋₆ alkyl, C₂₋₆ alkenyl, C₂₋₆ alkynyl, aryl, heteroaryl, arylalkyl, heteroarylalkyl, cycloalkyl, cycloalkylalkyl, heterocycloalkyl or heterocycloalkylalkyl;
R³, R⁴, R⁵, and R⁶ are each, independently, H, C₁₋₆ alkyl, C₂₋₆ alkenyl, C₂₋₆ alkynyl, halo, C₁₋₄ haloalkyl, CN, NO₂, OR¹², SR¹², C(O)R¹³, C(O)OR¹², C(O)NR¹⁴R¹⁵, NR¹⁴R¹⁵, NR¹⁴CONHR¹⁵, NR¹⁴C(O)R¹³, NR¹⁴C(O)OR¹², S(O)R¹³, S(O)₂R¹³, S(O)NR¹⁴R¹⁵, SO₂NR¹⁴R¹⁵;
R⁷ is H, C₁₋₆ alkyl, C₂₋₆ alkenyl, C₂₋₆ alkynyl, halo, C₁₋₄ haloalkyl, OH, C₁₋₄ alkoxy, C₁₋₄ haloalkoxy, CN, NO₂, C(O)—(C₁₋₄ alkyl), C(O)OH, C(O)O—(C₁₋₄ alkyl), C(O)NH₂, C(O)NH(C₁₋₄ alkyl), C(O)N(C₄ alkyl)₂, S(O)₂NH₂, S(O)₂NH(C₁₋₄ alkyl), S(O)₂N(C₁₋₄ alkyl)₂, S(O)₂—(C₁₋₄ alkyl), NH₂, NH(C₁₋₄ alkyl), or N(C₁₋₄ alkyl)₂;
R⁸ is H, C₁₋₄ alkyl, C₂₋₄ alkenyl, C₂₋₄ alkynyl, OH or C₁₋₄ alkoxy;
R⁹ is H, halo, C₁₋₄ alkyl, C₁₋₄ haloalkyl, C₂₋₄ alkenyl, C₂₋₄ alkynyl, OH, C₁₋₄ alkoxy or C₁₋₄ haloalkoxy;
R¹⁰ and R¹¹ are each, independently, H, C₁₋₆ alkyl, C₂₋₆ alkenyl, C₂₋₆ alkynyl, aryl, cycloalkyl, arylalkyl, cycloalkylalkyl, COR^{a}, SOR^{a}, or SO₂R^{a} wherein each of said C₁₋₆ alkyl, C₂₋₆ alkenyl, C₂₋₆ alkynyl, aryl, cycloalkyl, arylalkyl, or cycloalkylalkyl is optionally substituted by 1, 2 or 3 substitutents slected from halo, C₁₋₄ alkyl, C₁₋₄ haloalkyl, OH, C₁₋₄ alkoxy, C₁₋₄ haloalkoxy, amino, C₁₋₄ alkylamino, C₂₋₈ dialkylamino, aminocarbonyl, C₁₋₄ alkylaminocarbonyl, or C₂₋₈ dialkylaminocarbonyl, CN and NO₂;
or R¹⁰ and R¹¹ together with the N atom to which they are attached form a heterocycloalkyl group optionally substituted by 1, 2 or 3 substitutents slected from halo, C₁₋₄ alkyl, C₁₋₄ haloalkyl, OH, C₁₋₄ alkoxy, C₁₋₄ haloalkoxy, amino, C₁₋₄ alkylamino, C₂₋₈ dialkylamino, aminocarbonyl, C₁₋₄ alkylaminocarbonyl, or C₂₋₈ dialkylaminocarbonyl;
R¹² and R¹³ are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
R¹⁴ and R¹⁵ are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R¹⁴ and R¹⁵ together with the N atom to which they are attached form a heterocyclyl group;
R^{a} is H, C₁₋₆ alkyl, C₁₋₆ haloalkyl, C₂₋₆ alkenyl, C₂₋₆ alkynyl, aryl, cycloalkyl, arylalkyl, cycloalkylalkyl, heteroaryl, heterocycloalkyl, heteroarylalkyl, heterocycloalkylalkyl, NH₂, NH(C₁₋₆ alkyl), N(C₁₋₆ alkyl)₂, NH(carbocyclyl), N(carbocyclyl)₂, NH(carbocyclylalkyl) or N(carbocyclylalkyl)₂;
with the proviso that when Ring A is: 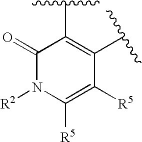
Ring B is: 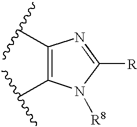
D¹ is CR^{1a};
D² is N or CR^{1b};
D³ is CR^{1c}; and
D⁴ is CR^{1d};
then W¹ is O, S, NR¹¹, SO, SO₂, SONR¹¹, SO₂NR¹¹, or NR¹¹CONR¹².

2. The compound of claim 1 wherein Ring A is

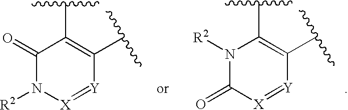

3. The compound of claim 2 wherein R² is H.

4. The compound of claim 2 wherein R² is H, X is CH and Y is CH.

5. The compound of claim 1 wherein Ring A is

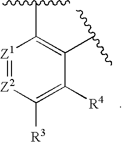

6. The compound of claim 1 wherein Ring A is

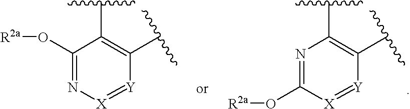

7. The compound of claim 6 wherein R^{2a} is C₁₋₆ alkyl.

8. The compound of claim 6 wherein at least one of X and Y is N.

9. The compound of claim 1 wherein Ring B is

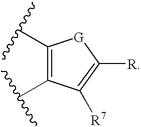

10. The compound of claim 9 wherein G is O or S.

11. The compound of claim 9 wherein G is NR⁸.

12. The compound of claim 9 wherein R is H, C₁₋₆ alkyl, aryl, heteroaryl, cycloalkyl, heterocycloalkyl, or NR¹⁰R¹¹.

13. The compound of claim 1 wherein Ring B is

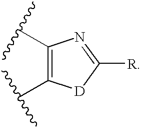

14. The compound of claim 13 wherein D is S.

15. The compound of claim 13 wherein D is O.

16. The compound of claim 13 wherein D is NR⁸.

17. The compound of claim 13 wherein R is H, C₁₋₆ alkyl, aryl, heteroaryl, cycloalkyl, heterocycloalkyl, or NR¹⁰R¹¹.

18. The compound of claim 13 wherein R is (C₁₋₆ alkyl)-W²—W³—W⁴, O—W²—W³—W⁴, S—W²—W³—W⁴, NR¹¹—W²—W³—W⁴, or —W²—W³—W⁴.

19. The compound of claim 13 wherein D is S or O and R is O—W²—W³—W⁴, S—W²—W³—W⁴ or NR¹¹—W²—W³—W⁴.

20. The compound of claim 1 wherein Ring B is

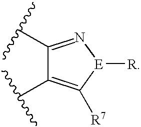

21. The compound of claim 20 wherein E is N.

22. The compound of claim 20 wherein R⁷ is H.

23. The compound of claim 20 wherein R is H, C₁₋₆ alkyl, aryl, heteroaryl, cycloalkyl, heterocycloalkyl, or NR¹⁰R¹¹.

24. The compound of claim 1 wherein Ring B is

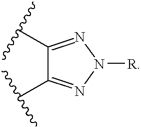

25. The compound of claim 1 wherein Ring B is:

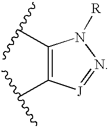

26. The compound of claim 25 wherein J is N.

27. The compound of claim 25 where J is CR⁷.

28. The compound of claim 25 wherein R is H, C₁₋₆ alkyl, aryl, heteroaryl, cycloalkyl, heterocycloalkyl.

29. The compound of claim 25 wherein R is (C₁₋₆ alkyl)-W²—W³—W⁴, CO—W²—W³—W⁴, COO—W²—W³—W⁴, CONR¹¹—W²—W³—W⁴ or SO₂—W²—W³—W⁴.

30. The compound of claim 1 wherein Ring B is

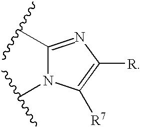

31. The compound of claim 30 wherein R is C₁₋₆ alkyl, C₂₋₆ alkenyl, C₂₋₆ alkynyl, cycloalkyl, or heterocycloalkyl, each optionally substituted by 1, 2, 3, 4 or 5 halo, OH, CN, C₁₋₄ alkoxy, ═NH, ═NOH, ═NO—(C₁₋₄ alkyl), C₁₋₄ haloalkyl, C₁₋₄ haloalkoxy, COOH, COO—(C₁₋₄ alkyl), amino, C₁₋₄ alkylamino or C₂₋₈ dialkylamino;

32. The compound of claim 1 wherein D² is CR^{1b} and CR^{1b} is F, Cl, Br or I.

33. The compound of claim 1 wherein at least one of D¹, D², D³, and D⁴ is N.

34. The compound of claim 1 having the Formula Ia:

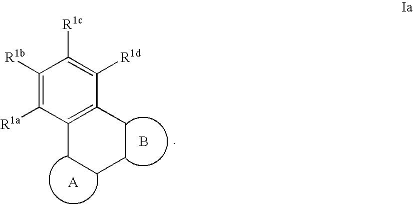

35. The compound of claim 1 wherein R is —W¹—W²—W³—W⁴; and W¹ is absent, C₁₋₆ alkyl, O, S, NR¹¹, SO, or SO₂.

36. The compound of claim 1 wherein R is —W¹—W²—W³—W⁴; and W¹ is absent, and W² is aryl, cycloalkyl, heteroaryl or heterocycloalkyl, each optionally substituted by 1, 2, 3 or 4 halo, CN, NO₂, OH, ═NH, ═NOH, ═NO—(C₁₋₄ alkyl), C₁₋₄ haloalkyl, C₁₋₄ alkoxy, C₄ haloalkoxy, amino, C₁₋₄ alkylamino or C₂₋₈ dialkylamino.

37. The compound of claim 1 selected from:
9-Fluoro-2-piperidin-1-ylbenzo[h][1,3]thiazolo[5,4-f]isoquinolin-7(6H)-one;
2-(tert-Butylamino)-9-fluorobenzo[h][1,3]thiazolo[5,4-f]isoquinolin-7(6H)-one;
9-Fluoro-2-[(3-methoxypropyl)amino]benzo[h][1,3]thiazolo[5,4-f]isoquinolin-7(6H)-one;
9-Fluoro-2-(4-methylpiperazin-1-yl)benzo[h][1,3]thiazolo[5,4-f]-isoquinolin-7(6H)-one;
2-(Dimethylamino)-9-fluorobenzo[h][1,3]thiazolo[5,4-f]isoquinolin-7(6H)-one;
2-(Benzylamino)-9-fluorobenzo[h][1,3]thiazolo[5,4-f]isoquinolin-7(6H)-one;
2-Anilino-9-fluorobenzo[h][1,3]thiazolo[5,4-f]isoquinolin-7(6H)-one;
9-Fluoro-2,6-dihydro-7H-benzo [h]pyrazolo[4,3-f]isoquinolin-7-one;
9-Fluoro-2-piperidin-1-ylbenzo[h][1,3]oxazolo[5,4-f]isoquinolin-7(6H)-one;
2-tert-Butyl-9-fluoro-3H-benzo[h]imidazo[4,5-f]quinoline;
2-tert-Butyl-9-fluoro-3H-benzo[h]imidazo[4,5-f]quinoline 7-oxide;
2-tert-Butyl-9-fluoro-7-methoxy-3H-benzo[f]imidazo[4,5-h]phthalazin-3-ol trifluoroacetic acid;
2-tert-Butyl-9-fluoro-7-methoxy-3H-1,3,5,6-tetraaza-cyclopenta[l]phenanthrene;
2-tert-Butyl-9-fluoro-3,6-dihydro-1,3,5,6-tetraaza-cyclopenta[l]phenanthren-7-one;
2-tert-Butyl-9-fluoro-3H-benzo[f]imidazo[4,5-h]phthalazine-3,7-diol;
2-tert-Butyl-9-fluoro-7-methoxy-3H-benzo[f]imidazo[4,5-h]quinazolin-5-amine;
5-Amino-2-tert-butyl-9-fluoro-3H-benzo[f]imidazo[4,5-h]quinazolin-7-ol;
2-tert-Butyl-9-fluoro-7-methoxy-3H-benzo[f]imidazo[4,5-h]quinazoline;
2-tert-Butyl-9-fluoro-3H-benzo[f]imidazo[4,5-h]quinazolin-7-ol;
2-tert-Butyl-9-fluoro-3H-benzo[h]imidazo[4,5-f]isoquinolin-3-ol;
2-tert-Butyl-9-fluoro-3H-benzo[h]imidazo[4,5-f]isoquinoline;
2-tert-Butyl-9-fluoro-3H-benzo[h]imidazo[4,5-f]isoquinoline 6-oxide;
9-Fluoro-2-[hydroxy(pyridin-3-yl)methyl]benzo[h][1,3]thiazolo[5,4-f]isoquinolin-7(6H)-one;
9-Fluoro-2-[4-(1H-imidazol-4-ylcarbonyl)piperazin-1-yl]benzo[h][1,3]thiazolo[5,4-f]isoquinolin-7(6H)-one;
2-Ethyl-9-fluoro-3,6-dihydro-7H-benzo[f]imidazo[4,5-h]quinazolin-7-one;
trans-2-(9-fluoro-7-oxo-6,7-dihydro-3H-benzo[f]imidazo[4,5-h]quinazolin-2-yl)cyclopropanecarboxamide;
1-(9-fluoro-7-oxo-6,7-dihydro-3H-benzo[f]imidazo[4,5-h]quinazolin-2-yl)cyclopropanecarboxylic acid;
2-[2-(dimethylamino)-1,1-dimethylethyl]-9-fluoro-3,6-dihydro-7H-benzo[f]imidazo[4,5-h]quinazolin-7-one;
4-ethyl-4-(9-fluoro-7-oxo-6,7-dihydro-3H-benzo[f]imidazo[4,5-h]quinazolin-2-yl)hexanenitrile;
4-ethyl-4-(9-fluoro-7-oxo-6,7-dihydro-3H-benzo[f]imidazo[4,5-h]quinazolin-2-yl)hexanamide;
2-(4-amino-1,1-dimethylbutyl)-9-fluoro-3,6-dihydro-7H-benzo[f]imidazo[4,5-h]quinazolin-7-one;
benzyl [1-(9-fluoro-7-oxo-6,7-dihydro-3H-benzo[f]imidazo[4,5-h]quinazolin-2-yl)-1-methylethyl]carbamate;
benzyl [2-(9-fluoro-7-oxo-6,7-dihydro-3H-benzo[f]imidazo[4,5-h]quinazolin-2-yl)-2-methylpropyl]carbamate;
[2-(9-fluoro-7-oxo-6,7-dihydro-3H-benzo[f]imidazo[4,5-h]quinazolin-2-yl)-2-methylpropoxy]acetonitrile;
2-(1-amino-1-methylethyl)-9-fluoro-3,6-dihydro-7H-benzo[f]imidazo[4,5-h]quinazolin-7-one;
4-(9-fluoro-7-oxo-6,7-dihydro-3H-benzo[f]imidazo[4,5-h]quinazolin-2-yl)butanenitrile;
N-[1-(9-fluoro-7-oxo-6,7-dihydro-3H-benzo[f]imidazo[4,5-h]quinazolin-2-yl)-1-methylethyl]acetamide;
benzyl 4-(9-fluoro-7-oxo-6,7-dihydro-3H-benzo[f]imidazo[4,5-h]quinazolin-2-yl)piperidine-1-carboxylate;
3-(9-fluoro-7-oxo-6,7-dihydro-3H-benzo[f]imidazo[4,5-h]quinazolin-2-yl)propanenitrile;
N-[2-(9-fluoro-7-oxo-6,7-dihydro-3H-benzo[f]imidazo[4,5-h]quinazolin-2-yl)-2-methylpropyl]urea;
4-(9-fluoro-7-oxo-6,7-dihydro-3H-benzo [f]imidazo[4,5-h]quinazolin-2-yl)-4-methylpentanenitrile;
2-(1-acetylpiperidin-4-yl)-9-fluoro-3,6-dihydro-7H-benzo[f]imidazo[4,5-h]quinazolin-7-one;
9-fluoro-2-(trans-4-hydroxycyclohexyl)-3,6-dihydro-7H-benzo[f]imidazo[4,5-h]quinazolin-7-one;
9-fluoro-2-(cis-4-hydroxycyclohexyl)-3,6-dihydro-7H-benzo[f]imidazo[4,5-h]quinazolin-7-one;
3-(9-fluoro-7-oxo-6,7-dihydro-3H-benzo [f]imidazo[4,5-h]quinazolin-2-yl)-3-methylbutanenitrile;
2-(Ethylthio)-9-fluoro-3,6-dihydro-7H-benzo[h]imidazo[4,5-f]isoquinolin-7-one;
2-(Ethylsulfinyl)-9-fluoro-3,6-dihydro-7H-benzo[h]imidazo[4,5-f]isoquinolin-7-one;
2-(Ethylsulfonyl)-9-fluoro-3,6-dihydro-7H-benzo[h]imidazo[4,5-f]isoquinolin-7-one;
2-[(9-Fluoro-7-oxo-6,7-dihydro-3H-benzo[h]imidazo[4,5-f]isoquinolin-2-yl)thio]-4-hydroxybutanamide;
9-Fluoro-2-[(2-hydroxycyclohexyl)thio]-3,6-dihydro-7H-benzo[h]imidazo[4,5-f]isoquinolin-7-one;
2-[(3,5-Dimethyl-1H-pyrazol-4-yl)thio]-9-fluoro-3,6-dihydro-7H-benzo[h]imidazo[4,5-f]isoquinolin-7-one;
9-Fluoro-2-{[(5-hydroxy-1H-pyrazol-3-yl)methyl]thio}-3,6-dihydro-7H-benzo[h]imidazo[4,5-f]isoquinolin-7-one;
9-Fluoro-2-[(2,3,5,6-tetrafluoropyridin-4-yl)thio]-3,6-dihydro-7H-benzo[h]imidazo[4,5-f]isoquinolin-7-one;
2-[(2,6-Diamino-3,5-difluoropyridin-4-yl)thio]-9-fluoro-3,6-dihydro-7H-benzo[h]imidazo[4,5-f]isoquinolin-7-one;
2-[(2-Amino-3,5,6-trifluoropyridin-4-yl)thio]-9-fluoro-3,6-dihydro-7H-benzo[h]imidazo[4,5-f]isoquinolin-7-one;
2-(benzylthio)-9-fluoro-3,6-dihydro-7H-benzo[h]imidazo[4,5-f]isoquinolin-7-one;
2-(benzylsulfinyl)-9-fluoro-3,6-dihydro-7H-benzo[h]imidazo[4,5-f]isoquinolin-7-one;
2-(benzylsulfonyl)-9-fluoro-3,6-dihydro-7H-benzo[h]imidazo[4,5-f]isoquinolin-7-one;
3-{[(9-fluoro-7-oxo-6,7-dihydro-3H-benzo[h]imidazo[4,5-f]isoquinolin-2-yl)thio]methyl}benzonitrile;
9-fluoro-2-[(2-methoxyethyl)thio]-3,6-dihydro-7H-benzo[h]imidazo[4,5-f]isoquinolin-7-one;
9-fluoro-2-[(2-oxotetrahydrofuran-3-yl)thio]-3,6-dihydro-7H-benzo[h]imidazo[4,5-f]isoquinolin-7-one;
2-{2-[(9-fluoro-7-oxo-6,7-dihydro-3H-benzo[h]imidazo[4,5-f]isoquinolin-2-yl)thio]ethyl}-1H-isoindole-1,3(2H)-dione;
9-fluoro-2-[(pyridin-3-ylmethyl)thio]-3,6-dihydro-7H-benzo[h]imidazo[4,5-f]isoquinolin-7-one;
2-[(1-ethylpropyl)thio]-9-fluoro-3,6-dihydro-7H-benzo[h]imidazo[4,5-f]isoquinolin-7-one;
2-[1-ethylpropyl)sulfinyl]-9-fluoro-3,6-dihydro-7H-benzo[h]imidazo[4,5-f]isoquinolin-7-one;
2-[(1-ethylpropyl)sulfonyl]-9-fluoro-3,6-dihydro-7H-benzo[h]imidazo[4,5-f]isoquinolin-7-one;
2-[(3,5-dimethoxybenzyl)thio]-9-fluoro-3,6-dihydro-7H-benzo[h]imidazo[4,5-f]isoquinolin-7-one;
ethyl [(9-fluoro-7-oxo-6,7-dihydro-3H-benzo[h]imidazo[4,5-f]isoquinolin-2-yl)thio]acetate;
9-fluoro-2-(isopropylthio)-3,6-dihydro-7H-benzo[h]imidazo[4,5-f]isoquinolin-7-one;
[(9-fluoro-7-oxo-6,7-dihydro-3H-benzo[h]imidazo[4,5-f]isoquinolin-2-yl)thio]acetic acid;
9-fluoro-2-[(1-phenylethyl)thio]-3,6-dihydro-7H-benzo[h]imidazo[4,5-f]isoquinolin-7-one;
[(9-fluoro-7-oxo-6,7-dihydro-3H-benzo[h]imidazo[4,5-f]isoquinolin-2-yl)thio]acetonitrile;
[(9-fluoro-7-oxo-6,7-dihydro-3H-benzo[h]imidazo[4,5-f]isoquinolin-2-yl)sulfinyl]acetonitrile;
2-[(9-fluoro-7-oxo-6,7-dihydro-3H-benzo[h]imidazo[4,5-f]isoquinolin-2-yl)thio]-N-phenylpropanamide;
9-fluoro-2-[(3-hydroxypropyl)thio]-3,6-dihydro-7H-benzo[h]imidazo[4,5-f]isoquinolin-7-one;
9-fluoro-2-[(3,3,3-trifluoro-2-oxopropyl)thio]-3,6-dihydro-7H-benzo[h]imidazo[4,5-f]isoquinolin-7-one;
ethyl 4-[(9-fluoro-7-oxo-6,7-dihydro-3H-benzo[h]imidazo[4,5-f]isoquinolin-2-yl)thio]butanoate;
2-[(2-aminoethyl)thio]-9-fluoro-3,6-dihydro-7H-benzo[h]imidazo[4,5-f]isoquinolin-7-one;
2-[(cyclohexylmethyl)thio]-9-fluoro-3,6-dihydro-7H-benzo[h]imidazo[4,5-f]isoquinolin-7-one;
2-[(9-fluoro-7-oxo-6,7-dihydro-3H-benzo[h]imidazo[4,5-f]isoquinolin-2-yl)thio]acetamide;
2-(cyclohexylthio)-9-fluoro-3,6-dihydro-7H-benzo[h]imidazo[4,5-f]isoquinolin-7-one;
2-[(2,2-dimethoxyethyl)thio]-9-fluoro-3,6-dihydro-7H-benzo[h]imidazo[4,5-f]isoquinolin-7-one;
2-[(3,3-dimethyl-2-oxobutyl)thio]-9-fluoro-3,6-dihydro-7H-benzo[h]imidazo[4,5-f]isoquinolin-7-one;
ethyl 2-[(9-fluoro-7-oxo-6,7-dihydro-3H-benzo[h]imidazo[4,5-f]isoquinolin-2-yl)thio]propanoate;
4-[(9-fluoro-7-oxo-6,7-dihydro-3H-benzo[h]imidazo[4,5-f]isoquinolin-2-yl)thio]butanenitrile;
ethyl 2-[(9-fluoro-7-oxo-6,7-dihydro-3H-benzo[h]imidazo[4,5-f]isoquinolin-2-yl)thio]butanoate;
2-[(9-fluoro-7-oxo-6,7-dihydro-3H-benzo[h]imidazo[4,5-f]isoquinolin-2-yl)thio]-4-hydroxy-N,N-dimethylbutanamide;
methyl 3-{[(9-fluoro-7-oxo-6,7-dihydro-3H-benzo[h]imidazo[4,5-f]isoquinolin-2-yl)thio]methyl}benzoate;
9-fluoro-2-[(tetrahydro-2H-pyran-2-ylmethyl)thio]-3,6-dihydro-7H-benzo[h]imidazo[4,5-f]isoquinolin-7-one;
4-[(9-fluoro-7-oxo-6,7-dihydro-3H-benzo[h]imidazo[4,5-f]isoquinolin-2-yl)thio]butanoic acid
4-[(9-fluoro-7-oxo-6,7-dihydro-3H-benzo[h]imidazo[4,5-f]isoquinolin-2-yl)thio]butanamide;
9-fluoro-7-oxo-6,7-dihydro-3H-benzo[h]imidazo[4,5-f]isoquinolin-2-yl thiocyanate;
2-[(9-fluoro-7-oxo-6,7-dihydro-3H-benzo[h]imidazo[4,5-f]isoquinolin-2-yl)thio]propanenitrile;
9-fluoro-2-[(2-hydroxy-2-methylpropyl)thio]-3,6-dihydro-7H-benzo[h]imidazo[4,5-f]isoquinolin-7-one;
9-fluoro-2-[(3-hydroxy-2,2-dimethylpropyl)thio]-3,6-dihydro-7H-benzo[h]imidazo[4,5-f]isoquinolin-7-one;
9-fluoro-2-[(2-oxocyclopentyl)thio]-3,6-dihydro-7H-benzo[h]imidazo[4,5-f]isoquinolin-7-one;
2-[(1,3-dioxolan-2-ylmethyl)thio]-9-fluoro-3,6-dihydro-7H-benzo[h]imidazo[4,5-f]isoquinolin-7-one;
N-ethyl-2-[(9-fluoro-7-oxo-6,7-dihydro-3H-benzo[h]imidazo[4,5-f]isoquinolin-2-yl)thio]-4-hydroxybutanamide;
9-fluoro-2-[(2-hydroxyethyl)thio]-3,6-dihydro-7H-benzo[h]imidazo[4,5-f]isoquinolin-7-one;
9-fluoro-2-(piperidin-4-ylthio)-3,6-dihydro-7H-benzo[h]imidazo[4,5-f]isoquinolin-7-one;
3-[(9-fluoro-7-oxo-6,7-dihydro-3H-benzo[h]imidazo[4,5-f]isoquinolin-2-yl)thio]propanenitrile;
9-fluoro-2-[(3-oxocyclohex-1-en-1-yl)thio]-3,6-dihydro-7H-phenanthro[9,10-d]imidazol-7-one;
9-fluoro-2-(pyridin-4-ylthio)-3,6-dihydro-7H-benzo[h]imidazo[4,5-f]isoquinolin-7-one;
9-fluoro-2-(1H-pyrazol-4-ylthio)-3,6-dihydro-7H-benzo[h]imidazo[4,5-f]isoquinolin-7-one;
9-fluoro-2-[(2-hydroxy-2-methylpropyl)sulfinyl]-3,6-dihydro-7H-benzo[h]imidazo[4,5-f]isoquinolin-7-one;
3-[(9-fluoro-7-oxo-6,7-dihydro-3H-benzo[h]imidazo[4,5-f]isoquinolin-2-yl)sulfinyl]propanenitrile;
9-fluoro-2-[(2-hydroxy-3,3-dimethylbutyl)thio]-3,6-dihydro-7H-benzo[h]imidazo[4,5-f]isoquinolin-7-one;
9-fluoro-2-[(2-oxopropyl)thio]-3,6-dihydro-7H-benzo[h]imidazo[4,5-f]isoquinolin-7-one,
9-fluoro-2-(pyridin-4-ylsulfinyl)-3,6-dihydro-7H-benzo[h]imidazo[4,5-f]isoquinolin-7-one;
4-[(9-fluoro-7-oxo-6,7-dihydro-3H-benzo[h]imidazo[4,5-f]isoquinolin-2-yl)sulfinyl]butanenitrile;
9-fluoro-2-(pyrimidin-5-ylthio)-3,6-dihydro-7H-benzo[h]imidazo[4,5-f]isoquinolin-7-one;
ethyl 4-[(9-fluoro-7-oxo-6,7-dihydro-3H-benzo[h]imidazo[4,5-f]isoquinolin-2-yl)thio]-3-oxobutanoate;
3-[(9-fluoro-7-oxo-6,7-dihydro-3H-benzo[h]imidazo[4,5-f]isoquinolin-2-yl)thio]-2-methylpropanenitrile;
3-[(9-fluoro-7-oxo-6,7-dihydro-3H-benzo[h]imidazo[4,5-f]isoquinolin-2-yl)sulfinyl]-2-methylpropanenitrile;
9-fluoro-2-[(2-hydroxypropyl)thio]-3,3a,6,11b-tetrahydro-7H-benzo[h]imidazo[4,5-f]isoquinolin-7-one;
9-fluoro-2-(isoxazol-4-ylthio)-3,6-dihydro-7H-benzo[h]imidazo[4,5-f]isoquinolin-7-one
3-[(9-fluoro-7-oxo-6,7-dihydro-3H-benzo[h]imidazo[4,5-f]isoquinolin-2-yl)thio]propanamide;
2-[(3,5-dichloropyridin-4-yl)thio]-9-fluoro-3,6-dihydro-7H-benzo[h]imidazo[4,5-f]isoquinolin-7-one;
2-[(4,6-dimethoxy-1,3,5-triazin-2-yl)thio]-9-fluoro-3,6-dihydro-7H-benzo[h]imidazo[4,5-f]isoquinolin-7-one;
9-fluoro-2-(pyrimidin-2-ylthio)-3,6-dihydro-7H-benzo[h]imidazo[4,5-f]isoquinolin-7-one;
2-[(2,2-dimethylpropyl)thio]-9-fluoro-3,6-dihydro-7H-benzo[h]imidazo[4,5-f]isoquinolin-7-one;
9-fluoro-2-(isobutylthio)-3,6-dihydro-7H-benzo[h]imidazo[4,5-f]isoquinolin-7-one;
2-[(2,2-dimethylpropyl)sulfinyl]-9-fluoro-3,6-dihydro-7H-benzo[h]imidazo[4,5-f]isoquinolin-7-one;
2-(cyclohexylsulfinyl)-9-fluoro-3,6-dihydro-7H-benzo[h]imidazo[4,5-f]isoquinolin-7-one;
9-fluoro-2-(pyrazin-2-ylthio)-3,6-dihydro-7H-benzo[h]imidazo[4,5-f]isoquinolin-7-one;
9-fluoro-2-[(3-fluoropyridin-4-yl)thio]-3,6-dihydro-7H-benzo[h]imidazo[4,5-f]isoquinolin-7-one;
2-[(6-chloropyrimidin-4-yl)thio]-9-fluoro-3,6-dihydro-7H-benzo[h]imidazo[4,5-f]isoquinolin-7-one;
2-[(2,6-dimethoxypyrimidin-4-yl)thio]-9-fluoro-3,6-dihydro-7H-benzo[h]imidazo[4,5-f]isoquinolin-7-one;
2-[(2,6-dichloropyridin-4-yl)thio]-9-fluoro-3,6-dihydro-7H-benzo[h]imidazo[4,5-f]isoquinolin-7-one;
2-[(6-chloropyridazin-3-yl)thio]-9-fluoro-3,6-dihydro-7H-benzo[h]imidazo[4,5-f]isoquinolin-7-one;
2-[(3-amino-6-bromopyrazin-2-yl)thio]-9-fluoro-3,6-dihydro-7H-benzo[h]imidazo[4,5-f]isoquinolin-7-one;
9-fluoro-2-(quinolin-4-ylthio)-3,6-dihydro-7H-benzo[h]imidazo[4,5-f]isoquinolin-7-one;
2-[(6-chloropyrazin-2-yl)thio]-9-fluoro-3,6-dihydro-7H-benzo[h]imidazo[4,5-f]isoquinolin-7-one;
2-[(2,6-dichloropyrimidin-4-yl)thio]-9-fluoro-3,6-dihydro-7H-benzo[h]imidazo[4,5-f]isoquinolin-7-one;
9-fluoro-2-[(1-oxidopyridin-4-yl)thio]-3,6-dihydro-7H-benzo[h]imidazo[4,5-f]isoquinolin-7-one;
2-[(2,6-diaminopyrimidin-4-yl)thio]-9-fluoro-3,6-dihydro-7H-benzo[h]imidazo[4,5-f]isoquinolin-7-one;
9-fluoro-2-[(1-methyl-1H-pyrazol-4-yl)thio]-3,6-dihydro-7H-benzo[h]imidazo[4,5-f]isoquinolin-7-one;
3-[(9-fluoro-7-oxo-6,7-dihydro-3H-benzo[h]imidazo[4,5-f]isoquinolin-2-yl)thio]pyrazine-2-carbonitrile;
9-fluoro-2-[(2-methylpyrimidin-5-yl)thio]-3,6-dihydro-7H-benzo[h]imidazo[4,5-f]isoquinolin-7-one;
2-[(5-chloro-3-hydroxypyridazin-4-yl)thio]-9-fluoro-3,6-dihydro-7H-benzo[h]imidazo[4,5-f]isoquinolin-7-one;
2-[(3,5-dichloropyridazin-4-yl)thio]-9-fluoro-3,6-dihydro-7H-benzo[h]imidazo[4,5-f]isoquinolin-7-one;
9-fluoro-2-[(1-oxidopyridin-2-yl)thiol]-3,6-dihydro-7H-benzo[h]imidazo[4,5-f]isoquinolin-7-one;
9-fluoro-2-[(1H-tetrazol-5-ylmethyl)thio]-3,6-dihydro-7H-benzo[h]imidazo[4,5-f]isoquinolin-7-one trifluoroacetate;
9-fluoro-2-[(6-hydroxypyridin-3-yl)thio]-3,6-dihydro-7H-benzo[h]imidazo[4,5-f]isoquinolin-7-one;
2-[(2-amino-6-chloropyridin-4-yl)thio]-9-fluoro-3,6-dihydro-7H-benzo[h]imidazo[4,5-f]isoquinolin-7-one trifluoroacetate;
2-[(6-aminopyrimidin-4-yl)thio]-9-fluoro-3,6-dihydro-7H-benzo[h]imidazo[4,5-f]isoquinolin-7-one;
2-[(6-aminopyrimidin-4-yl)thio]-9-fluoro-3,6-dihydro-7H-benzo[h]imidazo[4,5-f]isoquinolin-7-one;
9-fluoro-2-(pyridin-3-ylthio)-3,6-dihydro-7H-benzo[h]imidazo[4,5-f]isoquinolin-7-one trifluoroacetate;
9-fluoro-2-(phenylthio)-3,6-dihydro-7H-benzo[h]imidazo[4,5-f]isoquinolin-7-one trifluoroacetate;
2-[(2-chloropyrimidin-4-yl)thio]-9-fluoro-3,6-dihydro-7H-benzo[h]imidazo[4,5-f]isoquinolin-7-one;
9-fluoro-2-(1H-tetrazol-5-ylthio)-3,6-dihydro-7H-benzo[h]imidazo[4,5-f]isoquinolin-7-one;
2-[(5-bromopyridin-3-yl)thio]-9-fluoro-3,6-dihydro-7H-benzo[h]imidazo[4,5-f]isoquinolin-7-one trifluoroacetate;
2-[(3-aminophenyl)thio]-9-fluoro-3,6-dihydro-7H-benzo[h]imidazo[4,5-f]isoquinolin-7-one trifluoroacetate;
4-[(9-fluoro-7-oxo-6,7-dihydro-3H-benzo[h]imidazo[4,5-f]isoquinolin-2-yl)thio]pyridine-2-carbonitrile;
9-fluoro-2-[(5-methoxypyridin-3-yl)thio]-3,6-dihydro-7H-benzo[h]imidazo[4,5-f]isoquinolin-7-one trifluoroacetate;
2-[(2-aminopyrimidin-4-yl)thio]-9-fluoro-3,6-dihydro-7H-benzo[h]imidazo[4,5-f]isoquinolin-7-one;
5-[(9-fluoro-7-oxo-6,7-dihydro-3H-benzo[h]imidazo[4,5-f]isoquinolin-2-yl)thio]-N-(2-hydroxyethyl)nicotinamide trifluoroacetate (salt);
methyl 4-chloro-5-[(9-fluoro-7-oxo-6,7-dihydro-3H-benzo[h]imidazo[4,5-f]isoquinolin-2-yl)thio]pyridine-2-carboxylate;
9-fluoro-2-[(6-fluoropyridin-3-yl)thio]-3,6-dihydro-7H-benzo[h]imidazo[4,5-f]isoquinolin-7-one trifluoroacetate;
2-[(6-aminopyridin-3-yl)thio]-9-fluoro-3,6-dihydro-7H-benzo[h]imidazo[4,5-f]isoquinolin-7-one trifluoroacetate;
9-fluoro-2-[(6-methoxypyridin-3-yl)thio]-3,6-dihydro-7H-benzo[h]imidazo[4,5-f]isoquinolin-7-one trifluoroacetate;
9-fluoro-2-[(2-fluoropyridin-4-yl)thio]-3,6-dihydro-7H-benzo[h]imidazo[4,5-f]isoquinolin-7-one;
9-fluoro-2-(quinolin-3-ylthio)-3,6-dihydro-7H-benzo[h]imidazo[4,5-f]isoquinolin-7-one trifluoroacetate;
5-[(9-fluoro-7-oxo-6,7-dihydro-3H-benzo[h]imidazo[4,5-f]isoquinolin-2-yl)thio]nicotinamide trifluoroacetate;
2-[(2-aminopyridin-4-yl)thio]-9-fluoro-3,6-dihydro-7H-benzo[h]imidazo[4,5-f]isoquinolin-7-one;
9-fluoro-2-[(2-methoxypyridin-4-yl)thio]-3,6-dihydro-7H-benzo[h]imidazo[4,5-f]isoquinolin-7-one;
9-fluoro-2-{[5-(morpholin-4-ylcarbonyl)pyridin-3-yl]thio}-3,6-dihydro-7H-benzo[h]imidazo[4,5-f]isoquinolin-7-one trifluoroacetate;
5-[(9-fluoro-7-oxo-6,7-dihydro-3H-benzo[h]imidazo[4,5-f]isoquinolin-2-yl)thio]-N-[2-(tetrahydro-2H-pyran-4-yl)ethyl]nicotinamide;
9-fluoro-2-({5-[(4-methylpiperazin-1-yl)carbonyl]pyridin-3-yl}thio)-3,6-dihydro-7H-benzo[h]imidazo[4,5-f]isoquinolin-7-one;
9-fluoro-2-({2-[(2-morpholin-4-ylethyl)amino]pyridin-4-yl}thio)-3,6-dihydro-7H-benzo[hjimidazo[4,5-f]isoquinolin-7-one;
2-{[2-(dimethylamino)pyridin-4-yl]thio}-9-fluoro-3,6-dihydro-7H-benzo[h]imidazo[4,5-f]isoquinolin-7-one;
9-fluoro-2-({2-[(2-hydroxyethyl)amino]pyridin-4-yl}thio)-3,6-dihydro-7H-benzo[h]imidazo[4,5-f]isoquinolin-7-one;
methyl 4-[(9-fluoro-7-oxo-6,7-dihydro-3H-benzo[h]imidazo[4,5-f]isoquinolin-2-yl)thio]pyridine-2-carboxylate;
9-fluoro-2-(isoquinolin-4-ylthio)-3,6-dihydro-7H-benzo[h]imidazo[4,5-f]isoquinolin-7-one trifluoroacetate;
9-fluoro-2-({6-[(2-hydroxyethyl)amino]pyridin-3-yl}thio)-3,6-dihydro-7H-benzo[h]imidazo[4,5-f]isoquinolin-7-one trifluoroacetate;
9-fluoro-2-({6-[(2-morpholin-4-ylethyl)amino]pyridin-3-yl}thio)-3,6-dihydro-7H-benzo[h]imidazo[4,5-f]isoquinolin-7-one trifluoroacetate;
4-[(9-fluoro-7-oxo-6,7-dihydro-3H-benzo[h]imidazo[4,5-f]isoquinolin-2-yl)thio]benzamide;
2-[(5-aminopyridin-3-yl)thio]-9-fluoro-3,6-dihydro-7H-benzo[h]imidazo[4,5-f]isoquinolin-7-one;
9-fluoro-2-{[4-(1H-imidazol-1-yl)phenyl]thio}-3,6-dihydro-7H-benzo[h]imidazo[4,5-f]isoquinolin-7-one trifluoroacetate;
9-fluoro-2-[(3-hydroxyphenyl)thio]-3,6-dihydro-7H-benzo[h]imidazo[4,5-f]isoquinolin-7-one trifluoroacetate (salt);
4-[(9-fluoro-7-oxo-6,7-dihydro-3H-benzo[h]imidazo[4,5-f]isoquinolin-2-yl)thio]-N-(2-hydroxyethyl)benzamide trifluoroacetate (salt);
3-[(9-fluoro-7-oxo-6,7-dihydro-3H-benzo[h]imidazo[4,5-f]isoquinolin-2-yl)thio]-N-(2-hydroxyethyl)benzamide trifluoroacetate (salt);
3-[(9-fluoro-7-oxo-6,7-dihydro-3H-benzo[h]imidazo[4,5-f]isoquinolin-2-yl)thio]benzonitrile trifluoroacetate;
9-fluoro-2-[(5-morpholin-4-ylpyridin-3-yl)thio]-3,6-dihydro-7H-benzo[h]imidazo[4,5-f]isoquinolin-7-one trifluoroacetate;
{3-[(9-fluoro-7-oxo-6,7-dihydro-3H-benzo[h]imidazo[4,5-f]isoquinolin-2-yl)thio]phenoxy}acetonitrile trifluoroacetate;
N-{3-[(9-fluoro-7-oxo-6,7-dihydro-3H-benzo[h]imidazo[4,5-f]isoquinolin-2-yl)thio]phenyl}methanesulfonamide trifluoroacetate;
2-cyano-N-{3-[(9-fluoro-7-oxo-6,7-dihydro-3H-benzo[h]imidazo[4,5-f]isoquinolin-2-yl)thio]phenyl}acetamide trifluoroacetate;
N′-{3-[(9-fluoro-7-oxo-6,7-dihydro-3H-benzo[h]imidazo[4,5-f]isoquinolin-2-yl)thio]phenyl}-N,N-dimethylsulfamide;
N-{3-[(9-fluoro-7-oxo-6,7-dihydro-3H-benzo[h]imidazo[4,5-f]isoquinolin-2-yl)thio]phenyl}ethanesulfonamide;
2-(dimethylamino)-N-{3-[(9-fluoro-7-oxo-6,7-dihydro-3H-benzo[h]imidazo[4,5-f]isoquinolin-2-yl)thio]phenyl}acetamide trifluoroacetate;
N-ethyl-N′-{3-[(9-fluoro-7-oxo-6,7-dihydro-3H-benzo[h]imidazo[4,5-f]isoquinolin-2-yl)thio]phenyl}urea trifluoroacetate;
2-tert-butyl-10-fluorobenzo[c]imidazo[1,2-a]-1,6-naphthyridin-8(7H)-one;
10-Fluoro-2-(4-hydroxycyclohexyl)benzo[c]imidazo[1,2-a]-1,6-naphthyridin-8(7H)-one;
trans-4-(10-Fluoro-8-oxo-7,8-dihydrobenzo[c]imidazo[1,2-a]-1,6-naphthyridin-2-yl)cyclohexyl (dimethylamino)acetate;
10-fluoro-2-(1-methylcyclopropyl)benzo[c]imidazo[1,2-a]-1,6-naphthyridin-8(7H)-one;
2-cyclopropyl-10-fluorobenzo[c]imidazo[1,2-a]-1,6-naphthyridin-8(7H)-one;
2-cyclobutyl-10-fluorobenzo[c]imidazo[1,2-a]-1,6-naphthyridin-8(7H)-one;
2-ethyl-10-fluorobenzo[c]imidazo[1,2-a]-1,6-naphthyridin-8(7H)-one;
ethyl 2-ethyl-2-(10-fluoro-8-oxo-7,8-dihydrobenzo[c]imidazo[1,2-a]-1,6-naphthyridin-2-yl)butanoate;
2-[1-ethyl-1-(hydroxymethyl)propyl]-10-fluorobenzo[c]imidazo[1,2-a]-1,6-naphthyridin-8(7H)-one;
2-(1-ethylpropyl)-10-fluorobenzo[c]imidazo[1,2-a]-1,6-naphthyridin-8(7H)-one;
10-fluoro-2-(4-oxocyclohexyl)benzo[c]imidazo[1,2-a]-1,6-naphthyridin-8(7H)-one;
10-fluoro-2-methylbenzo[c]imidazo[1,2-a]-1,6-naphthyridin-8(7H)-one;
10-fluoro-2-(cis-4-methoxycyclohexyl)benzo[c]imidazo[1,2-a]-1,6-naphthyridin-8(7H)-one;
10-fluoro-2-(trans-4-methoxycyclohexyl)benzo[c]imidazo[1,2-a]-1,6-naphthyridin-8(7H)-one;
10-fluoro-2-[4-(hydroxyimino)cyclohexyl]benzo[c]imidazo[1,2-a]-1,6-naphthyridin-8(7H)-one;
2-(1-acetylpiperidin-4-yl)-10-fluorobenzo[c]imidazo[1,2-a]-1,6-naphthyridin-8(7H)-one;
2-amino-9-fluorobenzo[h][1,3]thiazolo[5,4-f]isoquinolin-7(6H)-one;
9-fluoro-2-(4-hydroxypiperidin-1-yl)benzo[h][1,3]thiazolo[5,4-f]isoquinolin-7(6H)-one;
9-fluoro-2-{[(1S)-1-(hydroxymethyl)-3-methylbutyl]amino}benzo[h][1,3]thiazolo[5,4-f]isoquinolin-7(6H)-one;
9-fluoro-2-{[(1R)-2-hydroxy-1-phenylethyl]amino}benzo[h][1,3]thiazolo[5,4-f]isoquinolin-7(6H)-one;
(2S)-2-[(9-fluoro-7-oxo-6,7-dihydrobenzo[h][1,3]thiazolo[5,4-f]isoquinolin-2-yl)amino]-4-methoxy-N-methylbutanamide;
9-fluoro-2-(4-oxopiperidin-1-yl)benzo[h][1,3]thiazolo[5,4-f]isoquinolin-7(6H)-one;
9-fluoro-2-{[(1R)-1-(hydroxymethyl)-3-methylbutyl]amino}benzo[h][1,3]thiazolo[5,4-f]isoquinolin-7(6H)-one;
9-fluoro-2-(3-hydroxypiperidin-1-yl)benzo[h][1,3]thiazolo[5,4-f]isoquinolin-7(6H)-one;
9-fluoro-2-{[(1S)-2-hydroxy-1-phenylethyl]amino}benzo[h][1,3]thiazolo[5,4-f]isoquinolin-7(6H)-one;
9-fluoro-2-(3-hydroxypyrrolidin-1-yl)benzo[h][1,3]thiazolo[5,4-f]isoquinolin-7(6H)-one;
9-fluorobenzo[h][1,3]thiazolo[5,4-f]isoquinolin-7(6H)-one;
9-fluoro-2-(1-hydroxy-4-oxocyclohexyl)benzo[h][1,3]thiazolo[5,4-f]isoquinolin-7(6H)-one;
2-[4-(ethoxyimino)piperidin-1-yl]-9-fluorobenzo[h][1,3]thiazolo[5,4-f]isoquinolin-7(6H)-one;
9-fluoro-2-[4-(hydroxyimino)piperidin-1-yl]benzo[h][1,3]thiazolo[5,4-f]isoquinolin-7(6H)-one;
4-(9-fluoro-7-oxo-6,7-dihydrobenzo[h][1,3]thiazolo[5,4-f]isoquinolin-2-yl)butanenitrile;
2-(1,4-dihydroxycyclohexyl)-9-fluorobenzo[h][1,3]thiazolo[5,4-f]isoquinolin-7(6H)-one;
2-(1-acetyl-4-hydroxypiperidin-4-yl)-9-fluorobenzo[h][1,3]thiazolo[5,4-f]isoquinolin-7(6H)-one;
9-fluoro-2-(4-hydroxy-1-isobutyrylpiperidin-4-yl)benzo[h][1,3]thiazolo[5,4-f]isoquinolin-7(6H)-one;
9-fluoro-2-isonicotinoylbenzo[h][1,3]thiazolo[5,4-f]isoquinolin-7(6H)-one;
9-fluoro-2-(1-oxidoisonicotinoyl)benzo[h][1,3]thiazolo[5,4-f]isoquinolin-7(6H)-one;
9-fluoro-2-phenylbenzo[h][1,3]thiazolo[5,4-f]isoquinolin-7(6H)-one;
2-ethyl-9-fluorobenzo[h][1,3]thiazolo[5,4-f]isoquinolin-7(6H)-one;
9-fluoro-2-morpholin-4-ylbenzo[h][1,3]thiazolo[5,4-f]isoquinolin-7(6H)-one;
9-fluoro-2-[(pyridin-3-ylmethyl)amino]benzo[h][1,3]thiazolo[5,4-f]isoquinolin-7(6H)-one;
9-fluoro-2-[(3-morpholin-4-ylpropyl)amino]benzo[h][1,3]thiazolo[5,4-f]isoquinolin-7(6H)-one;
9-fluoro-2-[(3-methoxybenzyl)amino]benzo[h][1,3]thiazolo[5,4-f]isoquinolin-7(6H)-one;
9-fluoro-2-{[(1R)-1-(3-methoxyphenyl)ethyl]amino}benzo[h][1,3]thiazolo[5,4-f]isoquinolin-7(6H)-one;
9-fluoro-2-{[(1S)-1-(3-methoxyphenyl)ethyl]amino}benzo[h][1,3]thiazolo[5,4-f]isoquinolin-7(6H)-one;
9-fluoro-2-[(3-hydroxypropyl)amino]benzo[h][1,3]thiazolo[5,4-f]isoquinolin-7(6H)-one;
9-fluoro-2-[(2-hydroxyethyl)(methyl)amino]benzo[h][1,3]thiazolo[5,4-f]isoquinolin-7(6H)-one;
9-fluoro-2-{[2-(4-hydroxyphenyl)ethyl]amino}benzo [h][1,3]thiazolo[5,4-f]isoquinolin-7(6H)-one;
9-fluoro-2-[(2-methoxy-1-methylethyl)amino]benzo[h][1,3]thiazolo[5,4-f]isoquinolin-7(6H)-one;
N,N-diethyl-1-(9-fluoro-7-oxo-6,7-dihydrobenzo[h][1,3]thiazolo[5,4-f]isoquinolin-2-yl)piperidine-3-carboxamide;
9-fluoro-2-[(3-phenylpropyl)amino]benzo[h][1,3]thiazolo[5,4-f]isoquinolin-7(6H)-one;
9-fluoro-2-{[(1R)-1-(4-methoxyphenyl)ethyl]amino}benzo[h][1,3]thiazolo[5,4-f]isoquinolin-7(6H)-one;
9-fluoro-2-{[(1S)-1-(4-methoxyphenyl)ethyl]amino}benzo[h][1,3]thiazolo[5,4-f]isoquinolin-7(6H)-one;
9-fluoro-2-(pyridin-3-ylamino)benzo [h][1,3]thiazolo[5,4-f]isoquinolin-7(6H)-one;
9-fluoro-2-(4-pyridin-2-ylpiperazin-1-yl)benzo[h][1,3]thiazolo[5,4-f]isoquinolin-7(6H)-one;
9-fluoro-2-{[3-(1H-imidazol-1-yl)propyl]amino}benzo[h][1,3]thiazolo[5,4-f]isoquinolin-7(6H)-one;
9-fluoro-2-[(2-morpholin-4-ylethyl)amino]benzo[h][1,3]thiazolo[5,4-f]isoquinolin-7(6H)-one;
9-fluoro-2-[(2-methoxyethyl)amino]benzo[h][1,3]thiazolo[5,4-f]isoquinolin-7(6H)-one;
9-fluoro-2-[(tetrahydrofuran-2-ylmethyl)amino]benzo[h][1,3]thiazolo[5,4-f]isoquinolin-7(6H)-one;
9-fluoro-2-[(2-pyridin-2-ylethyl)amino]benzo[h][1,3]thiazolo[5,4-f]isoquinolin-7(6H)-one;
9-fluoro-2-{[2-(1H-imidazol-4-yl)ethyl]amino}benzo[h][1,3]thiazolo[5,4-f]isoquinolin-7(6H)-one;
9-fluoro-2-[4-(2-hydroxyethyl)piperazin-1-yl]benzo[h][1,3]thiazolo[5,4-f]isoquinolin-7(6H)-one;
2-{[3-(dimethylamino)propyl]amino}-9-fluorobenzo[h][1,3]thiazolo[5,4-f]isoquinolin-7(6H)-one;
9-fluoro-2-(4-hydroxy-4-pyridin-2-ylpiperidin-1-yl)benzo[h][1,3]thiazolo[5,4-f]isoquinolin-7(6H)-one;
2-[bis(2-methoxyethyl)amino]-9-fluorobenzo[h][1,3]thiazolo[5,4-f]isoquinolin-7(6H)-one;
(9-fluoro-7-oxo-6,7-dihydrobenzo[h][1,3]thiazolo[5,4-f]isoquinolin-2-yl)acetonitrile;
2-{benzyl[2-(dimethylamino)ethyl]amino}-9-fluorobenzo[h][1,3]thiazolo[5,4-f]isoquinolin-7(6H)-one;
9-fluoro-2-{[3-(trifluoromethyl)phenyl]amino}benzo[h][1,3]thiazolo[5,4-f]isoquinolin-7(6H)-one;
9-fluoro-2-[(2-methoxyphenyl)amino]benzo[h][1,3]thiazolo[5,4-f]isoquinolin-7(6H)-one;
9-fluoro-2-(propylamino)benzo[h][1,3]thiazolo[5,4-f]isoquinolin-7(6H)-one;
9-fluoro-2-(isopropylamino)benzo[h][1,3]thiazolo[5,4-f]isoquinolin-7(6H)-one;
3-[(9-fluoro-7-oxo-6,7-dihydrobenzo[h][1,3]thiazolo[5,4-f]isoquinolin-2-yl)amino]benzonitrile;
9-fluoro-2-[(3-hydroxyphenyl)amino]benzo[h][1,3]thiazolo[5,4-f]isoquinolin-7(6H)-one;
9-fluoro-2-[(trans-4-hydroxy-4-pyridin-2-ylcyclohexyl)amino]benzo[h][1,3]thiazolo[5,4-f]isoquinolin-7(6H)-one;
9-fluoro-2-[cis-4-hydroxy-3-(hydroxymethyl)piperidin-1-yl]benzo[h][1,3]thiazolo[5,4-f]isoquinolin-7(6H)-one;
9-fluoro-2-[trans-4-hydroxy-3-(hydroxymethyl)piperidin-1-yl]benzo[h][1,3]thiazolo[5,4-f]isoquinolin-7(6H)-one;
9-fluoro-2-[(2-hydroxyethyl)amino]benzo[h][1,3]thiazolo[5,4-f]isoquinolin-7(6H)-one;
9-fluoro-2-[(4-hydroxybenzyl)amino]benzo[h][1,3]thiazolo[5,4-f]isoquinolin-7(6H)-one;
9-fluoro-2-[4-(hydroxymethyl)piperidin-1-yl]benzo[h][1,3]thiazolo[5,4-f]isoquinolin-7(6H)-one;
9-fluoro-2-[3-(hydroxymethyl)piperidin-1-yl]benzo[h][1,3]thiazolo[5,4-f]isoquinolin-7(6H)-one;
9-fluoro-2-[(2-hydroxy-1-methylethyl)amino]benzo [h][1,3]thiazolo[5,4-f]isoquinolin-7(6H)-one;
9-fluoro-2-[(2S)-2-(hydroxymethyl)pyrrolidin-1-yl]benzo[h][1,3]thiazolo[5,4-f]isoquinolin-7(6H)-one;
9-fluoro-2-[(2R)-2-(hydroxymethyl)pyrrolidin-1-yl]benzo[h][1,3]thiazolo[5,4-f]isoquinolin-7(6H)-one;
2-(4-acetylpiperazin-1-yl)-9-fluorobenzo[h][1,3]thiazolo[5,4-f]isoquinolin-7(6H)-one;
9-fluoro-2-piperazin-1-ylbenzo[h][1,3]thiazolo[5,4-f]isoquinolin-7(6H)-one;
2-{4-[(dimethylamino)acetyl]piperazin-1-yl}-9-fluorobenzo[h][1,3]thiazolo[5,4-f]isoquinolin-7(6H)-one;
9-fluoro-2-[4-(2-hydroxyethyl)piperidin-1-yl]benzo[h][1,3]thiazolo[5,4-f]isoquinolin-7(6H)-one;
9-fluoro-2-{4-[(2R)-2-hydroxypropyl]piperazin-1-yl}benzo[h][1,3]thiazolo[5,4-f]isoquinolin-7(6H)-one;
2-[(2,3-dihydroxypropyl)amino]-9-fluorobenzo[h][1,3]thiazolo[5,4-f]isoquinolin-7(6H)-one;
4-(9-fluoro-7-oxo-6,7-dihydrobenzo[h][1,3]thiazolo[5,4-f]isoquinolin-2-yl)piperazine-1-carboxamide;
methyl 4-(9-fluoro-7-oxo-6,7-dihydrobenzo[h][1,3]thiazolo[5,4-f]isoquinolin-2-yl)piperazine-1-carboxylate;
ethyl 4-(9-fluoro-7-oxo-6,7-dihydrobenzo[h][1,3]thiazolo[5,4-f]isoquinolin-2-yl)piperazine-1-carboxylate;
4-(9-fluoro-7-oxo-6,7-dihydrobenzo[h][1,3]thiazolo[5,4-f]isoquinolin-2-yl)piperazine-1-carbaldehyde;
9-fluoro-2-[4-(1H-tetrazol-5-ylacetyl)piperazin-1-yl]benzo[h][1,3]thiazolo[5,4-f]isoquinolin-7(6H)-one;
9-fluoro-2-(pyridin-2-ylamino)benzo[h][1,3]thiazolo[5,4-f]isoquinolin-7(6H)-one;
9-fluoro-2-(pyridin-4-ylamino)benzo[h][1,3]thiazolo[5,4-f]isoquinolin-7(6H)-one;
9-fluoro-2-[(3R,4R)-3-methoxy-4-(methylamino)pyrrolidin-1-yl]benzo[h][1,3]thiazolo[5,4-f]isoquinolin-7(6H)-one;
9-fluoro-2-[(3S,4R)-3-methoxy-4-(methylamino)pyrrolidin-1-yl]benzo[h][1,3]thiazolo[5,4-f]isoquinolin-7(6H)-one;
benzyl (3R,4R)-3-[(9-fluoro-7-oxo-6,7-dihydrobenzo[h][1,3]thiazolo[5,4-f]isoquinolin-2-yl)(methyl)amino]-4-methoxypyrrolidine-1-carboxylate;
2-(1-ethyl-1-hydroxypropyl)-9-fluorobenzo[h][1,3]thiazolo[5,4-f]isoquinolin-7(6H)-one;
9-fluoro-2-(hydroxymethyl)benzo[h][1,3]thiazolo[5,4-f]isoquinolin-7(6H)-one;
2-(ethoxymethyl)-9-fluorobenzo[h][1,3]thiazolo[5,4-f]isoquinolin-7(6H)-one;
3-(9-fluoro-7-oxo-6,7-dihydrobenzo[h][1,3]thiazolo[5,4-f]isoquinolin-2-yl)propanenitrile;
[1-(9-fluoro-7-oxo-6,7-dihydrobenzo[h][1,3]thiazolo[5,4-f]isoquinolin-2-yl)piperidin-4-yl]acetonitrile;
1-(9-fluoro-7-oxo-6,7-dihydrobenzo[h][1,3]thiazolo[5,4-f]isoquinolin-2-yl)piperidine-4-carbonitrile;
3-[(9-fluoro-7-oxo-6,7-dihydrobenzo[h][1,3]thiazolo[5,4-f]isoquinolin-2-yl)methoxy]propanenitrile;
9-fluoro-2-{4-[2-(1H-1,2,4-triazol-1-yl)ethyl]piperidin-1-yl}benzo[h][1,3]thiazolo[5,4-f]isoquinolin-7(6H)-one;
9-fluoro-2-[4-(1H-1,2,4-triazol-1-ylmethyl)piperidin-1-yl]benzo[h][1,3]thiazolo[5,4-f]isoquinolin-7(6H)-one;
9-fluoro-2-[4-(1H-imidazol-1-ylmethyl)piperidin-1-yl]benzo[h][1,3]thiazolo[5,4-f]isoquinolin-7(6H)-one;
3-[1-(9-fluoro-7-oxo-6,7-dihydrobenzo[h][1,3]thiazolo[5,4-f]isoquinolin-2-yl)piperidin-4-yl]propanenitrile;
9-fluoro-2-{4-[2-(1H-imidazol-1-yl)ethyl]piperidin-1-yl}benzo[h][1,3]thiazolo[5,4-f]isoquinolin-7(6H)-one;
2-{4-[(diethylamino)methyl]piperidin-1-yl}-9-fluorobenzo[h][1,3]thiazolo[5,4-f]isoquinolin-7(6H)-one;
9-fluoro-2-(pyridin-3-ylmethyl)benzo[h][1,3]thiazolo[5,4-f]isoquinolin-7(6H)-one;
9-fluoro-2-(pyridin-2-ylmethyl)benzo[h][1,3]thiazolo[5,4-f]isoquinolin-7(6H)-one;
9-fluoro-2-[(1-oxidopyridin-3-yl)methyl]benzo[h][1,3]thiazolo[5,4-f]isoquinolin-7(6H)-one;
9-fluoro-2-(pyridin-4-ylmethyl)benzo[h][1,3]thiazolo[5,4-f]isoquinolin-7(6H)-one;
9-fluoro-2-[hydroxy(6-methoxypyridin-3-yl)methyl]benzo[h][1,3]thiazolo[5,4-f]isoquinolin-7(6H)-one;
9-fluoro-2-[3-hydroxy-1-(pyridin-4-ylmethyl)piperidin-3-yl]benzo[h][1,3]thiazolo[5,4-f]isoquinolin-7(6H)-one;
2-(1-acetyl-3-hydroxypiperidin-3-yl)-9-fluorobenzo[h][1,3]thiazolo[5,4-f]isoquinolin-7(6H)-one;
9-fluoro-2-[(4-hydroxypiperidin-1-yl)methyl]benzo[h][1,3]thiazolo[5,4-f]isoquinolin-7(6H)-one;
2-[(4-acetylpiperazin-1-yl)carbonyl]-9-fluorobenzo[h][1,3]thiazolo[5,4-f]isoquinolin-7(6H)-one;
9-fluoro-2-β-hydroxy-1-(methylsulfonyl)piperidin-3-yl]benzo[h][1,3]thiazolo[5,4-f]isoquinolin-7(6H)-one;
9-fluoro-2-(1-hydroxy-1-pyridin-3-ylethyl)benzo[h][1,3]thiazolo[5,4-f]isoquinolin-7(6H)-one;
9-fluoro-2-(1-hydroxy-1-pyridin-4-ylethyl)benzo[h][1,3]thiazolo[5,4-f]isoquinolin-7(6H)-one;
9-fluoro-2-[1-hydroxy-1-(1-oxidopyridin-3-yl)ethyl]benzo[h][1,3]thiazolo[5,4-f]isoquinolin-7(6H)-one;
9-fluoro-2-[1-hydroxy-1-(1-oxidopyridin-4-yl)ethyl]benzo[h][1,3]thiazolo[5,4-f]isoquinolin-7(6H)-one;
2-{4-[(dimethylamino)methyl]piperidin-1-yl}-9-fluorobenzo[h][1,3]thiazolo[5,4-f]isoquinolin-7(6H)-one;
9-fluoro-2-(pyridine-3-yloxy)benzo[h][1,3]thiazolo[5,4-f]isoquinolin-7(6H)-one;
9-fluoro-2-(pyridin-4-ylthio)benzo[h][1,3]thiazolo[5,4-f]isoquinolin-7(6H)-one;
9-fluoro-2-[(trans-4-hydroxycyclohexyl)amino]benzo[h][1,3]thiazolo[5,4-f]isoquinolin-7(6H)-one;
9-fluoro-2-(4-pyrazin-2-ylpiperazin-1-yl)benzo[h][1,3]thiazolo[5,4-f]isoquinolin-7(6H)-one;
4-(9-fluoro-7-oxo-6,7-dihydrobenzo[h][1,3]thiazolo[5,4-f]isoquinolin-2-yl)-N-methylpiperazine-1-carboxamide;
9-fluoro-2-[4-(1H-pyrazol-3-ylcarbonyl)piperazin-1-yl]benzo[h][1,3]thiazolo[5,4-f]isoquinolin-7(6H)-one;
9-fluoro-2-[4-(1H-pyrazol-4-ylcarbonyl)piperazin-1-yl]benzo[h][1,3]thiazolo[5,4-f]isoquinolin-7(6H)-one;
9-fluoro-2-[4-(1H-imidazol-2-ylcarbonyl)piperazin-1-yl]benzo[h][1,3]thiazolo[5,4-f]isoquinolin-7(6H)-one;
2-[4-(aminoacetyl)piperazin-1-yl]-9-fluorobenzo[h][1,3]thiazolo[5,4-f]isoquinolin-7(6H)-one;
2-[4-(azetidin-3-ylcarbonyl)piperazin-1-yl]-9-fluorobenzo[h][1,3]thiazolo[5,4-f]isoquinolin-7(6H)-one;
N-(tert-butyl)-4-(9-fluoro-7-oxo-6,7-dihydrobenzo[h][1,3]thiazolo[5,4-f]isoquinolin-2-yl)piperazine-1-carboxamide;
9-fluoro-2-{4-[(2S)-2-hydroxypropyl]piperazin-1-yl}benzo[h][1,3]thiazolo[5,4-f]isoquinolin-7(6H)-one;
3-[4-(9-fluoro-7-oxo-6,7-dihydrobenzo[h][1,3]thiazolo[5,4-f]isoquinolin-2-yl)piperazin-1-yl]-3-oxopropanenitrile;
9-fluoro-2-[4-(methylsulfonyl)piperazin-1-yl]benzo[h][1,3]thiazolo[5,4-f]isoquinolin-7(6H)-one;
4-(9-fluoro-7-oxo-6,7-dihydrobenzo[h][1,3]thiazolo[5,4-f]isoquinolin-2-yl)-N,N-dimethylpiperazine-1-carboxamide;
1-(9-fluoro-7-oxo-6,7-dihydrobenzo[h][1,3]thiazolo[5,4-f]isoquinolin-2-yl)piperidine-4-carboxylic acid;
9-fluoro-2-{4-hydroxy-4-[(methylamino)methyl]piperidin-1-yl}benzo[h][1,3]thiazolo[5,4-f]isoquinolin-7(6H)-one;
9-fluoro-2-[4-hydroxy-4-(1H-1,2,4-triazol-1-ylmethyl)piperidin-1-yl]benzo[h][1,3]thiazolo[5,4-f]isoquinolin-7(6H)-one;
9-fluoro-2-[4-hydroxy-4-(hydroxymethyl)piperidin-1-yl]benzo[h][1,3]thiazolo[5,4-f]isoquinolin-7(6H)-one;
9-fluoro-2-piperidin-3-ylbenzo[h][1,3]thiazolo[5,4-f]isoquinolin-7(6H)-one;
2-(1-acetylpiperidin-3-yl)-9-fluorobenzo[h][1,3]thiazolo[5,4-f]isoquinolin-7(6H)-one;
9-fluoro-2-[1-(methylsulfonyl)piperidin-3-yl]benzo[h][1,3]thiazolo[5,4-f]isoquinolin-7(6H)-one;
9-fluoro-2-[1-(pyridin-4-ylmethyl)piperidin-3-yl]benzo[h][1,3]thiazolo[5,4-f]isoquinolin-7(6H)-one;
3-[3-(9-fluoro-7-oxo-6,7-dihydrobenzo[h][1,3]thiazolo[5,4-f]isoquinolin-2-yl)piperidin-1-yl]-3-oxopropanenitrile;
9-fluoro-2-{1-[(2S)-2-hydroxypropyl]piperidin-3-yl}benzo[h][1,3]thiazolo[5,4-f]isoquinolin-7(6H)-one;
2-{1-[(dimethylamino)acetyl]piperidin-3-yl}-9-fluorobenzo[h][1,3]thiazolo[5,4-f]isoquinolin-7(6H)-one;
9-fluoro-2-[4-(morpholin-4-ylmethyl)piperidin-1-yl]benzo[h][1,3]thiazolo[5,4-f]isoquinolin-7(6H)-one;
9-fluoro-2-[4-(2-morpholin-4-ylethyl)piperidin-1-yl]benzo[h][1,3]thiazolo[5,4-f]isoquinolin-7(6H)-one;
4-(9-fluoro-7-oxo-6,7-dihydrobenzo[h][1,3]oxazolo[5,4-f]isoquinolin-2-yl)butanenitrile;
9-fluoro-2-[3-(1H-1,2,4-triazol-1-yl)propyl]benzo[h][1,3]oxazolo[5,4-f]isoquinolin-7(6H)-one;
9-fluoro-2-{[(1R)-1-phenylethyl]amino}benzo[h][1,3]oxazolo[5,4-f]isoquinolin-7(6H)-one;
9-fluoro-2-{[(1S)-1-phenylethyl]amino}benzo[h][1,3]oxazolo[5,4-f]isoquinolin-7(6H)-one;
9-fluoro-2-[(2-methoxybenzyl)amino]benzo [h][1,3]oxazolo[5,4-f]isoquinolin-7(6H)-one;
9-fluoro-2-[(4-methoxybenzyl)amino]benzo[h][1,3]oxazolo[5,4-f]isoquinolin-7(6H)-one;
9-fluoro-2-[(3-methoxybenzyl)amino]benzo[h][1,3]oxazolo[5,4-f]isoquinolin-7(6H)-one;
9-fluoro-2-(4-oxopiperidin-1-yl)benzo[h][1,3]oxazolo[5,4-f]isoquinolin-7(6H)-one;
9-fluoro-2-(4-hydroxypiperidin-1-yl)benzo[h][1,3]oxazolo[5,4-f]isoquinolin-7(6H)-one;
9-fluoro-2-{[1-(methylsulfonyl)piperidin-4-yl]amino}benzo[h][1,3]oxazolo[5,4-f]isoquinolin-7(6H)-one;
2-[(1-acetylpiperidin-4-yl)amino]-9-fluorobenzo[h][1,3]oxazolo[5,4-f]isoquinolin-7(6H)-one;
9-fluoro-2-[hydroxy(pyridin-3-yl)methyl]benzo[h][1,3]oxazolo[5,4-f]isoquinolin-7(6H)-one;
2-(4-acetylpiperazin-1-yl)-9-fluorobenzo[h][1,3]oxazolo[5,4-f]isoquinolin-7(6H)-one;
4-(9-fluoro-7-oxo-6,7-dihydrobenzo[h][1,3]oxazolo[5,4-f]isoquinolin-2-yl)piperazine-1-carboxamide;
2-[(4-acetylpiperazin-1-yl)methyl]-9-fluorobenzo[h][1,3]oxazolo[5,4-f]isoquinolin-7(6H)-one;
9-fluoro-2-[(trans-4-hydroxycyclohexyl)amino]benzo[h][1,3]oxazolo[5,4-f]isoquinolin-7(6H)-one;
9-fluoro-2-(pyridin-3-yloxy)benzo[h][1,3]oxazolo[5,4-f]isoquinolin-7(6H)-one;
2-[(1-acetylpiperidin-3-yl)amino]-9-fluorobenzo[h][1,3]oxazolo[5,4-f]isoquinolin-7(6H)-one;
9-fluoro-2-{[1-(methylsulfonyl)piperidin-3-yl]amino}benzo[h][1,3]oxazolo[5,4-f]isoquinolin-7(6H)-one;
9-fluoro-2-[4-(1,3-thiazol-2-yl)piperazin-1-yl]benzo[h][1,3]oxazolo[5,4-f]isoquinolin-7(6H)-one;
2-(trans-4-Hydroxycyclohexyl)imidazo[1,2-a]pyrido[4,3-c]-1,6-naphthyridin-8(7H)-one;
2-(cis-4-Hydroxycyclohexyl)imidazo[1,2-a]pyrido[4,3-c]-1,6-naphthyridin-8(7H)-one;
4-(8-Oxo-7,8-dihydroimidazo[1,2-a]pyrido[4,3-c]-1,6-naphthyridin-2-yl)piperidine-1-carbaldehyde;
2-Piperidin-4-ylimidazo[1,2-a]pyrido[4,3-c]-1,6-naphthyridin-8(7H)-one;
2-[1-(Cyclopropylmethyl)piperidin-4-yl]imidazo[1,2-a]pyrido[4,3-c]-1,6-naphthyridin-8(7H)-one;
2-[1-(Propyl)piperidin-4-yl]imidazo[1,2-a]pyrido[4,3-c]-1,6-naphthyridin-8(7H)-one;
2-tert-Butylimidazo[1,2-a]pyrido[4,3-c]-1,6-naphthyridin-8(7H)-one;
2-(4-Hydroxypiperidin-1-yl)-1,9-dihydro-8H-imidazo[4,5-f]-2,8-phenanthrolin-8-one;
2-(4-hydroxypiperidin-1-yl)[1,3]thiazolo[4,5-f]-2,9-phenanthrolin-8(9H)-one;
2-(isopropylamino)[1,3]thiazolo[4,5-f]-2,9-phenanthrolin-8(9H)-one;
2-[(3-methoxypropyl)amino][1,3]thiazolo[4,5-f]-2,9-phenanthrolin-8(9H)-one;
2-{[(R)-1-(hydroxymethyl)-3-methylbutyl]amino}[1,3]thiazolo[4,5-f]-2,9-phenanthrolin-8(9H)-one;
2-{[1-(methylsulfonyl)piperidin-4-yl]oxy}[1,3]thiazolo[4,5-f]-2,9-phenanthrolin-8(9H)-one;
2-{[(1R)-1-(hydroxymethyl)butyl]amino}[1,3]thiazolo[4,5-f]-2,9-phenanthrolin-8(9H)-one;
2-{[(1R)-1-(hydroxymethyl)propyl]amino}[1,3]thiazolo[4,5-f]-2,9-phenanthrolin-8(9H)-one;
2-{[(1R)-1-(hydroxymethyl)-2-methylpropyl]amino}[1,3]thiazolo[4,5-f]-2,9-phenanthrolin-8(9H)-one;
2-{[(1R,2R)-2-hydroxycyclohexyl]amino}[1,3]thiazolo[4,5-f]-2,9-phenanthrolin-8(9H)-one;
2-{[(1R,2R)-2-hydroxycyclopentyl]amino}[1,3]thiazolo[4,5-f]-2,9-phenanthrolin-8(9H)-one;
2-{[(1R)-1-(hydroxymethyl)-2,2-dimethylpropyl]amino}[1,3]thiazolo[4,5-f]-2,9-phenanthrolin-8(9H)-one;
2-(4-{[3-(2-oxopyrrolidin-1-yl)propyl]amino}piperidin-1-yl)[1,3]thiazolo[4,5-f]-2,9-phenanthrolin-8(9H)-one;
2-{[(1S,2S)-1-(hydroxymethyl)-2-methylbutyl]amino}[1,3]thiazolo[4,5-f]-2,9-phenanthrolin-8(9H)-one;
2-{4-[(3-morpholin-4-ylpropyl)amino]piperidin-1-yl}[1,3]thiazolo[4,5-f]-2,9-phenanthrolin-8(9H)-one;
2-(dimethylamino)-N-[1-(8-oxo-8,9-dihydro[1,3]thiazolo[4,5-f]-2,9-phenanthrolin-2-yl)piperidin-4-yl]acetamide;
2-(4-hydroxypiperidin-1-yl)[1,3]thiazolo[4,5-f]-2,9-phenanthrolin-8(9H)-one;
2-(propylamino)[1,3]thiazolo[4,5-f]-2,9-phenanthrolin-8(9H)-one;
2-(4-acetylpiperazin-1-yl)[1,3]thiazolo[4,5-f]-2,9-phenanthrolin-8(9H)-one;
2-piperazin-1-yl[1,3]thiazolo[4,5-f]-2,9-phenanthrolin-8(9H)-one;
2-{4-[(2S)-2-hydroxypropyl]piperazin-1-yl}[1,3]thiazolo[4,5-f]-2,9-phenanthrolin-8(9H)-one;
2-[4-(1H-imidazol-5-ylacetyl)piperazin-1-yl][1,3]thiazolo[4,5-f]-2,9-phenanthrolin-8(9H)-one;
2-{4-[(2R)-2-hydroxypropyl]piperazin-1-yl}[1,3]thiazolo[4,5-f]-2,9-phenanthrolin-8(9H)-one;
2-[4-(2-hydroxyethyl)piperazin-1-yl][1,3]thiazolo[4,5-f]-2,9-phenanthrolin-8(9H)-one;
2-{[(2R)-2-amino-3,3-dimethylbutyl]oxy}[1,3]thiazolo[4,5-f]-2,9-phenanthrolin-8(9H)-one;
2-{[(S)-1-(hydroxymethyl)-2,2-dimethylpropyl]amino}[1,3]thiazolo[4,5-f]-2,9-phenanthrolin-8(9H)-one;
N-[1-(8-oxo-8,9-dihydro[1,3]thiazolo[4,5-f]-2,9-phenanthrolin-2-yl)piperidin-4-yl]-3-pyridin-3-ylpropanamide;
2-[4-(2-morpholin-4-ylethoxy)piperidin-1-yl][1,3]thiazolo[4,5-f]-2,9-phenanthrolin-8(9H)-one;
2-{4-[(2-morpholin-4-ylethoxy)methyl]piperidin-1-yl}[1,3]thiazolo[4,5-f]-2,9-phenanthrolin-8(9H)-one;
2-(4-{methyl[3-(2-oxopyrrolidin-1-yl)propyl]amino}piperidin-1-yl)[1,3]thiazolo[4,5-f]-2,9-phenanthrolin-8(9H)-one;
2-[4-(morpholin-4-ylmethyl)piperidin-1-yl][1,3]thiazolo[4,5-f]-2,9-phenanthrolin-8(9H)-one;
2-[4-(2-morpholin-4-ylethyl)piperidin-1-yl][1,3]thiazolo[4,5-f]-2,9-phenanthrolin-8(9H)-one;
2-(4-hydroxypiperidin-1-yl)[1,3]oxazolo[4,5-f]-2,9-phenanthrolin-8(9H)-one;
2-(4-oxopiperidin-1-yl)[1,3]oxazolo[4,5-f]-2,9-phenanthrolin-8(9H)-one;
2-(4-hydroxy-4-methylpiperidin-1-yl)[1,3]oxazolo[4,5-f]-2,9-phenanthrolin-8(9H)-one;
9-fluoro-2-(pyridin-4-ylmethoxy)-3,6-dihydro-7H-benzo[h]imidazo[4,5-f]isoquinolin-7-one;
9-fluoro-2-[(pyridin-3-ylmethyl)amino]-3,6-dihydro-7H-benzo[h]imidazo[4,5-f]isoquinolin-7-one;
9-fluoro-2-(3-piperidin-1-ylpropoxy)-1,6-dihydro-7H-benzo[h]imidazo[4,5-f]isoquinolin-7-one;
9-fluoro-2-[(3-morpholin-4-ylpropyl)amino]-1,6-dihydro-7H-benzo[h]imidazo[4,5-f]isoquinolin-7-one;
9-fluoro-2-4-hydroxy-4-[(2-morpholin-4-ylethoxy)methyl]piperidin-1-yl-1,6-dihydro-7H-benzo[h]imidazo[4,5-f]isoquinolin-7-one;
9-fluoro-2-(pyridin-3-yloxy)-3,6-dihydro-7H-benzo[h]imidazo[4,5-f]isoquinolin-7-one;
9-fluoro-2-isopropoxy-3,6-dihydro-7H-benzo[h]imidazo[4,5-f]isoquinolin-7-one;
9-fluoro-2-(pyridin-3-ylmethoxy)-3,6-dihydro-7H-benzo[h]imidazo[4,5-f]isoquinolin-7-one;
9-fluoro-2-(4-oxopyridin-1 (4H)-yl)-3,6-dihydro-7H-benzo[h]imidazo[4,5-f]isoquinolin-7-one;
9-fluoro-2-[(cis-4-hydroxycyclohexyl)oxy]-3,6-dihydro-7H-benzo[h]imidazo[4,5-f]isoquinolin-7-one;
9-fluoro-2-[(trans-4-hydroxycyclohexyl)oxy]-3,6-dihydro-7H-benzo[h]imidazo[4,5-f]isoquinolin-7-one;
9-fluoro-2-(pyridin-2-ylmethoxy)-3,6-dihydro-7H-benzo[h]imidazo[4,5-f]isoquinolin-7-one;
9-fluoro-2-[(trans-4-hydroxycyclohexyl)amino]-3,6-dihydro-7H-benzo[h]imidazo[4,5-f]isoquinolin-7-one;
9-fluoro-2-[(pyridin-4-ylmethyl)amino]-3,6-dihydro-7H-benzo[h]imidazo[4,5-f]isoquinolin-7-one;
9-fluoro-2-[(1-oxidopyridin-2-yl)methoxy]-3,6-dihydro-7H-benzo[h]imidazo[4,5-f]isoquinolin-7-one;
9-fluoro-2-[(1-oxidopyridin-3-yl)methoxy]-3,6-dihydro-7H-benzo[h]imidazo[4,5-f]isoquinolin-7-one;
9-fluoro-2-[(1R,3R)-3-hydroxycyclohexyl]oxy-3,6-dihydro-7H-benzo[h]imidazo[4,5-f]isoquinolin-7-one;
cis- and trans-9-fluoro-2-[3-hydroxycyclohexyl]oxy-3,6-dihydro-7H-benzo[h]imidazo[4,5-f]isoquinolin-7-one;
9-fluoro-2-[1-(methylsulfonyl)piperidin-4-yl]oxy-3,6-dihydro-7H-benzo[h]imidazo[4,5-f]isoquinolin-7-one;
2-[(1-acetylpiperidin-4-yl)oxy]-9-fluoro-3,6-dihydro-7H-benzo[h]imidazo[4,5-f]isoquinolin-7-one;
3-4-[(9-fluoro-7-oxo-6,7-dihydro-3H-benzo[h]imidazo[4,5-f]isoquinolin-2-yl)oxy]piperidin-1-yl-3-oxopropanenitrile;
9-fluoro-2-[(trans-4-hydroxycyclohexyl)(methyl)amino]-3,6-dihydro-7H-benzo[h]imidazo[4,5-f]isoquinolin-7-one;
9-fluoro-2-(3-morpholin-4-ylpropoxy)-1,6-dihydro-7H-benzo[h]imidazo[4,5-f]isoquinolin-7-one;
9-fluoro-2-[3-(2-oxopyrrolidin-1-yl)propyl]amino-1,6-dihydro-7H-benzo[h]imidazo[4,5-f]isoquinolin-7-one;
2-(4-{[bis(2-methoxyethyl)amino]methyl}piperidin-1-yl)-9-fluoro-1,6-dihydro-7H-benzo[h]imidazo[4,5-f]isoquinolin-7-one;
9-fluoro-2-{4-[(2-morpholin-4-ylethoxy)methyl]piperidin-1-yl}-1,6-dihydro-7H-benzo[h]imidazo[4,5-f]isoquinolin-7-one;
2-[(cyclopropylmethyl)(3-morpholin-4-ylpropyl)amino]-9-fluoro-1,6-dihydro-7H-benzo[h]imidazo[4,5-f]isoquinolin-7-one;
9-fluoro-2-4-hydroxy-4-[(3-piperidin-1-ylpropoxy)methyl]piperidin-1-yl-1,6-dihydro-7H-benzo[h]imidazo[4,5-f]isoquinolin-7-one;
9-fluoro-2-4-hydroxy-4-[(3-morpholin-4-ylpropoxy)methyl]piperidin-1-yl-1,6-dihydro-7H-benzo[h]imidazo[4,5-f]isoquinolin-7-one;
10-fluoro-2-[4-(hydroxyimino)cyclohexyl]benzo[c]imidazo[1,2-a]-1,6-naphthyridin-8(7H)-one;
9-fluoro-2-4-[(2-morpholin-4-ylethoxy)imino]piperidin-1-yl-1,6-dihydro-7H-benzo[h]imidazo[4,5-f]isoquinolin-7-one;
4-ethyl-4-(9-fluoro-7-hydroxy-3H-benzo[f]imidazo[4,5-h]phthalazin-2-yl)hexanenitrile;
4-ethyl-4-(9-fluoro-7-oxo-6,7-dihydro-3H-benzo[f]imidazo[4,5-h]phthalazin-2-yl)hexanoic acid;
4-ethyl-4-(9-fluoro-7-hydroxy-3H-benzo[f]imidazo[4,5-h]phthalazin-2-yl)hexanamide;
methyl 2-(9-fluoro-7-hydroxy-3H-benzo[f]imidazo[4,5-h]phthalazin-2-yl)-2-methylpropanoate;
2-(9-fluoro-7-hydroxy-3H-benzo [f]imidazo[4,5-h]phthalazin-2-yl)-2-methylpropanoic acid;
9-fluoro-2-(2-hydroxy-1,1-dimethylethyl)-3H-benzo[f]imidazo[4,5-h]phthalazin-7-ol;
4-(9-fluoro-7-oxo-6,7-dihydro-3H-benzo[f]imidazo[4,5-h]phthalazin-2-yl)-4-methylpentanenitrile;
(2E)-4-(9-fluoro-7-oxo-6,7-dihydro-3H-benzo[f]imidazo[4,5-h]phthalazin-2-yl)-4-methylpent-2-enenitrile;
9-fluoro-2-[4-(methylthio)phenyl]-3,6-dihydro-7H-benzo[f]imidazo[4,5-h]phthalazin-7-one;
9-fluoro-2-[4-(methylsulfinyl)phenyl]-3,6-dihydro-7H-benzo[f]imidazo[4,5-h]phthalazin-7-one;
4-(9-fluoro-7-hydroxy-3H-benzo [f]imidazo[4,5-h]phthalazin-2-yl)-4-methylpentanamide;
3-(9-fluoro-7-hydroxy-3H-benzo [f]imidazo[4,5-h]phthalazin-2-yl)propanenitrile;
9-fluoro-2-(2-pyridin-2-ylethyl)-3H-benzo[f]imidazo[4,5-h]phthalazin-7-ol;
9-fluoro-2-(pyridin-4-ylmethyl)-3,6-dihydro-7H-benzo[f]imidazo[4,5-h]phthalazin-7-one;
9-fluoro-2-(pyridin-3-ylmethyl)-3,6-dihydro-7H-benzo[f]imidazo[4,5-h]phthalazin-7-one;
2-[4-(benzyloxy)cyclohexyl]-9-fluoro-3,6-dihydro-7H-benzo[f]imidazo[4,5-h]phthalazin-7-one;
9-fluoro-2-(4-hydroxycyclohexyl)-3,6-dihydro-7H-benzo[f]imidazo[4,5-h]phthalazin-7-one;
methyl 2-(9-fluoro-7-hydroxybenzo[f][1,3]oxazolo[5,4-h]phthalazin-2-yl)-2-methylpropanoate;
benzyl [4-(9-fluoro-7-hydroxybenzo[f][1,3]oxazolo[5,4-h]phthalazin-2-yl)-4-methylpentyl]carbamate;
2-(4-amino-1,1-dimethylbutyl)-9-fluorobenzo[f][1,3]oxazolo[5,4-h]phthalazin-7-ol;
2-(9-fluoro-7-hydroxybenzo[f][1,3]oxazolo[5,4-h]phthalazin-2-yl)-2-methylpropanoic acid;
2-tert-butyl-1,9-dihydro-8H-imidazo[4,5-f]-2,8-phenanthrolin-8-one;
2-tert-butyl-3,6-dihydro-7H-imidazo[4,5-f]-1,9-phenanthrolin-7-one;
2-cyclohexyl-1,9-dihydro-8H-imidazo[4,5-f]-2,8-phenanthrolin-8-one;
3-(8-oxo-8,9-dihydro-1H-imidazo[4,5-f]-2,8-phenanthrolin-2-yl)propanenitrile;
2-(trans-4-hydroxycyclohexyl)-1,9-dihydro-8H-imidazo[4,5-f]-2,8-phenanthrolin-8-one;
2-[1-(methylsulfonyl)piperidin-4-yl]-1,9-dihydro-8H-imidazo[4,5-f]-2,8-phenanthrolin-8-one;
3-oxo-3-[4-(8-oxo-8,9-dihydro-1H-imidazo[4,5-f]-2,8-phenanthrolin-2-yl)piperidin-1-yl]propanenitrile;
3-methyl-3-(8-oxo-8,9-dihydro-1H-imidazo[4,5-f]-2,8-phenanthrolin-2-yl)butanenitrile;
2-tert-butyl-1,9-dihydro-8H-imidazo[4,5-f]-2,7-phenanthrolin-8-one;
2-cyclohexyl-1,9-dihydro-8H-imidazo[4,5-f]-2,7-phenanthrolin-8-one;
2-cyclopentyl-1,9-dihydro-8H-imidazo[4,5-f]-2,7-phenanthrolin-8-one;
2-tert-butyl-1,9-dihydro-8H-imidazo[4,5-f]-2,8-phenanthrolin-8-one 5-oxide; and
3-methyl-3-(5-oxido-8-oxo-8,9-dihydro-1H-imidazo[4,5-f]-2,8-phenanthrolin-2-yl)butanenitrile; or pharmaceutically acceptable salt thereof.

38. A composition comprising a compound of claim 1 and a pharmaceutically acceptable carrier.

39. A method of modulating an activity of JAK comprising contacting JAK with a compound of claim 1.

40. The method of claim 39 wherein said JAK is JAK1, JAK2, JAK3 or TYK2.

41. The method of claim 39 wherein said JAK is a naturally occurring JAK variant.

42. The method of claim 39 wherein said modulating is inhibiting.

43. A method of treating a disease in a patient, where said disease is associated with JAK activity, comprising administering to said patient a therapeutically effective amount of a compound of claim 1.

44. The method of claim 43 wherein said disease is allograft rejection or graft versus host disease.

45. The method of claim 43 wherein said disease is an autoimmune diesase.

46. The method of claim 45 wherein said autoimmune disease is multiple sclerosis, rheumatoid arthritis, juvenile arthritis, type I diabetes, lupus, psoriasis, inflammatory bowel disease, Crohn's disease, or autoimmune thyroid disorder.

47. The method of claim 43 wherein said disease is a viral disease.

48. The method of claim 47 wherein said viral disease is Epstein Barr Virus (EBV), Hepatitis B, Hepatitis C, HIV, HTLV 1, Varicell-Zoster Virus (VZV) or Human Papilloma Virus (HPV).

49. The method of claim 43 wherein said disease is cancer.

50. The method of claim 49 wherein said cancer is prostate cancer, lymphoma, leukemia, or multiple myeloma.

51. The method of claim 43 wherein said disease is a myeloproliferative disorder.

52. A method of treating psoriasis comprising administering to patient suffering from psoriasis a therapeutically effective amount of a compound of claim 1.

53. The method of claim 52 wherein said compound is administered topically.

54. A composition comprising a compound of claim 1 and a pharmaceutically acceptable carrier wherein said composition is suitable for topical administration.
